(19) United States
(12) Patent Application Publication
(10) Pub. No.: US 2023/0357770 A1
(43) Pub. Date: Nov. 9, 2023

(54) COMPOUNDS AND METHODS FOR REDUCING APOE EXPRESSION

(71) Applicant: Ionis Pharmaceuticals, Inc., Carlsbad, CA (US)

(72) Inventors: Tracy A. Cole, Encinitas, CA (US); Swagatam Mukhopadhyay, Oceanside, CA (US); Huynh-Hoa Bui, San Diego, CA (US); Priyam Singh, San Diego, CA (US); Holly Kordasiewicz, San Diego, CA (US); Susan M. Freier, San Diego, CA (US); Hien Thuy Zhao, San Diego, CA (US)

(73) Assignee: Ionis Pharmaceuticals, Inc., Carlsbad, CA (US)

(21) Appl. No.: 18/246,468

(22) PCT Filed: Sep. 23, 2021
(86) PCT No.: PCT/US2021/051822

§ 371 Date: Mar. 23, 2023

Related U.S. Application Data
(60) Provisional application No. 63/083,006, filed on Sep. 24, 2020.

(51) Int. Cl. C12N 15/113; C12N 9/22; A61K 38/46; A61K 31/713; A61P 25/28

(52) U.S. Cl. CPC C12N 15/113; C12N 9/22; C12Y 301/26004; A61K 38/465; A61K 31/713; A61P 25/28; C12N 2310/14; C12N 2310/321; C12N 2310/313; C12N 2310/3233; C12N 2310/3341; C12N 2310/341; C12N 2310/3231; C12N 2310/3519; C12N 2310/346; C12N 2310/345

(57) ABSTRACT

Provided are compounds, methods, and pharmaceutical compositions for reducing the amount or activity of APOE RNA in a cell or animal, and in certain instances reducing the amount of APOE protein in a cell or animal Such compounds, methods, and pharmaceutical compositions are useful to ameliorate at least one symptom or hallmark of a neurodegenerative disease. Such symptoms and hallmarks include cognitive impairment, progressive memory loss, behavioral abnormality, dementia, difficulty performing daily activities, amyloid plaques, neurofibrillary tangles, and neuroinflammation.

### SEQUENCE LISTING

The present application is being filed along with a Sequence Listing in electronic format. The Sequence Listing is provided as a file entitled BIOL0394WOSEQ_ST25.txt, created on Sep. 23, 2021, which is 597 KB in size. The information in the electronic format of the sequence listing is incorporated herein by reference in its entirety.

### FIELD

Provided are compounds, methods, and pharmaceutical compositions for reducing the amount or activity of APOE RNA in a cell or animal, and in certain instances reducing the amount of APOE protein in a cell or animal. Certain such compounds, methods, and pharmaceutical compositions are useful to ameliorate at least one symptom or hallmark of a neurodegenerative disease. Such symptoms and hallmarks include cognitive impairment, progressive memory loss, behavioral abnormality, dementia, difficulty performing daily activities, amyloid plaques, neurofibrillary tangles, and neuroinflammation. Such neurodegenerative diseases include Alzheimer's Disease.

### BACKGROUND

Alzheimer's Disease (AD) is the most common cause of age-associated dementia, affecting an estimated 5.7 million Americans a year (Alzheimer's Association. 2018 Alzheimer's Disease Facts and Figures. Alzheimer's Dement. 2018; 14(3):367-429). Symptoms of AD include cognitive impairment, a decline in memory and language skills, behavioral and psychological symptoms such as apathy and lack of motivation, gait disturbances and seizures, and dementia. Hallmarks of AD include the presence of amyloid plaques and neurofibrillary tangles in the brains of patients. Amyloid plaques are toxic aggregates composed mainly of peptides that are encoded by the amyloid precursor protein gene, APP. Neurofibrillary tangles are hyperphosphorylated, insoluble aggregates of tau proteins.

Apolipoprotein E (APOE) is a fat-binding protein that is associated with lipoprotein particles. APOE is produced mainly by the liver. APOE is also produced in the brain by astrocytes, where it plays a role in transporting cholesterol to neurons via APOE receptors. There are three main APOE alleles that encode three different APOE protein isoforms: APOE-ε2, APOE-ε3, and APOE-ε4. These alleles encode for APOE protein variants having different combinations of amino acids at positions 112 and 158 of the APOE protein: APOE-ε2 (Cys112, Cys158), APOE-ε3 (Cys112, Arg158), and APOE-ε4 (Arg112, Arg158). These amino acid differences result in variable APOE structure and function.

APOE has been linked to pathological hallmarks of AD (e.g., amyloid plaques and neurofibrillary tangles), and to pathways including synaptic plasticity, lipid transport, glucose metabolism, mitochondrial function, and vascular integrity. Polymorphisms in the APOE promoter result in increased APOE promoter activity and APOE protein levels, and an increased risk for developing AD. The APOE4 allele, encoding isoform ε4, is the strongest genetic risk factor for late-onset Alzheimer's disease. Patients homozygous for the APOE4 allele account for about 16% of AD population.

Currently there is a lack of acceptable options for treating neurodegenerative diseases such as AD. It is therefore an object herein to provide compounds, methods, and pharmaceutical compositions for the treatment of such diseases.

### SUMMARY OF THE INVENTION

Provided herein are compounds, methods and pharmaceutical compositions for reducing the amount or activity of APOE RNA, and in certain embodiments reducing the amount of APOE protein in a cell or animal. In certain embodiments, the animal has a neurodegenerative disease. In certain embodiments, the animal has Alzheimer's Disease (AD). In certain embodiments, compounds useful for reducing expression of APOE RNA are oligomeric compounds. In certain embodiments, compounds useful for reducing expression of APOE RNA are modified oligonucleotides.

Also provided are methods useful for ameliorating at least one symptom or hallmark of a neurodegenerative disease. In certain embodiments, the neurodegenerative disease is Alzheimer's Disease. In certain embodiments, the symptom or hallmark includes cognitive impairment, progressive memory loss, behavioral abnormality, dementia, difficulty performing daily activities, amyloid plaques, neurofibrillary tangles, and neuroinflammation.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methyl cytosine” means a cytosine modified with a methyl group attached to the 5 position. A 5-methyl cytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to an animal.

As used herein, “amyloid plaque” means an aggregate of peptides that are encoded by a human amyloid precursor protein gene, APP.

As used herein, “animal” and “subject” are used interchangeably and the terms mean a human or non-human animal.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is cognitive impairment, progressive memory loss, behavioral abnormality, dementia, difficulty performing daily activities, amyloid plaques, neurofibrillary tangles, and neuroinflammation.

As used herein, “behavioral abnormality” means a behavior exhibited by a subject that is atypical or out of the ordinary for the subject. In certain embodiments, the behavior abnormality is selected from depression, anxiety, panic, phobia, paranoia, and a combination thereof. In certain embodiments, the behavior abnormality is a dysfunctional or deviant behavior (e.g., causes distress for others).

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “blunt” or “blunt ended” in reference to a duplex formed by two oligonucleotides mean that there are no terminal unpaired nucleotides (i.e. no overhanging nucleotides). One or both ends of a double-stranded RNAi agent can be blunt.

As used herein, “cell-targeting moiety” means a conjugate group or portion of a conjugate group that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cleavable moiety” means a bond or group of atoms that is cleaved under physiological conditions, for example, inside a cell, an animal, or a human. As used herein, “cognitive impairment” means confusion, poor motor coordination, loss of short-term memory, loss of long-term memory, identity confusion, impaired judgment, or any combination thereof.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Certain modified nucleobases that pair with natural nucleobases or with other modified nucleobases are known in the art. For example, inosine can pair with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt modified sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “daily activities” mean one or more activities selected from dressing, eating, walking, bathing, cooking, shopping, cleaning and exercising.

As used herein, “dementia” means any combination of symptoms selected from memory loss, difficulty communicating, disorientation, difficulty reasoning, difficulty planning, discoordination, compromised motor function, confusion, disorientation, depression, anxiety, paranoia, agitation, and hallucination.

As used herein, “double-stranded” means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNase H cleavage. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE modified nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

“Lipid nanoparticle” or “LNP” is a vesicle comprising a lipid layer encapsulating a pharmaceutically active molecule, such as a nucleic acid molecule, e.g., an RNAi or a plasmid from which an RNAi is transcribed. LNPs are described in, for example, U.S. Pat. Nos. 6,858,225, 6,815,432, 8,158,601, and 8,058,069, the entire contents of which are hereby incorporated herein by reference.

As used herein, “non-bicyclic modified sugar moiety” means a modified sugar moiety that comprises a modification, such as a substituent, that does not form a bridge between two atoms of the sugar to form a second ring.

As used herein, “neuroinflammation” means inflammation of the peripheral nervous system or the central nervous system. In certain embodiments, the amount of a cytokine in the cerebrospinal fluid of a subject with neuroinflammation is significantly greater than the amount of the cytokine in the cerebrospinal fluid of a subject that does not have neuroinflammation. In certain embodiments, a subject with neuroinflammation has Alzheimer's Disease. In certain embodiments, a subject that does not have neuroinflammation does not have Alzheimer's Disease.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned.

As used herein, “MOE” means O-methoxyethyl. “2′-MOE” or “2′-MOE modified sugar” or “2′-MOE modified sugar moiety” means a 2′-OCH₂CH₂OCH₃ group (or a 2′-O(CH₂)₂—OCH₃ group) in place of the 2′-OH group of a ribosyl sugar moiety. Unless otherwise indicated, a 2′-MOE modified sugar moiety is in the β-D-ribosyl configuration. As used herein, “2′-MOE modified nucleoside” means a nucleoside comprising a 2′-MOE modified sugar moiety (or a 2′-O(CH₂)₂—OCH₃ ribosyl modified sugar moiety).

As used herein, “2′-OMe” means a 2′-OCH₃ group in place of the 2′-OH group of a ribosyl sugar moiety. A “2′-O-methyl sugar moiety” or “2′-OMe sugar moiety” or “2′-OMe modified sugar moiety” or “2′-O methylribosyl sugar” means a sugar moiety with a 2′-OCH₃ group in place of the 2′-OH group of a ribosyl sugar moiety. Unless otherwise indicated, a 2′-OMe sugar moiety is in the β-D-ribosyl configuration.

As used herein, “2′-OMe nucleoside” means a nucleoside comprising a 2′-OMe modified sugar moiety.

As used herein, “2′-F” means a 2′-fluoro group in place of the 2′-OH group of a ribosyl sugar moiety. A “2′-F sugar moiety” or “2′-F modified sugar moiety” or “2′-fluororibosyl sugar” means a sugar moiety with a 2′-F group in place of the 2′-OH group of a ribosyl sugar moiety. Unless otherwise indicated, a 2′-F sugar moiety is in the β-D-ribosyl configuration.

As used herein, “2′-F nucleoside” or “2′-F modified nucleoside” means a nucleoside comprising a 2′-F modified sugar moiety.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of function or structure, including loss of neuronal function and death of neurons. In certain embodiments, the neurodegenerative disease is Alzheimer's Disease. In certain embodiments, the neurodegenerative disease is Alzheimer's Disease in Down Syndrome patients. In certain embodiments, the neurodegenerative disease is Cerebral Amyloid Angiopathy.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methyl cytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “nucleoside overhang” refers to unpaired nucleotides at either or both ends of a duplex formed by hybridization of an antisense RNAi oligonucleotide and a sense RNAi oligonucleotide.

As used herein, “modified nucleoside” means a nucleoside comprising a modified nucleobase and/or a modified sugar moiety.

As used herein, “linked nucleosides” are nucleosides that are connected in a contiguous sequence (i.e., no additional nucleosides are presented between those that are linked).

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. Thus, each nucleoside of an unmodified oligonucleotide is a DNA or RNA nucleoside and each internucleoside linkage is a phosphodiester linkage.

As used herein, “neurofibrillary tangles” mean hyperphosphorylated insoluble aggregates of tau protein. Tau protein is encoded by a human MAPT gene.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a polymer of linked nucleosides connected via internucleoside linkages, wherein each nucleoside and internucleoside linkage may be modified or unmodified. Unless otherwise indicated, oligonucleotides consist of 8-50 linked nucleosides. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide. An “oligonucleotide duplex” means a duplex formed by two paired oligonucleotides having complementary nucleobase sequences. Each oligo of an oligonucleotide duplex is a “duplexed oligonucleotide” or a “double-stranded oligonucleotide.”

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “progressive memory loss” means occurrences of forgetfulness (e.g., inability to remember events, names, and facts) that increase in frequency over time.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNAi agent” means an antisense compound that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount and/or activity of a target nucleic acid. The term RNAi agent excludes antisense compounds that act through RNase H.

As used herein, “RNAi oligonucleotide” means an antisense RNAi oligonucleotide or a sense RNAi oligonucleotide.

As used herein, “antisense RNAi oligonucleotide” means an oligonucleotide comprising a region that is complementary to a target sequence, and which includes at least one chemical modification suitable for RNAi.

As used herein, “sense RNAi oligonucleotide” means an oligonucleotide comprising a region that is complementary to a region of an antisense RNAi oligonucleotide, and which is capable of forming a duplex with such antisense RNAi oligonucleotide. A duplex formed by an antisense RNAi oligonucleotide and a sense RNAi oligonucleotide is referred to as a double-stranded RNAi agent (dsRNAi) or a short interfering RNA (siRNA).

As used herein, “RNase H compound” means an antisense compound that acts, at least in part, through RNase H to modulate a target nucleic acid and/or protein encoded by a target nucleic acid. In certain embodiments, RNase H compounds are single-stranded. In certain embodiments, RNase H compounds are double-stranded. RNase H compounds may comprise conjugate groups and/or terminal groups. In certain embodiments, an RNase H compound modulates the amount or activity of a target nucleic acid. The term RNase H compound excludes antisense compounds that act principally through RISC/Ago2.

As used herein, “antisense RNase H oligonucleotide” means an oligonucleotide comprising a region that is complementary to a target sequence, and which includes at least one chemical modification suitable for RNase H-mediated nucleic acid reduction.

As used herein, “self-complementary” in reference to an oligonucleotide means an oligonucleotide that at least partially hybridizes to itself.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard in vitro assay,” with respect to means the assay described in Example 1 or Example 10 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

### Certain Embodiments

The present disclosure provides the following non-limiting numbered embodiments:

Embodiment 1. An oligomeric compound comprising a modified oligonucleotide consisting of 12 to 30 linked nucleosides wherein the nucleobase sequence of the modified oligonucleotide is at least 80% complementary to an equal length portion of an APOE RNA, and wherein the modified oligonucleotide comprises at least one modification selected from a modified sugar and a modified internucleoside linkage.

Embodiment 2. An oligomeric compound comprising a modified oligonucleotide consisting of 12 to 30 linked nucleosides, wherein the nucleobase sequence of the modified oligonucleotide comprises at least 12, at least 13, at least 14, at least 15, at least 16, at least 17, at least 18, at least 19, or at least 20 nucleobases of any of SEQ ID NOS: 20-2551 or 2934; wherein the modified oligonucleotide comprises at least one modification selected from a modified sugar and a modified internucleoside linkage.

Embodiment 3. An oligomeric compound comprising a modified oligonucleotide consisting of 12 to 30 linked nucleosides, wherein the nucleobase sequence of the modified oligonucleotide comprises at least 12, at least 13, at least 14, at least 15, at least 16, at least 17, at least 18, at least 19, at least 20, at least 21, at least 22, or at least 23 nucleobases of any of SEQ ID NOS: 2552-2742 or 2935-2944; wherein the modified oligonucleotide comprises at least one modification selected from a modified sugar and a modified internucleoside linkage.

Embodiment 4. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:
an equal length portion of nucleobases 1155-1178 of SEQ ID NO: 2;
an equal length portion of nucleobases 1207-1230 of SEQ ID NO: 2; or
an equal length portion of nucleobases 1259-1295 of SEQ ID NO: 2;
wherein the modified oligonucleotide comprises at least one modification selected from a modified sugar and a modified internucleoside linkage.

Embodiment 5. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:
an equal length portion of nucleobases 1135-1166 of SEQ ID NO: 2; or
an equal length portion of nucleobases 1255-1294 of SEQ ID NO: 2;
wherein the modified oligonucleotide comprises at least one modification selected from a modified sugar and a modified internucleoside linkage.

Embodiment 6. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of an equal length portion of nucleobases 1255-1295 of SEQ ID NO: 2, wherein the modified oligonucleotide comprises at least one modification selected from a modified sugar and a modified internucleoside linkage.

Embodiment 7. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of any of the nucleobase sequences of:
SEQ ID NOS: 70, 71, 169, 170, 447, 448, 543, 552, 553, 919, 1061, 1132, 1938, 1991, 2066, 2154, 2226, 2259, 2324, 2417, or 2486;
SEQ ID NOS: 460, 461, 462, 563, 564, 565, 566, 990, 1469, 1572, 1653, 1746, 1914, 1955, 2026, 2110, 2211, 2247, 2344, 2393, or 2481; or
SEQ ID NOS: 77, 475, 476, 477, 478, 479, 480, 481, 482, 483, 578, 579, 580, 581, 582, 622, 623, 624, 625, 626, 627, 1063, 1193, 1231, 1232, 1300, 1305, 1378, 1409, 1493, 1526, 1564, 1576, 1678, 1679, 1695, 1827, 1870, 1921, 1928, 1950, 1982, 2012, 2046, 2051, 2074, 2088, 2118, 2158, 2169, 2208, 2223, 2232, 2255, 2321, 2343, 2380, 2436, 2449, or 2451.

Embodiment 8. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of any of the nucleobase sequences of:
SEQ ID NOS: 2600, 2601, 2604, 2605, 2606, 2607, 2608, 2609, 2610, or 2613; or
SEQ ID NOS: 2720, 2721, 2722, 2726, 2727, 2729, 2730, 2731, 2732, 2733, 2734, 2735, 2736, 2737, 2738, or 2740.

Embodiment 9. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of any of the nucleobase sequences of SEQ ID NOs: 76, 77, 473, 474, 475, 476, 477, 478, 479, 480, 481, 482, 483, 576, 578, 579, 580, 581, 582, 622, 623, 624, 625, 626, 627, 1063, 1193, 1231, 1232, 1300, 1305, 1378, 1409, 1493, 1526, 1564, 1576, 1678, 1679, 1695, 1701, 1792, 1827, 1870, 1886, 1906, 1921, 1928, 1950, 1982, 2012, 2046, 2051, 2074, 2088, 2118, 2158, 2169, 2208, 2223, 2232, 2255, 2321, 2343, 2370, 2380, 2436, 2449, 2490, 2451, 2720, 2721, 2722, 2725, 2726, 2727, 2729, 2730, 2731, 2732, 2733, 2734, 2735, 2736, 2737, 2738, 2740, 2935 or 2936.

Embodiment 10. The oligomeric compound of any of embodiments 1-9, wherein the nucleobase sequence of the modified oligonucleotide is at least 80%, 85%, 90%, 95%, or 100% complementary to any of the nucleobase sequences of SEQ ID NOs: 1-6 when measured across the entire nucleobase sequence of the modified oligonucleotide.

Embodiment 11. The oligomeric compound of any of embodiments 1-10, wherein at least one nucleoside of the modified oligonucleotide is a modified nucleoside.

Embodiment 12. The oligomeric compound of embodiment 11, wherein at least one modified nucleoside of the modified oligonucleotide comprises a modified sugar moiety.

Embodiment 13. The oligomeric compound of embodiment 12, wherein the modified sugar moiety comprises a bicyclic sugar moiety.

Embodiment 14. The oligomeric compound of embodiment 13, wherein the bicyclic sugar moiety comprises a 2′-4′ bridge selected from —O—CH₂— and —O—CH(CH₃)—.

Embodiment 15. The oligomeric compound of any of embodiments 11-14, wherein at least one modified nucleoside of the modified oligonucleotide comprises a non-bicyclic modified sugar moiety.

Embodiment 16. The oligomeric compound of embodiment 15, wherein at least one modified nucleoside of the modified oligonucleotide comprises a bicyclic sugar moiety having a 2′-4′ bridge and at least one nucleoside comprising a non-bicyclic modified sugar moiety.

Embodiment 17. The oligomeric compound of embodiment 15 or 16, wherein the non-bicyclic modified sugar moiety is a 2′-O(CH₂)₂—OCH₃ ribosyl modified sugar moiety, a 2′-OMe modified sugar moiety, or a 2′-F modified sugar moiety.

Embodiment 18. The oligomeric compound of any of embodiments 1-17, wherein the modified oligonucleotide comprises at least one modified nucleoside comprising a sugar surrogate.

Embodiment 19. The oligomeric compound of embodiment 18, wherein at least one modified nucleoside of the modified oligonucleotide comprises a sugar surrogate selected from morpholino and PNA.

Embodiment 20. The oligomeric compound of any of embodiments 1-19, wherein the modified oligonucleotide comprises at least one modified internucleoside linkage.

Embodiment 21. The oligomeric compound of embodiment 20, wherein each internucleoside linkage of the modified oligonucleotide is a modified internucleoside linkage.

Embodiment 22. The oligomeric compound of embodiment 20 or 21, wherein at least one internucleoside linkage is a phosphorothioate internucleoside linkage.

Embodiment 23. The oligomeric compound of embodiment 20 or 22, wherein the modified oligonucleotide comprises at least one phosphodiester internucleoside linkage.

Embodiment 24. The oligomeric compound of any of embodiments 20, 22, or 23, wherein each internucleoside linkage is independently selected from a phosphodiester internucleoside linkage or a phosphorothioate internucleoside linkage.

Embodiment 25. The oligomeric compound of any of embodiments 20 or 22-24, wherein at least 4, at least 5, at least 6, at least 7, at least 8, at least 9, at least 10, at least 11, at least 12, at least 13, at least 14, at least 15, at least 16, at least 17, at least 18, or at least 19 internucleoside linkages of the modified oligonucleotide are phosphorothioate internucleoside linkages.

Embodiment 26. The oligomeric compound of any of embodiments 20-25, wherein each internucleoside linkage is a phosphorothioate internucleoside linkage.

Embodiment 27. The oligomeric compound of any of embodiments 20 or 22-26, wherein the internucleoside linkage motif of the modified oligonucleotide is selected from: 5′-sssssssssssssssssss-3′, 5′-soossssssssssooss-3′, 5′-sooosssssssssssooss-3′, 5′-soossssssssssos-3′, 5′-ssoooooooooooooooooss-3′, 5′-ssooooooooooooooooss-3′, 5′-soooossssssssssooss-3′, 5′-sossssssssssssssoss-3′, 5′-soosssssssssooss-3′, and 5′-sooooossssssssssoss-3′; wherein each ‘o’ represents a phosphodiester internucleoside linkage and each ‘s’ represents a phosphorothioate internucleoside linkage.

Embodiment 28. The oligomeric compound of any of embodiments 1-27, wherein the modified oligonucleotide comprises a modified nucleobase.

Embodiment 29. The oligomeric compound of embodiment 28, wherein the modified nucleobase is a 5-methyl cytosine.

Embodiment 30. The oligomeric compound of any of embodiments 1-29, wherein the oligomeric compound comprises a modified oligonucleotide consisting of 12-22, 12-20, 14-18, 14-20, 15-17, 15-25, 16-20, 16-18, 18-22, 18-25, 18-20, 20-25, or 21-23 linked nucleosides, or a pharmaceutically acceptable salt thereof.

Embodiment 31. The oligomeric compound of embodiment 30, which is a pharmaceutically acceptable salt comprising one or more cations selected from sodium, potassium, calcium, and magnesium.

Embodiment 32. The oligomeric compound of any of embodiments 1-31, wherein the modified oligonucleotide consists of 16 or 18 linked nucleosides.

Embodiment 33. The oligomeric compound of any of embodiments 1-31, wherein the modified oligonucleotide consists of 20 linked nucleosides.

Embodiment 34. The oligomeric compound of any of embodiments 1-31, wherein the modified oligonucleotide consists of 21 linked nucleosides.

Embodiment 35. The oligomeric compound of any of embodiments 1-31, wherein the modified oligonucleotide consists of 23 linked nucleosides.

Embodiment 36. The oligomeric compound of any of embodiments 1-35, wherein the oligomeric compound is an RNase H compound.

Embodiment 37. The oligomeric compound of embodiment 36, wherein the modified oligonucleotide is a gapmer.

Embodiment 38. The oligomeric compound of any of embodiments 1-37, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprising a 2′-substituted sugar moiety.

Embodiment 39. The oligomeric compound of any of embodiments 1-34 or 36-37, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

Embodiment 40. The oligomeric compound of embodiment 39, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises either a cEt modified sugar moiety or a 2′-O(CH₂)₂—OCH₃ ribosyl modified sugar moiety, and each of the central region nucleosides comprises a 2′-β-D-deoxyribosyl sugar moiety.

Embodiment 41. The oligomeric compound of embodiment 39 or embodiment 40, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises a 2′-O(CH₂)₂—OCH₃ ribosyl modified sugar moiety, and each of the central region nucleosides comprises a 2′-β-D-deoxyribosyl sugar moiety.

Embodiment 42. The oligomeric compound of any of embodiments 1-35, wherein the oligomeric compound is an RNAi agent.

Embodiment 43. The oligomeric compound of any of embodiments 1-42, wherein the oligomeric compound comprises an antisense RNAi oligonucleotide comprising a targeting region comprising at least 15 contiguous nucleobases, wherein the targeting region is at least 90% complementary to an equal-length portion of an APOE RNA.

Embodiment 44. The oligomeric compound of embodiment 43, wherein the targeting region of the antisense RNAi oligonucleotide is at least 95% complementary or is 100% complementary to the equal length portion of an APOE RNA.

Embodiment 45. The oligomeric compound of any of embodiments 43-44, wherein the targeting region of the antisense RNAi oligonucleotide comprises at least 19, 20, 21, or 25 contiguous nucleobases.

Embodiment 46. The oligomeric compound of any of embodiments 43-45, wherein the APOE RNA has the nucleobase sequence of any of SEQ ID NOs: 1-6.

Embodiment 47. The oligomeric compound of any of embodiments 43-46, wherein at least one nucleoside of the antisense RNAi oligonucleotide comprises a modified sugar moiety selected from: 2′-F, 2′-OMe, 2′-O(CH₂)₂—OCH₃, 2′-NMA, LNA, and cEt; or a sugar surrogate selected from GNA, and UNA.

Embodiment 48. The oligomeric compound of any of embodiments 43-47, wherein each nucleoside of the antisense RNAi oligonucleotide comprises a modified sugar moiety or a sugar surrogate.

Embodiment 49. The oligomeric compound of any of embodiments 43-48, wherein at least 80%, at least 90%, or 100% of the nucleosides of the antisense RNAi oligonucleotide comprises a modified sugar moiety selected from 2′-F and 2′-OMe.

Embodiment 50. The oligomeric compound of any of embodiments 43-49, comprising a stabilized phosphate group attached to the 5′ position of the 5′-most nucleoside of the antisense RNAi oligonucleotide.

Embodiment 51. The oligomeric compound of embodiment 50, wherein the stabilized phosphate group comprises a cyclopropyl phosphonate or an (E)-vinyl phosphonate.

Embodiment 52. The oligomeric compound of any of embodiments 1-51, wherein the oligomeric compound is a single-stranded oligomeric compound.

Embodiment 53. The oligomeric compound of any of embodiments 1-52, consisting of the modified oligonucleotide or the RNAi antisense oligonucleotide.

Embodiment 54. The oligomeric compound of any of embodiments 1-53, comprising a conjugate group comprising a conjugate moiety and a conjugate linker.

Embodiment 55. The oligomeric compound of embodiment 54, wherein the conjugate linker consists of a single bond.

Embodiment 56. The oligomeric compound of embodiment 54, wherein the conjugate linker is cleavable.

Embodiment 57. The oligomeric compound of embodiment 54, wherein the conjugate linker comprises 1-3 linker-nucleosides.

Embodiment 58. The oligomeric compound of any of embodiments 54-57, wherein the conjugate group is attached to the 5′-end of the modified oligonucleotide or the antisense RNAi oligonucleotide.

Embodiment 59. The oligomeric compound of any of embodiments 54-57, wherein the conjugate group is attached to the 3′-end of the modified oligonucleotide or the antisense RNAi oligonucleotide.

Embodiment 60. The oligomeric compound of any of embodiments 1-59, comprising a terminal group.

Embodiment 61. The oligomeric compound of any of embodiments 1-56 or 58-60, wherein the oligomeric compound does not comprise linker-nucleosides.

Embodiment 62. An oligomeric duplex, comprising a first oligomeric compound comprising an antisense RNAi oligonucleotide of any of embodiments 43-61 and a second oligomeric compound comprising a sense RNAi oligonucleotide consisting of 17 to 30 linked nucleosides, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide.

Embodiment 63. The oligomeric duplex of embodiment 62, wherein the sense RNAi oligonucleotide consists of 18-25, 20-25, or 21-23 linked nucleosides.

Embodiment 64. The oligomeric duplex of embodiment 62, wherein the sense RNAi oligonucleotide consists of 21 or 23 linked nucleosides.

Embodiment 65. The oligomeric duplex of any of embodiments 62-64, wherein 1-4 3′-most nucleosides of the antisense or the sense RNAi oligonucleotide are overhanging nucleosides.

Embodiment 66. The oligomeric duplex of any of embodiments 62-65, wherein 1-4 5′-most nucleosides of the antisense or sense RNAi oligonucleotide are overhanging nucleosides.

Embodiment 67. The oligomeric duplex of any of embodiments 62-64, wherein the duplex is blunt ended at the 3′-end of the antisense RNAi oligonucleotide.

Embodiment 68. The oligomeric duplex of any of embodiments 62-64, wherein the duplex is blunt ended at the 5′-end of the antisense RNAi oligonucleotide.

Embodiment 69. The oligomeric duplex of any of embodiments 62-68, wherein at least one nucleoside of the sense RNAi oligonucleotide comprises a modified sugar moiety selected from: 2′-F, 2′-OMe, LNA, cEt, or a sugar surrogate selected from GNA, and UNA.

Embodiment 70. The oligomeric duplex of embodiment 69, wherein each nucleoside of the sense RNAi oligonucleotide comprises a modified sugar moiety or a sugar surrogate.

Embodiment 71. The oligomeric duplex of embodiment 70, wherein at least 80%, at least 90%, or 100% of the nucleosides of the sense RNAi oligonucleotide comprises a modified sugar moiety selected from 2′-F and 2′-OMe.

Embodiment 72. The oligomeric duplex of any of embodiments 62-71, wherein at least one nucleoside of the sense RNAi oligonucleotide comprises a modified nucleobase.

Embodiment 73. The oligomeric duplex of any of embodiments 62-72, wherein at least one internucleoside linkage of the sense RNAi oligonucleotide is a modified internucleoside linkage.

Embodiment 74. The oligomeric duplex of embodiment 73, wherein at least one internucleoside linkage of the sense RNAi oligonucleotide is a phosphorothioate internucleoside linkage.

Embodiment 75. The oligomeric duplex of any of embodiments 62-74, wherein the oligomeric duplex comprises 1-5 abasic sugar moieties attached to one or both ends of the antisense or sense RNA oligonucleotide.

Embodiment 76. The oligomeric duplex of any of embodiments 62-75, consisting of the antisense RNAi oligonucleotide and the sense RNAi oligonucleotide.

Embodiment 77. The oligomeric duplex of any of embodiments 62-75, wherein the second oligomeric compound comprises a conjugate group comprising a conjugate moiety and a conjugate linker.

Embodiment 78. The oligomeric duplex of embodiment 77, wherein the conjugate linker consists of a single bond.

Embodiment 79. The oligomeric duplex of embodiment 78, wherein the conjugate linker is cleavable.

Embodiment 80. The oligomeric duplex of embodiment 78, wherein the conjugate linker comprises 1-3 linker-nucleosides.

Embodiment 81. The oligomeric duplex of any of embodiments 78-80, wherein the conjugate group is attached to the 5′-end of the sense RNAi oligonucleotide.

Embodiment 82. The oligomeric duplex of any of embodiments 78-80, wherein the conjugate group is attached to the 3′-end of the sense RNAi oligonucleotide.

Embodiment 83. The oligomeric duplex of any of embodiments 78-80, wherein the conjugate group is attached via the 2′ position of a ribosyl sugar moiety at an internal position of the sense RNAi oligonucleotide.

Embodiment 84. The oligomeric compound of any of embodiments 54-59 or the oligomeric duplex of any of embodiments 77-83, wherein at least one conjugate group comprises a C₁₆ alkyl group.

Embodiment 85. The oligomeric duplex of embodiment 62, wherein the second oligomeric compound comprises a terminal group.

Embodiment 86. An antisense agent comprising an antisense compound, wherein the antisense compound is the oligomeric compound of any of embodiments 1-61.

Embodiment 87. The antisense agent of embodiment 86, wherein the antisense agent is the oligomeric duplex of any of embodiments 62-85.

Embodiment 88. The antisense agent of embodiment 86 or embodiment 87, wherein the antisense agent is:
i) an RNase H agent capable of reducing the amount of APOE nucleic acid through the activation of RNase H; or
ii) an RNAi agent capable of reducing the amount of APOE nucleic acid through the activation of RISC/Ago2.

Embodiment 89. The antisense agent of any of embodiments 86-88, wherein the antisense agent comprises a conjugate group, wherein the conjugate group comprises a cell-targeting moiety.

Embodiment 90. A chirally enriched population of oligomeric compounds of any of embodiments 1-61, wherein the population is enriched for modified oligonucleotides comprising at least one particular phosphorothioate internucleoside linkage having a particular stereochemical configuration.

Embodiment 91. The chirally enriched population of embodiment 90, wherein the population is enriched for modified oligonucleotides comprising at least one particular phosphorothioate internucleoside linkage having the (Sp) or (Rp) configuration.

Embodiment 92. The chirally enriched population of embodiment 90, wherein the population is enriched for modified oligonucleotides having a particular, independently selected stereochemical configuration at each phosphorothioate internucleoside linkage.

Embodiment 93. The chirally enriched population of embodiment 90, wherein the population is enriched for modified oligonucleotides having the (Rp) configuration at one particular phosphorothioate internucleoside linkage and the (Sp) configuration at each of the remaining phosphorothioate internucleoside linkages.

Embodiment 94. The chirally enriched population of embodiment 90, wherein the population is enriched for modified oligonucleotides having at least 3 contiguous phosphorothioate internucleoside linkages in the Sp, Sp, and Rp configurations, in the 5′ to 3′ direction.

Embodiment 95. A population of oligomeric compounds of any of embodiments 1-61, wherein all of the phosphorothioate internucleoside linkages of the modified oligonucleotide are stereorandom.

Embodiment 96. A pharmaceutical composition comprising an oligomeric compound of any of embodiments 1-61, an oligomeric duplex of any of embodiments 62-85, an antisense agent of any of embodiments 86-89, or a population of any of embodiments 90-95, and a pharmaceutically acceptable carrier or diluent.

Embodiment 97. The pharmaceutical composition of embodiment 96, wherein the pharmaceutically acceptable diluent is artificial cerebral spinal fluid (aCSF), sterile saline, or PBS.

Embodiment 98. The pharmaceutical composition of embodiment 97, wherein the pharmaceutical composition consists essentially of the modified oligonucleotide, the oligomeric duplex, the antisense agent, or the population and PBS or aCSF.

Embodiment 99. A method comprising administering to a subject an oligomeric compound of any of embodiments 1-61, an oligomeric duplex of any of embodiments 62-85, an antisense agent of any of embodiments 86-89, a population of any of embodiments 90-95, or a pharmaceutical composition of any of embodiments 96-98.

Embodiment 100. A method of treating a disease associated with APOE comprising administering to a subject having or at risk for developing a disease associated with APOE a therapeutically effective amount of an oligomeric compound of any of embodiments 1-61, an oligomeric duplex of any of embodiments 62-85, an antisense agent of any of embodiments 86-89, a population of any of embodiments 90-95, or a pharmaceutical composition according to any of embodiments 96-98; and thereby treating the disease associated with APOE.

Embodiment 101. The method of embodiment 100, wherein the APOE-associated disease is Alzheimer's Disease.

Embodiment 102. The method of any of embodiments 99-101, wherein at least one symptom or hallmark of the APOE-associated disease is ameliorated.

Embodiment 103. The method of embodiment 102, wherein the symptom or hallmark is cognitive impairment, progressive memory loss, behavioral abnormality, dementia, difficulty performing daily activities, amyloid plaques, neurofibrillary tangles, or neuroinflammation.

Embodiment 104. The method of any of embodiments 100-103, wherein administering an oligomeric compound of any of embodiments 1-61, an oligomeric duplex of any of embodiments 62-85, an antisense agent of any of embodiments 86-89, a population of any of embodiments 90-95, or a pharmaceutical composition according to any of embodiments 96-98 reduces cognitive impairment, behavioral abnormality, dementia, difficulty performing daily activities, amyloid plaques, neurofibrillary tangles, or neuroinflammation, or slows memory loss in the subject.

Embodiment 105. The method of any of embodiments 99-104, wherein the subject is human.

Embodiment 106. A method of reducing expression of APOE in a cell comprising contacting the cell with an oligomeric compound of any of embodiments 1-61, an oligomeric duplex of any of embodiments 62-85, an antisense agent of any of embodiments 86-89, a population of any of embodiments 90-95, or a pharmaceutical composition according to any of embodiments 96-98.

Embodiment 107. The method of embodiment 106, wherein the cell is a neuron or a glial cell, optionally wherein the cell is an astrocyte or microglial cell.

Embodiment 108. The method of embodiment 106 or embodiment 107, wherein the cell is a human cell.

Embodiment 109. Use of an oligomeric compound of any of embodiments 1-61, an oligomeric duplex of any of embodiments 62-85, an antisense agent of any of embodiments 86-89, a population of any of embodiments 90-95, or a pharmaceutical composition according to any of embodiments 96-98 for treating a disease associated with APOE.

Embodiment 110. Use of an oligomeric compound of any of embodiments 1-61, an oligomeric duplex of any of embodiments 62-85, an antisense agent of any of embodiments 86-89, a population of any of embodiments 90-95, or a pharmaceutical composition according to any of embodiments 96-98 in the manufacture of a medicament for treating a disease associated with APOE.

Embodiment 111. The use of embodiment 109 or embodiment 110, wherein the APOE-associated disease is Alzheimer's Disease.

I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. In certain embodiments, provided herein are RNAi agents comprising antisense RNAi oligonucleotides complementary to APOE and optionally sense RNAi oligonucleotides complementary to the antisense RNAi oligonucleotides. Antisense RNAi oligonucleotides and sense RNAi oligonucleotides typically comprise at least one modified nucleoside and/or at least one modified internucleoside linkage. Certain modified nucleosides and modified internucleoside linkages suitable for use in modified oligonucleotides are described below.

A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modifed nucleobases may be incorporated into antisense RNAi oligonucleotides and/or sense RNAi oligonucleotides.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)2OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)2SCH3, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH2)2ON(CH3)2 (“DMAOE”), 2′-OCH2OCH2N(CH2)2 (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl). In certain embodiments, non-bicyclic modified sugar moieties comprise a substituent group at the 4′-position. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, ethyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂N(CH₃)₂, O(CH₂)₂O(CH₂)₂N(CH₃)₂, O(CH₂)2ON(CH3)2 (“DMAOE”), OCH2OCH2N(CH2)2 (“DMAEOE”) and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. In certain embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, R^{a}, and R_{b}, is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Ra)(Rb)]n-, —[C(Ra)(Rb)]n-O—, C(Ra)═C(Rb)—, C(Ra)═N—, C(═NRa)—, —C(═O)—, —C(═S)—, —O—, —Si(Ra)2-, —S(═O)x-, and N(Ra)—;
wherein:
x is 0, 1, or 2;
n is 1, 2, 3, or 4;
each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

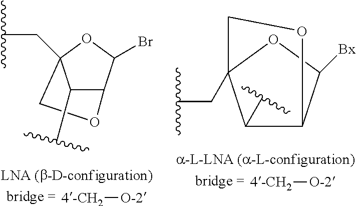

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

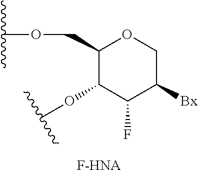

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

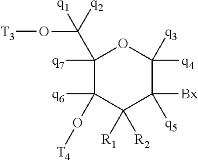

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

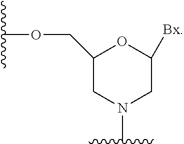

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the RNAi oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

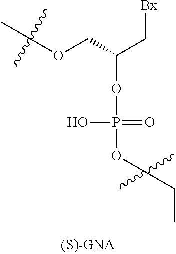

where Bx represents any nucleobase.

Many other bicyclic and tricyclic sugar and sugar surrogatsare known in the art that can be used in modified nucleosides.

2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N—2, N—6 and 0-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

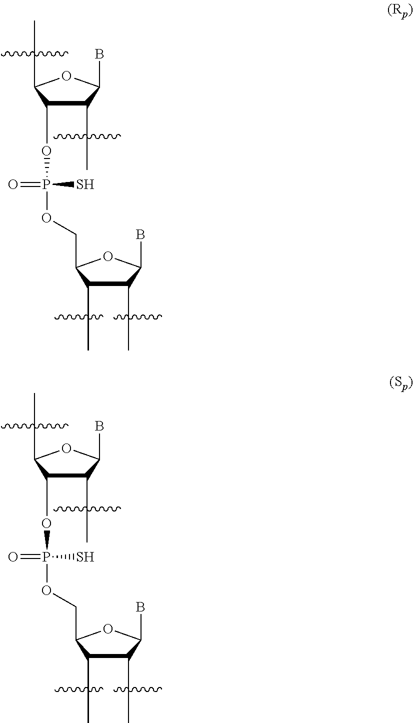

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′—CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides (such as antisense RNAi oligonucleotides and/or sense RNAi oligonucleotides) comprise one or more inverted nucleoside, as shown below:

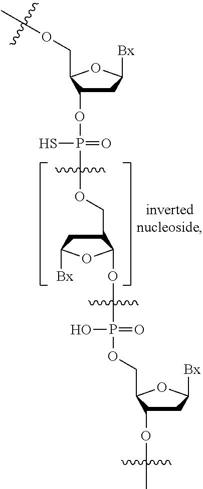

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

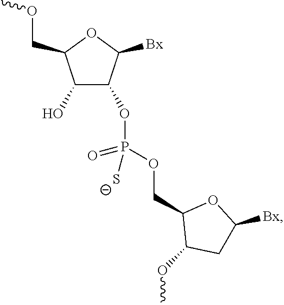

wherein each Bx represents any nucleobase.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Uniformly Modified Oligonucleotides

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

Herein, the lengths (number of nucleosides) of the three regions of a gapmer may be provided using the notation [# of nucleosides in the 5′-wing]—[# of nucleosides in the gap]—[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE modified nucleosides in the 5′-wing, 10 linked 2′-β-D-deoxynucleosides in the gap, and 5 linked 2′-MOE modified nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 6-10-4 MOE gapmer consists of 6 linked 2′-MOE modified nucleosides in the 5′-wing, 10 linked 2′-β-D-deoxynucleosides in the gap, and 4 linked 2′-MOE modified nucleosides in the 3′-wing. A 5-8-5 gapmer consists of 5 linked nucleosides comprising a modified sugar moiety in the 5′-wing, 8 linked 2′-β-D-deoxynucleosides in the gap, and 5 linked nucleosides comprising a modified sugar moiety in the 3′-wing. A 5-8-5 mixed gapmer has at least two different modified sugar moieties in the 5′- and/or the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers. In certain embodiments, modified oligonucleotides are 6-10-4 MOE gapmers. In certain embodiments, modified oligonucleotides are 5-8-5 MOE gapmers or 5-8-5 mixed gapmers.

In certain embodiments, modified oligonucleotides are 5-10-5 MOE gapmers that consist of 5 linked 2′-MOE modified nucleosides in the 5′-wing, 10 linked 2′-β-D-deoxynucleosides in the gap, and 5 linked 2′-MOE modified nucleosides in the 3′-wing. In certain embodiments, modified nucleosides have a sugar motif of eeeeeddddddddddeeeee, where each “e” represents a nucleoside comprising a 2′-MOE modified sugar moiety, and each “d” represents a nucleoside comprising a 2′-β-D-deoxyribosyl sugar moiety.

In certain embodiments, modified oligonucleotides are 3-10-3 cEt gapmers that consist of 3 linked cEt nucleosides in the 5′-wing, 10 linked 2′-β-D-deoxynucleosides in the gap, and 3 linked cEt nucleosides in the 3′-wing. In certain embodiments, modified nucleosides have a sugar motif of kkkddddddddddkkk, wherein each “k” represents a nucleoside comprising a cEt modified sugar moiety, and each “d” represents a nucleoside comprising a 2′-β-D-deoxyribosyl sugar moiety.

In certain embodiments, modified oligonucleotides are 6-10-4 MOE gapmers that consist of 6 linked 2′-MOE modified nucleosides in the 5′-wing, 10 linked 2′-β-D-deoxynucleosides in the gap, and 4 linked 2′-MOE modified nucleosides in the 3′-wing. In certain embodiments, modified nucleosides have a sugar motif of eeeeeeddddddddddeeee, where each “e” represents a nucleoside comprising a 2′-MOE modified sugar moiety, and each “d” represents a nucleoside comprising a 2′-β-D-deoxyribosyl sugar moiety.

In certain embodiments, modified oligonucleotides are 5-8-5 MOE gapmers that consist of 5 linked 2′-MOE modified nucleosides in the 5′-wing, 8 linked 2′-β-D-deoxynucleosides in the gap, and 5 linked 2′-MOE modified nucleosides in the 3′-wing. In certain embodiments, modified nucleosides have a sugar motif of eeeeeddddddddeeeee, where each “e” represents a nucleoside comprising a 2′-MOE modified sugar moiety, and each “d” represents a nucleoside comprising a 2′-β-D-deoxyribosyl sugar moiety.

In certain embodiments, modified oligonucleotides are 5-8-5 mixed gapmers that consist of 5 linked 2′-MOE modified nucleosides in the 5′-wing, 8 linked 2′-β-D-deoxynucleosides in the gap, and a mixture of cEt and 2′-MOE modified nucleosides in the 3′-wing. In certain embodiments, modified nucleosides have a sugar motif of eeeeeddddddddkkeee, where each “e” represents a nucleoside comprising a 2′-MOE modified sugar moiety, each “d” represents a nucleoside comprising a 2′-β-D-deoxyribosyl sugar moiety, and each “k” represents a nucleoside comprising a cEt modified sugar moiety. In certain embodiments, modified nucleosides have a sugar motif of eeeeeddddddddkeeee, where each “e” represents a nucleoside comprising a 2′-MOE modified sugar moiety, each “d” represents a nucleoside comprising a 2′-β-D-deoxyribosyl sugar moiety, and each “k” represents a nucleoside comprising a cEt modified sugar moiety.

### Antisense RNAi Oligonucleotides

In certain embodiments, the sugar moiety of at least one nucleoside of an antisense RNAi oligonucleotide is a modified sugar moiety.

In certain such embodiments, at least one nucleoside of the antisense RNAi oligonucleotide comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain embodiments, at least 21 nucleosides comprise 2′-OMe modified sugar moieties. In certain such embodiments, the remainder of the nucleosides are 2′-F modified.

In certain embodiments, at least one nucleoside of the antisense RNAi oligonucleotide comprises a 2′-F modified sugar moiety. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than one nucleoside comprises a 2′-F modified sugar. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, antisense RNAi oligonucleotides have a block of 2-4 contiguous 2′-F modified nucleosides. In certain embodiments, 4 nucleosides of an antisense RNAi oligonucleotide are 2′-F modified nucleosides and 3 of those 2′-F modified nucleosides are contiguous. In certain such embodiments, the remainder of the nucleosides are 2′-OMe modified.

In certain embodiments, at least one nucleoside of the antisense RNAi oligonucleotide comprises a 2′-OMe modified sugar moiety and at least one nucleoside comprises a 2′-F modified sugar moiety. In certain embodiments, at least 1, 2, 3, 4, 5, 6, 7, 8, 9 or 10 nucleosides comprises a 2′-OMe modified sugar moiety and at least 1, 2, 3, 4, 5, 6, 7, 8, 9 or 10 nucleosides comprises a 2′-F modified sugar moiety. In certain embodiments, the antisense RNAi oligonucleotide comprises a sugar motif of fyf or yfy, wherein each “f” represents a 2′-F modified sugar moiety and each “y” represents a 2′-OMe modified sugar moiety. In certain embodiments, the antisense RNAi oligonucleotide has a sugar motif of yfyfyfyfyfyfyfyfyfyfyyy, wherein each “f” represents a 2′-F modified sugar moiety and each “y” represents a 2′-OMe modified sugar moiety.

In certain embodiments, at least one nucleoside of the antisense RNAi oligonucleotide comprises a 2′-MOE modified sugar moiety. In certain embodiments, the antisense RNAi oligonucleotide further comprises one or more 2′-OMe modified sugar moieties and one or more 2′-F modified sugar moieties. In certain embodiments, at least 1, 2, 3, 4, 5, 6, 7, 8, 9, 10, 11, 12, 13, 14, 15, 16, 17, or 18 nucleosides comprises a 2′-OMe modified sugar moiety. In certain embodiments, at least 1, 2, 3, or 4 nucleosides comprises a 2′-F modified sugar moiety. In certain embodiments, the antisense RNAi oligonucleotide has a sugar motif of efyyyfyyyyyyyfyfyyyyyyy, wherein ‘e’ represents a 2′-MOE modified sugar moiety, each “y” represents a 2′-O-methylribosyl sugar, and each “f” represents a 2′-fluororibosyl sugar.

### Sense RNAi Oligonucleotides

In certain embodiments, the sugar moiety of at least one nucleoside of a sense RNAi oligonucleotides is a modified sugar moiety.

In certain such embodiments, at least one nucleoside of the sense RNAi oligonucleotide comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain such embodiments, at least 21 nucleosides comprise 2′-OMe modified sugar moieties.

In certain embodiments, at least one nucleoside of the sense RNAi oligonucleotide comprises a 2′-F modified sugar moiety. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than nucleoside comprises a 2′-F modified sugar moiety. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, sense RNAi oligonucleotides have a block of 2-4 contiguous 2′-F modified nucleosides. In certain embodiments, 4 nucleosides of a sense RNAi oligonucleotide are 2′-F modified nucleosides and 3 of those 2′-F modified nucleosides are contiguous. In certain such embodiments the remainder of the nucleosides are 2′OMe modified.

In certain embodiments, at least one nucleoside of the sense RNAi oligonucleotide comprises a 2′-OMe modified sugar moiety and at least one nucleoside comprises a 2′-F modified sugar moiety. In certain embodiments, at least 1, 2, 3, 4, 5, 6, 7, 8, 9, 10, 11, 12, 13, 14, 15, 16, or 17 nucleosides comprises a 2′-OMe modified sugar moiety and at least 1, 2, 3, 4, 5, 6, 7, 8, 9 or 10 nucleosides comprises a 2′-F modified sugar moiety. In certain embodiments, the sense RNAi oligonucleotide comprises a sugar motif of fyf or yfy, wherein each “f” represents a 2′-F modified sugar moiety and each “y” represents a 2′-OMe modified sugar moiety. In certain embodiments, the sense RNAi oligonucleotide has a sugar motif of fyfyfyfyfyfyfyfyfyfyf, wherein each “f” represents a 2′-F modified sugar moiety and each “y” represents a 2′-OMe modified sugar moiety. In certain embodiments, the sense RNAi oligonucleotide has a sugar motif of yyyyyyfyfffyyyyyyyyyy, wherein each “y” represents a 2′-O-methylribosyl sugar, and each “f” represents a 2′-fluororibosyl sugar.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### Gapmer Oligonucleotides

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from: a 2-thiopyrimidine and a 5-propynepyrimidine.

### Antisense RNAi Oligonucleotides

In certain embodiments, one nucleoside of an antisense RNAi oligonucleotide is a UNA. In certain embodiments, one nucleoside of an antisense RNAi oligonucleotide is a GNA. In certain embodiments, 1-4 nucleosides of an antisense RNAi oligonucleotide is/are DNA. In certain such embodiments, the 1-4 DNA nucleosides are at one or both ends of the antisense RNAi oligonucleotide.

### Sense RNAi Oligonucleotides

In certain embodiments, one nucleoside of a sense RNAi oligonucleotide is a UNA. In certain embodiments, one nucleoside of a sense RNAi oligonucleotide is a GNA. In certain embodiments, 1-4 nucleosides of a sense RNAi oligonucleotide is/are DNA. In certain such embodiments, the 1-4 DNA nucleosides are at one or both ends of the sense RNAi oligonucleotide.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═0). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

### Gapmer Oligonucleotides

In certain embodiments, the sugar motif of a modified oligonucleotide is a gapmer and the internucleoside linkages within the gap are all modified. In certain embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified nucleotides have an internucleoside linkage motif of sososssssssssosss, wherein each “s” represents a phosphorothioate internucleoside linkage and each “o” represents a phosphodiester internucleoside linkage. In certain embodiments, modified nucleotides have an internucleoside linkage motif of sooosssssssssooss, wherein each “s” represents a phosphorothioate internucleoside linkage and each “o” represents a phosphodiester internucleoside linkage. In certain embodiments, modified nucleotides have an internucleoside linkage motif of sooosssssssssooss, wherein each “s” represents a phosphorothioate internucleoside linkage and each “o” represents a phosphodiester internucleoside linkage. In certain embodiments, modified nucleotides have an internucleoside linkage motif of soossssssssssooss, wherein each “s” represents a phosphorothioate internucleoside linkage and each “o” represents a phosphodiester internucleoside linkage. In certain embodiments, modified nucleotides have an internucleoside linkage motif of soooossssssssssooss, wherein each “s” represents a phosphorothioate internucleoside linkage and each “o” represents a phosphodiester internucleoside linkage. In certain embodiments, modified nucleotides have an internucleoside linkage motif of sossssssssssssssoss, wherein each “s” represents a phosphorothioate internucleoside linkage and each “o” represents a phosphodiester internucleoside linkage. In certain embodiments, modified nucleotides have an internucleoside linkage motif of soossssssssssos, wherein each “s” represents a phosphorothioate internucleoside linkage and each “o” represents a phosphodiester internucleoside linkage. In certain embodiments, modified nucleotides have an internucleoside linkage motif of soosssssssssooss, wherein each “s” represents a phosphorothioate internucleoside linkage and each “o” represents a phosphodiester internucleoside linkage. In certain embodiments, modified nucleotides have an internucleoside linkage motif of sooosssssssssssooss, wherein each “s” represents a phosphorothioate internucleoside linkage and each “o” represents a phosphodiester internucleoside linkage. In certain embodiments, modified nucleotides have an internucleoside linkage motif of sooooossssssssssoss, wherein each “s” represents a phosphorothioate internucleoside linkage and each “o” represents a phosphodiester internucleoside linkage.

### Antisense RNAi Oligonucleotides

In certain embodiments, at least one linkage of the antisense RNAi oligonucleotide is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages. In certain embodiments, antisense RNAi oligonucleotides have an internucleoside linkage motif of ssooooooooooooooooooss, wherein each “s” represents a phosphorothioate internucleoside linkage and each “o” represents a phosphodiester internucleoside linkage.

In certain embodiments, at least one linkage of the antisense RNAi oligonucleotide is an inverted linkage.

### Sense RNAi Oligonucleotides

In certain embodiments, at least one linkage of the sense RNAi oligonucleotides is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages. In certain embodiments, sense RNAi oligonucleotides have an internucleoside linkage motif of ssooooooooooooooooss, wherein each “s” represents a phosphorothioate internucleoside linkage and each “o” represents a phosphodiester internucleoside linkage.

In certain embodiments, at least one linkage of the sense RNAi oligonucleotides is an inverted linkage.

C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### Antisense RNAi Oligonucleotides

In certain embodiments, antisense RNAi oligonucleotides consist of 17-30 linked nucleosides. In certain embodiments, antisense RNAi oligonucleotides consist of 17-25 linked nucleosides. In certain embodiments, antisense RNAi oligonucleotides consist of 17-23 linked nucleosides. In certain embodiments, antisense RNAi oligonucleotides consist of 17-21 linked nucleosides. In certain embodiments, antisense RNAi oligonucleotides consist of 18-30 linked nucleosides. In certain embodiments, antisense RNAi oligonucleotides consist of 20-30 linked nucleosides. In certain embodiments, antisense RNAi oligonucleotides consist of 21-30 linked nucleosides. In certain embodiments, antisense RNAi oligonucleotides consist of 23-30 linked nucleosides. In certain embodiments, antisense RNAi oligonucleotides consist of 18-25 linked nucleosides. In certain embodiments, antisense RNAi oligonucleotides consist of 20-22 linked nucleosides. In certain embodiments, antisense RNAi oligonucleotides consist of 21-23 linked nucleosides. In certain embodiments, antisense RNAi oligonucleotides consist of 23-24 linked nucleosides. In certain embodiments, antisense RNAi oligonucleotides consist of 20 linked nucleosides. In certain embodiments, antisense RNAi oligonucleotides consist of 21 linked nucleosides. In certain embodiments, antisense RNAi oligonucleotides consist of 22 linked nucleosides. In certain embodiments, antisense RNAi oligonucleotides consist of 23 linked nucleosides.

### Sense RNAi Oligonucleotides

In certain embodiments, sense RNAi oligonucleotides consist of 17-30 linked nucleosides. In certain embodiments, sense RNAi oligonucleotides consist of 17-25 linked nucleosides. In certain embodiments, sense RNAi oligonucleotides consist of 17-23 linked nucleosides. In certain embodiments, sense RNAi oligonucleotides consist of 17-21 linked nucleosides. In certain embodiments, sense RNAi oligonucleotides consist of 18-30 linked nucleosides. In certain embodiments, sense RNAi oligonucleotides consist of 20-30 linked nucleosides. In certain embodiments, sense RNAi oligonucleotides consist of 21-30 linked nucleosides. In certain embodiments, sense RNAi oligonucleotides consist of 23-30 linked nucleosides. In certain embodiments, sense RNAi oligonucleotides consist of 18-25 linked nucleosides. In certain embodiments, sense RNAi oligonucleotides consist of 20-22 linked nucleosides. In certain embodiments, sense RNAi oligonucleotides consist of 21-23 linked nucleosides. In certain embodiments, sense RNAi oligonucleotides consist of 23-24 linked nucleosides. In certain embodiments, sense RNAi oligonucleotides consist of 20 linked nucleosides. In certain embodiments, sense RNAi oligonucleotides consist of 21 linked nucleosides. In certain embodiments, sense RNAi oligonucleotides consist of 22 linked nucleosides. In certain embodiments, sense RNAi oligonucleotides consist of 23 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain RNAi Agents

RNAi agents comprise an antisense RNAi oligonucleotide and optionally a sense RNAi oligonucleotide. RNAi agents may also comprise terminal groups and/or conjugate groups which may be attached to the antisense RNAi oligonucleotide or the sense RNAi oligonucleotide (when present).

Duplexes

RNAi agents comprising an antisense RNAi oligonucleotide and a sense RNAi oligonucleotide form a duplex, because the sense RNAi oligonucleotide comprises an antisense-hybridizing region that is complementary to the antisense RNAi oligonucleotide. In certain embodiments, each nucleobase of the antisense RNAi oligonucleotide and the sense RNAi oligonucleotide are complementary to one another. In certain embodiments, the two RNAi oligonucleotides have at least one mismatch relative to one another.

In certain embodiments, the antisense hybridizing region constitutes the entire length of the sense RNAi oligonucleotide and the antisense RNAi oligonucleotide. In certain embodiments, one or both of the antisense RNAi oligonucleotide and the sense RNAi oligonucleotide comprise additional nucleosides at one or both ends that do not hybridize (overhanging nucleosides). In certain embodiments, overhanging nucleosides are DNA. In certain embodiments, overhanging nucleosides are linked to each other (where there is more than one) and to the first non-overhanging nucleoside with phosphorothioate linkages.

B. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer. In certain embodiments, the modified oligonucleotide is an antisense RNAi oligonucleotide. In certain embodiments, the modified oligonucleotide is a sense RNAi oligonucleotide.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, a conjugate group is a lipid having the following structure:

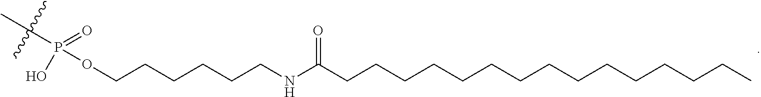 [SMILES: *P(=O)(O)OCCCCCCNC(=O)CCCCCCCCCCCCCCC]

In certain embodiments, a conjugate group is a lipid having the following structure:

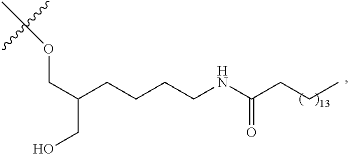

which is also referred to herein as 3nC7-C16. “3nC7-C16” represents a palmitate moiety linked to a 3′-C7 amino modifier and is attached to the 3′-nucleoside of an RNAi oligonucleotide (e.g., the 3′-nucleoside of a sense oligonucleotide or the antisense oligonucleotide) via a phosphodiester linkage.

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is 2′-deoxyadenosine.

3. Cell-Targeting Moieties

In certain embodiments, a conjugate group comprises a cell-targeting moiety. In certain embodiments, a conjugate group has the general formula:

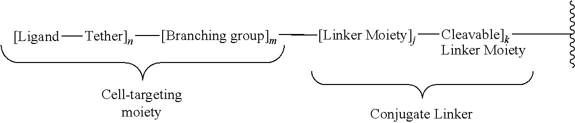

wherein n is from 1 to about 3, m is 0 when n is 1, m is 1 when n is 2 or greater, j is 1 or 0, and k is 1 or O.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

C. Certain Terminal Groups

In certain embodiments, oligomeric compounds comprise one or more terminal groups. In certain such embodiments, modified oligonucleotides comprise a phosphorus-containing group at the 5′-end of the modified oligonucleotide. In certain embodiments, the phosphorus-containing group is at the 5′-end of the antisense RNAi oligonucleotide and/or the sense RNAi oligonucleotide. In certain embodiments, the terminal group is a phosphate stabilized phosphate group. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos) or 5′-deoxy-5′-C-malonyl. When the 5′-end phosphorus-containing group is 5′-end vinylphosphonate, the 5′VP can be either 5′-E-VP isomer (i.e., trans-vinylphosphonate), 5′-Z-VP isomer (i.e., cis-vinylphosphonate), or mixtures thereof. Although such phosphate group can be attached to any modified oligonucleotide, it has particularly been shown that attachment of such a group to an antisense RNAi oligonucleotide improves activity of certain RNAi agents. See, e.g., Prakash et al., Nucleic Acids Res., 43(6):2993-3011, 2015; Elkayam, et al., Nucleic Acids Res., 45(6):3528-3536, 2017; Parmar, et al. ChemBioChem, 17(11)985-989; 2016; Harastzi, et al., Nucleic Acids Res., 45(13):7581-7592, 2017. In certain embodiments, the phosphate stabilizing group is 5′-cyclopropyl phosphonate. See e.g., WO/2018/027106.

In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

D. Certain Specific RNAi Motifs

RNAi agents can be described by motif or by specific features.

In certain embodiments, the RNAi agents described herein comprise:
(a) a sense RNAi oligonucleotide having:
   (i) a length of 21 nucleotides;
   (ii) a conjugate attached to the 3′-end; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense RNAi oligonucleotide having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleoside linkages between nucleoside positions 21 and 22, and between nucleoside positions 22 and 23 (counting from the 5′ end);
   wherein the two nucleotides at the 3′end of the antisense RNAi oligonucleotide are overhanging nucleosides, and the end of the RNAi agent duplex constituting the 5′-end of the antisense RNAi oligonucleotide and the 3′-end of the sense RNAi oligonucleotide is blunt (i.e., neither oligonucleotide has overhang nucleoside at that end and instead the hybridizing region of the sense RNAi oligonucleotide includes the 3′-most nucleoside of the sense RNAi oligonucleotide and that nucleoside hybridizes with the 5′-most nucleoside of the antisense oligonucleotide).

In certain embodiments, the RNAi agents described herein comprise:
(a) a sense RNAi oligonucleotide having:
   (i) a length of 21 nucleotides;
   (ii) a conjugate attached to the 3′-end;
   (iii) 2′-F modifications at positions 1, 3, 5, 7, 9 to 11, 13, 17, 19, and 21, and 2′-OMe modifications at positions 2, 4, 6, 8, 12, 14, 16, 18, and 20 (counting from the 5′ end); and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached to the 3′-end;
   (iii) 2′-OMe modifications at positions 1 to 6, 8, 10, and 12 to 21, and 2′-F modifications at positions 7 and 9, and a deoxynucleotide at position 11 (counting from the 5′ end); and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3, 7, 9, 11, 13, 15, 17, and 19 to 23, and 2′F modifications at positions 2, 4 to 6, 8, 10, 12, 14, 16, and 18 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached to the 3′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 5, 7, 8, 10 to 13, 15, and 17 to 23, and 2′F modifications at positions 2, 6, 9, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached to the 3′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 5, 7, 10 to 13, 15, and 17 to 23, and 2′F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 19 nucleotides;
   (ii) a conjugate attached to the 3′-end;
   (iii) 2′-OMe modifications at positions 1 to 4, 6, and 10 to 19, and 2′-F modifications at positions 5, and 7 to 9; and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 21 nucleotides;
   (ii) 2′-OMe modifications at positions 1, 3 to 5, 7, 10 to 13, 15, and 17 to 21, and 2′F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleoside positions 19 and 20, and between nucleoside positions 20 and 21 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached at position 6 (counting from the 5′ end);
   (iii) 2′-F modifications at positions 7 and 9 to 11, and 2′-OMe modifications at positions 1 to 5, 8, and 12 to 21 (counting from the 5′ end); and
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense RNAi oligonucleotide having:
   (i) a length of 23 nucleotides;
   (ii) 2′-OMe modifications at positions 1, 3 to 5, 7, 10 to 13, 15, and 17 to 23, and 2′F modifications at positions 2, 6, 8, 9, 14, and 16 (counting from the 5′ end);
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
   (iv) a stabilized phosphate group attached to the 5′ position of the 5′-most nucleoside;
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached to the 3′-end;
   (iii) 2′-F modifications at positions 7 and 9 to 11, and 2′-OMe modifications at positions 1 to 6, 8, and 12 to 21 (counting from the 5′ end);
   (iv) phosphorothioate internucleoside linkages between nucleoside positions 1 and 2 and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 5, 7 to 13, 15, and 17 to 23 an (S)-GNA modification at position 6, and 2′F modifications at positions 2, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached to the 3′-end;
   (iii) 2′-F modifications at positions 7 and 9 to 11, and 2′-OMe modifications at positions 1 to 6, 8, and 12 to 21 (counting from the 5′ end);
   (iv) phosphorothioate internucleoside linkages between nucleoside positions 1 and 2 and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 6, 8 to 13, 15, and 17 to 23 an (S)-GNA modification at position 7, and 2′F modifications at positions 2, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached at position 6 (counting from the 5′ end); and
   (iii) 2′-F modifications at positions 7 and 9 to 11, and 2′-OMe modifications at positions 1 to 5, 8, and 12 to 21 (counting from the 5′ end);
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense RNAi oligonucleotide having:
   (i) a length of 23 nucleotides;
   (ii) 2′-OMe modifications at positions 1, 3 to 5, 7 to 13, 15, and 17 to 23 an (S)-GNA modification at position 6, and 2′F modifications at positions 2, 14, and 16 (counting from the 5′ end);
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
   (iv) a stabilized phosphate group attached to the 5′ position of the 5′-most nucleoside;
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi agents described herein comprise:
(a) a sense RNAi oligonucleotide having:
   (i) a length of 21 nucleotides;
   (ii) a conjugate attached at position 6 (counting from the 5′ end);
   (iii) 2′-F modifications at positions 7 and 9 to 11, and 2′-OMe modifications at positions 1 to 5, 8, and 12 to 21 (counting from the 5′ end); and
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense RNAi oligonucleotide having:
   (i) a length of 23 nucleotides;
   (ii) 2′-OMe modifications at positions 1, 3 to 6, 8 to 13, 15, and 17 to 23 an (S)-GNA modification at position 7, and 2′F modifications at positions 2, 14, and 16 (counting from the 5′ end);
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
   (iv) a stabilized phosphate group attached to the 5′ position of the 5′-most nucleoside;
   wherein the two nucleotides at the 3′end of the antisense RNAi oligonucleotide are overhanging nucleosides, and the end of the RNAi agent duplex constituting the 5′-end of the antisense RNAi oligonucleotide and the 3′-end of the sense RNAi oligonucleotide is blunt (i.e., neither oligonucleotide has overhang nucleoside at that end and instead the hybridizing region of the sense RNAi oligonucleotide includes the 3′-most nucleoside of the sense RNAi oligonucleotide and that nucleoside hybridizes with the 5′-most nucleoside of the antisense oligonucleotide).

In certain embodiments, the RNAi agents described herein comprise:
(a) a sense RNAi oligonucleotide having:
   (i) a length of 21 nucleotides;
   (ii) a conjugate attached to the 5′-end;
   (iii) 2′-OMe modifications at positions 1 to 8, and 12 to 21, and 2′-F modifications at positions 9 to 11; and
   (iv) inverted abasic sugar moieties attached to both the 5′-most and 3′-most nucleosides;
and
(b) an antisense RNAi oligonucleotide having:
   (i) a length of 21 nucleotides;
   (ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 3 and 4, and between nucleoside positions 20 and 21 (counting from the 5′ end).

In certain embodiments, the RNAi agents described herein comprise:
(a) a sense RNAi oligonucleotide having:
   (i) a length of 21 nucleotides;
   (ii) a conjugate attached to the 5′-end;
   (iii) 2′-OMe modifications at positions 1 to 8, and 12 to 21, and 2′-F modifications at positions 9 to 11;
   (iv) a phosphorothioate internucleoside linkage between nucleoside positions 1 and 2 (counting from the 5′ end); and
   (v) an inverted abasic sugar moiety attached to the 3′-most nucleoside;
and
(b) an antisense RNAi oligonucleotide having:
   (i) a length of 21 nucleotides;
   (ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 3 and 4, and between nucleoside positions 20 and 21 (counting from the 5′ end).

In certain embodiments, the RNAi agents described herein comprise:
(a) a sense RNAi oligonucleotide having:
   (i) a length of 19 nucleotides;
   (ii) a conjugate attached to the 5′-end;
   (iii) 2′-OMe modifications at positions 2, 4, 6, 8, 10, 12, 14, 16, 18, and 20, and 2′-F modifications at positions 1, 3, 5, 7, 9, 11, 13, 15, 17, 19, and 21; and
   (iv) phosphorothioate internucleoside linkages between nucleoside positions 17 and 18, and between nucleoside positions 18 and 19 (counting from the 5′ end);
and
(b) an antisense RNAi oligonucleotide having:
   (i) a length of 19 nucleotides;
   (ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 17 and 18, and between nucleoside positions 18 and 19 (counting from the 5′ end).

In any of the above embodiments, the conjugate at the 3′-end of the sense RNAi oligonucleotide may comprise a targeting moiety. In certain embodiments, the targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, the RNAi agent comprises a 21 nucleotide sense RNAi oligonucleotide and a 23 nucleotide antisense RNAi oligonucleotide, wherein the sense RNAi oligonucleotide contains at least one motif of three contiguous 2′-F modified nucleosides at positions 9, 10, 11 from the 5′-end; the antisense RNAi oligonucleotide contains at least one motif of three 2′-O-methyl modifications on three consecutive nucleotides at positions 11, 12, 13 from the 5′ end, wherein one end of the RNAi agent is blunt, while the other end comprises a 2 nucleotide overhang. Preferably, the 2 nucleotide overhang is at the 3′-end of the antisense RNAi oligonucleotide.

In certain embodiments, when the 2 nucleotide overhang is at the 3′-end of the antisense RNAi oligonucleotide, there may be two phosphorothioate internucleoside linkages between the terminal three nucleotides, wherein two of the three nucleotides are the overhang nucleotides, and the third nucleotide is a paired nucleotide next to the overhang nucleotide. In certain embodiments, the RNAi agent additionally has two phosphorothioate internucleoside linkages between the terminal three nucleotides at both the 5′-end of the sense RNAi oligonucleotide and at the 5′-end of the antisense RNAi oligonucleotide. In certain embodiments, every nucleotide in the sense RNAi oligonucleotide and the antisense RNAi oligonucleotide of the RNAi agent is a modified nucleotide. In certain embodiments, each nucleotide is independently modified with a 2′-O-methyl or 3′-fluoro, e.g. in an alternating motif. Optionally, the RNAi agent comprises a conjugate.

In certain embodiments, every nucleotide in the sense RNAi oligonucleotide and antisense RNAi oligonucleotide of the RNAi agent, including the nucleotides that are part of the motifs, may be modified. Each nucleotide may be modified with the same or different modification, which can include one or more alteration of one or both of the non-linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In certain embodiments, each nucleoside of the sense RNAi oligonucleotide and antisense RNAi oligonucleotide is independently modified with LNA, cEt, UNA, HNA, CeNA, 2′-MOE, 2′-OMe, 2′-O-allyl, 2′-C-allyl, 2′-deoxy, 2′-hydroxyl, or 2′-fluoro. The RNAi agent can contain more than one modification. In certain embodiments, each nucleoside of the sense RNAi oligonucleotide and antisense RNAi oligonucleotide is independently modified with 2′-O-methyl or 2′-F. In certain embodiments, the modification is a 2′-NMA modification.

The term “alternating motif” as used herein refers to a motif having one or more modifications, each modification occurring on alternating nucleotides of one RNAi oligonucleotide. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense RNAi oligonucleotide or antisense RNAi oligonucleotide can be selected from several possibilities of modifications within the alternating motif such as “ABABAB . . . ”, “ACACAC . . . ” “BDBDBD . . . ” or “CDCDCD . . . ,” etc.

In certain embodiments, the modification pattern for the alternating motif on the sense RNAi oligonucleotide relative to the modification pattern for the alternating motif on the antisense RNAi oligonucleotide is shifted. The shift may be such that the group of modified nucleotides of the sense RNAi oligonucleotide corresponds to a group of differently modified nucleotides of the antisense RNAi oligonucleotide and vice versa. For example, the sense RNAi oligonucleotide when paired with the antisense RNAi oligonucleotide in the RNAi duplex, the alternating motif in the sense RNAi oligonucleotide may start with “ABABAB” from 5′-3′ of the RNAi oligonucleotide and the alternating motif in the antisense RNAi oligonucleotide may start with “BABABA” from 5′-3 ‘of the RNAi oligonucleotide within the duplex region. As another example, the alternating motif in the sense RNAi oligonucleotide may start with “AABBAABB” from 5’-3′ of the RNAi oligonucleotide and the alternating motif in the antisense RNAi oligonucleotide may start with “BBAABBAA” from 5′-3′ of the RNAi oligonucleotide within the duplex region, so that there is a complete or partial shift of the modification 10 patterns between the sense RNAi oligonucleotide and the antisense RNAi oligonucleotide.

In certain embodiments, the RNAi agent comprising the pattern of the alternating motif of 2′-O-methyl modification and 2′-F modification on the sense RNAi oligonucleotide initially has a shift relative to the pattern of the alternating motif of 2′-O-methyl modification and 2′-F modification on the antisense RNAi oligonucleotide initially, i.e., the 2′-O-methyl modified nucleotide on the sense RNAi oligonucleotide base pairs with a 2′-F modified nucleotides on the antisense RNAi oligonucleotide and vice versa. The 1 position of the sense RNAi oligonucleotide may start with the 2′-F modification, and the 1 position of the antisense RNAi oligonucleotide may start with a 2′-O-methyl modification.

The introduction of one or more motifs of three identical modifications on three consecutive nucleotides to the sense RNAi oligonucleotide and/or antisense RNAi oligonucleotide interrupts the initial modification pattern present in the sense RNAi oligonucleotide and/or antisense RNAi oligonucleotide. This interruption of the modification pattern of the sense and/or antisense RNAi oligonucleotide by introducing one or more motifs of three identical modifications on three consecutive nucleotides to the sense and/or antisense RNAi oligonucleotide surprisingly enhances the gene silencing activity to the target gene. In one embodiment, when the motif of three identical modifications on three consecutive 25 nucleotides is introduced to any of the RNAi oligonucleotide s, the modification of the nucleotide next to the motif is a different modification than the modification of the motif. For example, the portion of the sequence containing the motif is “ . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications. Alternatively, Na and/or Nb may be present or absent when there is a wing modification present.

In certain embodiments, the sense RNAi oligonucleotide may be represented by formula (I):

5′nₚ-Nₐ—(XXX)i-N_{b}—YYY—N_{b}—(ZZZ)ᵣNₐ-n_{q}3′ (I)
wherein:
i and j are each independently 0 or 1;
p and q are each independently 0-6;
each Nₐ independently represents 0-25 linked nucleosides comprising at least two differently modified nucleosides;
each N_{b} independently represents 0-10 linked nucleosides;
each nₚ and n_{q} independently represent an overhanging nucleoside;
wherein N_{b} and Y do not have the same modification; and
XXX, YYY, and ZZZ each independently represent modified nucleosides where each X nucleoside has the same modification; each Y nucleoside has the same modification; and each Z nucleoside has the same modification. In certain embodiments, each Y comprises a 2′-F modification.

In certain embodiments, the Nₐ and N_{b} comprise modifications of alternating patterns.

In certain embodiments, the YYY motif occurs at or near the cleavage site of the target nucleic acid. For example, when the RNAi agent has a duplex region of 17-23 nucleotides in length, the YYY motif can occur at or near the vicinity of the cleavage site (e.g., can occur at positions 6, 7, 8; 7, 8, 9; 8, 9, 10; 9, 10, 11; 10, 11, 12; or 11, 12, 13) of the sense RNAi oligonucleotide, the count starting from the 1^{st} nucleotide from the 5′-end; or optionally, the count starting at the 1^{st} paired nucleotide within the duplex region, from the 5′-end.

In certain embodiments, the antisense RNAi oligonucleotide of the RNAi may be represented by the formula:

5′n_{q}-Nₐ′—(Z′Z′Z′)ₖ—N_{b}′—Y′Y′Y′—N_{b}′—(X′X′X′)ₗ—N′ₐ-nₚ3′ (II)
wherein:
k and l are each independently 0 or 1;
p′ and q′ are each independently 0-6;
each Nₐ′ independently represents 0-25 linked nucleotides comprising at least two differently modified nucleotides;
each N_{b}′ independently represents 0-10 linked nucleotides;
each nₚ′ and n_{q}′ independently represent an overhanging nucleoside;
wherein N_{b}′ and Y′ do not have the same modification; and
X′X′X′, Y′Y′Y′, and Z′Z′Z′ each independently represent modified nucleosides where each X′ nucleoside has the same modification; each Y′ nucleoside has the same modification; and each Z′ nucleoside has the same modification. In certain embodiments, each Y′ comprises a 2′-F modification. In certain embodiments, each Y′ comprises a 2′-OMe modification.

In certain embodiments, the Nₐ′ and/or N_{b}′ comprise modifications of alternating patterns.

In certain embodiments, the Y′Y′Y′ motif occurs at or near the cleavage site of the target nucleic acid. For example, when the RNAi agent has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense RNAi oligonucleotide, with the count starting from the 1^{st} nucleotide from the 5′-end; or, optionally, the count starting at the 1^{st} paired nucleotide within the duplex region, from the 5′-end. Preferably, the Y′Y′Y′ motif occurs at positions 11, 12, 13.

In certain embodiments, k is 1 and l is 0, or k is 0 and l is 1, or both k and l are 1.

The antisense RNAi oligonucleotide can therefore be represented by the following formulas:

5′n_{q}′—Nₐ′—Z′Z′Z′—N_{b}′—Y′Y′Y′—Nₐ′-nₚ′3′ (IIb);

5′n_{q}′—Nₐ′—Y′Y′Y′—N_{b}′—X′X′X′-nₚ′3′ (IIc); or

5′n_{q}′—Nₐ′—Z′Z′Z′—N_{b}′—Y′Y′Y′—N_{b}′—X′X′X′—Nₐ′-nₚ′3′ (IId).

When the antisense RNAi oligonucleotide is represented by formula IIb, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense RNAi oligonucleotide is represented by formula IIc, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense RNAi oligonucleotide is represented by formula IId, N_{b}′ represents 0-10, 0-7, 0-5, 0-4, 0-2, or 0 linked nucleosides. Each Nₐ′ independently represents 2-20, 2-15, or 2-10 linked nucleosides. Preferably, N_{b}′ is 0, 1, 2, 3, 4, 5, or 6.

In certain embodiments, k is 0 and 1 is 0 and the antisense RNAi oligonucleotide may be represented by the formula:

5′nₚ′—Nₐ′—Y′Y′Y′—Nₐ′-n_{q}′3′ (Ia).

When the antisense RNAi oligonucleotide is represented by formula IIa, each Nₐ′ independently represents 2-20, 2-15, or 2-10 linked nucleosides.

Each X′, Y′, and Z′ may be the same or different from each other.

Each nucleotide of the sense RNAi oligonucleotide and antisense RNAi oligonucleotide may be independently modified with LNA, UNA, cEt, HNA, CeNA, 2′-methoxyethyl, 2′-O-allyl, 2′-C-allyl, 2′-hydroxyl, or 2′-fluoro. For example, each nucleotide of the sense RNAi oligonucleotide and antisense RNAi oligonucleotide is independently modified with, 2′-O-methyl or 2′-fluoro. Each X, Y, Z, X′, Y′, and Z′, in particular, may represent a 2′-O-methyl modification or 2′-fluoro modification. In certain embodiments, the modification is a 2′-NMA modification.

In certain embodiments, the sense RNAi oligonucleotide of the RNAi agent may contain YYY motif occurring at 9, 10, and 11 positions of the RNAi oligonucleotide when the duplex region is 21 nucleotides, the count starting from the 1^{st} nucleotide from the 5′-end, or optionally, the count starting at the 1^{st} paired nucleotide within the duplex region, from the 5′-end; and Y represents 2′-F modification. The sense RNAi oligonucleotide may additionally contain XXX motif or ZZZ motifs as wing modifications at the opposite end of the duplex region; and XXX and ZZZ each independently represents a 2′-O-methyl modification or 2′-fluoro modification.

In certain embodiments, the antisense RNAi oligonucleotide may contain Y′Y′Y′ motif occurring at positions 11, 12, 13 of the RNAi oligonucleotide, the count starting from the 1^{st} nucleotide from the 5′-end, or optionally, the count starting at the 1^{st} paired nucleotide within the duplex region, from the 5′-end; and Y′ represents 2′-O-methyl modification. The antisense RNAi oligonucleotide may additionally contain X′X′X′ motif or Z′Z′Z′ motif as wing modifications at the opposite end of the duplex region; and X′X′X′ or Z′Z′Z′ each independently represents a 2′-O-methyl modification or 2′-fluoro modification.

The sense RNAi oligonucleotide represented by any one of the above formulas Ia, Ib, Ic, and Id forms a duplex with an antisense RNAi oligonucleotide being represented by any one of the formulas IIa, IIb, IIc, and Hd, respectively.

Accordingly, the RNAi agents described herein may comprise a sense RNAi oligonucleotide and an antisense RNAi oligonucleotide, each RNAi oligonucleotide having 14 to 30 nucleotides, the RNAi duplex represented by formula (III):

Sense: 5′nₚ-Nₐ—(XXX)ᵢ—N_{b}—YYY—N_{b}—(ZZZ)ⱼ—Nₐ-n_{q}3′

Antisense: 3′nₚ′—Nₐ′—(X′X′X′)ₖ—N_{b}′—Y′Y′Y′—N_{b}′—(Z′Z′Z′)ᵢ—Nₐ′-n_{q}′5′
wherein:
j, k, and l are each independently 0 or 1;
p, p′, q, and q′ are each independently 0-6;
each Nₐ and Nₐ′ independently represents 0-25 linked nucleosides, each sequence comprising at least two differently modified nucleotides;
each N_{b} and N_{b}′ independently represents 0-10 linked nucleosides;
wherein each nₚ′, nₚ, n_{q}′ and n_{q}, each of which may or may not be present, independently represents an overhang nucleotide; and
XXX, YYY, X′X′X′, Y′Y′Y′, and Z′Z′Z′ each independently represent one motif of three identical modifications on three consecutive nucleotides.

In certain embodiments, i is 0 and j is 0; or i is 1 and j is 0; ori is 0 and j is 1; or both i and j are 0; or both i and j are 1. In another embodiment, k is 0 and l is 0; or k is 1 and l is 0, or k is 0 and l is 1; or both k and l are 0; or both k and l are 1.

Exemplary combinations of the sense RNAi oligonucleotide and antisense RNAi oligonucleotide forming a RNAi duplex include the formulas below:

5′nₚ-Nₐ—YYY—Nₐ-n_{q}3′

3′np′—Nₐ′—Y′Y′Y′—Nₐ′n_{q}′5′ (IIIa)

5′nₚ-Nₐ—YYY—N_{b}—ZZZ—Nₐ-n_{q}3′

3′nₚ′-Nₐ′—Y′Y′Y′—N_{b}′—Z′Z′Z′—Nₐ′n_{q}′5′ (IIIb)

5′nₚ-Nₐ—XXX—N_{b}—YYY—Nₐ-n_{q}3′

3′nₚ′—Nₐ′—X′X′X′—N_{b}′—Y′Y′Y′—Nₐ′-n_{q}′5′ (IIIc)

5′nₚ-Nₐ—XXX—N_{b}—YYY—N_{b}—ZZZ—Nₐ-n_{q}3′

3′nₚ′—Nₐ—X′X′X′—N_{b}′—Y′Y′Y′—N_{b}′—Z′Z′Z′—Nₐ-n_{q}′5′ (IIId)

When the RNAi agent is represented with formula IIIa, each Nₐ independently represents 2-20, 2-15, or 2-10 linked nucleosides.

When the RNAi agent is represented with formula IIIb, each N_{b} independently represents 1-10, 1-7, 1-5, or 1-4 linked nucleosides. Each Nₐ independently represents 2-20, 2-15, or 2-10 linked nucleosides.

When the RNAi agent is represented with formula IIIc, each N_{b}, N_{b}′ independently represents 0-10, 0-7, 0-10, 0-7, 0-5, 0-4, 0-2, or 0 linked nucleosides. Each Nₐ independently represents 2-20, 2-15, or 2-10 linked nucleosides.

When the RNAi agent is represented with formula IIId, each N_{b}, N_{b}′ independently represents 0-10, 0-7, 0-10, 0-7, 0-5, 0-4, 0-2, or 0 linked nucleosides. Each Nₐ, Nₐ′ independently 2-20, 2-15, or 2-10 linked nucleosides. Each Nₐ, Nₐ′, N_{b}, N_{b}′ independently comprises modifications of alternating pattern.

Each of X, Y, and Z in formulas III, IIIa, IIIb, IIIc, and IIId may be the same or different from each other.

When the RNAi agent is represented by formula III, IIIa, IIIb, IIIc, and/or IIId, at least one of the Y nucleotides may form a base pair with one of the Y′ nucleotides. Alternatively, at least two of the Y nucleotides may form base pairs with the corresponding Y′ nucleotides; or all three of the Y nucleotides may form base pairs with the corresponding Y′ nucleotides.

When the RNAi agent is represented by formula IIIb or IIId, at least one of the Z nucleotides may form a base pair with one of the Z′ nucleotides. Alternatively, at least two of the Z nucleotides may form base pairs with the corresponding Z′ nucleotides; or all three of the Z nucleotides may form base pairs with the corresponding Z′ nucleotides.

When the RNAi agent is represented by formula IIIc or IIId, at least one of the X nucleotides may form a base pair with one of the X′ nucleotides. Alternatively, at least two of the X nucleotides may form base pairs with the corresponding X′ nucleotides; or all three of the X nucleotides may form base pairs with the corresponding X′ nucleotides.

In certain embodiments, the modification of the Y nucleotide is different than the modification on the Y′ nucleotide, the modification on the Z nucleotide is different than the modification on the Z′ nucleotide, and/or the modification on the X nucleotide is different than the modification on the X′ nucleotide.

In certain embodiments, when the RNAi agent is represented by the formula IIId, the Nₐ modifications are 2′-O-methyl or 2′-fluoro modifications. In another embodiment, when the RNAi agent is represented by formula IIId, the Nₐ modifications are 2′-O-methyl or 2′-fluoro modifications and nₚ′>0 and at least one nₚ′ is linked to a neighboring nucleotide via phosphorothioate linkage. In other embodiments, when the RNAi agent is represented by formula IIId, the Nₐ modifications are 2′-O-methyl or 2′-fluoro modifications, nₚ′>0 and at least one nₚ′ is linked to a neighboring nucleotide via phosphorothioate linkage, and the sense RNAi oligonucleotide is conjugated to one or more cell targeting group attached through a bivalent or trivalent branched linker. In certain embodiments, when the RNAi agent is represented by formula Hid, the Nₐ modifications are 2′-O-methyl or 2′-fluoro modifications, nₚ′>0 and at least one nₚ′ is linked to a neighboring nucleotide via phosphorothioate linkage, the sense RNAi oligonucleotide comprises at least one phosphorothioate linkage and the sense RNAi oligonucleotide is conjugated to one or more cell targeting group attached through a bivalent or trivalent branched linker.

In certain embodiments, when the RNAi agent is represented by the formula IIIa, the Nₐ modifications are 2′-O-methyl or 2′-fluoro modifications and nₚ′>0 and at least one nₚ′ is linked to a neighboring nucleotide via phosphorothioate linkage, the sense RNAi oligonucleotide comprises at least one phosphorothioate linkage and the sense RNAi oligonucleotide is conjugated to one or more cell targeting group attached through a bivalent or trivalent branched linker.

In certain embodiments, the modification is a 2′-NMA modification.

In certain embodiments, the antisense strand may comprise a stabilized phosphate group attached to the 5′ position of the 5′-most nucleoside. In certain embodiments, the stabilized phosphate group comprises an (E)-vinyl phosphonate. In certain embodiments, the stabilized phosphate group comprises a cyclopropyl phosphonate.

In certain embodiments, the antisense strand may comprise a seed-pairing destabilizing modification. In certain embodiments, the seed-pairing destabilizing modification is located at position 6 (counting from the 5′ end). In certain embodiments, the seed-pairing destabilizing modification is located at position 7 (counting from the 5′ end). In certain embodiments, the seed-pairing destabilizing modification is a GNA sugar surrogate. In certain embodiments, the seed-pairing destabilizing modification is an (S)-GNA. In certain embodiments, the seed-pairing destabilizing modification is a UNA. In certain embodiments, the seed-pairing destabilizing modification is a morpholino.

In certain embodiments, the sense strand may comprise an inverted abasic sugar moiety attached to the 5′-most nucleoside. In certain embodiments, the sense strand may comprise an inverted abasic sugar moiety attached to the 3′-most nucleoside. In certain embodiments, the sense strand may comprise inverted abasic sugar moieties attached to both the 5′-most and 3′-most nucleosides.

In certain embodiments, the sense strand may comprise a conjugate attached at position 6 (counting from the 5′ end). In certain embodiments, the conjugate is attached at the 2′ position of the nucleoside. In certain embodiments the conjugate is a C₁₆ lipid conjugate. In certain embodiments, the modified nucleoside at position 6 of the sense strand has a 2′-O-hexadecyl modified sugar moiety.

IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in a standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi agents. RNAi agents may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain embodiments, the target non-coding RNA is selected from: a long non-coding RNA, a short non-coding RNA, an intronic RNA molecule.

A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

### Gapmer Oligonucleotides

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### Antisense RNAi Oligonucleotides

In certain embodiments, antisense RNAi oligonucleotides comprise one or more mismatched nucleobases relative to the target nucleic acid. In certain embodiments, RNAi activity against the target is reduced by such mismatch, but activity against a non-target is reduced by a greater amount. Thus, in certain embodiments selectivity of the antisense RNAi oligonucleotides is improved.

In certain embodiments, antisense RNAi oligonucleotides comprise a targeting region complementary to the target nucleic acid. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides. In certain embodiments, the targeting region constitutes 70%, 80%, 85%, 90%, or 95% of the nucleosides of the antisense RNAi oligonucleotide. In certain embodiments, the targeting region constitutes all of the nucleosides of the antisense RNAi oligonucleotide. In certain embodiments, the targeting region of the antisense RNAi oligonucleotide is at least 99%, 95%, 90%, 85%, or 80% complementary to the target nucleic acid. In certain embodiments, the targeting region of the antisense RNAi oligonucleotide is 100% complementary to the target nucleic acid.

### Sense RNAi Oligonucleotides

In certain embodiments, RNAi agents comprise a sense RNAi oligonucleotide. In such embodiments, sense RNAi oligonucleotides comprise an antisense hybridizing region complementary to the antisense RNAi oligonucleotide. In certain embodiments, the antisense hybridizing region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides. In certain embodiments, the antisense hybridizing region constitutes 70%, 80%, 85%, 90%, or 95% of the nucleosides of the sense RNAi oligonucleotide. In certain embodiments, the antisense hybridizing region constitutes all of the nucleosides of the sense RNAi oligonucleotide. In certain embodiments, the antisense hybridizing region of the sense RNAi oligonucleotide is at least 99%, 95%, 90%, 85%, or 80% complementary to the antisense RNAi oligonucleotide. In certain embodiments, the antisense hybridizing region of the sense RNAi oligonucleotide is 100% complementary to the antisense RNAi oligonucleotide.

The hybridizing region of a sense RNAi oligonucleotide hybridizes with the antisense RNAi oligonucleotide to form a duplex region. In certain embodiments, such duplex region consists of 7 hybridized pairs of nucleosides (one of each pair being on the antisense RNAi oligonucleotide and the other of each pair bien on the sense RNAi oligonucleotide). In certain embodiments, a duplex region comprises least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 hybridized pairs. In certain embodiments, each nucleoside of antisense RNAi oligonucleotide is paired in the duplex region (i.e., the antisense RNAi oligonucleotide has no overhanging nucleosides). In certain embodiments, the antisense RNAi oligonucleotide includes unpaired nucleosides at the 3′-end and/or the 5′end (overhanging nucleosides). In certain embodiments, each nucleoside of sense RNAi oligonucleotide is paired in the duplex region (i.e., the sense RNAi oligonucleotide has no overhanging nucleosides). In certain embodiments, the sense RNAi oligonucleotide includes unpaired nucleosides at the 3′-end and/or the 5′end (overhanging nucleosides). In certain embodiments, duplexes formed by the antisense RNAi oligonucleotide and the sense RNAi oligonucleotide do not include any overhangs at one or both ends. Such ends without overhangs are referred to as blunt. In certain embodiments wherein the antisense RNAi oligonucleotide has overhanging nucleosides, one or more of those overhanging nucleosides are complementary to the target nucleic acid. In certain embodiments wherein the antisense RNAi oligonucleotide has overhanging nucleosides, one or more of those overhanging nucleosides are not complementary to the target nucleic acid.

B. APOE

In certain embodiments, oligomeric compounds comprise or consist of an oligonucleotide comprising a region that is complementary to a target nucleic acid, wherein the target nucleic acid is an APOE nucleic acid. In certain embodiments, the APOE nucleic acid has the sequence set forth as SEQ ID NO: 1 (GENBANK Accession No. NC_000019.10, truncated from nucleotides 44903001 to 44912000). In certain embodiments, the APOE nucleic acid has the sequence set forth as SEQ ID NO: 2 (GENBANK Accession No. NM_001302688.1). In certain embodiments, the APOE nucleic acid has the sequence set forth as SEQ ID NO: 3 (GENBANK Accession No. AU126799.1). In certain embodiments, the APOE nucleic acid has the sequence set forth as SEQ ID NO: 4 (GENBANK Accession No. BI602495.1). In certain embodiments, the APOE nucleic acid has the sequence set forth as SEQ ID NO: 5 (the complement of GENBANK Accession No. CA306379.1). In certain embodiments, the APOE nucleic acid has the sequence set forth as SEQ ID NO: 6 (GENBANK Accession No. NM_000041.2 with T-->C at pos 471 to result in APOE4 mutant mRNA).

In certain embodiments, contacting a cell with an oligomeric compound complementary to SEQ ID NO: 1, SEQ ID NO: 2, SEQ ID NO: 3, SEQ ID NO: 4, or SEQ ID NO: 5 reduces the amount of APOE RNA, and in certain embodiments reduces the amount of APOE protein in the cell. In certain embodiments, administering an oligomeric compound complementary to SEQ ID NO: 1, SEQ ID NO: 2, SEQ ID NO: 3, SEQ ID NO: 4, or SEQ ID NO: 5 to a subject in need thereof results in reduced amyloid plaques and/or reduced neurofibrillary tangles in the brain of the subject as compared to the amount of amyloid plaques and/or neurofibrillary tangles in the brain of the subject before administering. In certain embodiments, administering an oligomeric compound complementary to SEQ ID NO: 1, SEQ ID NO: 2, SEQ ID NO: 3, SEQ ID NO: 4, or SEQ ID NO: 5 to a subject in need thereof results in less amyloid plaques and/or less neurofibrillary tangles in the brain of the subject as compared to the amount of amyloid plaques and/or neurofibrillary tangles in the brain of a control subject not receiving the oligomeric compound. In certain embodiments, the oligomeric compound consists of a modified oligonucleotide. In certain embodiments, the oligomeric compound consists of a modified oligonucleotide and a conjugate group. In certain embodiments, the oligomeric compound is paired with an additional oligomeric compound in an oligomeric duplex. In certain embodiments, the oligomeric duplex comprises a conjugate group.

C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system. Such tissues include the brain, cortex, spinal cord, and the hippocampus.

VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

In certain embodiments, where a modified oligonucleotide or an oligomeric compound is in solution comprising sodium (e.g., saline), the modified oligonucleotide or oligomeric compound may be partially or fully de-protonated and in association with sodium ions. However, the mass of the protons is nevertheless counted toward the weight of the dose, and the mass of the sodium ions is not counted toward the weight of the dose. Thus, for example, a dose, or dosage unit, of 10 mg of a number of fully protonated molecules that weighs 10 mg. This would be equivalent to 10.58 mg of solvent-free, sodium acetate-free, anhydrous sodiated Compound No. 699467 or 10.65 mg of solvent-free, sodium acetate-free, anhydrous sodiated Compound No. 1381709.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

VII. Certain Hotspot Regions

1. Nucleobases 1155-1178 of SEQ ID NO: 2

In certain embodiments, nucleobases 1155-1178 of SEQ ID NO: 2 comprise a hotspot region. In certain embodiments, modified oligonucleotides are complementary to a portion of nucleobases 1155-1178 of SEQ ID NO: 2. In certain embodiments, the modified oligonucleotides are 16 to 20 nucleobases in length. In certain embodiments, modified oligonucleotides are gapmers. In certain embodiments, the gapmers are cEt gapmers. In certain embodiments, the gapmers are MOE gapmers. In certain embodiments, the internucleoside linkages of the modified nucleotides are phosphorothioate internucleoside linkages and phosphodiester linkages, of a combination thereof.

The nucleobase sequences of SEQ ID NOs: 70, 71, 169, 170, 447, 448, 543, 552, 553, 919, 1061, 1132, 1938, 1991, 2066, 2154, 2226, 2259, 2324, 2417, and 2486 are complementary to nucleobases 1155-1178 of SEQ ID NO: 2.

The nucleobase sequences of Compound NOs: 426048, 426049, 689013, 689014, 689015, 689016, 689118, 708021, 708022, 729682, 729683, 942586, 942587, 942588, 1516539, 1516559, 1516570, 1516656, 1516771, 1516839, 1516862, 1516866, and 1516877 are complementary to nucleobases 1155-1178 of SEQ ID NO: 2.

In certain embodiments, modified oligonucleotides complementary to a portion of nucleobases 1155-1178 of SEQ ID NO: 2 achieve at least 46% reduction of APOE RNA in a standard in vitro assay. In certain embodiments, modified oligonucleotides complementary to a portion of nucleobases 1155-1178 of SEQ ID NO: 2 achieve an average of 83.4% reduction of APOE RNA in a standard in vitro assay.

2. Nucleobases 1207-1230 of SEQ ID NO: 2

In certain embodiments, nucleobases 1207-1230 of SEQ ID NO: 2 comprise a hotspot region. In certain embodiments, modified oligonucleotides are complementary to a portion of nucleobases 1207-1230 of SEQ ID NO: 2. In certain embodiments, the modified oligonucleotides are 16 to 20 nucleobases in length. In certain embodiments, modified oligonucleotides are gapmers. In certain embodiments, the gapmers are cEt gapmers. In certain embodiments, the gapmers are MOE gapmers. In certain embodiments, the internucleoside linkages of the modified nucleotides are phosphorothioate internucleoside linkages and phosphodiester linkages, of a combination thereof.

The nucleobase sequences of SEQ ID NOs: 460, 461, 462, 563, 564, 565, 566, 990, 1469, 1572, 1653, 1746, 1914, 1955, 2026, 2110, 2211, 2247, 2344, 2393, and 2481 are complementary to nucleobases 1207-1230 of SEQ ID NO: 2.

The nucleobase sequences of Compound NOs: 689028, 689029, 689030, 729693, 729694, 729695, 729696, 942591, 1516614, 1516652, 1516784, 1516831, 1516890, 1516931, 1516936, 1516973, 1517107, 1517281, 1517604, 1517622, and 1517806 are complementary to nucleobases 1207-1230 of SEQ ID NO: 2.

In certain embodiments, modified oligonucleotides complementary to a portion of nucleobases 1207-1230 of SEQ ID NO: 2 achieve at least 30% reduction of APOE RNA in a standard in vitro assay. In certain embodiments, modified oligonucleotides complementary to a portion of nucleobases 1207-1230 of SEQ ID NO: 2 achieve an average of 60.9% reduction of APOE RNA in a standard in vitro assay.

3. Nucleobases 1259-1295 of SEQ ID NO: 2

In certain embodiments, nucleobases 1259-1295 of SEQ ID NO: 2 comprise a hotspot region. In certain embodiments, modified oligonucleotides are complementary to a portion of nucleobases 1259-1295 of SEQ ID NO: 2. In certain embodiments, the modified oligonucleotides are 16 to 20 nucleobases in length. In certain embodiments, modified oligonucleotides are gapmers. In certain embodiments, the gapmers are cEt gapmers. In certain embodiments, the gapmers are MOE gapmers. In certain embodiments, the internucleoside linkages of the modified nucleotides are phosphorothioate internucleoside linkages and phosphodiester linkages, of a combination thereof.

The nucleobase sequences of SEQ ID NOs: 77, 475, 476, 477, 478, 479, 480, 481, 482, 483, 578, 579, 580, 581, 582, 622, 623, 624, 625, 626, 627, 1063, 1193, 1231, 1232, 1300, 1305, 1378, 1409, 1493, 1526, 1564, 1576, 1678, 1679, 1695, 1827, 1870, 1921, 1928, 1950, 1982, 2012, 2046, 2051, 2074, 2088, 2118, 2158, 2169, 2208, 2223, 2232, 2255, 2321, 2343, 2380, 2436, 2449, and 2451 are complementary to nucleobases 1259-1295 of SEQ ID NO: 2.

The nucleobase sequences of Compound NOs: 689043, 689044, 689045, 689046, 689047, 689048, 689049, 689050, 689051, 708024, 729709, 729710, 729711, 729712, 729713, 729714, 729715, 729716, 729717, 729718, 729719, 942598, 1516533, 1516549, 1516553, 1516613, 1516617, 1516658, 1516672, 1516681, 1516726, 1516740, 1516743, 1516758, 1516778, 1516834, 1516921, 1516932, 1516974, 1517003, 1517024, 1517063, 1517082, 1517130, 1517220, 1517383, 1517427, 1517508, 1517519, 1517578, 1517652, 1517770, 1517837, 1517841, 1517842, 1517849, 1517853, 1517874, 1517885, and 1517891 are complementary to nucleobases 1259-1295 of SEQ ID NO: 2.

In certain embodiments, modified oligonucleotides complementary to a portion of nucleobases 1259-1295 of SEQ ID NO: 2 achieve at least 29% reduction of APOE RNA in a standard in vitro assay. In certain embodiments, modified oligonucleotides complementary to a portion of nucleobases 1259-1295 of SEQ ID NO: 2 achieve an average of 72.4% reduction of APOE RNA in a standard in vitro assay.

4. Nucleobases 1135-1166 of SEQ ID NO: 2

In certain embodiments, nucleobases 1135-1166 of SEQ ID NO: 2 comprise a hotspot region. In certain embodiments, oligomeric compounds or oligomeric duplexes comprise modified oligonucleotides that are complementary within nucleobases 1135-1166 of SEQ ID NO: 2. In certain embodiments, modified oligonucleotides are 23 nucleobases in length. In certain embodiments, modified oligonucleotides are antisense RNAi oligonucleotides. In certain embodiments, the antisense RNAi oligonucleotide has a sugar motif (from 5′ to 3′) of: mfinfmfmfinfinfmfinfmfinfmmm; wherein “m” represents a 2′-O methylribosyl sugar, and the “f” represents a 2′-fluororibosyl sugar; and a linkage motif (from 5′ to 3′) of: ssoooooooooooooooooss; wherein ‘o’ represents a phosphodiester internucleoside linkage and ‘s’ represents a phosphorothioate internucleoside linkage.

The nucleobase sequences of SEQ ID NOs: 2600, 2601, 2604, 2605, 2606, 2607, 2608, 2609, 2610, and 2613 are complementary within nucleobases 1135-1166 of SEQ ID NO: 2.

RNAi compounds 1518282, 1518283, 1518286, 1518299, 1518300, 1518301, 1518302, 1518303, 1518304, and 1518319 comprise an antisense RNAi oligonucleotide that is complementary within nucleobases 1135-1166 of SEQ ID NO: 2.

In certain embodiments, modified oligonucleotides complementary within nucleobases 1135-1166 of SEQ ID NO: 2 achieve at least 45% reduction of APOE RNA in a standard in vitro assay. In certain embodiments, modified oligonucleotides complementary within nucleobases 1135-1166 of SEQ ID NO: 2 achieve an average of 73.1% reduction of APOE RNA in a standard in vitro assay.

5. Nucleobases 1255-1294 of SEQ ID NO: 2

In certain embodiments, nucleobases 1255-1294 of SEQ ID NO: 2 comprise a hotspot region. In certain embodiments, oligomeric compounds or oligomeric duplexes comprise modified oligonucleotides that are complementary within nucleobases 1255-1294 of SEQ ID NO: 2. In certain embodiments, modified oligonucleotides are 23 nucleobases in length. In certain embodiments, modified oligonucleotides are antisense RNAi oligonucleotides. In certain embodiments, the antisense RNAi oligonucleotide has a sugar motif (from 5′ to 3′) of: mfmfmfmfmfmfmfmfmfmfmmm; wherein “m” represents a 2′-O methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and ‘s’ represents a phosphorothioate internucleoside linkage.

The nucleobase sequences of SEQ ID NOs: 2720, 2721, 2722, 2726, 2727, 2729, 2730, 2731, 2732, 2733, 2734, 2735, 2736, 2737, 2738, and 2740 are complementary within nucleobases 1255-1294 of SEQ ID NO: 2.

RNAi compounds 1518642, 1518643, 1518644, 1518659, 1518660, 1518661, 1518663, 1518664, 1518677, 1518678, 1518679, 1518680, 1518681, 1518682, 1518695, 1518697, and 1518699 comprise an antisense RNAi oligonucleotide that is complementary within nucleobases 1255-1294 of SEQ ID NO: 2.

In certain embodiments, modified oligonucleotides complementary within nucleobases 1255-1294 of SEQ ID NO: 2 achieve at least 88% reduction of APOE RNA in a standard in vitro assay. In certain embodiments, modified oligonucleotides complementary within nucleobases 1255-1294 of SEQ ID NO: 2 achieve an average of 94.9% reduction of APOE RNA in a standard in vitro assay.

6. Nucleobases 1255-1295 of SEQ ID NO: 2

In certain embodiments, nucleobases 1255-1295 of SEQ ID NO: 2 comprise a hotspot region. In certain embodiments, oligomeric compounds or oligomeric duplexes comprise modified oligonucleotides that are complementary within nucleobases 1255-1295 of SEQ ID NO: 2.

In certain embodiments, modified oligonucleotides are 23 nucleobases in length. In certain embodiments, modified oligonucleotides are antisense RNAi oligonucleotides. In certain embodiments, the antisense RNAi oligonucleotide has a sugar motif (from 5′ to 3′) of: mfmfmfmfmfmfmfmfmfmfmmm; wherein “m” represents a 2′-O methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and ‘s’ represents a phosphorothioate internucleoside linkage. In certain embodiments, the antisense RNAi oligonucleotide has a sugar motif (from 5′ to 3′) of: efmmmfmmmmmmmfmfmmmmmmm; wherein ‘e’ represents a 2′-MOE modified sugar moiety, each “m” represents a 2′-O-methylribosyl sugar, and each “f” represents a 2′-fluororibosyl sugar; and an internucleoside linkage motif (from 5′ to 3′) of: ssooooooooooooooooooss; wherein ‘o’ represents a phosphodiester internucleoside linkage and ‘s’ represents a phosphorothioate internucleoside linkage.

In certain embodiments, the modified oligonucleotides are 16 to 20 nucleobases in length. In certain embodiments, modified oligonucleotides are gapmers. In certain embodiments, the gapmers are cEt gapmers. In certain embodiments, the gapmers are MOE gapmers. In certain embodiments, the internucleoside linkages of the modified nucleotides are phosphorothioate internucleoside linkages and phosphodiester linkages, of a combination thereof.

The nucleobase sequences of SEQ ID NOs: 76, 77, 473, 474, 475, 476, 477, 478, 479, 480, 481, 482, 483, 576, 578, 579, 580, 581, 582, 622, 623, 624, 625, 626, 627, 1063, 1193, 1231, 1232, 1300, 1305, 1378, 1409, 1493, 1526, 1564, 1576, 1678, 1679, 1695, 1701, 1792, 1827, 1870, 1886, 1906, 1921, 1928, 1950, 1982, 2012, 2046, 2051, 2074, 2088, 2118, 2158, 2169, 2208, 2223, 2232, 2255, 2321, 2343, 2370, 2380, 2436, 2449, 2490, 2451, 2720, 2721, 2722, 2725, 2726, 2727, 2729, 2730, 2731, 2732, 2733, 2734, 2735, 2736, 2737, 2738, 2740, 2935, or 2936 are complementary within nucleobases 1255-1294 of SEQ ID NO: 2.

RNAi compounds 1518642, 1518643, 1518644, 1518659, 1518660, 1518661, 1518663, 1518664, 1518677, 1518678, 1518679, 1518680, 1518681, 1518682, 1518695, 1518697, and 1518699 comprise an antisense RNAi oligonucleotide that is complementary within nucleobases 1255-1294 of SEQ ID NO: 2. The nucleobase sequences of Compound Nos: 689043, 689044, 689045, 689046, 689047, 689048, 689049, 689050, 689051, 708024, 729709, 729710, 729711, 729712, 729713, 729714, 729715, 729716, 729717, 729718, 729719, 942598, 1516533, 1516549, 1516553, 1516613, 1516617, 1516658, 1516672, 1516681, 1516726, 1516740, 1516743, 1516758, 1516778, 1516834, 1516921, 1516932, 1516974, 1517003, 1517024, 1517063, 1517082, 1517130, 1517220, 1517383, 1517427, 1517508, 1517519, 1517578, 1517652, 1517770, 1517837, 1517841, 1517842, 1517849, 1517853, 1517874, 1517885, 1517891, 1642901, and 1644690 are complementary to nucleobases 1259-1295 of SEQ ID NO: 2.

In certain embodiments, modified oligonucleotides complementary within nucleobases 1255-1295 of SEQ ID NO: 2 achieve at least 37% reduction of APOE RNA in a standard in vitro assay. In certain embodiments, modified oligonucleotides complementary within nucleobases 1255-1295 of SEQ ID NO: 2 achieve an average of 70% reduction of APOE RNA in a standard in vitro assay.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of 5-10-5 MOE Full Phosphorothioate Modified Oligonucleotides on Human APOE RNA In Vitro, Single Dose

Modified oligonucleotides complementary to human APOE nucleic acid were designed and tested for their single dose effects on APOE RNA in vitro. The modified oligonucleotides were tested in a series of experiments that had the same culture conditions.

The modified oligonucleotides in the table below are 5-10-5 MOE gapmers with full phosphorothioate internucleoside linkages. The gapmers are 20 nucleosides in length, wherein the central gap segment consists of ten 2′-β-D-deoxynucleosides, and wherein the 5′ and 3′ wing segments each consist of five 2′-MOE modified nucleosides. The sugar motif for the gapmers is (from 5′ to 3′): eeeeeddddddddddeeeee; wherein ‘d’ represents a 2′-β-D-deoxyribosyl sugar, and ‘e’ represents a 2′-MOE modified sugar moiety. The internucleoside linkage motif for the gapmers is (from 5′ to 3′): sssssssssssssssssss; wherein each ‘s’ represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NC_000019.10, truncated from nucleotides 44903001 to 44912000), to SEQ ID NO: 2 (GENBANK Accession No. NM_001302688.1), to SEQ ID NO: 3 (GENBANK Accession No. AU126799.1), to SEQ ID NO: 4 (GENBANK Accession No. BI602495.1), to SEQ ID NO: 5 (the complement of GENBANK Accession No. CA306379.1), or to any combination of these SEQ ID NOs. ‘N/A’ indicates that the modified oligonucleotide is not 100% complementary to that particular target nucleic acid sequence.

Cultured HepG2 cells were treated with modified oligonucleotide at a concentration of 100 nM using Lipofectin at a density of 10,000 cells per well. After a treatment period of approximately 24 hours, total RNA was isolated from the cells and APOE RNA levels were measured by quantitative real-time RTPCR. APOE RNA levels were measured by human primer-probe set RTS3073 (forward sequence TGGGTCGCTTTTGGGATTAC, designated herein as SEQ ID NO: 10; reverse sequence CCATCAGCGCCCTCAGTT, designated herein as SEQ ID NO: 11; probe sequence CTGCTCAGCTCCCAGGTCACCCA, designated herein as SEQ ID NO: 12). APOE RNA levels were normalized to total RNA content, as measured by RIBOGREEN®. Results are presented in the tables below as percent APOE RNA relative to untreated control cells (% UTC). Each table represents results from an individual assay plate. The values marked with an “†” indicate that the modified oligonucleotide is complementary to the amplicon region of the primer probe set. Additional assays may be used to measure the potency and efficacy of the modified oligonucleotides complementary to the amplicon region.

TABLE 1
Reduction of APOE RNA by 5-10-5 MOE gapmers with full phosphorothioate internucleoside
linkages at a concentration of 100 nM in HepG2 cells plated at 10,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 425997 | 2782 | 2801 | 34 | 53 | GAGTAGGACTCAAGGATCCC | 108 | 20 |
| 425999 | 3625 | 3644 | 199 | 218 | GCAGCCCACAGAACCTTCAT | 122 | 21 |
| 426000 | 3628 | 3647 | 202 | 221 | AACGCAGCCCACAGAACCTT | 88 | 22 |
| 426001 | 3636 | 3655 | 210 | 229 | TGACCAGCAACGCAGCCCAC | 79 | 23 |
| 426002 | 3642 | 3661 | 216 | 235 | GGAATGTGACCAGCAACGCA | 66 | 24 |
| 426003 | 3649 | 3668 | 223 | 242 | CCTGCCAGGAATGTGACCAG | 26 | 25 |
| 426004 | N/A | N/A | 226 | 245 | CATCCTGCCAGGAATGTGAC | 72 | 26 |
| 426005 | N/A | N/A | 235 | 254 | TTGGCCTGGCATCCTGCCAG | 81 | 27 |
| 426006 | 4759 | 4778 | 241 | 260 | TCCACCTTGGCCTGGCATCC | 84 | 28 |
| 426007 | 4844 | 4863 | 326 | 345 | ACCCAGTGCCAGTTCCCAGC | 79† | 29 |
| 426008 | 4854 | 4873 | 336 | 355 | CCCAAAAGCGACCCAGTGCC | 73† | 30 |
| 426009 | 4861 | 4880 | 343 | 362 | AGGTAATCCCAAAAGCGACC | 60† | 31 |
| 426010 | 4864 | 4883 | 346 | 365 | CGCAGGTAATCCCAAAAGCG | 47† | 32 |
| 426011 | 4872 | 4891 | 354 | 373 | GCACCCAGCGCAGGTAATCC | 33† | 33 |
| 426012 | 4875 | 4894 | 357 | 376 | TCTGCACCCAGCGCAGGTAA | 53† | 34 |
| 426013 | 4879 | 4898 | 361 | 380 | AGTGTCTGCACCCAGCGCAG | 14† | 35 |
| 426014 | 4886 | 4905 | 368 | 387 | CTCAGACAGTGTCTGCACCC | 62† | 36 |
| 426015 | 4893 | 4912 | 375 | 394 | GCACCTGCTCAGACAGTGTC | 57† | 37 |
| 426016 | 4921 | 4940 | 403 | 422 | GTGACCTGGGAGCTGAGCAG | 42† | 38 |
| 426017 | 4932 | 4951 | 414 | 433 | TCAGTTCCTGGGTGACCTGG | 19† | 39 |
| 426018 | N/A | N/A | 419 | 438 | CGCCCTCAGTTCCTGGGTGA | 68† | 40 |
| 426019 | 5559 | 5578 | 461 | 480 | CGATTTGTAGGCCTTCAACT | 32 | 41 |
| 426020 | 5565 | 5584 | 467 | 486 | CAGTTCCGATTTGTAGGCCT | 25 | 42 |
| 426021 | 5618 | 5637 | 520 | 539 | TCCTTGGACAGCCGTGCCCG | 56 | 43 |
| 426022 | 5682 | 5701 | 584 | 603 | CTGCACCAGGCGGCCGCACA | 66 | 44 |
| 426023 | 5688 | 5707 | 590 | 609 | GCGGTACTGCACCAGGCGGC | 116 | 45 |
| 426024 | 5691 | 5710 | 593 | 612 | GCCGCGGTACTGCACCAGGC | 60 | 46 |
| 426025 | 5715 | 5734 | 617 | 636 | CTGGCCGAGCATGGCCTGCA | 96 | 47 |
| 426026 | 5765 | 5784 | 667 | 686 | CGCAGCTTGCGCAGGTGGGA | 82 | 48 |
| 426027 | 5775 | 5794 | 677 | 696 | GAGCCGCTTACGCAGCTTGC | 76 | 49 |
| 426028 | 5799 | 5818 | 701 | 720 | CTGCAGGTCATCGGCATCGC | 24 | 50 |
| 426029 | 5804 | 5823 | 706 | 725 | CGCTTCTGCAGGTCATCGGC | 35 | 51 |
| 426030 | 5825 | 5844 | 727 | 746 | CCGGCCTGGTACACTGCCAG | 75 | 52 |
| 426031 | 5828 | 5847 | 730 | 749 | GCCCCGGCCTGGTACACTGC | 70 | 53 |
| 426032 | 5832 | 5851 | 734 | 753 | GCGGGCCCCGGCCTGGTACA | 87 | 54 |
| 426033 | 5919 | 5938 | 821 | 840 | GCCCACAGTGGCGGCCCGCA | 84 | 55 |
| 426034 | 5922 | 5941 | 824 | 843 | GGAGCCCACAGTGGCGGCCC | 115 | 56 |
| 426035 | 5925 | 5944 | 827 | 846 | CAGGGAGCCCACAGTGGCGG | 106 | 57 |
| 426036 | 5928 | 5947 | 830 | 849 | GGCCAGGGAGCCCACAGTGG | 97 | 58 |
| 426037 | 5934 | 5953 | 836 | 855 | CTGGCCGGCCAGGGAGCCCA | 111 | 59 |
| 426038 | 6009 | 6028 | 911 | 930 | GCGGGTCCGGCTGCCCATCT | 61 | 60 |
| 426039 | 6062 | 6081 | 964 | 983 | TCCAGCTTGGCGCGCACCTC | 37 | 61 |
| 426040 | 6103 | 6122 | 1005 | 1024 | GGAAGGCCTCGGCCTGCAGG | 92 | 62 |
| 426041 | 6118 | 6137 | 1020 | 1039 | TCTTGAGGCGGGCCTGGAAG | 91 | 63 |
| 426042 | 6127 | 6146 | 1029 | 1048 | CGAACCAGCTCTTGAGGCGG | 117 | 64 |
| 426043 | 6150 | 6169 | 1052 | 1071 | CTGCATGTCTTCCACCAGGG | 37 | 65 |
| 426044 | 6153 | 6172 | 1055 | 1074 | GCGCTGCATGTCTTCCACCA | 50 | 66 |
| 426045 | 6197 | 6216 | 1099 | 1118 | GTGCCCACGGCAGCCTGCAC | 90 | 67 |
| 426046 | 6230 | 6249 | 1132 | 1151 | CAGTGATTGTCGCTGGGCAC | 136 | 68 |
| 426047 | 6234 | 6253 | 1136 | 1155 | CGTTCAGTGATTGTCGCTGG | 31 | 69 |
| 426048 | 6254 | 6273 | 1156 | 1175 | CGCATGGCTGCAGGCTTCGG | 5 | 70 |
| 426049 | 6257 | 6276 | 1159 | 1178 | GGTCGCATGGCTGCAGGCTT | 10 | 71 |
| 426050 | 6336 | 6355 | 1238 | 1257 | ACCCCAGGAGGACGGCTGGG | 32 | 72 |
| 426051 | 6340 | 6359 | 1242 | 1261 | GTCCACCCCAGGAGGACGGC | 69 | 73 |
| 426052 | 6343 | 6362 | 1245 | 1264 | AGGGTCCACCCCAGGAGGAC | 37 | 74 |
| 426053 | 6347 | 6366 | 1249 | 1268 | AACTAGGGTCCACCCCAGGA | 214 | 75 |
| 426054 | 6353 | 6372 | 1255 | 1274 | TTATTAAACTAGGGTCCACC | 56 | 76 |
| 426055 | 6371 | 6390 | 1273 | 1292 | GTGAAACTTGGTGAATCTTT | 30 | 77 |
| 426062 | 2888 | 2907 | 140 | 159 | CCCAGGGTCCCAGCTCTTTC | 163 | 78 |
| 426067 | 2892 | 2911 | 144 | 163 | GGTTCCCAGGGTCCCAGCTC | 87 | 79 |
| 426068 | 2912 | 2931 | N/A | N/A | GAGACTACCTGGAGGCCAGG | 96 | 80 |
| 426069 | 3167 | 3186 | N/A | N/A | ACCGTGTCGCTGCCCCTGGC | 99 | 81 |
| 426070 | 3656 | 3675 | N/A | N/A | CCCCATACCTGCCAGGAATG | 130 | 82 |
| 426071 | 4272 | 4291 | N/A | N/A | TGTGTGCCCCAGGCAGGGCT | 113 | 83 |
| 426072 | 4937 | 4956 | N/A | N/A | TCACCTCAGTTCCTGGGTGA | 91† | 84 |
| 426073 | 4976 | 4995 | N/A | N/A | GCCGCCCACCAGGAGGGTCA | 105† | 85 |
| 426074 | 7222 | 7241 | N/A | N/A | GCAGGCCGGGCTCGGAGCCC | 107 | 86 |

TABLE 2
Reduction of APOE RNA by 5-10-5 MOE gapmers with full phosphorothioate internucleoside
linkages at a concentration of 100 nM in HepG2 cells plated at 10,000 cells per well

| | SEQ | SEQ | SEQ | SEQ | SEQ | SEQ | | | |
|---|---|---|---|---|---|---|---|---|---|
| | ID No: | ID No: | ID No: | ID No: | ID No: | ID No: | | APOE | |
| Compound | 3 Start | 3 Stop | 4 Start | 4 Stop | 5 Start | 5 Stop | Sequence | (% | SEQ |
| Number | Site | Site | Site | Site | Site | Site | (5′ to 3′) | UTC) | ID NO |
| 425998 | 40 | 59 | N/A | N/A | N/A | N/A | TGTGATTGGC | 85 | 87 |
| | | | | | | | CAGTCGGCTC | | |
| 426056 | 103 | 122 | N/A | N/A | N/A | N/A | TGGCTGGCAT | 106 | 88 |
| | | | | | | | CCTGCCAGGA | | |
| 426057 | 534 | 553 | N/A | N/A | N/A | N/A | TACGCAGTTG | 123 | 89 |
| | | | | | | | CGCAGGTGGG | | |
| 426058 | 543 | 562 | N/A | N/A | N/A | N/A | AGGAGCCGTT | 109 | 90 |
| | | | | | | | ACGCAGTTGC | | |
| 426060 | 731 | 750 | N/A | N/A | N/A | N/A | ATTAAACTTA | 133 | 91 |
| | | | | | | | GGGTCCACTC | | |
| 426061 | 734 | 753 | N/A | N/A | N/A | N/A | TTTATTAAACT | 73 | 92 |
| | | | | | | | TAGGGTCCA | | |
| 426063 | N/A | N/A | 747 | 766 | N/A | N/A | TGTTCCACCA | 125 | 93 |
| | | | | | | | GGGCCCCAGG | | |
| 426064 | N/A | N/A | 777 | 796 | N/A | N/A | GGAGCCCACA | 135 | 94 |
| | | | | | | | GTGCCGGCCG | | |
| 426065 | N/A | N/A | N/A | N/A | 98 | 117 | CACTTCTGCA | 67 | 95 |
| | | | | | | | GGTCATCGGC | | |
| 426066 | N/A | N/A | N/A | N/A | 103 | 122 | CCAGGCACTT | 121 | 96 |
| | | | | | | | CTGCAGGTCA | | |

### Example 2: Effect of 5-8-5 MOE Mixed Backbone Modified Oligonucleotides on Human APOE RNA In Vitro, Single Dose

Modified oligonucleotides complementary to human APOE nucleic acid were designed and tested for their single dose effects on APOE RNA in vitro. The modified oligonucleotides were tested in a series of experiments that had similar culture conditions. The modified oligonucleotides in the tables below are 5-8-5 MOE gapmers with mixed PO/PS internucleoside linkages. The gapmers are 18 nucleosides in length, wherein the central gap segment consists of eight 2′-β-D-deoxynucleosides, and wherein the 5′ and 3′ wing segments each consist of five 2′-MOE modified nucleosides. The sugar motif for the gapmers is (from 5′ to 3′): eeeeeddddddddeeeee; wherein ‘d’ represents a 2′-β-D-deoxyribosyl sugar, and ‘e’ represents a 2′-MOE modified sugar moiety. The internucleoside linkage motif for the gapmers is (from 5′ to 3′): soossssssssssooss; wherein each ‘o’ represents a phosphodiester internucleoside linkage and each ‘s’ represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), to SEQ ID NO: 3 (described herein above), to SEQ ID NO: 4 (described herein above), to SEQ ID NO: 5 (described herein above), to SEQ ID NO: 6 (GENBANK Accession No. NM_000041.2 with T-->C at pos 471 to result in APOE4 mutant mRNA), or to any combination of these SEQ ID NOs. ‘N/A’ indicates that the modified oligonucleotide is not 100% complementary to that particular target nucleic acid sequence.

Cultured Hep3B or HepG2 cells were treated with modified oligonucleotide at a concentration of 2000 or 4000 nM by electroporation at a density of either 5,000 or 20,000 cells per well, as indicated in the tables below. After a treatment period of approximately 24 hours, total RNA was isolated from the cells and APOE RNA levels were measured by quantitative real-time RTPCR. APOE RNA levels were measured by human primer-probe set RTS3073 (described herein in Example 1). APOE RNA levels were normalized to total RNA content, as measured by RIBOGREEN®. Results are presented in the tables below as percent APOE RNA relative to untreated control cells (% UTC). Each table represents results from an individual assay plate. The values marked with an “†” indicate that the modified oligonucleotide is complementary to the amplicon region of the primer probe set. Additional assays may be used to measure the potency and efficacy of the modified oligonucleotides complementary to the amplicon region. In Tables 3-8 below, Compound 426048 (described herein above) was included as a reference.

TABLE 3
Reduction of APOE RNA by 5-8-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 42 | 70 |
| 688661 | 2783 | 2800 | 35 | 52 | AGTAGGACTCAAGGATCC | 103 | 97 |
| 688662 | 2785 | 2802 | 37 | 54 | TGAGTAGGACTCAAGGAT | 108 | 98 |
| 688663 | 2787 | 2804 | 39 | 56 | GCTGAGTAGGACTCAAGG | 108 | 99 |
| 688664 | 2789 | 2806 | 41 | 58 | GGGCTGAGTAGGACTCAA | 108 | 100 |
| 688665 | 2805 | 2822 | 57 | 74 | TCCTTCACCTCCGCTGGG | 120 | 101 |
| 688666 | 2807 | 2824 | 59 | 76 | CGTCCTTCACCTCCGCTG | 109 | 102 |
| 688671 | 3601 | 3618 | 175 | 192 | GCCTGTGATTGGCCAGTC | 92 | 103 |
| 688672 | 3603 | 3620 | 177 | 194 | CTGCCTGTGATTGGCCAG | 70 | 104 |
| 688673 | 3605 | 3622 | 179 | 196 | TCCTGCCTGTGATTGGCC | 96 | 105 |
| 688674 | 3607 | 3624 | 181 | 198 | CTTCCTGCCTGTGATTGG | 96 | 106 |
| 688675 | 3609 | 3626 | 183 | 200 | ATCTTCCTGCCTGTGATT | 100 | 107 |
| 688676 | 3613 | 3630 | 187 | 204 | CTTCATCTTCCTGCCTGT | 95 | 108 |
| 688677 | 3615 | 3632 | 189 | 206 | ACCTTCATCTTCCTGCCT | 110 | 109 |
| 688678 | 3617 | 3634 | 191 | 208 | GAACCTTCATCTTCCTGC | 93 | 110 |
| 688679 | 3619 | 3636 | 193 | 210 | CAGAACCTTCATCTTCCT | 96 | 111 |
| 688680 | 3621 | 3638 | 195 | 212 | CACAGAACCTTCATCTTC | 88 | 112 |
| 688681 | 3623 | 3640 | 197 | 214 | CCCACAGAACCTTCATCT | 78 | 113 |
| 688682 | 3625 | 3642 | 199 | 216 | AGCCCACAGAACCTTCAT | 80 | 114 |
| 688683 | 3627 | 3644 | 201 | 218 | GCAGCCCACAGAACCTTC | 101 | 115 |
| 688684 | 3629 | 3646 | 203 | 220 | ACGCAGCCCACAGAACCT | 97 | 116 |
| 688685 | 3631 | 3648 | 205 | 222 | CAACGCAGCCCACAGAAC | 102 | 117 |
| 688686 | 3633 | 3650 | 207 | 224 | AGCAACGCAGCCCACAGA | 94 | 118 |
| 688687 | 3635 | 3652 | 209 | 226 | CCAGCAACGCAGCCCACA | 68 | 119 |
| 688688 | 3637 | 3654 | 211 | 228 | GACCAGCAACGCAGCCCA | 80 | 120 |
| 688689 | 3639 | 3656 | 213 | 230 | GTGACCAGCAACGCAGCC | 109 | 121 |
| 688690 | 3641 | 3658 | 215 | 232 | ATGTGACCAGCAACGCAG | 113 | 122 |
| 688691 | 3645 | 3662 | 219 | 236 | AGGAATGTGACCAGCAAC | 93 | 123 |
| 688692 | 3647 | 3664 | 221 | 238 | CCAGGAATGTGACCAGCA | 100 | 124 |
| 688693 | 3649 | 3666 | 223 | 240 | TGCCAGGAATGTGACCAG | 105 | 125 |
| 688694 | 3651 | 3668 | 225 | 242 | CCTGCCAGGAATGTGACC | 123 | 126 |
| 688695 | N/A | N/A | 227 | 244 | ATCCTGCCAGGAATGTGA | 122 | 127 |
| 688696 | N/A | N/A | 229 | 246 | GCATCCTGCCAGGAATGT | 112 | 128 |
| 688697 | N/A | N/A | 231 | 248 | TGGCATCCTGCCAGGAAT | 112 | 129 |
| 688698 | N/A | N/A | 233 | 250 | CCTGGCATCCTGCCAGGA | 99 | 130 |
| 688699 | N/A | N/A | 235 | 252 | GGCCTGGCATCCTGCCAG | 90 | 131 |
| 688700 | 4757 | 4774 | 239 | 256 | CCTTGGCCTGGCATCCTG | 105 | 132 |
| 688701 | 4759 | 4776 | 241 | 258 | CACCTTGGCCTGGCATCC | 132 | 133 |
| 688702 | 4761 | 4778 | 243 | 260 | TCCACCTTGGCCTGGCAT | 132 | 134 |
| 688703 | 4763 | 4780 | 245 | 262 | GCTCCACCTTGGCCTGGC | 121 | 135 |
| 688704 | 4765 | 4782 | 247 | 264 | TTGCTCCACCTTGGCCTG | 112 | 136 |
| 688705 | 4767 | 4784 | 249 | 266 | GCTTGCTCCACCTTGGCC | 119 | 137 |
| 688706 | 4769 | 4786 | 251 | 268 | CCGCTTGCTCCACCTTGG | 111 | 138 |
| 688707 | 4773 | 4790 | 255 | 272 | TCCACCGCTTGCTCCACC | 118 | 139 |
| 688708 | 4775 | 4792 | 257 | 274 | TCTCCACCGCTTGCTCCA | 120 | 140 |
| 688709 | 4777 | 4794 | 259 | 276 | TGTCTCCACCGCTTGCTC | 155 | 141 |
| 688710 | 4779 | 4796 | 261 | 278 | TCTGTCTCCACCGCTTGC | 104 | 142 |
| 688711 | 4781 | 4798 | 263 | 280 | GCTCTGTCTCCACCGCTT | 53 | 143 |
| 688712 | 4783 | 4800 | 265 | 282 | CGGCTCTGTCTCCACCGC | 68 | 144 |
| 688713 | 4785 | 4802 | 267 | 284 | TCCGGCTCTGTCTCCACC | 81 | 145 |
| 688714 | 4787 | 4804 | 269 | 286 | GCTCCGGCTCTGTCTCCA | 71 | 146 |
| 688715 | 4791 | 4808 | 273 | 290 | TCGGGCTCCGGCTCTGTC | 110 | 147 |
| 688716 | 4794 | 4811 | 276 | 293 | AGCTCGGGCTCCGGCTCT | 83 | 148 |
| 688717 | 4796 | 4813 | 278 | 295 | GCAGCTCGGGCTCCGGCT | 88 | 149 |
| 688718 | 4798 | 4815 | 280 | 297 | GCGCAGCTCGGGCTCCGG | 134 | 150 |
| 688719 | 4800 | 4817 | 282 | 299 | TGGCGCAGCTCGGGCTCC | 90 | 151 |
| 688720 | 4802 | 4819 | 284 | 301 | GCTGGCGCAGCTCGGGCT | 98 | 152 |
| 688721 | 4804 | 4821 | 286 | 303 | CTGCTGGCGCAGCTCGGG | 86 | 153 |
| 688722 | 4806 | 4823 | 288 | 305 | GTCTGCTGGCGCAGCTCG | 113 | 154 |
| 688723 | 4808 | 4825 | 290 | 307 | CGGTCTGCTGGCGCAGCT | 97 | 155 |
| 688724 | 4810 | 4827 | 292 | 309 | CTCGGTCTGCTGGCGCAG | 103 | 156 |
| 688725 | 4812 | 4829 | 294 | 311 | CACTCGGTCTGCTGGCGC | 96 | 157 |
| 688726 | 4814 | 4831 | 296 | 313 | GCCACTCGGTCTGCTGGC | 100 | 158 |
| 688727 | 4816 | 4833 | 298 | 315 | CTGCCACTCGGTCTGCTG | 112 | 159 |
| 688728 | 4818 | 4835 | 300 | 317 | CTCTGCCACTCGGTCTGC | 75 | 160 |
| 688729 | 4820 | 4837 | 302 | 319 | CGCTCTGCCACTCGGTCT | 98 | 161 |
| 688730 | 4822 | 4839 | 304 | 321 | GCCGCTCTGCCACTCGGT | 86 | 162 |
| 688731 | 4824 | 4841 | 306 | 323 | TGGCCGCTCTGCCACTCG | 100 | 163 |
| 688732 | 4826 | 4843 | 308 | 325 | GCTGGCCGCTCTGCCACT | 109 | 164 |
| 688733 | 4828 | 4845 | 310 | 327 | GCGCTGGCCGCTCTGCCA | 125 | 165 |
| 688734 | 4842 | 4859 | 324 | 341 | AGTGCCAGTTCCCAGCGC | 127† | 166 |
| 688735 | 4844 | 4861 | 326 | 343 | CCAGTGCCAGTTCCCAGC | 136† | 167 |
| 688736 | 4848 | 4865 | 330 | 347 | CGACCCAGTGCCAGTTCC | 79† | 168 |
| 689014 | 6255 | 6272 | 1157 | 1174 | GCATGGCTGCAGGCTTCG | 38 | 169 |
| 689016 | 6258 | 6275 | 1160 | 1177 | GTCGCATGGCTGCAGGCT | 28 | 170 |

TABLE 4
Reduction of APOE RNA by 5-8-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 36 | 70 |
| 688737 | 4850 | 4867 | 332 | 349 | AGCGACCCAGTGCCAGTT | 42† | 171 |
| 688738 | 4852 | 4869 | 334 | 351 | AAAGCGACCCAGTGCCAG | 59† | 172 |
| 688739 | 4854 | 4871 | 336 | 353 | CAAAAGCGACCCAGTGCC | 60† | 173 |
| 688740 | 4856 | 4873 | 338 | 355 | CCCAAAAGCGACCCAGTG | 32† | 174 |
| 688741 | 4858 | 4875 | 340 | 357 | ATCCCAAAAGCGACCCAG | 39† | 175 |
| 688742 | 4860 | 4877 | 342 | 359 | TAATCCCAAAAGCGACCC | 51† | 176 |
| 688743 | 4862 | 4879 | 344 | 361 | GGTAATCCCAAAAGCGAC | 44† | 177 |
| 688744 | 4866 | 4883 | 348 | 365 | CGCAGGTAATCCCAAAAG | 31† | 178 |
| 688745 | 4868 | 4885 | 350 | 367 | AGCGCAGGTAATCCCAAA | 32† | 179 |
| 688746 | 4870 | 4887 | 352 | 369 | CCAGCGCAGGTAATCCCA | 17† | 180 |
| 688747 | 4872 | 4889 | 354 | 371 | ACCCAGCGCAGGTAATCC | 13† | 181 |
| 688748 | 4874 | 4891 | 356 | 373 | GCACCCAGCGCAGGTAAT | 43† | 182 |
| 688749 | 4876 | 4893 | 358 | 375 | CTGCACCCAGCGCAGGTA | 28† | 183 |
| 688750 | 4878 | 4895 | 360 | 377 | GTCTGCACCCAGCGCAGG | 24† | 184 |
| 688751 | 4881 | 4898 | 363 | 380 | AGTGTCTGCACCCAGCGC | 24† | 185 |
| 688752 | 4882 | 4899 | 364 | 381 | CAGTGTCTGCACCCAGCG | 41† | 186 |
| 688753 | 4884 | 4901 | 366 | 383 | GACAGTGTCTGCACCCAG | 42† | 187 |
| 688754 | 4886 | 4903 | 368 | 385 | CAGACAGTGTCTGCACCC | 81† | 188 |
| 688755 | 4888 | 4905 | 370 | 387 | CTCAGACAGTGTCTGCAC | 49† | 189 |
| 688756 | 4890 | 4907 | 372 | 389 | TGCTCAGACAGTGTCTGC | 57† | 190 |
| 688757 | 4894 | 4911 | 376 | 393 | CACCTGCTCAGACAGTGT | 43† | 191 |
| 688758 | 4896 | 4913 | 378 | 395 | TGCACCTGCTCAGACAGT | 40† | 192 |
| 688759 | 4898 | 4915 | 380 | 397 | CCTGCACCTGCTCAGACA | 12† | 193 |
| 688760 | 4900 | 4917 | 382 | 399 | CTCCTGCACCTGCTCAGA | 17† | 194 |
| 688761 | 4902 | 4919 | 384 | 401 | TCCTCCTGCACCTGCTCA | 11† | 195 |
| 688762 | 4904 | 4921 | 386 | 403 | GCTCCTCCTGCACCTGCT | 16† | 196 |
| 688763 | 4906 | 4923 | 388 | 405 | CAGCTCCTCCTGCACCTG | 13† | 197 |
| 688764 | 4908 | 4925 | 390 | 407 | AGCAGCTCCTCCTGCACC | 46† | 198 |
| 688765 | 4912 | 4929 | 394 | 411 | GCTGAGCAGCTCCTCCTG | 21† | 199 |
| 688766 | 4914 | 4931 | 396 | 413 | GAGCTGAGCAGCTCCTCC | 52† | 200 |
| 688767 | 4916 | 4933 | 398 | 415 | GGGAGCTGAGCAGCTCCT | 76† | 201 |
| 688768 | 4918 | 4935 | 400 | 417 | CTGGGAGCTGAGCAGCTC | 31† | 202 |
| 688769 | 4922 | 4939 | 404 | 421 | TGACCTGGGAGCTGAGCA | 51† | 203 |
| 688770 | 4924 | 4941 | 406 | 423 | GGTGACCTGGGAGCTGAG | 20† | 204 |
| 688771 | 4932 | 4949 | 414 | 431 | AGTTCCTGGGTGACCTGG | 43† | 205 |
| 688772 | 4934 | 4951 | 416 | 433 | TCAGTTCCTGGGTGACCT | 82† | 206 |
| 688773 | 4936 | 4953 | 418 | 435 | CCTCAGTTCCTGGGTGAC | 61† | 207 |
| 688774 | N/A | N/A | 420 | 437 | GCCCTCAGTTCCTGGGTG | 35† | 208 |
| 688775 | N/A | N/A | 422 | 439 | GCGCCCTCAGTTCCTGGG | 10† | 209 |
| 688776 | 5545 | 5562 | 447 | 464 | AACTCCTTCATGGTCTCG | 85 | 210 |
| 688777 | 5549 | 5566 | 451 | 468 | CTTCAACTCCTTCATGGT | 115 | 211 |
| 688778 | 5551 | 5568 | 453 | 470 | GCCTTCAACTCCTTCATG | 101 | 212 |
| 688779 | 5553 | 5570 | 455 | 472 | AGGCCTTCAACTCCTTCA | 67 | 213 |
| 688780 | 5555 | 5572 | 457 | 474 | GTAGGCCTTCAACTCCTT | 84 | 214 |
| 688781 | 5559 | 5576 | 461 | 478 | ATTTGTAGGCCTTCAACT | 72 | 215 |
| 688782 | 5561 | 5578 | 463 | 480 | CGATTTGTAGGCCTTCAA | 45 | 216 |
| 688783 | 5563 | 5580 | 465 | 482 | TCCGATTTGTAGGCCTTC | 51 | 217 |
| 688784 | 5565 | 5582 | 467 | 484 | GTTCCGATTTGTAGGCCT | 57 | 218 |
| 688785 | 5567 | 5584 | 469 | 486 | CAGTTCCGATTTGTAGGC | 98 | 219 |
| 688786 | 5569 | 5586 | 471 | 488 | TCCAGTTCCGATTTGTAG | 102 | 220 |
| 688787 | 5571 | 5588 | 473 | 490 | CCTCCAGTTCCGATTTGT | 51 | 221 |
| 688788 | 5573 | 5590 | 475 | 492 | TTCCTCCAGTTCCGATTT | 68 | 222 |
| 688789 | 5575 | 5592 | 477 | 494 | TGTTCCTCCAGTTCCGAT | 83 | 223 |
| 688790 | 5577 | 5594 | 479 | 496 | GTTGTTCCTCCAGTTCCG | 70 | 224 |
| 688791 | 5579 | 5596 | 481 | 498 | CAGTTGTTCCTCCAGTTC | 115 | 225 |
| 688792 | 5581 | 5598 | 483 | 500 | GTCAGTTGTTCCTCCAGT | 82 | 226 |
| 688793 | 5583 | 5600 | 485 | 502 | GGGTCAGTTGTTCCTCCA | 62 | 227 |
| 688794 | 5599 | 5616 | 501 | 518 | GTCTCCTCCGCCACCGGG | 89 | 228 |
| 688795 | 5616 | 5633 | 518 | 535 | TGGACAGCCGTGCCCGCG | 58 | 229 |
| 688796 | 5618 | 5635 | 520 | 537 | CTTGGACAGCCGTGCCCG | 79 | 230 |
| 688797 | 5620 | 5637 | 522 | 539 | TCCTTGGACAGCCGTGCC | 80 | 231 |
| 688798 | 5622 | 5639 | 524 | 541 | GCTCCTTGGACAGCCGTG | 58 | 232 |
| 688799 | 5624 | 5641 | 526 | 543 | CAGCTCCTTGGACAGCCG | 92 | 233 |
| 688800 | 5626 | 5643 | 528 | 545 | TGCAGCTCCTTGGACAGC | 80 | 234 |
| 688801 | 5628 | 5645 | 530 | 547 | CCTGCAGCTCCTTGGACA | 92 | 235 |
| 688802 | 5632 | 5649 | 534 | 551 | GCCGCCTGCAGCTCCTTG | 69 | 236 |
| 688803 | 5634 | 5651 | 536 | 553 | GCGCCGCCTGCAGCTCCT | 80 | 237 |
| 688804 | 5636 | 5653 | 538 | 555 | CTGCGCCGCCTGCAGCTC | 71 | 238 |
| 688805 | 5638 | 5655 | 540 | 557 | GCCTGCGCCGCCTGCAGC | 99 | 239 |
| 688806 | 5640 | 5657 | 542 | 559 | GGGCCTGCGCCGCCTGCA | 81 | 240 |
| 688807 | 5642 | 5659 | 544 | 561 | CCGGGCCTGCGCCGCCTG | 88 | 241 |
| 688808 | 5644 | 5661 | 546 | 563 | AGCCGGGCCTGCGCCGCC | 67 | 242 |
| 688809 | 5648 | 5665 | 550 | 567 | GCCCAGCCGGGCCTGCGC | 114 | 243 |
| 688810 | 5650 | 5667 | 552 | 569 | GCGCCCAGCCGGGCCTGC | 65 | 244 |
| 688811 | 5652 | 5669 | 554 | 571 | CCGCGCCCAGCCGGGCCT | 58 | 245 |
| 688812 | 5654 | 5671 | 556 | 573 | GTCCGCGCCCAGCCGGGC | 75 | 246 |
| 689014 | 6255 | 6272 | 1157 | 1174 | GCATGGCTGCAGGCTTCG | 44 | 169 |
| 689016 | 6258 | 6275 | 1160 | 1177 | GTCGCATGGCTGCAGGCT | 27 | 170 |

TABLE 5
Reduction of APOE RNA by 5-8-5 MOE gapmers with mixed PO/PS internucleoside linkages
at a concentration of 2000 nM in HepG2 cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 21 | 70 |
| 688813 | 5656 | 5673 | 558 | 575 | ATGTCCGCGCCCAGCCGG | 144 | 247 |
| 688814 | 5658 | 5675 | 560 | 577 | CCATGTCCGCGCCCAGCC | 112 | 248 |
| 688815 | 5660 | 5677 | 562 | 579 | CTCCATGTCCGCGCCCAG | 98 | 249 |
| 688816 | 5662 | 5679 | 564 | 581 | TCCTCCATGTCCGCGCCC | 102 | 250 |
| 688817 | 5664 | 5681 | 566 | 583 | CGTCCTCCATGTCCGCGC | 122 | 251 |
| 688818 | 5680 | 5697 | 582 | 599 | ACCAGGCGGCCGCACACG | 73 | 252 |
| 688819 | 5682 | 5699 | 584 | 601 | GCACCAGGCGGCCGCACA | 67 | 253 |
| 688820 | 5684 | 5701 | 586 | 603 | CTGCACCAGGCGGCCGCA | 126 | 254 |
| 688821 | 5686 | 5703 | 588 | 605 | TACTGCACCAGGCGGCCG | 131 | 255 |
| 688822 | 5688 | 5705 | 590 | 607 | GGTACTGCACCAGGCGGC | 119 | 256 |
| 688823 | 5690 | 5707 | 592 | 609 | GCGGTACTGCACCAGGCG | 108 | 257 |
| 688824 | 5692 | 5709 | 594 | 611 | CCGCGGTACTGCACCAGG | 89 | 258 |
| 688825 | 5694 | 5711 | 596 | 613 | CGCCGCGGTACTGCACCA | 79 | 259 |
| 688826 | 5698 | 5715 | 600 | 617 | ACCTCGCCGCGGTACTGC | 114 | 260 |
| 688827 | 5700 | 5717 | 602 | 619 | GCACCTCGCCGCGGTACT | 124 | 261 |
| 688828 | 5704 | 5721 | 606 | 623 | GCCTGCACCTCGCCGCGG | 110 | 262 |
| 688829 | 5706 | 5723 | 608 | 625 | TGGCCTGCACCTCGCCGC | 163 | 263 |
| 688830 | 5708 | 5725 | 610 | 627 | CATGGCCTGCACCTCGCC | 74 | 264 |
| 688831 | 5710 | 5727 | 612 | 629 | AGCATGGCCTGCACCTCG | 63 | 265 |
| 688832 | 5712 | 5729 | 614 | 631 | CGAGCATGGCCTGCACCT | 68 | 266 |
| 688833 | 5714 | 5731 | 616 | 633 | GCCGAGCATGGCCTGCAC | 86 | 267 |
| 688834 | 5716 | 5733 | 618 | 635 | TGGCCGAGCATGGCCTGC | 142 | 268 |
| 688835 | 5720 | 5737 | 622 | 639 | GCTCTGGCCGAGCATGGC | 112 | 269 |
| 688836 | 5722 | 5739 | 624 | 641 | GTGCTCTGGCCGAGCATG | 94 | 270 |
| 688837 | 5724 | 5741 | 626 | 643 | CGGTGCTCTGGCCGAGCA | 128 | 271 |
| 688838 | 5726 | 5743 | 628 | 645 | CTCGGTGCTCTGGCCGAG | 74 | 272 |
| 688839 | 5728 | 5745 | 630 | 647 | TCCTCGGTGCTCTGGCCG | 127 | 273 |
| 688840 | 5730 | 5747 | 632 | 649 | GCTCCTCGGTGCTCTGGC | 98 | 274 |
| 688841 | 5732 | 5749 | 634 | 651 | CAGCTCCTCGGTGCTCTG | 147 | 275 |
| 688842 | 5734 | 5751 | 636 | 653 | CGCAGCTCCTCGGTGCTC | 134 | 276 |
| 688843 | 5736 | 5753 | 638 | 655 | CCCGCAGCTCCTCGGTGC | 104 | 277 |
| 688844 | 5738 | 5755 | 640 | 657 | CACCCGCAGCTCCTCGGT | 92 | 278 |
| 688845 | 5740 | 5757 | 642 | 659 | CGCACCCGCAGCTCCTCG | 110 | 279 |
| 688846 | 5742 | 5759 | 644 | 661 | GGCGCACCCGCAGCTCCT | 84 | 280 |
| 688847 | 5744 | 5761 | 646 | 663 | GAGGCGCACCCGCAGCTC | 102 | 281 |
| 688848 | 5746 | 5763 | 648 | 665 | GCGAGGCGCACCCGCAGC | 61 | 282 |
| 688849 | 5748 | 5765 | 650 | 667 | AGGCGAGGCGCACCCGCA | 125 | 283 |
| 688850 | 5750 | 5767 | 652 | 669 | GGAGGCGAGGCGCACCCG | 93 | 284 |
| 688851 | 5752 | 5769 | 654 | 671 | TGGGAGGCGAGGCGCACC | 126 | 285 |
| 688852 | 5754 | 5771 | 656 | 673 | GGTGGGAGGCGAGGCGCA | 113 | 286 |
| 688853 | 5756 | 5773 | 658 | 675 | CAGGTGGGAGGCGAGGCG | 114 | 287 |
| 688854 | 5758 | 5775 | 660 | 677 | CGCAGGTGGGAGGCGAGG | 133 | 288 |
| 688855 | 5762 | 5779 | 664 | 681 | CTTGCGCAGGTGGGAGGC | 78 | 289 |
| 688856 | 5764 | 5781 | 666 | 683 | AGCTTGCGCAGGTGGGAG | 131 | 290 |
| 688857 | 5768 | 5785 | 670 | 687 | ACGCAGCTTGCGCAGGTG | 71 | 291 |
| 688858 | 5770 | 5787 | 672 | 689 | TTACGCAGCTTGCGCAGG | 120 | 292 |
| 688859 | 5772 | 5789 | 674 | 691 | GCTTACGCAGCTTGCGCA | 78 | 293 |
| 688860 | 5774 | 5791 | 676 | 693 | CCGCTTACGCAGCTTGCG | 143 | 294 |
| 688861 | 5776 | 5793 | 678 | 695 | AGCCGCTTACGCAGCTTG | 79 | 295 |
| 688862 | 5778 | 5795 | 680 | 697 | GGAGCCGCTTACGCAGCT | 131 | 296 |
| 688863 | 5782 | 5799 | 684 | 701 | CGGAGGAGCCGCTTACGC | 131 | 297 |
| 688864 | 5784 | 5801 | 686 | 703 | CGCGGAGGAGCCGCTTAC | 96 | 298 |
| 688865 | 5786 | 5803 | 688 | 705 | ATCGCGGAGGAGCCGCTT | 109 | 299 |
| 688866 | 5788 | 5805 | 690 | 707 | GCATCGCGGAGGAGCCGC | 107 | 300 |
| 688867 | 5790 | 5807 | 692 | 709 | CGGCATCGCGGAGGAGCC | 82 | 301 |
| 688868 | 5792 | 5809 | 694 | 711 | ATCGGCATCGCGGAGGAG | 122 | 302 |
| 688869 | 5796 | 5813 | 698 | 715 | GGTCATCGGCATCGCGGA | 131 | 303 |
| 688870 | 5798 | 5815 | 700 | 717 | CAGGTCATCGGCATCGCG | 104 | 304 |
| 688871 | 5801 | 5818 | 703 | 720 | CTGCAGGTCATCGGCATC | 123 | 305 |
| 688872 | 5802 | 5819 | 704 | 721 | TCTGCAGGTCATCGGCAT | 91 | 306 |
| 688873 | 5804 | 5821 | 706 | 723 | CTTCTGCAGGTCATCGGC | 102 | 307 |
| 688874 | 5806 | 5823 | 708 | 725 | CGCTTCTGCAGGTCATCG | 101 | 308 |
| 688875 | 5821 | 5838 | 723 | 740 | TGGTACACTGCCAGGCGC | 87 | 309 |
| 688876 | 5823 | 5840 | 725 | 742 | CCTGGTACACTGCCAGGC | 148 | 310 |
| 688877 | 5825 | 5842 | 727 | 744 | GGCCTGGTACACTGCCAG | 104 | 311 |
| 688878 | 5827 | 5844 | 729 | 746 | CCGGCCTGGTACACTGCC | 137 | 312 |
| 688879 | 5829 | 5846 | 731 | 748 | CCCCGGCCTGGTACACTG | 81 | 313 |
| 688880 | 5831 | 5848 | 733 | 750 | GGCCCCGGCCTGGTACAC | 81 | 314 |
| 688881 | 5833 | 5850 | 735 | 752 | CGGGCCCCGGCCTGGTAC | 87 | 315 |
| 688882 | 5835 | 5852 | 737 | 754 | CGCGGGCCCCGGCCTGGT | 104 | 316 |
| 688883 | 5837 | 5854 | 739 | 756 | CTCGCGGGCCCCGGCCTG | 105 | 317 |
| 688884 | 5839 | 5856 | 741 | 758 | CCCTCGCGGGCCCCGGCC | 126 | 318 |
| 688885 | 5841 | 5858 | 743 | 760 | CGCCCTCGCGGGCCCCGG | 88 | 319 |
| 688886 | 5856 | 5873 | 758 | 775 | TGAGGCCGCGCTCGGCGC | 62 | 320 |
| 688887 | 5858 | 5875 | 760 | 777 | GCTGAGGCCGCGCTCGGC | 100 | 321 |
| 688888 | 5860 | 5877 | 762 | 779 | GCGCTGAGGCCGCGCTCG | 87 | 322 |
| 689014 | 6255 | 6272 | 1157 | 1174 | GCATGGCTGCAGGCTTCG | 13 | 169 |
| 689016 | 6258 | 6275 | 1160 | 1177 | GTCGCATGGCTGCAGGCT | 15 | 170 |

TABLE 6
Reduction of APOE RNA by 5-8-5 MOE gapmers with mixed PO/PS internucleoside linkages
at a concentration of 2000 nM in HepG2 cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 23 | 70 |
| 688889 | 5898 | 5915 | 800 | 817 | GGCCCTGTTCCACCAGGG | 145 | 323 |
| 688890 | 5900 | 5917 | 802 | 819 | GCGGCCCTGTTCCACCAG | 149 | 324 |
| 688891 | 5902 | 5919 | 804 | 821 | ACGCGGCCCTGTTCCACC | 98 | 325 |
| 688892 | 5904 | 5921 | 806 | 823 | GCACGCGGCCCTGTTCCA | 107 | 326 |
| 688893 | 5906 | 5923 | 808 | 825 | CCGCACGCGGCCCTGTTC | 163 | 327 |
| 688894 | 5908 | 5925 | 810 | 827 | GCCCGCACGCGGCCCTGT | 128 | 328 |
| 688895 | 5910 | 5927 | 812 | 829 | CGGCCCGCACGCGGCCCT | 79 | 329 |
| 688896 | 5912 | 5929 | 814 | 831 | GGCGGCCCGCACGCGGCC | 103 | 330 |
| 688897 | 5914 | 5931 | 816 | 833 | GTGGCGGCCCGCACGCGG | 118 | 331 |
| 688898 | 5916 | 5933 | 818 | 835 | CAGTGGCGGCCCGCACGC | 143 | 332 |
| 688899 | 5918 | 5935 | 820 | 837 | CACAGTGGCGGCCCGCAC | 97 | 333 |
| 688900 | 5920 | 5937 | 822 | 839 | CCCACAGTGGCGGCCCGC | 65 | 334 |
| 688901 | 5922 | 5939 | 824 | 841 | AGCCCACAGTGGCGGCCC | 96 | 335 |
| 688902 | 5924 | 5941 | 826 | 843 | GGAGCCCACAGTGGCGGC | 92 | 336 |
| 688903 | 5928 | 5945 | 830 | 847 | CCAGGGAGCCCACAGTGG | 87 | 337 |
| 688904 | 5930 | 5947 | 832 | 849 | GGCCAGGGAGCCCACAGT | 114 | 338 |
| 688905 | 5932 | 5949 | 834 | 851 | CCGGCCAGGGAGCCCACA | 125 | 339 |
| 688906 | 5934 | 5951 | 836 | 853 | GGCCGGCCAGGGAGCCCA | 72 | 340 |
| 688907 | 5936 | 5953 | 838 | 855 | CTGGCCGGCCAGGGAGCC | 128 | 341 |
| 688908 | 5938 | 5955 | 840 | 857 | GGCTGGCCGGCCAGGGAG | 95 | 342 |
| 688909 | 5941 | 5958 | 843 | 860 | AGCGGCTGGCCGGCCAGG | 64 | 343 |
| 688910 | 5943 | 5960 | 845 | 862 | GTAGCGGCTGGCCGGCCA | 105 | 344 |
| 688911 | 5945 | 5962 | 847 | 864 | CTGTAGCGGCTGGCCGGC | 117 | 345 |
| 688912 | 5947 | 5964 | 849 | 866 | TCCTGTAGCGGCTGGCCG | 110 | 346 |
| 688913 | 5949 | 5966 | 851 | 868 | GCTCCTGTAGCGGCTGGC | 63 | 347 |
| 688914 | 5951 | 5968 | 853 | 870 | CCGCTCCTGTAGCGGCTG | 109 | 348 |
| 688915 | 5955 | 5972 | 857 | 874 | GGGCCCGCTCCTGTAGCG | 90 | 349 |
| 688916 | 5957 | 5974 | 859 | 876 | CTGGGCCCGCTCCTGTAG | 148 | 350 |
| 688917 | 5959 | 5976 | 861 | 878 | GCCTGGGCCCGCTCCTGT | 98 | 351 |
| 688918 | 5961 | 5978 | 863 | 880 | AGGCCTGGGCCCGCTCCT | 111 | 352 |
| 688919 | 5963 | 5980 | 865 | 882 | CCAGGCCTGGGCCCGCTC | 137 | 353 |
| 688920 | 5965 | 5982 | 867 | 884 | CCCCAGGCCTGGGCCCGC | 111 | 354 |
| 688921 | 5967 | 5984 | 869 | 886 | CGCCCCAGGCCTGGGCCC | 102 | 355 |
| 688922 | 5971 | 5988 | 873 | 890 | CGCTCGCCCCAGGCCTGG | 117 | 356 |
| 688923 | 5973 | 5990 | 875 | 892 | GCCGCTCGCCCCAGGCCT | 90 | 357 |
| 688924 | 5975 | 5992 | 877 | 894 | CAGCCGCTCGCCCCAGGC | 108 | 358 |
| 688925 | 5977 | 5994 | 879 | 896 | CGCAGCCGCTCGCCCCAG | 108 | 359 |
| 688926 | 5979 | 5996 | 881 | 898 | CGCGCAGCCGCTCGCCCC | 63 | 360 |
| 688927 | 5981 | 5998 | 883 | 900 | CGCGCGCAGCCGCTCGCC | 129 | 361 |
| 688928 | 5983 | 6000 | 885 | 902 | CGCGCGCGCAGCCGCTCG | 102 | 362 |
| 688929 | 5985 | 6002 | 887 | 904 | TCCGCGCGCGCAGCCGCT | 67 | 363 |
| 688930 | 5987 | 6004 | 889 | 906 | CATCCGCGCGCGCAGCCG | 74 | 364 |
| 688931 | 5989 | 6006 | 891 | 908 | TCCATCCGCGCGCGCAGC | 106 | 365 |
| 688932 | 5991 | 6008 | 893 | 910 | CCTCCATCCGCGCGCGCA | 119 | 366 |
| 688933 | 5993 | 6010 | 895 | 912 | CTCCTCCATCCGCGCGCG | 104 | 367 |
| 688934 | 5995 | 6012 | 897 | 914 | ATCTCCTCCATCCGCGCG | 118 | 368 |
| 688935 | 5997 | 6014 | 899 | 916 | CCATCTCCTCCATCCGCG | 114 | 369 |
| 688936 | 5999 | 6016 | 901 | 918 | GCCCATCTCCTCCATCCG | 103 | 370 |
| 688937 | 6003 | 6020 | 905 | 922 | GGCTGCCCATCTCCTCCA | 101 | 371 |
| 688938 | 6005 | 6022 | 907 | 924 | CCGGCTGCCCATCTCCTC | 83 | 372 |
| 688939 | 6007 | 6024 | 909 | 926 | GTCCGGCTGCCCATCTCC | 68 | 373 |
| 688940 | 6009 | 6026 | 911 | 928 | GGGTCCGGCTGCCCATCT | 101 | 374 |
| 688941 | 6011 | 6028 | 913 | 930 | GCGGGTCCGGCTGCCCAT | 106 | 375 |
| 688942 | 6013 | 6030 | 915 | 932 | TCGCGGGTCCGGCTGCCC | 133 | 376 |
| 688943 | 6015 | 6032 | 917 | 934 | GGTCGCGGGTCCGGCTGC | 134 | 377 |
| 688944 | 6017 | 6034 | 919 | 936 | GCGGTCGCGGGTCCGGCT | 88 | 378 |
| 688945 | 6019 | 6036 | 921 | 938 | AGGCGGTCGCGGGTCCGG | 105 | 379 |
| 688946 | 6021 | 6038 | 923 | 940 | CCAGGCGGTCGCGGGTCC | 102 | 380 |
| 688947 | 6023 | 6040 | 925 | 942 | GTCCAGGCGGTCGCGGGT | 64 | 381 |
| 688948 | 6040 | 6057 | 942 | 959 | ACCTGCTCCTTCACCTCG | 94 | 382 |
| 688949 | 6042 | 6059 | 944 | 961 | CCACCTGCTCCTTCACCT | 129 | 383 |
| 688950 | 6044 | 6061 | 946 | 963 | CGCCACCTGCTCCTTCAC | 99 | 384 |
| 688951 | 6046 | 6063 | 948 | 965 | TCCGCCACCTGCTCCTTC | 168 | 385 |
| 688952 | 6048 | 6065 | 950 | 967 | CCTCCGCCACCTGCTCCT | 69 | 386 |
| 688953 | 6050 | 6067 | 952 | 969 | CACCTCCGCCACCTGCTC | 130 | 387 |
| 688954 | 6052 | 6069 | 954 | 971 | CGCACCTCCGCCACCTGC | 59 | 388 |
| 688955 | 6054 | 6071 | 956 | 973 | CGCGCACCTCCGCCACCT | 88 | 389 |
| 688956 | 6056 | 6073 | 958 | 975 | GGCGCGCACCTCCGCCAC | 86 | 390 |
| 688957 | 6058 | 6075 | 960 | 977 | TTGGCGCGCACCTCCGCC | 122 | 391 |
| 688958 | 6060 | 6077 | 962 | 979 | GCTTGGCGCGCACCTCCG | 115 | 392 |
| 688959 | 6062 | 6079 | 964 | 981 | CAGCTTGGCGCGCACCTC | 83 | 393 |
| 688960 | 6066 | 6083 | 968 | 985 | CCTCCAGCTTGGCGCGCA | 138 | 394 |
| 688961 | 6068 | 6085 | 970 | 987 | CTCCTCCAGCTTGGCGCG | 53 | 395 |
| 688962 | 6070 | 6087 | 972 | 989 | TGCTCCTCCAGCTTGGCG | 83 | 396 |
| 688963 | 6072 | 6089 | 974 | 991 | CCTGCTCCTCCAGCTTGG | 55 | 397 |
| 688964 | 6074 | 6091 | 976 | 993 | GGCCTGCTCCTCCAGCTT | 84 | 398 |
| 689014 | 6255 | 6272 | 1157 | 1174 | GCATGGCTGCAGGCTTCG | 21 | 169 |
| 689016 | 6258 | 6275 | 1160 | 1177 | GTCGCATGGCTGCAGGCT | 21 | 170 |

TABLE 7
Reduction of APOE RNA by 5-8-5 MOE gapmers with mixed PO/PS internucleoside linkages
at a concentration of 2000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 19 | 70 |
| 688965 | 6076 | 6093 | 978 | 995 | TGGGCCTGCTCCTCCAGC | 92 | 399 |
| 688966 | 6078 | 6095 | 980 | 997 | GCTGGGCCTGCTCCTCCA | 76 | 400 |
| 688967 | 6080 | 6097 | 982 | 999 | CTGCTGGGCCTGCTCCTC | 90 | 401 |
| 688968 | 6082 | 6099 | 984 | 1001 | ATCTGCTGGGCCTGCTCC | 90 | 402 |
| 688969 | 6084 | 6101 | 986 | 1003 | GTATCTGCTGGGCCTGCT | 79 | 403 |
| 688970 | 6101 | 6118 | 1003 | 1020 | GGCCTCGGCCTGCAGGCG | 84 | 404 |
| 688971 | 6103 | 6120 | 1005 | 1022 | AAGGCCTCGGCCTGCAGG | 92 | 405 |
| 688972 | 6105 | 6122 | 1007 | 1024 | GGAAGGCCTCGGCCTGCA | 96 | 406 |
| 688973 | 6107 | 6124 | 1009 | 1026 | CTGGAAGGCCTCGGCCTG | 88 | 407 |
| 688974 | 6109 | 6126 | 1011 | 1028 | GCCTGGAAGGCCTCGGCC | 78 | 408 |
| 688975 | 6111 | 6128 | 1013 | 1030 | GGGCCTGGAAGGCCTCGG | 82 | 409 |
| 688976 | 6113 | 6130 | 1015 | 1032 | GCGGGCCTGGAAGGCCTC | 75 | 410 |
| 688977 | 6115 | 6132 | 1017 | 1034 | AGGCGGGCCTGGAAGGCC | 77 | 411 |
| 688978 | 6117 | 6134 | 1019 | 1036 | TGAGGCGGGCCTGGAAGG | 81 | 412 |
| 688979 | 6119 | 6136 | 1021 | 1038 | CTTGAGGCGGGCCTGGAA | 79 | 413 |
| 688980 | 6123 | 6140 | 1025 | 1042 | AGCTCTTGAGGCGGGCCT | 78 | 414 |
| 688981 | 6125 | 6142 | 1027 | 1044 | CCAGCTCTTGAGGCGGGC | 84 | 415 |
| 688982 | 6127 | 6144 | 1029 | 1046 | AACCAGCTCTTGAGGCGG | 85 | 416 |
| 688983 | 6129 | 6146 | 1031 | 1048 | CGAACCAGCTCTTGAGGC | 117 | 417 |
| 688984 | 6131 | 6148 | 1033 | 1050 | CTCGAACCAGCTCTTGAG | 66 | 418 |
| 688985 | 6133 | 6150 | 1035 | 1052 | GGCTCGAACCAGCTCTTG | 76 | 419 |
| 688986 | 6150 | 6167 | 1052 | 1069 | GCATGTCTTCCACCAGGG | 72 | 420 |
| 688987 | 6152 | 6169 | 1054 | 1071 | CTGCATGTCTTCCACCAG | 80 | 421 |
| 688988 | 6154 | 6171 | 1056 | 1073 | CGCTGCATGTCTTCCACC | 69 | 422 |
| 688989 | 6169 | 6186 | 1071 | 1088 | AGCCCGGCCCACTGGCGC | 76 | 423 |
| 688990 | 6171 | 6188 | 1073 | 1090 | CCAGCCCGGCCCACTGGC | 82 | 424 |
| 688991 | 6173 | 6190 | 1075 | 1092 | CACCAGCCCGGCCCACTG | 74 | 425 |
| 688992 | 6175 | 6192 | 1077 | 1094 | TCCACCAGCCCGGCCCAC | 108 | 426 |
| 688993 | 6177 | 6194 | 1079 | 1096 | TCTCCACCAGCCCGGCCC | 223 | 427 |
| 688994 | 6179 | 6196 | 1081 | 1098 | CTTCTCCACCAGCCCGGC | 75 | 428 |
| 688995 | 6181 | 6198 | 1083 | 1100 | ACCTTCTCCACCAGCCCG | 101 | 429 |
| 688996 | 6185 | 6202 | 1087 | 1104 | CTGCACCTTCTCCACCAG | 99 | 430 |
| 688997 | 6187 | 6204 | 1089 | 1106 | GCCTGCACCTTCTCCACC | 89 | 431 |
| 688998 | 6189 | 6206 | 1091 | 1108 | CAGCCTGCACCTTCTCCA | 80 | 432 |
| 688999 | 6191 | 6208 | 1093 | 1110 | GGCAGCCTGCACCTTCTC | 57 | 433 |
| 689000 | 6193 | 6210 | 1095 | 1112 | ACGGCAGCCTGCACCTTC | 90 | 434 |
| 689001 | 6195 | 6212 | 1097 | 1114 | CCACGGCAGCCTGCACCT | 74 | 435 |
| 689002 | 6197 | 6214 | 1099 | 1116 | GCCCACGGCAGCCTGCAC | 93 | 436 |
| 689003 | 6199 | 6216 | 1101 | 1118 | GTGCCCACGGCAGCCTGC | 51 | 437 |
| 689004 | 6201 | 6218 | 1103 | 1120 | TGGTGCCCACGGCAGCCT | 79 | 438 |
| 689005 | 6203 | 6220 | 1105 | 1122 | GCTGGTGCCCACGGCAGC | 78 | 439 |
| 689006 | 6205 | 6222 | 1107 | 1124 | GCGCTGGTGCCCACGGCA | 61 | 440 |
| 689007 | 6227 | 6244 | 1129 | 1146 | ATTGTCGCTGGGCACAGG | 88 | 441 |
| 689008 | 6229 | 6246 | 1131 | 1148 | TGATTGTCGCTGGGCACA | 83 | 442 |
| 689009 | 6231 | 6248 | 1133 | 1150 | AGTGATTGTCGCTGGGCA | 56 | 443 |
| 689010 | 6233 | 6250 | 1135 | 1152 | TCAGTGATTGTCGCTGGG | 100 | 444 |
| 689011 | 6235 | 6252 | 1137 | 1154 | GTTCAGTGATTGTCGCTG | 91 | 445 |
| 689012 | 6252 | 6269 | 1154 | 1171 | TGGCTGCAGGCTTCGGCG | 72 | 446 |
| 689013 | 6254 | 6271 | 1156 | 1173 | CATGGCTGCAGGCTTCGG | 32 | 447 |
| 689014 | 6255 | 6272 | 1157 | 1174 | GCATGGCTGCAGGCTTCG | 50 | 169 |
| 689015 | 6257 | 6274 | 1159 | 1176 | TCGCATGGCTGCAGGCTT | 19 | 448 |
| 689016 | 6258 | 6275 | 1160 | 1177 | GTCGCATGGCTGCAGGCT | 33 | 170 |
| 689017 | 6260 | 6277 | 1162 | 1179 | GGGTCGCATGGCTGCAGG | 41 | 449 |
| 689018 | 6286 | 6303 | 1188 | 1205 | GGAGGCAGGAGGCACGGG | 91 | 450 |
| 689019 | 6288 | 6305 | 1190 | 1207 | GCGGAGGCAGGAGGCACG | 82 | 451 |
| 689020 | 6290 | 6307 | 1192 | 1209 | GCGCGGAGGCAGGAGGCA | 65 | 452 |
| 689021 | 6292 | 6309 | 1194 | 1211 | CTGCGCGGAGGCAGGAGG | 69 | 453 |
| 689022 | 6294 | 6311 | 1196 | 1213 | GGCTGCGCGGAGGCAGGA | 86 | 454 |
| 689023 | 6296 | 6313 | 1198 | 1215 | CAGGCTGCGCGGAGGCAG | 56 | 455 |
| 689024 | 6298 | 6315 | 1200 | 1217 | TGCAGGCTGCGCGGAGGC | 92 | 456 |
| 689025 | 6300 | 6317 | 1202 | 1219 | GCTGCAGGCTGCGCGGAG | 75 | 457 |
| 689026 | 6302 | 6319 | 1204 | 1221 | CCGCTGCAGGCTGCGCGG | 78 | 458 |
| 689027 | 6304 | 6321 | 1206 | 1223 | TCCCGCTGCAGGCTGCGC | 53 | 459 |
| 689028 | 6306 | 6323 | 1208 | 1225 | TCTCCCGCTGCAGGCTGC | 37 | 460 |
| 689029 | 6308 | 6325 | 1210 | 1227 | GGTCTCCCGCTGCAGGCT | 42 | 461 |
| 689030 | 6310 | 6327 | 1212 | 1229 | AGGGTCTCCCGCTGCAGG | 70 | 462 |
| 689031 | 6312 | 6329 | 1214 | 1231 | ACAGGGTCTCCCGCTGCA | 68 | 463 |
| 689032 | 6314 | 6331 | 1216 | 1233 | GGACAGGGTCTCCCGCTG | 61 | 464 |
| 689033 | 6336 | 6353 | 1238 | 1255 | CCCAGGAGGACGGCTGGG | 86 | 465 |
| 689034 | 6338 | 6355 | 1240 | 1257 | ACCCCAGGAGGACGGCTG | 105 | 466 |
| 689035 | 6340 | 6357 | 1242 | 1259 | CCACCCCAGGAGGACGGC | 80 | 467 |
| 689036 | 6342 | 6359 | 1244 | 1261 | GTCCACCCCAGGAGGACG | 82 | 468 |
| 689037 | 6344 | 6361 | 1246 | 1263 | GGGTCCACCCCAGGAGGA | 97 | 469 |
| 689038 | 6346 | 6363 | 1248 | 1265 | TAGGGTCCACCCCAGGAG | 66 | 470 |
| 689039 | 6348 | 6365 | 1250 | 1267 | ACTAGGGTCCACCCCAGG | 79 | 471 |
| 689040 | 6352 | 6369 | 1254 | 1271 | TTAAACTAGGGTCCACCC | 61 | 472 |
| 689041 | 6354 | 6371 | 1256 | 1273 | TATTAAACTAGGGTCCAC | 79 | 473 |
| 689042 | 6356 | 6373 | 1258 | 1275 | TTTATTAAACTAGGGTCC | 58 | 474 |

TABLE 8
Reduction of APOE RNA by 5-8-5 MOE gapmers with mixed PO/PS internucleoside linkages
at a concentration of 2000 nM in HepG2 cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 37 | 70 |
| 689014 | 6255 | 6272 | 1157 | 1174 | GCATGGCTGCAGGCTTCG | 33 | 169 |
| 689016 | 6258 | 6275 | 1160 | 1177 | GTCGCATGGCTGCAGGCT | 14 | 170 |
| 689043 | 6358 | 6375 | 1260 | 1277 | TCTTTATTAAACTAGGGT | 23 | 475 |
| 689044 | 6360 | 6377 | 1262 | 1279 | AATCTTTATTAAACTAGG | 39 | 476 |
| 689045 | 6362 | 6379 | 1264 | 1281 | TGAATCTTTATTAAACTA | 103 | 477 |
| 689046 | 6364 | 6381 | 1266 | 1283 | GGTGAATCTTTATTAAAC | 12 | 478 |
| 689047 | 6366 | 6383 | 1268 | 1285 | TTGGTGAATCTTTATTAA | 33 | 479 |
| 689048 | 6368 | 6385 | 1270 | 1287 | ACTTGGTGAATCTTTATT | 28 | 480 |
| 689049 | 6370 | 6387 | 1272 | 1289 | AAACTTGGTGAATCTTTA | 41 | 481 |
| 689050 | 6372 | 6389 | 1274 | 1291 | TGAAACTTGGTGAATCTT | 29 | 482 |
| 689051 | 6374 | 6391 | 1276 | 1293 | CGTGAAACTTGGTGAATC | 22 | 483 |
| 689052 | 3652 | 3669 | N/A | N/A | ACCTGCCAGGAATGTGAC | 113 | 484 |
| 689053 | 3667 | 3684 | N/A | N/A | AAGCCCCGCCCCCATACC | 69 | 485 |
| 689054 | 3708 | 3725 | N/A | N/A | TGAGGTGAGGATGAGAGG | 101 | 486 |
| 689055 | 3737 | 3754 | N/A | N/A | CAGGGTCTGCCTGAATGG | 101 | 487 |
| 689056 | 3759 | 3776 | N/A | N/A | AGAAGCCTCAGAAGAGGG | 116 | 488 |
| 689057 | 3774 | 3791 | N/A | N/A | GCCAGGAAGCAGCACAGA | 130 | 489 |
| 689058 | 3789 | 3806 | N/A | N/A | AAATCGCTGTTCAGAGCC | 107 | 490 |
| 689059 | 3809 | 3826 | N/A | N/A | ACCGAGGCCCAGAGAGCG | 128 | 491 |
| 689060 | 3831 | 3848 | N/A | N/A | TCCTATCTCAAGGATGGG | 125 | 492 |
| 689061 | 3846 | 3863 | N/A | N/A | AAAACAACTTCTAACTCC | 102 | 493 |
| 689062 | 4119 | 4136 | N/A | N/A | TCGAAACGGGCAGATCAC | 56 | 494 |
| 689063 | 4172 | 4189 | N/A | N/A | AACTCCCAGCCAGGTGCG | 117 | 495 |
| 689064 | 4187 | 4204 | N/A | N/A | GCATTAGAAACCTCTAAC | 101 | 496 |
| 689065 | 4202 | 4219 | N/A | N/A | CTATCTGCCTGCAATGCA | 82 | 497 |
| 689066 | 4232 | 4249 | N/A | N/A | AGATCACAGCTGCCCCGT | 109 | 498 |
| 689067 | 4247 | 4264 | N/A | N/A | GGTGATGGAGAATAAAGA | 118 | 499 |
| 689068 | 4266 | 4283 | N/A | N/A | CCAGGCAGGGCTGTGTGG | 89 | 500 |
| 689069 | 4281 | 4298 | N/A | N/A | GTGTCCTTGTGTGCCCCA | 54 | 501 |
| 689070 | 4296 | 4313 | N/A | N/A | AAAAGCATGTATTGAGTG | 56 | 502 |
| 689071 | 4460 | 4477 | N/A | N/A | GCACAGGTGTGTGGCACC | 70 | 503 |
| 689072 | 4536 | 4553 | N/A | N/A | CTGAACATGGTTCACTGC | 103 | 504 |
| 689073 | 4586 | 4603 | N/A | N/A | GTATTTATAAACAGGGTC | 96 | 505 |
| 689074 | 4601 | 4618 | N/A | N/A | CTTGGAAAGCATTATGTA | 130 | 506 |
| 689075 | 4616 | 4633 | N/A | N/A | GAGTCGGTTTAATCACTT | 85 | 507 |
| 689076 | 4643 | 4660 | N/A | N/A | GGAGCCATGGTGGGCAGG | 69 | 508 |
| 689077 | 4658 | 4675 | N/A | N/A | CACAAATGCTTCTTTGGA | 68 | 509 |
| 689078 | 4673 | 4690 | N/A | N/A | ACACAGAAGGTGCTCCAC | 111 | 510 |
| 689079 | 4693 | 4710 | N/A | N/A | GGCATCTAGTACCTAGGG | 81 | 511 |
| 689080 | 4708 | 4725 | N/A | N/A | TTCTGACCCCGTCCAGGC | 65 | 512 |
| 689081 | 4730 | 4747 | N/A | N/A | AGTTCAAGGTGGGTCAGG | 83 | 513 |
| 689082 | 4745 | 4762 | N/A | N/A | ATCCTGTGTGGAACAAGT | 77 | 514 |
| 689083 | 4937 | 4954 | N/A | N/A | ACCTCAGTTCCTGGGTGA | 76† | 515 |
| 689084 | 4963 | 4980 | N/A | N/A | GGTCAAGGGCCAGGATGG | 93† | 516 |
| 689085 | 4978 | 4995 | N/A | N/A | GCCGCCCACCAGGAGGGT | 132† | 517 |
| 689086 | 5004 | 5021 | N/A | N/A | ATGAAACCTGGACCTGGG | 126† | 518 |
| 689087 | 5027 | 5044 | N/A | N/A | CAAGACTTAGCGACAGGG | 110† | 519 |
| 689088 | 5042 | 5059 | N/A | N/A | GAGACCCAGGCCCCCCAA | 120† | 520 |
| 689089 | 5057 | 5074 | N/A | N/A | AAGCTAGAACCAGCAGAG | 82† | 521 |
| 689090 | 5073 | 5090 | N/A | N/A | CAGAAATGGGAAGAGGAA | 46† | 522 |
| 689091 | 5088 | 5105 | N/A | N/A | GCTAAAGCCAGGAGTCAG | 116† | 523 |
| 689092 | 5103 | 5120 | N/A | N/A | AGAGAATTCCAGAGAGCT | 100† | 524 |
| 689093 | 5118 | 5135 | N/A | N/A | AGACAAAGCTGAGAGAGA | 103† | 525 |
| 689094 | 5136 | 5153 | N/A | N/A | TCAGAAGGGAAGAGAGAG | 95† | 526 |
| 689095 | 5151 | 5168 | N/A | N/A | GTGTGAGAGACTGAGTCA | 102† | 527 |
| 689096 | 5166 | 5183 | N/A | N/A | ACAGAGCCAGGACGAGTG | 65† | 528 |
| 689097 | 5181 | 5198 | N/A | N/A | TAGGGAAGGACAGAGACA | 75† | 529 |
| 689098 | 5196 | 5213 | N/A | N/A | TCTATATAAAAGAGCTAG | 108† | 530 |
| 689099 | 5213 | 5230 | N/A | N/A | GACCCCATCTCTCTGTCT | 56† | 531 |
| 689100 | 5323 | 5340 | N/A | N/A | TAGGAGGCCGGGCAAGGT | 88† | 532 |
| 689101 | 5338 | 5355 | N/A | N/A | AGACGAAGAAGGAGCTAG | 99† | 533 |
| 689102 | 5353 | 5370 | N/A | N/A | AGAGGGCAGAGGCAGAGA | 42† | 534 |
| 689103 | 5368 | 5385 | N/A | N/A | GCAGAGAGCAGATGCAGA | 80† | 535 |
| 689104 | 5392 | 5409 | N/A | N/A | CGAGAGAAGGAGACAGAG | 104† | 536 |
| 689105 | 5436 | 5453 | N/A | N/A | GAGCCAGAGAGACCCAAG | 102† | 537 |
| 689106 | 5490 | 5507 | N/A | N/A | TCGCACAGTGGGAGGCGG | 61† | 538 |
| 689107 | 5515 | 5532 | N/A | N/A | CTGCGGCCGAGAGGGCGG | 90† | 539 |
| 689108 | 2831 | 2848 | 83 | 100 | TTCTCACCGGCTCCTGGG | 99 | 540 |
| 689109 | 2864 | 2881 | 116 | 133 | CCCCTGAGCTCATCCCCG | 128 | 541 |
| 689117 | 6236 | 6253 | 1138 | 1155 | CGTTCAGTGATTGTCGCT | 86 | 542 |
| 689118 | 6256 | 6273 | 1158 | 1175 | CGCATGGCTGCAGGCTTC | 8 | 543 |

TABLE 9
Reduction of APOE RNA by 5-8-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 689014 | 6255 | 6272 | 1157 | 1174 | GCATGGCTGCAGGCTTCG | 43 | 169 |
| 689015 | 6257 | 6274 | 1159 | 1176 | TCGCATGGCTGCAGGCTT | 34 | 448 |
| 689046 | 6364 | 6381 | 1266 | 1283 | GGTGAATCTTTATTAAAC | 27 | 478 |
| 689047 | 6366 | 6383 | 1268 | 1285 | TTGGTGAATCTTTATTAA | 65 | 479 |
| 689048 | 6368 | 6385 | 1270 | 1287 | ACTTGGTGAATCTTTATT | 35 | 480 |
| 689049 | 6370 | 6387 | 1272 | 1289 | AAACTTGGTGAATCTTTA | 36 | 481 |
| 689050 | 6372 | 6389 | 1274 | 1291 | TGAAACTTGGTGAATCTT | 37 | 482 |
| 689051 | 6374 | 6391 | 1276 | 1293 | CGTGAAACTTGGTGAATC | 40 | 483 |
| 729667 | 6237 | 6254 | 1139 | 1156 | GCGTTCAGTGATTGTCGC | 77 | 544 |
| 729669 | 6239 | 6256 | 1141 | 1158 | CGGCGTTCAGTGATTGTC | 61 | 545 |
| 729671 | 6241 | 6258 | 1143 | 1160 | TTCGGCGTTCAGTGATTG | 86 | 546 |
| 729673 | 6243 | 6260 | 1145 | 1162 | GCTTCGGCGTTCAGTGAT | 77 | 547 |
| 729675 | 6245 | 6262 | 1147 | 1164 | AGGCTTCGGCGTTCAGTG | 83 | 548 |
| 729677 | 6247 | 6264 | 1149 | 1166 | GCAGGCTTCGGCGTTCAG | 72 | 549 |
| 729679 | 6249 | 6266 | 1151 | 1168 | CTGCAGGCTTCGGCGTTC | 70 | 550 |
| 729681 | 6251 | 6268 | 1153 | 1170 | GGCTGCAGGCTTCGGCGT | 105 | 551 |
| 729682 | 6253 | 6270 | 1155 | 1172 | ATGGCTGCAGGCTTCGGC | 52 | 552 |
| 729683 | 6259 | 6276 | 1161 | 1178 | GGTCGCATGGCTGCAGGC | 54 | 553 |
| 729684 | 6287 | 6304 | 1189 | 1206 | CGGAGGCAGGAGGCACGG | 83 | 554 |
| 729685 | 6289 | 6306 | 1191 | 1208 | CGCGGAGGCAGGAGGCAC | 75 | 555 |
| 729686 | 6291 | 6308 | 1193 | 1210 | TGCGCGGAGGCAGGAGGC | 82 | 556 |
| 729687 | 6293 | 6310 | 1195 | 1212 | GCTGCGCGGAGGCAGGAG | 78 | 557 |
| 729688 | 6295 | 6312 | 1197 | 1214 | AGGCTGCGCGGAGGCAGG | 64 | 558 |
| 729689 | 6297 | 6314 | 1199 | 1216 | GCAGGCTGCGCGGAGGCA | 72 | 559 |
| 729690 | 6299 | 6316 | 1201 | 1218 | CTGCAGGCTGCGCGGAGG | 85 | 560 |
| 729691 | 6301 | 6318 | 1203 | 1220 | CGCTGCAGGCTGCGCGGA | 83 | 561 |
| 729692 | 6303 | 6320 | 1205 | 1222 | CCCGCTGCAGGCTGCGCG | 83 | 562 |
| 729693 | 6305 | 6322 | 1207 | 1224 | CTCCCGCTGCAGGCTGCG | 59 | 563 |
| 729694 | 6307 | 6324 | 1209 | 1226 | GTCTCCCGCTGCAGGCTG | 56 | 564 |
| 729695 | 6309 | 6326 | 1211 | 1228 | GGGTCTCCCGCTGCAGGC | 67 | 565 |
| 729696 | 6311 | 6328 | 1213 | 1230 | CAGGGTCTCCCGCTGCAG | 63 | 566 |
| 729697 | 6313 | 6330 | 1215 | 1232 | GACAGGGTCTCCCGCTGC | 69 | 567 |
| 729698 | 6337 | 6354 | 1239 | 1256 | CCCCAGGAGGACGGCTGG | 85 | 568 |
| 729699 | 6339 | 6356 | 1241 | 1258 | CACCCCAGGAGGACGGCT | 95 | 569 |
| 729700 | 6341 | 6358 | 1243 | 1260 | TCCACCCCAGGAGGACGG | 96 | 570 |
| 729701 | 6343 | 6360 | 1245 | 1262 | GGTCCACCCCAGGAGGAC | 101 | 571 |
| 729702 | 6345 | 6362 | 1247 | 1264 | AGGGTCCACCCCAGGAGG | 87 | 572 |
| 729703 | 6347 | 6364 | 1249 | 1266 | CTAGGGTCCACCCCAGGA | 92 | 573 |
| 729704 | 6349 | 6366 | 1251 | 1268 | AACTAGGGTCCACCCCAG | 73 | 574 |
| 729706 | 6351 | 6368 | 1253 | 1270 | TAAACTAGGGTCCACCCC | 84 | 575 |
| 729707 | 6353 | 6370 | 1255 | 1272 | ATTAAACTAGGGTCCACC | 63 | 576 |
| 729708 | 6355 | 6372 | 1257 | 1274 | TTATTAAACTAGGGTCCA | 59 | 577 |
| 729709 | 6357 | 6374 | 1259 | 1276 | CTTTATTAAACTAGGGTC | 30 | 578 |
| 729710 | 6359 | 6376 | 1261 | 1278 | ATCTTTATTAAACTAGGG | 46 | 579 |
| 729711 | 6361 | 6378 | 1263 | 1280 | GAATCTTTATTAAACTAG | 36 | 580 |
| 729712 | 6363 | 6380 | 1265 | 1282 | GTGAATCTTTATTAAACT | 45 | 581 |
| 729719 | 6376 | 6393 | 1278 | 1295 | TGCGTGAAACTTGGTGAA | 45 | 582 |
| 1517923 | 6263 | 6280 | 1165 | 1182 | GTGGGGTCGCATGGCTGC | 62 | 583 |
| 1517925 | 6321 | 6338 | 1223 | 1240 | GGGGCGGGGACAGGGTCT | 98 | 584 |
| 1517934 | 6273 | 6290 | 1175 | 1192 | ACGGGGTGGCGTGGGGTC | 103 | 585 |
| 1517936 | 6333 | 6350 | 1235 | 1252 | AGGAGGACGGCTGGGGCG | 117 | 586 |
| 1517938 | 6329 | 6346 | 1231 | 1248 | GGACGGCTGGGGCGGGGA | 107 | 587 |
| 1517940 | 3338 | 3355 | N/A | N/A | CAAATTCCATCCCCCCAC | 102 | 588 |
| 1517958 | 3431 | 3448 | N/A | N/A | CCCATTCCCTATTTAACT | 97 | 589 |
| 1517963 | 6279 | 6296 | 1181 | 1198 | GGAGGCACGGGGTGGCGT | 101 | 590 |
| 1517974 | 3517 | 3534 | N/A | N/A | CCGGCCTCACCCCAGGTT | 99 | 591 |
| 1517975 | 3425 | 3442 | N/A | N/A | CCCTATTTAACTCCCTCC | 94 | 592 |
| 1517981 | 3271 | 3288 | N/A | N/A | TCTCCCTTCACATTCTAA | 92 | 593 |
| 1517986 | 6315 | 6332 | 1217 | 1234 | GGGACAGGGTCTCCCGCT | 74 | 594 |
| 1517987 | 6277 | 6294 | 1179 | 1196 | AGGCACGGGGTGGCGTGG | 88 | 595 |
| 1517997 | 6325 | 6342 | 1227 | 1244 | GGCTGGGGCGGGGACAGG | 100 | 596 |
| 1518001 | 6267 | 6284 | 1169 | 1186 | TGGCGTGGGGTCGCATGG | 66 | 597 |
| 1518004 | 3284 | 3301 | N/A | N/A | TCGCATTCCTCATTCTCC | 96 | 598 |
| 1518008 | 6323 | 6340 | 1225 | 1242 | CTGGGGCGGGGACAGGGT | 108 | 599 |
| 1518010 | 6335 | 6352 | 1237 | 1254 | CCAGGAGGACGGCTGGGG | 96 | 600 |
| 1518014 | 6317 | 6334 | 1219 | 1236 | CGGGGACAGGGTCTCCCG | 103 | 601 |
| 1518029 | 6269 | 6286 | 1171 | 1188 | GGTGGCGTGGGGTCGCAT | 79 | 602 |
| 1518030 | 6265 | 6282 | 1167 | 1184 | GCGTGGGGTCGCATGGCT | 49 | 603 |
| 1518039 | 6261 | 6278 | 1163 | 1180 | GGGGTCGCATGGCTGCAG | 69 | 604 |
| 1518041 | 6285 | 6302 | 1187 | 1204 | GAGGCAGGAGGCACGGGG | 99 | 605 |
| 1518046 | 6271 | 6288 | 1173 | 1190 | GGGGTGGCGTGGGGTCGC | 67 | 606 |
| 1518050 | 6283 | 6300 | 1185 | 1202 | GGCAGGAGGCACGGGGTG | 101 | 607 |
| 1518052 | 6319 | 6336 | 1221 | 1238 | GGCGGGGACAGGGTCTCC | 88 | 608 |
| 1518056 | 6327 | 6344 | 1229 | 1246 | ACGGCTGGGGCGGGGACA | 96 | 609 |
| 1518059 | 5552 | 5569 | 454 | 471 | GGCCTTCAACTCCTTCAT | 88 | 610 |
| 1518061 | 6275 | 6292 | 1177 | 1194 | GCACGGGGTGGCGTGGGG | 95 | 611 |
| 1518063 | 6281 | 6298 | 1183 | 1200 | CAGGAGGCACGGGGTGGC | 89 | 612 |
| 1518064 | 6331 | 6348 | 1233 | 1250 | GAGGACGGCTGGGGCGGG | 102 | 613 |

TABLE 10
Reduction of APOE RNA by 5-8-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 689012 | 6252 | 6269 | 1154 | 1171 | TGGCTGCAGGCTTCGGCG | 89 | 446 |
| 689013 | 6254 | 6271 | 1156 | 1173 | CATGGCTGCAGGCTTCGG | 39 | 447 |
| 689016 | 6258 | 6275 | 1160 | 1177 | GTCGCATGGCTGCAGGCT | 39 | 170 |
| 689017 | 6260 | 6277 | 1162 | 1179 | GGGTCGCATGGCTGCAGG | 64 | 449 |
| 689018 | 6286 | 6303 | 1188 | 1205 | GGAGGCAGGAGGCACGGG | 80 | 450 |
| 689019 | 6288 | 6305 | 1190 | 1207 | GCGGAGGCAGGAGGCACG | 78 | 451 |
| 689020 | 6290 | 6307 | 1192 | 1209 | GCGCGGAGGCAGGAGGCA | 90 | 452 |
| 689021 | 6292 | 6309 | 1194 | 1211 | CTGCGCGGAGGCAGGAGG | 82 | 453 |
| 689022 | 6294 | 6311 | 1196 | 1213 | GGCTGCGCGGAGGCAGGA | 71 | 454 |
| 689023 | 6296 | 6313 | 1198 | 1215 | CAGGCTGCGCGGAGGCAG | 80 | 455 |
| 689024 | 6298 | 6315 | 1200 | 1217 | TGCAGGCTGCGCGGAGGC | 85 | 456 |
| 689025 | 6300 | 6317 | 1202 | 1219 | GCTGCAGGCTGCGCGGAG | 80 | 457 |
| 689026 | 6302 | 6319 | 1204 | 1221 | CCGCTGCAGGCTGCGCGG | 97 | 458 |
| 689027 | 6304 | 6321 | 1206 | 1223 | TCCCGCTGCAGGCTGCGC | 59 | 459 |
| 689028 | 6306 | 6323 | 1208 | 1225 | TCTCCCGCTGCAGGCTGC | 63 | 460 |
| 689029 | 6308 | 6325 | 1210 | 1227 | GGTCTCCCGCTGCAGGCT | 64 | 461 |
| 689030 | 6310 | 6327 | 1212 | 1229 | AGGGTCTCCCGCTGCAGG | 73 | 462 |
| 689031 | 6312 | 6329 | 1214 | 1231 | ACAGGGTCTCCCGCTGCA | 78 | 463 |
| 689032 | 6314 | 6331 | 1216 | 1233 | GGACAGGGTCTCCCGCTG | 73 | 464 |
| 689033 | 6336 | 6353 | 1238 | 1255 | CCCAGGAGGACGGCTGGG | 102 | 465 |
| 689034 | 6338 | 6355 | 1240 | 1257 | ACCCCAGGAGGACGGCTG | 92 | 466 |
| 689035 | 6340 | 6357 | 1242 | 1259 | CCACCCCAGGAGGACGGC | 95 | 467 |
| 689036 | 6342 | 6359 | 1244 | 1261 | GTCCACCCCAGGAGGACG | 98 | 468 |
| 689037 | 6344 | 6361 | 1246 | 1263 | GGGTCCACCCCAGGAGGA | 99 | 469 |
| 689038 | 6346 | 6363 | 1248 | 1265 | TAGGGTCCACCCCAGGAG | 96 | 470 |
| 689039 | 6348 | 6365 | 1250 | 1267 | ACTAGGGTCCACCCCAGG | 90 | 471 |
| 689040 | 6352 | 6369 | 1254 | 1271 | TTAAACTAGGGTCCACCC | 83 | 472 |
| 689041 | 6354 | 6371 | 1256 | 1273 | TATTAAACTAGGGTCCAC | 57 | 473 |
| 689042 | 6356 | 6373 | 1258 | 1275 | TTTATTAAACTAGGGTCC | 81 | 474 |
| 689043 | 6358 | 6375 | 1260 | 1277 | TCTTTATTAAACTAGGGT | 29 | 475 |
| 689044 | 6360 | 6377 | 1262 | 1279 | AATCTTTATTAAACTAGG | 46 | 476 |
| 689045 | 6362 | 6379 | 1264 | 1281 | TGAATCTTTATTAAACTA | 71 | 477 |
| 689046 | 6364 | 6381 | 1266 | 1283 | GGTGAATCTTTATTAAAC | 35 | 478 |
| 689117 | 6236 | 6253 | 1138 | 1155 | CGTTCAGTGATTGTCGCT | 81 | 542 |
| 689118 | 6256 | 6273 | 1158 | 1175 | CGCATGGCTGCAGGCTTC | 32 | 543 |
| 729668 | 6238 | 6255 | 1140 | 1157 | GGCGTTCAGTGATTGTCG | 70 | 614 |
| 729670 | 6240 | 6257 | 1142 | 1159 | TCGGCGTTCAGTGATTGT | 81 | 615 |
| 729672 | 6242 | 6259 | 1144 | 1161 | CTTCGGCGTTCAGTGATT | 81 | 616 |
| 729674 | 6244 | 6261 | 1146 | 1163 | GGCTTCGGCGTTCAGTGA | 73 | 617 |
| 729676 | 6246 | 6263 | 1148 | 1165 | CAGGCTTCGGCGTTCAGT | 80 | 618 |
| 729678 | 6248 | 6265 | 1150 | 1167 | TGCAGGCTTCGGCGTTCA | 73 | 619 |
| 729680 | 6250 | 6267 | 1152 | 1169 | GCTGCAGGCTTCGGCGTT | 83 | 620 |
| 729705 | 6350 | 6367 | 1252 | 1269 | AAACTAGGGTCCACCCCA | 93 | 621 |
| 729713 | 6365 | 6382 | 1267 | 1284 | TGGTGAATCTTTATTAAA | 58 | 622 |
| 729714 | 6367 | 6384 | 1269 | 1286 | CTTGGTGAATCTTTATTA | 36 | 623 |
| 729715 | 6369 | 6386 | 1271 | 1288 | AACTTGGTGAATCTTTAT | 50 | 624 |
| 729716 | 6371 | 6388 | 1273 | 1290 | GAAACTTGGTGAATCTTT | 48 | 625 |
| 729717 | 6373 | 6390 | 1275 | 1292 | GTGAAACTTGGTGAATCT | 41 | 626 |
| 729718 | 6375 | 6392 | 1277 | 1294 | GCGTGAAACTTGGTGAAT | 42 | 627 |
| 1517913 | 6278 | 6295 | 1180 | 1197 | GAGGCACGGGGTGGCGTG | 89 | 628 |
| 1517914 | 6328 | 6345 | 1230 | 1247 | GACGGCTGGGGCGGGGAC | 101 | 629 |
| 1517915 | 6316 | 6333 | 1218 | 1235 | GGGGACAGGGTCTCCCGC | 88 | 630 |
| 1517919 | 6272 | 6289 | 1174 | 1191 | CGGGGTGGCGTGGGGTCG | 105 | 631 |
| 1517931 | 3066 | 3083 | N/A | N/A | ATGGCTTACATCCCAGTC | 107 | 632 |
| 1517933 | 6282 | 6299 | 1184 | 1201 | GCAGGAGGCACGGGGTGG | 80 | 633 |
| 1517945 | 6334 | 6351 | 1236 | 1253 | CAGGAGGACGGCTGGGGC | 114 | 634 |
| 1517947 | 3340 | 3357 | N/A | N/A | TTCAAATTCCATCCCCCC | 99 | 635 |
| 1517955 | 6274 | 6291 | 1176 | 1193 | CACGGGGTGGCGTGGGGT | 94 | 636 |
| 1517960 | 6326 | 6343 | 1228 | 1245 | CGGCTGGGGCGGGGACAG | 119 | 637 |
| 1517965 | 6318 | 6335 | 1220 | 1237 | GCGGGGACAGGGTCTCCC | 88 | 638 |
| 1517968 | 6276 | 6293 | 1178 | 1195 | GGCACGGGGTGGCGTGGG | 83 | 639 |
| 1517971 | 6280 | 6297 | 1182 | 1199 | AGGAGGCACGGGGTGGCG | 96 | 640 |
| 1517973 | 6284 | 6301 | 1186 | 1203 | AGGCAGGAGGCACGGGGT | 98 | 641 |
| 1517990 | 6262 | 6279 | 1164 | 1181 | TGGGGTCGCATGGCTGCA | 58 | 642 |
| 1518000 | 6268 | 6285 | 1170 | 1187 | GTGGCGTGGGGTCGCATG | 87 | 643 |
| 1518002 | 6330 | 6347 | 1232 | 1249 | AGGACGGCTGGGGCGGGG | 105 | 644 |
| 1518006 | 6270 | 6287 | 1172 | 1189 | GGGTGGCGTGGGGTCGCA | 61 | 645 |
| 1518011 | 6266 | 6283 | 1168 | 1185 | GGCGTGGGGTCGCATGGC | 56 | 646 |
| 1518015 | 3429 | 3446 | N/A | N/A | CATTCCCTATTTAACTCC | 89 | 647 |
| 1518019 | 6320 | 6337 | 1222 | 1239 | GGGCGGGGACAGGGTCTC | 80 | 648 |
| 1518023 | 3283 | 3300 | N/A | N/A | CGCATTCCTCATTCTCCC | 95 | 649 |
| 1518024 | 3842 | 3859 | N/A | N/A | CAACTTCTAACTCCTATC | 119 | 650 |
| 1518032 | 3307 | 3324 | N/A | N/A | CCGGTTCCATCTCAGTCC | 124 | 651 |
| 1518036 | 6264 | 6281 | 1166 | 1183 | CGTGGGGTCGCATGGCTG | 81 | 652 |
| 1518043 | 6332 | 6349 | 1234 | 1251 | GGAGGACGGCTGGGGCGG | 113 | 653 |
| 1518045 | 6322 | 6339 | 1224 | 1241 | TGGGGCGGGGACAGGGTC | 99 | 654 |
| 1518057 | 3457 | 3474 | N/A | N/A | CAGCACATTTACCAAGCC | 111 | 655 |
| 1518062 | 6324 | 6341 | 1226 | 1243 | GCTGGGGCGGGGACAGGG | 90 | 656 |

TABLE 11
Reduction of APOE RNA by 5-8-5 MOE gapmers with mixed PO/PS internucleoside linkages
at a concentration of 4000 nM in Hep3B cells plated at 5000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 426048 | 6254 | 6273 | 1156 | 1175 | CGCATGGCTGCAGGCTTCGG | 56 | 70 |
| 688818 | 5680 | 5697 | 582 | 599 | ACCAGGCGGCCGCACACG | 83 | 252 |
| 688819 | 5682 | 5699 | 584 | 601 | GCACCAGGCGGCCGCACA | 120 | 253 |
| 688820 | 5684 | 5701 | 586 | 603 | CTGCACCAGGCGGCCGCA | 124 | 254 |
| 688874 | 5806 | 5823 | 708 | 725 | CGCTTCTGCAGGTCATCG | 83 | 308 |
| 688875 | 5821 | 5838 | 723 | 740 | TGGTACACTGCCAGGCGC | 117 | 309 |
| 689014 | 6255 | 6272 | 1157 | 1174 | GCATGGCTGCAGGCTTCG | 63 | 169 |
| 689015 | 6257 | 6274 | 1159 | 1176 | TCGCATGGCTGCAGGCTT | 49 | 448 |
| 689017 | 6260 | 6277 | 1162 | 1179 | GGGTCGCATGGCTGCAGG | 87 | 449 |
| 689029 | 6308 | 6325 | 1210 | 1227 | GGTCTCCCGCTGCAGGCT | 53 | 461 |
| 689046 | 6364 | 6381 | 1266 | 1283 | GGTGAATCTTTATTAAAC | 31 | 478 |
| 689118 | 6256 | 6273 | 1158 | 1175 | CGCATGGCTGCAGGCTTC | 11 | 543 |
| 693761 | 5667 | 5684 | 569 | 586 | ACACGTCCTCCATGTCCG | 81 | 657 |
| 693762 | 5668 | 5685 | 570 | 587 | CACACGTCCTCCATGTCC | 87 | 658 |
| 693763 | 5669 | 5686 | 571 | 588 | GCACACGTCCTCCATGTC | 104 | 659 |
| 693764 | 5670 | 5687 | 572 | 589 | CGCACACGTCCTCCATGT | 74 | 660 |
| 693765 | 5671 | 5688 | 573 | 590 | CCGCACACGTCCTCCATG | 89 | 661 |
| 693766 | 5672 | 5689 | 574 | 591 | GCCGCACACGTCCTCCAT | 74 | 662 |
| 693767 | 5673 | 5690 | 575 | 592 | GGCCGCACACGTCCTCCA | 85 | 663 |
| 693768 | 5674 | 5691 | 576 | 593 | CGGCCGCACACGTCCTCC | 66 | 664 |
| 693769 | 5675 | 5692 | 577 | 594 | GCGGCCGCACACGTCCTC | 66 | 665 |
| 693770 | 5676 | 5693 | 578 | 595 | GGCGGCCGCACACGTCCT | 69 | 666 |
| 693771 | 5677 | 5694 | 579 | 596 | AGGCGGCCGCACACGTCC | 94 | 667 |
| 693772 | 5678 | 5695 | 580 | 597 | CAGGCGGCCGCACACGTC | 106 | 668 |
| 693773 | 5679 | 5696 | 581 | 598 | CCAGGCGGCCGCACACGT | 101 | 669 |
| 693774 | 5681 | 5698 | 583 | 600 | CACCAGGCGGCCGCACAC | 85 | 670 |
| 693775 | 5683 | 5700 | 585 | 602 | TGCACCAGGCGGCCGCAC | 137 | 671 |
| 693776 | 5805 | 5822 | 707 | 724 | GCTTCTGCAGGTCATCGG | 85 | 672 |
| 693777 | 5807 | 5824 | 709 | 726 | GCGCTTCTGCAGGTCATC | 111 | 673 |
| 693778 | 5808 | 5825 | 710 | 727 | GGCGCTTCTGCAGGTCAT | 134 | 674 |
| 693779 | 5809 | 5826 | 711 | 728 | AGGCGCTTCTGCAGGTCA | 86 | 675 |
| 693780 | 5810 | 5827 | 712 | 729 | CAGGCGCTTCTGCAGGTC | 93 | 676 |
| 693781 | 5811 | 5828 | 713 | 730 | CCAGGCGCTTCTGCAGGT | 113 | 677 |
| 693782 | 5812 | 5829 | 714 | 731 | GCCAGGCGCTTCTGCAGG | 88 | 678 |
| 693783 | 5813 | 5830 | 715 | 732 | TGCCAGGCGCTTCTGCAG | 100 | 679 |
| 693784 | 5814 | 5831 | 716 | 733 | CTGCCAGGCGCTTCTGCA | 105 | 680 |
| 693785 | 5815 | 5832 | 717 | 734 | ACTGCCAGGCGCTTCTGC | 91 | 681 |
| 693786 | 5816 | 5833 | 718 | 735 | CACTGCCAGGCGCTTCTG | 95 | 682 |
| 693787 | 5817 | 5834 | 719 | 736 | ACACTGCCAGGCGCTTCT | 101 | 683 |
| 693788 | 5818 | 5835 | 720 | 737 | TACACTGCCAGGCGCTTC | 81 | 684 |
| 693789 | 5819 | 5836 | 721 | 738 | GTACACTGCCAGGCGCTT | 100 | 685 |
| 693790 | 5820 | 5837 | 722 | 739 | GGTACACTGCCAGGCGCT | 81 | 686 |
| 693791 | 5822 | 5839 | 724 | 741 | CTGGTACACTGCCAGGCG | 107 | 687 |

TABLE 12
Reduction of APOE RNA by 5-8-5 MOE gapmers
with mixed PO/PS internucleoside linkages
at a concentration of 4000 nM in Hep3B
cells plated at 5000 cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 5 | 5 | 6 | 6 | | APOE | SEQ |
| pound | Start | Stop | Start | Stop | Sequence | (% | ID |
| Number | Site | Site | Site | Site | (5′ to 3′) | UTC) | NO |
| 693792 | N/A | N/A | 454 | 471 | GCACGTCCTCCAT | 98 | 688 |
| | | | | | GTCCG | | |
| 693793 | N/A | N/A | 455 | 472 | CGCACGTCCTCCA | 136 | 689 |
| | | | | | TGTCC | | |
| 693794 | N/A | N/A | 456 | 473 | GCGCACGTCCTCC | 99 | 690 |
| | | | | | ATGTC | | |
| 693795 | N/A | N/A | 457 | 474 | CGCGCACGTCCTC | 81 | 691 |
| | | | | | CATGT | | |
| 693796 | N/A | N/A | 458 | 475 | CCGCGCACGTCCT | 112 | 692 |
| | | | | | CCATG | | |
| 693797 | N/A | N/A | 459 | 476 | GCCGCGCACGTCC | 104 | 693 |
| | | | | | TCCAT | | |
| 693798 | N/A | N/A | 460 | 477 | GGCCGCGCACGTC | 77 | 694 |
| | | | | | CTCCA | | |
| 693799 | N/A | N/A | 461 | 478 | CGGCCGCGCACGT | 98 | 695 |
| | | | | | CCTCC | | |
| 693800 | N/A | N/A | 462 | 479 | GCGGCCGCGCACG | 71 | 696 |
| | | | | | TCCTC | | |
| 693801 | N/A | N/A | 463 | 480 | GGCGGCCGCGCAC | 100 | 697 |
| | | | | | GTCCT | | |
| 693802 | N/A | N/A | 464 | 481 | AGGCGGCCGCGCA | 136 | 698 |
| | | | | | CGTCC | | |
| 693803 | N/A | N/A | 465 | 482 | CAGGCGGCCGCGC | 74 | 699 |
| | | | | | ACGTC | | |
| 693804 | N/A | N/A | 466 | 483 | CCAGGCGGCCGCG | 120 | 700 |
| | | | | | CACGT | | |
| 693805 | N/A | N/A | 467 | 484 | ACCAGGCGGCCGC | 103 | 701 |
| | | | | | GCACG | | |
| 693806 | N/A | N/A | 468 | 485 | CACCAGGCGGCCG | 105 | 702 |
| | | | | | CGCAC | | |
| 693807 | N/A | N/A | 469 | 486 | GCACCAGGCGGCC | 97 | 703 |
| | | | | | GCGCA | | |
| 693808 | N/A | N/A | 470 | 487 | TGCACCAGGCGGC | 78 | 704 |
| | | | | | CGCGC | | |
| 693809 | N/A | N/A | 471 | 488 | CTGCACCAGGCGG | 83 | 705 |
| | | | | | CCGCG | | |
| 693810 | 99 | 116 | N/A | N/A | ACTTCTGCAGGTC | 104 | 706 |
| | | | | | ATCGG | | |
| 693811 | 100 | 117 | N/A | N/A | CACTTCTGCAGGT | 80 | 707 |
| | | | | | CATCG | | |
| 693812 | 101 | 118 | N/A | N/A | GCACTTCTGCAGG | 111 | 708 |
| | | | | | TCATC | | |
| 693813 | 102 | 119 | N/A | N/A | GGCACTTCTGCAG | 112 | 709 |
| | | | | | GTCAT | | |
| 693814 | 103 | 120 | N/A | N/A | AGGCACTTCTGCA | 82 | 710 |
| | | | | | GGTCA | | |
| 693815 | 104 | 121 | N/A | N/A | CAGGCACTTCTGC | 89 | 711 |
| | | | | | AGGTC | | |
| 693816 | 105 | 122 | N/A | N/A | CCAGGCACTTCTG | 102 | 712 |
| | | | | | CAGGT | | |
| 693817 | 106 | 123 | N/A | N/A | GCCAGGCACTTCT | 76 | 713 |
| | | | | | GCAGG | | |
| 693818 | 107 | 124 | N/A | N/A | TGCCAGGCACTTC | 141 | 714 |
| | | | | | TGCAG | | |
| 693819 | 108 | 125 | N/A | N/A | CTGCCAGGCACTT | 121 | 715 |
| | | | | | CTGCA | | |
| 693820 | 109 | 126 | N/A | N/A | ACTGCCAGGCACT | 98 | 716 |
| | | | | | TCTGC | | |
| 693821 | 110 | 127 | N/A | N/A | CACTGCCAGGCAC | 98 | 717 |
| | | | | | TTCTG | | |
| 693822 | 111 | 128 | N/A | N/A | ACACTGCCAGGCA | 119 | 718 |
| | | | | | CTTCT | | |
| 693823 | 112 | 129 | N/A | N/A | TACACTGCCAGGC | 87 | 719 |
| | | | | | ACTTC | | |
| 693824 | 113 | 130 | N/A | N/A | GTACACTGCCAGG | 369 | 720 |
| | | | | | CACTT | | |
| 693825 | 114 | 131 | N/A | N/A | GGTACACTGCCAG | 81 | 721 |
| | | | | | GCACT | | |
| 693826 | 115 | 132 | N/A | N/A | TGGTACACTGCCA | 86 | 722 |
| | | | | | GGCAC | | |
| 693827 | 116 | 133 | N/A | N/A | CTGGTACACTGCC | 73 | 723 |
| | | | | | AGGCA | | |

TABLE 13
Reduction of APOE RNA by 5-8-5 MOE gapmers
with mixed PO/PS internucleoside linkages
at a concentration of 4000 nM in Hep3B
cells plated at 20,000 cells per well

| | SEQ | SEQ | | | |
|---|---|---|---|---|---|
| | ID | ID | | | |
| | No: | No: | | | |
| Com- | 3 | 3 | | APOE | SEQ |
| pound | Start | Stop | Sequence | (% | ID |
| Number | Site | Site | (5′ to 3′) | UTC) | NO |
| 688667 | 35 | 52 | GGCCAGTCGGCTCCTGGG | 75 | 724 |
| 688668 | 39 | 56 | GATTGGCCAGTCGGCTCC | 100 | 725 |
| 688669 | 41 | 58 | GTGATTGGCCAGTCGGCT | 78 | 726 |
| 688670 | 43 | 60 | CTGTGATTGGCCAGTCGG | 121 | 727 |

TABLE 14
Reduction of APOE RNA by 5-8-5 MOE gapmers
with mixed PO/PS internucleoside linkages
at a concentration of 2000 nM in HepG2
cells plated at 20,000 cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 3 | 3 | 4 | 4 | Sequence | APOE | SEQ |
| pound | Start | Stop | Start | Stop | (5′ to | (% | ID |
| Number | Site | Site | Site | Site | 3′) | UTC) | NO |
| 689110 | N/A | N/A | 768 | 785 | TGCCGGCCG | 50 | 728 |
| | | | | | CACCCGGCC | | |
| 689111 | N/A | N/A | 801 | 818 | TGTAGCGGC | 53 | 729 |
| | | | | | TGGCCCGGC | | |
| 689112 | N/A | N/A | 854 | 871 | CATTTCTCTC | 84 | 730 |
| | | | | | CATCCGCG | | |
| 689113 | N/A | N/A | 866 | 883 | GGTCCCGCT | 95 | 731 |
| | | | | | GCCCATTTC | | |
| 689114 | 155 | 172 | N/A | N/A | TCTGTGGCG | 56 | 732 |
| | | | | | CAGCTCGGG | | |
| 689115 | 257 | 274 | N/A | N/A | GAGCAGCTC | 68 | 733 |
| | | | | | CTCTGCACT | | |
| 689116 | 729 | 746 | N/A | N/A | AACTTAGGG | 121 | 734 |
| | | | | | TCCACTCCT | | |

### Example 3: Effect of 5-10-5 MOE Mixed Backbone Modified Oligonucleotides on Human APOE RNA In Vitro, Single Dose

Modified oligonucleotides complementary to human APOE nucleic acid were designed and tested for their single dose effects on APOE RNA in vitro. The modified oligonucleotides were tested in a series of experiments under the culture conditions indicated in the tables below.

The modified oligonucleotides in the tables below are 5-10-5 MOE gapmers with mixed PO/PS internucleoside linkages. The gapmers are 20 nucleosides in length, wherein the central gap segment consists often 2′-β-D-deoxynucleosides, and wherein the 5′ and 3′ wing segments each consist of five 2′-MOE modified nucleosides. The sugar motif for the gapmers is (from 5′ to 3′): eeeeeddddddddddeeeee; wherein ‘d’ represents a 2′-β-D-deoxyribosyl sugar, and ‘e’ represents a 2′-MOE modified sugar moiety. The internucleoside linkage motif for the gapmers is (from 5′ to 3′): sooosssssssssssooss; wherein each ‘o’ represents a phosphodiester internucleoside linkage and each ‘s’ represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

The modified oligonucleotides were tested either in cultured Hep3B cells or in primary transgenic mouse hepatocytes as indicated in the tables below. A transgenic mouse model was obtained from Taconic (model #1549). The transgenic mouse model has a C₅₇BL/6 genetic background and expresses the human apolipoprotein E4 isoform. Primary mouse hepatocytes were isolated from transgenic mouse livers and were treated with modified oligonucleotide at a concentration of 5000 nM by free uptake at a density of 15,000 cells per well. After a treatment period of approximately 24 hours, total RNA was isolated from the cells and APOE RNA levels were measured by quantitative real-time RTPCR. In the case of the experiments carried out on cultured Hep3B cells, the cells were treated with modified oligonucleotide at a concentration of 4000 nM by electroporation at a density of 20,000 cells per well. After a treatment period of approximately 24 hours, total RNA was isolated from the cells and APOE RNA levels were measured by quantitative real-time RTPCR.

APOE RNA levels were measured by human primer-probe set RTS3073 (described herein in Example 1). APOE RNA levels were normalized to total RNA content, as measured by RIBOGREEN®. Results are presented in the tables below as percent APOE RNA relative to untreated control cells (% UTC). Each table represents results from an individual assay plate. The values marked with an “t” indicate that the modified oligonucleotide is complementary to the amplicon region of the primer probe set. Additional assays may be used to measure the potency and efficacy of the modified oligonucleotides complementary to the amplicon region. In Tables 15-20 below, Compound 689046 (described herein above) was included for reference.

TABLE 15
Reduction of APOE RNA by 5-10-5 MOE gapmers
with mixed PO/PS internucleoside linkages
at a concentration of 5000 nM in transgenic
primary mouse hepatocytes plated at 15,000
cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 689046 | 6364 | 6381 | 1266 | 1283 | GGTGAATCTTTA | 8 | 478 |
| | | | | | TTAAAC | | |
| 708022 | 6257 | 6276 | 1159 | 1178 | GGTCGCATGGCT | 10 | 71 |
| | | | | | GCAGGCTT | | |
| 708024 | 6371 | 6390 | 1273 | 1292 | GTGAAACTTGGT | 16 | 77 |
| | | | | | GAATCTTT | | |
| 708027 | 6234 | 6253 | 1136 | 1155 | CGTTCAGTGATT | 40 | 69 |
| | | | | | GTCGCTGG | | |
| 942333 | 2749 | 2768 | 1 | 20 | TATAGGGCTCCC | 111 | 735 |
| | | | | | CCTGTCCC | | |
| 942339 | 2755 | 2774 | 7 | 26 | TCCAATTATAGG | 98 | 736 |
| | | | | | GCTCCCCC | | |
| 942345 | 2762 | 2781 | 14 | 33 | AGACTTGTCCAA | 109 | 737 |
| | | | | | TTATAGGG | | |
| 942351 | 2805 | 2824 | 57 | 76 | CGTCCTTCACCT | 82 | 738 |
| | | | | | CCGCTGGG | | |
| 942357 | 3638 | 3657 | 212 | 231 | TGTGACCAGCAA | 75 | 739 |
| | | | | | CGCAGCCC | | |
| 942363 | 3644 | 3663 | 218 | 237 | CAGGAATGTGAC | 70 | 740 |
| | | | | | CAGCAACG | | |
| 942369 | 4806 | 4825 | 288 | 307 | CGGTCTGCTGGC | 84 | 741 |
| | | | | | GCAGCTCG | | |
| 942375 | 4812 | 4831 | 294 | 313 | GCCACTCGGTCT | 93 | 742 |
| | | | | | GCTGGCGC | | |
| 942381 | 4850 | 4869 | 332 | 351 | AAAGCGACCCAG | 49† | 743 |
| | | | | | TGCCAGTT | | |
| 942387 | 4857 | 4876 | 339 | 358 | AATCCCAAAAGC | 28† | 744 |
| | | | | | GACCCAGT | | |
| 942393 | 4863 | 4882 | 345 | 364 | GCAGGTAATCCC | 38† | 745 |
| | | | | | AAAAGCGA | | |
| 942398 | 4869 | 4888 | 351 | 370 | CCCAGCGCAGGT | 18† | 746 |
| | | | | | AATCCCAA | | |
| 942403 | 4876 | 4895 | 358 | 377 | GTCTGCACCCAG | 18† | 747 |
| | | | | | CGCAGGTA | | |
| 942407 | 5562 | 5581 | 464 | 483 | TTCCGATTTGTA | 79 | 748 |
| | | | | | GGCCTTCA | | |
| 942412 | 5568 | 5587 | 470 | 489 | CTCCAGTTCCGA | 71 | 749 |
| | | | | | TTTGTAGG | | |
| 942418 | 5575 | 5594 | 477 | 496 | GTTGTTCCTCCA | 76 | 750 |
| | | | | | GTTCCGAT | | |
| 942424 | 5618 | 5637 | 520 | 539 | TCCTTGGACAGC | 120 | 43 |
| | | | | | CGTGCCCG | | |
| 942430 | 5662 | 5681 | 564 | 583 | CGTCCTCCATGT | 72 | 751 |
| | | | | | CCGCGCCC | | |
| 942436 | 5686 | 5705 | 588 | 607 | GGTACTGCACCA | 130 | 752 |
| | | | | | GGCGGCCG | | |
| 942442 | 5692 | 5711 | 594 | 613 | CGCCGCGGTACT | 91 | 753 |
| | | | | | GCACCAGG | | |
| 942448 | 5718 | 5737 | 620 | 639 | GCTCTGGCCGAG | 84 | 754 |
| | | | | | CATGGCCT | | |
| 942454 | 5725 | 5744 | 627 | 646 | CCTCGGTGCTCT | 77 | 755 |
| | | | | | GGCCGAGC | | |
| 942460 | 5747 | 5766 | 649 | 668 | GAGGCGAGGCGC | 100 | 756 |
| | | | | | ACCCGCAG | | |
| 942466 | 5765 | 5784 | 667 | 686 | CGCAGCTTGCGC | 87 | 48 |
| | | | | | AGGTGGGA | | |
| 942472 | 5772 | 5791 | 674 | 693 | CCGCTTACGCAG | 53 | 757 |
| | | | | | CTTGCGCA | | |
| 942478 | 5778 | 5797 | 680 | 699 | GAGGAGCCGCTT | 91 | 758 |
| | | | | | ACGCAGCT | | |
| 942484 | 5784 | 5803 | 686 | 705 | ATCGCGGAGGAG | 91 | 759 |
| | | | | | CCGCTTAC | | |
| 942490 | 5791 | 5810 | 693 | 712 | CATCGGCATCGC | 67 | 760 |
| | | | | | GGAGGAGC | | |
| 942496 | 5797 | 5816 | 699 | 718 | GCAGGTCATCGG | 66 | 761 |
| | | | | | CATCGCGG | | |
| 942501 | 5826 | 5845 | 728 | 747 | CCCGGCCTGGTA | 81 | 762 |
| | | | | | CACTGCCA | | |
| 942507 | 5833 | 5852 | 735 | 754 | CGCGGGCCCCGG | 83 | 763 |
| | | | | | CCTGGTAC | | |
| 942513 | 5902 | 5921 | 804 | 823 | GCACGCGGCCCT | 88 | 764 |
| | | | | | GTTCCACC | | |
| 942519 | 5908 | 5927 | 810 | 829 | CGGCCCGCACGC | 95 | 765 |
| | | | | | GGCCCTGT | | |
| 942525 | 5915 | 5934 | 817 | 836 | ACAGTGGCGGCC | 137 | 766 |
| | | | | | CGCACGCG | | |
| 942531 | 5921 | 5940 | 823 | 842 | GAGCCCACAGTG | 105 | 767 |
| | | | | | GCGGCCCG | | |
| 942537 | 5948 | 5967 | 850 | 869 | CGCTCCTGTAGC | 91 | 768 |
| | | | | | GGCTGGCC | | |
| 942543 | 5978 | 5997 | 880 | 899 | GCGCGCAGCCGC | 96 | 769 |
| | | | | | TCGCCCCA | | |
| 942549 | 6010 | 6029 | 912 | 931 | CGCGGGTCCGGC | 96 | 770 |
| | | | | | TGCCCATC | | |
| 942555 | 6016 | 6035 | 918 | 937 | GGCGGTCGCGGG | 86 | 771 |
| | | | | | TCCGGCTG | | |
| 942561 | 6056 | 6075 | 958 | 977 | TTGGCGCGCACC | 96 | 772 |
| | | | | | TCCGCCAC | | |
| 942567 | 6063 | 6082 | 965 | 984 | CTCCAGCTTGGC | 83 | 773 |
| | | | | | GCGCACCT | | |
| 942573 | 6125 | 6144 | 1027 | 1046 | AACCAGCTCTTG | 76 | 774 |
| | | | | | AGGCGGGC | | |
| 942579 | 6131 | 6150 | 1033 | 1052 | GGCTCGAACCAG | 90 | 775 |
| | | | | | CTCTTGAG | | |
| 942594 | 6349 | 6368 | 1251 | 1270 | TAAACTAGGGTC | 44 | 776 |
| | | | | | CACCCCAG | | |
| 942600 | 2833 | 2852 | 85 | 104 | GCGCTTCTCACC | 101 | 777 |
| | | | | | GGCTCCTG | | |
| 942606 | 2840 | 2859 | 92 | 111 | CCCGACTGCGCT | 114 | 778 |
| | | | | | TCTCACCG | | |
| 942612 | 2846 | 2865 | 98 | 117 | CGTGCCCCCGAC | 100 | 779 |
| | | | | | TGCGCTTC | | |
| 942618 | 2855 | 2874 | 107 | 126 | GCTCATCCCCGT | 59 | 780 |
| | | | | | GCCCCCGA | | |
| 942624 | 3448 | 3467 | N/A | N/A | TTTACCAAGCCG | 108 | 781 |
| | | | | | CCCCCAAC | | |
| 942630 | 3454 | 3473 | N/A | N/A | AGCACATTTACC | 78 | 782 |
| | | | | | AAGCCGCC | | |
| 942636 | 3495 | 3514 | N/A | N/A | CCTTCCAAGCCT | 101 | 783 |
| | | | | | TGTTGCAT | | |
| 942642 | 2926 | 2945 | N/A | N/A | CGAGTAGCTCTC | 85 | 784 |
| | | | | | CTGAGACT | | |
| 942648 | 2932 | 2951 | N/A | N/A | CGACCCCGAGTA | 73 | 785 |
| | | | | | GCTCTCCT | | |
| 942654 | 2938 | 2957 | N/A | N/A | CAAGCCCGACCC | 79 | 786 |
| | | | | | CGAGTAGC | | |
| 942660 | 3067 | 3086 | N/A | N/A | GCTATGGCTTAC | 104 | 787 |
| | | | | | ATCCCAGT | | |
| 942666 | 3096 | 3115 | N/A | N/A | TAAATGATAGTG | 75 | 788 |
| | | | | | ACAACTCG | | |
| 942672 | 3171 | 3190 | N/A | N/A | AGCTACCGTGTC | 84 | 789 |
| | | | | | GCTGCCCC | | |
| 942678 | 3177 | 3196 | N/A | N/A | ACGGCTAGCTAC | 78 | 790 |
| | | | | | CGTGTCGC | | |
| 942684 | 3191 | 3210 | N/A | N/A | AAGTTCTCCAAT | 70 | 791 |
| | | | | | CGACGGCT | | |
| 942690 | 3232 | 3251 | N/A | N/A | GCGCCGTGTTCC | 108 | 792 |
| | | | | | ATTTATGA | | |
| 942696 | 3306 | 3325 | N/A | N/A | GCCGGTTCCATC | 109 | 793 |
| | | | | | TCAGTCCC | | |
| 942702 | 3313 | 3332 | N/A | N/A | CCCCACCGCCGG | 101 | 794 |
| | | | | | TTCCATCT | | |
| 942708 | 3384 | 3403 | N/A | N/A | TCCCCAGGTCGG | 87 | 795 |
| | | | | | CCTCCATA | | |
| 942714 | 3557 | 3576 | N/A | N/A | ACCGCCAGTGAG | 96 | 796 |
| | | | | | GACTCCTC | | |
| 942720 | 3572 | 3591 | N/A | N/A | AGAAACTGTCAA | 73 | 797 |
| | | | | | TCAACCGC | | |
| 942726 | 3789 | 3808 | N/A | N/A | TCAAATCGCTGT | 121 | 798 |
| | | | | | TCAGAGCC | | |
| 942732 | 4220 | 4239 | N/A | N/A | TGCCCCGTGTCT | 88 | 799 |
| | | | | | GGTATTCA | | |
| 942738 | 4290 | 4309 | N/A | N/A | GCATGTATTGAG | 111 | 800 |
| | | | | | TGTCCTTG | | |
| 942744 | 4614 | 4633 | N/A | N/A | GAGTCGGTTTAA | 80 | 801 |
| | | | | | TCACTTGG | | |
| 942750 | 4700 | 4719 | N/A | N/A | CCCCGTCCAGGC | 117 | 802 |
| | | | | | ATCTAGTA | | |
| 942756 | 4710 | 4729 | N/A | N/A | GTCCTTCTGACC | 89 | 803 |
| | | | | | CCGTCCAG | | |
| 942762 | 4738 | 4757 | N/A | N/A | GTGTGGAACAAG | 81 | 804 |
| | | | | | TTCAAGGT | | |
| 942768 | 4980 | 4999 | N/A | N/A | TATAGCCGCCCA | 108† | 805 |
| | | | | | CCAGGAGG | | |
| 942774 | 4986 | 5005 | N/A | N/A | GGGAGGTATAGC | 62† | 806 |
| | | | | | CGCCCACC | | |
| 942780 | 5158 | 5177 | N/A | N/A | CCAGGACGAGTG | 113† | 807 |
| | | | | | TGAGAGAC | | |

TABLE 16
Reduction of APOE RNA by 5-10-5 MOE gapmers
with mixed PO/PS internucleoside linkages at
a concentration of 5000 nM in transgenic
primary mouse hepatocytes plated at 15,000
cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 689046 | 6364 | 6381 | 1266 | 1283 | GGTGAATCTTTA | 6 | 478 |
| | | | | | TTAAAC | | |
| 708024 | 6371 | 6390 | 1273 | 1292 | GTGAAACTTGGT | 14 | 77 |
| | | | | | GAATCTTT | | |
| 708026 | 4879 | 4898 | 361 | 380 | AGTGTCTGCACC | 7† | 35 |
| | | | | | CAGCGCAG | | |
| 708030 | 4864 | 4883 | 346 | 365 | CGCAGGTAATCC | 58† | 32 |
| | | | | | CAAAAGCG | | |
| 942334 | 2750 | 2769 | 2 | 21 | TTATAGGGCTCC | 110 | 808 |
| | | | | | CCCTGTCC | | |
| 942340 | 2756 | 2775 | 8 | 27 | GTCCAATTATAG | 115 | 809 |
| | | | | | GGCTCCCC | | |
| 942346 | 2763 | 2782 | 15 | 34 | CAGACTTGTCCA | 91 | 810 |
| | | | | | ATTATAGG | | |
| 942352 | 3628 | 3647 | 202 | 221 | AACGCAGCCCAC | 81 | 22 |
| | | | | | AGAACCTT | | |
| 942358 | 3639 | 3658 | 213 | 232 | ATGTGACCAGCA | 52 | 811 |
| | | | | | ACGCAGCC | | |
| 942364 | 4768 | 4787 | 250 | 269 | ACCGCTTGCTCC | 58 | 812 |
| | | | | | ACCTTGGC | | |
| 942370 | 4807 | 4826 | 289 | 308 | TCGGTCTGCTGG | 120 | 813 |
| | | | | | CGCAGCTC | | |
| 942376 | 4813 | 4832 | 295 | 314 | TGCCACTCGGTC | 185 | 814 |
| | | | | | TGCTGGCG | | |
| 942382 | 4851 | 4870 | 333 | 352 | AAAAGCGACCCA | 147† | 815 |
| | | | | | GTGCCAGT | | |
| 942388 | 4858 | 4877 | 340 | 359 | TAATCCCAAAAG | 33† | 816 |
| | | | | | CGACCCAG | | |
| 942399 | 4870 | 4889 | 352 | 371 | ACCCAGCGCAGG | 9† | 817 |
| | | | | | TAATCCCA | | |
| 942408 | 5563 | 5582 | 465 | 484 | GTTCCGATTTGT | 64 | 818 |
| | | | | | AGGCCTTC | | |
| 942413 | 5569 | 5588 | 471 | 490 | CCTCCAGTTCCG | 69 | 819 |
| | | | | | ATTTGTAG | | |
| 942419 | 5576 | 5595 | 478 | 497 | AGTTGTTCCTCC | 77 | 820 |
| | | | | | AGTTCCGA | | |
| 942425 | 5620 | 5639 | 522 | 541 | GCTCCTTGGACA | 62 | 821 |
| | | | | | GCCGTGCC | | |
| 942431 | 5680 | 5699 | 582 | 601 | GCACCAGGCGGC | 92 | 822 |
| | | | | | CGCACACG | | |
| 942437 | 5687 | 5706 | 589 | 608 | CGGTACTGCACC | 80 | 823 |
| | | | | | AGGCGGCC | | |
| 942443 | 5693 | 5712 | 595 | 614 | TCGCCGCGGTAC | 107 | 824 |
| | | | | | TGCACCAG | | |
| 942449 | 5719 | 5738 | 621 | 640 | TGCTCTGGCCGA | 84 | 825 |
| | | | | | GCATGGCC | | |
| 942455 | 5726 | 5745 | 628 | 647 | TCCTCGGTGCTC | 105 | 826 |
| | | | | | TGGCCGAG | | |
| 942461 | 5748 | 5767 | 650 | 669 | GGAGGCGAGGCG | 83 | 827 |
| | | | | | CACCCGCA | | |
| 942467 | 5766 | 5785 | 668 | 687 | ACGCAGCTTGCG | 98 | 828 |
| | | | | | CAGGTGGG | | |
| 942473 | 5773 | 5792 | 675 | 694 | GCCGCTTACGCA | 78 | 829 |
| | | | | | GCTTGCGC | | |
| 942479 | 5779 | 5798 | 681 | 700 | GGAGGAGCCGCT | 75 | 830 |
| | | | | | TACGCAGC | | |
| 942485 | 5785 | 5804 | 687 | 706 | CATCGCGGAGGA | 79 | 831 |
| | | | | | GCCGCTTA | | |
| 942491 | 5792 | 5811 | 694 | 713 | TCATCGGCATCG | 106 | 832 |
| | | | | | CGGAGGAG | | |
| 942497 | 5798 | 5817 | 700 | 719 | TGCAGGTCATCG | 51 | 833 |
| | | | | | GCATCGCG | | |
| 942502 | 5828 | 5847 | 730 | 749 | GCCCCGGCCTGG | 59 | 53 |
| | | | | | TACACTGC | | |
| 942508 | 5834 | 5853 | 736 | 755 | TCGCGGGCCCCG | 79 | 834 |
| | | | | | GCCTGGTA | | |
| 942514 | 5903 | 5922 | 805 | 824 | CGCACGCGGCCC | 54 | 835 |
| | | | | | TGTTCCAC | | |
| 942520 | 5910 | 5929 | 812 | 831 | GGCGGCCCGCAC | 57 | 836 |
| | | | | | GCGGCCCT | | |
| 942526 | 5916 | 5935 | 818 | 837 | CACAGTGGCGGC | 81 | 837 |
| | | | | | CCGCACGC | | |
| 942532 | 5943 | 5962 | 845 | 864 | CTGTAGCGGCTG | 87 | 838 |
| | | | | | GCCGGCCA | | |
| 942538 | 5949 | 5968 | 851 | 870 | CCGCTCCTGTAG | 82 | 839 |
| | | | | | CGGCTGGC | | |
| 942544 | 5993 | 6012 | 895 | 914 | ATCTCCTCCATC | 74 | 840 |
| | | | | | CGCGCGCG | | |
| 942550 | 6011 | 6030 | 913 | 932 | TCGCGGGTCCGG | 82 | 841 |
| | | | | | CTGCCCAT | | |
| 942556 | 6017 | 6036 | 919 | 938 | AGGCGGTCGCGG | 91 | 842 |
| | | | | | GTCCGGCT | | |
| 942562 | 6057 | 6076 | 959 | 978 | CTTGGCGCGCAC | 86 | 843 |
| | | | | | CTCCGCCA | | |
| 942568 | 6064 | 6083 | 966 | 985 | CCTCCAGCTTGG | 58 | 844 |
| | | | | | CGCGCACC | | |
| 942574 | 6126 | 6145 | 1028 | 1047 | GAACCAGCTCTT | 79 | 845 |
| | | | | | GAGGCGGG | | |
| 942580 | 6132 | 6151 | 1034 | 1053 | GGGCTCGAACCA | 111 | 846 |
| | | | | | GCTCTTGA | | |
| 942585 | 6252 | 6271 | 1154 | 1173 | CATGGCTGCAGG | 16 | 847 |
| | | | | | CTTCGGCG | | |
| 942589 | 6301 | 6320 | 1203 | 1222 | CCCGCTGCAGGC | 84 | 848 |
| | | | | | TGCGCGGA | | |
| 942595 | 6350 | 6369 | 1252 | 1271 | TTAAACTAGGGT | 47 | 849 |
| | | | | | CCACCCCA | | |
| 942601 | 2834 | 2853 | 86 | 105 | TGCGCTTCTCAC | 64 | 850 |
| | | | | | CGGCTCCT | | |
| 942607 | 2841 | 2860 | 93 | 112 | CCCCGACTGCGC | 75 | 851 |
| | | | | | TTCTCACC | | |
| 942613 | 2847 | 2866 | 99 | 118 | CCGTGCCCCCGA | 75 | 852 |
| | | | | | CTGCGCTT | | |
| 942619 | 2856 | 2875 | 108 | 127 | AGCTCATCCCCG | 35 | 853 |
| | | | | | TGCCCCCG | | |
| 942625 | 3449 | 3468 | N/A | N/A | ATTTACCAAGCC | 94 | 854 |
| | | | | | GCCCCCAA | | |
| 942631 | 3455 | 3474 | N/A | N/A | CAGCACATTTAC | 81 | 855 |
| | | | | | CAAGCCGC | | |
| 942637 | 3497 | 3516 | N/A | N/A | AGCCTTCCAAGC | 105 | 856 |
| | | | | | CTTGTTGC | | |
| 942643 | 2927 | 2946 | N/A | N/A | CCGAGTAGCTCT | 142 | 857 |
| | | | | | CCTGAGAC | | |
| 942649 | 2933 | 2952 | N/A | N/A | CCGACCCCGAGT | 56 | 858 |
| | | | | | AGCTCTCC | | |
| 942655 | 3030 | 3049 | N/A | N/A | CCCACCTTCTAG | 84 | 859 |
| | | | | | CGGGTCGG | | |
| 942661 | 3071 | 3090 | N/A | N/A | TCCTGCTATGGC | 123 | 860 |
| | | | | | TTACATCC | | |
| 942667 | 3149 | 3168 | N/A | N/A | GCTCCCCAGTTA | 64 | 861 |
| | | | | | TGGAGATC | | |
| 942673 | 3172 | 3191 | N/A | N/A | TAGCTACCGTGT | 95 | 862 |
| | | | | | CGCTGCCC | | |
| 942679 | 3178 | 3197 | N/A | N/A | GACGGCTAGCTA | 67 | 863 |
| | | | | | CCGTGTCG | | |
| 942685 | 3192 | 3211 | N/A | N/A | AAAGTTCTCCAA | 74 | 864 |
| | | | | | TCGACGGC | | |
| 942691 | 3248 | 3267 | N/A | N/A | CCAACCTCACAG | 81 | 865 |
| | | | | | TTAAGCGC | | |
| 942697 | 3307 | 3326 | N/A | N/A | CGCCGGTTCCAT | 93 | 866 |
| | | | | | CTCAGTCC | | |
| 942703 | 3314 | 3333 | N/A | N/A | TCCCCACCGCCG | 60 | 867 |
| | | | | | GTTCCATC | | |
| 942709 | 3385 | 3404 | N/A | N/A | ATCCCCAGGTCG | 59 | 868 |
| | | | | | GCCTCCAT | | |
| 942715 | 3558 | 3577 | N/A | N/A | AACCGCCAGTGA | 61 | 869 |
| | | | | | GGACTCCT | | |
| 942721 | 3652 | 3671 | N/A | N/A | ATACCTGCCAGG | 87 | 870 |
| | | | | | AATGTGAC | | |
| 942727 | 4198 | 4217 | N/A | N/A | ATCTGCCTGCAA | 68 | 871 |
| | | | | | TGCATTAG | | |
| 942733 | 4222 | 4241 | N/A | N/A | GCTGCCCCGTGT | 78 | 872 |
| | | | | | CTGGTATT | | |
| 942739 | 4298 | 4317 | N/A | N/A | GCGGAAAAGCAT | 65 | 873 |
| | | | | | GTATTGAG | | |
| 942745 | 4616 | 4635 | N/A | N/A | GGGAGTCGGTTT | 71 | 874 |
| | | | | | AATCACTT | | |
| 942751 | 4701 | 4720 | N/A | N/A | ACCCCGTCCAGG | 175 | 875 |
| | | | | | CATCTAGT | | |
| 942757 | 4711 | 4730 | N/A | N/A | GGTCCTTCTGAC | 79 | 876 |
| | | | | | CCCGTCCA | | |
| 942763 | 4974 | 4993 | N/A | N/A | CGCCCACCAGGA | 82† | 877 |
| | | | | | GGGTCAAG | | |
| 942769 | 4981 | 5000 | N/A | N/A | GTATAGCCGCCC | 96† | 878 |
| | | | | | ACCAGGAG | | |
| 942775 | 5030 | 5049 | N/A | N/A | CCCCCCAAGACT | 121† | 879 |
| | | | | | TAGCGACA | | |
| 942781 | 5490 | 5509 | N/A | N/A | TGTCGCACAGTG | 75† | 880 |
| | | | | | GGAGGCGG | | |

TABLE 17
Reduction of APOE RNA by 5-10-5 MOE gapmers
with mixed PO/PS internucleoside linkages at
a concentration of 5000 nM in transgenic
primary mouse hepatocytes plated at 15,000
cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 689046 | 6364 | 6381 | 1266 | 1283 | GGTGAATCTTTA | 10 | 478 |
| | | | | | TTAAAC | | |
| 708023 | 5799 | 5818 | 701 | 720 | CTGCAGGTCATC | 86 | 50 |
| | | | | | GGCATCGC | | |
| 708024 | 6371 | 6390 | 1273 | 1292 | GTGAAACTTGGT | 11 | 77 |
| | | | | | GAATCTTT | | |
| 708028 | 4932 | 4951 | 414 | 433 | TCAGTTCCTGGG | 41† | 39 |
| | | | | | TGACCTGG | | |
| 942335 | 2751 | 2770 | 3 | 22 | ATTATAGGGCTC | 81 | 881 |
| | | | | | CCCCTGTC | | |
| 942341 | 2757 | 2776 | 9 | 28 | TGTCCAATTATA | 91 | 882 |
| | | | | | GGGCTCCC | | |
| 942347 | 2768 | 2787 | 20 | 39 | GATCCCAGACTT | 91 | 883 |
| | | | | | GTCCAATT | | |
| 942353 | 3630 | 3649 | 204 | 223 | GCAACGCAGCCC | 63 | 884 |
| | | | | | ACAGAACC | | |
| 942359 | 3640 | 3659 | 214 | 233 | AATGTGACCAGC | 55 | 885 |
| | | | | | AACGCAGC | | |
| 942365 | 4769 | 4788 | 251 | 270 | CACCGCTTGCTC | 88 | 886 |
| | | | | | CACCTTGG | | |
| 942371 | 4808 | 4827 | 290 | 309 | CTCGGTCTGCTG | 101 | 887 |
| | | | | | GCGCAGCT | | |
| 942377 | 4846 | 4865 | 328 | 347 | CGACCCAGTGCC | 69† | 888 |
| | | | | | AGTTCCCA | | |
| 942383 | 4853 | 4872 | 335 | 354 | CCAAAAGCGACC | 47† | 889 |
| | | | | | CAGTGCCA | | |
| 942389 | 4859 | 4878 | 341 | 360 | GTAATCCCAAAA | 43† | 890 |
| | | | | | GCGACCCA | | |
| 942394 | 4865 | 4884 | 347 | 366 | GCGCAGGTAATC | 22† | 891 |
| | | | | | CCAAAAGC | | |
| 942400 | 4871 | 4890 | 353 | 372 | CACCCAGCGCAG | 24† | 892 |
| | | | | | GTAATCCC | | |
| 942409 | 5564 | 5583 | 466 | 485 | AGTTCCGATTTG | 65 | 893 |
| | | | | | TAGGCCTT | | |
| 942414 | 5570 | 5589 | 472 | 491 | TCCTCCAGTTCC | 55 | 894 |
| | | | | | GATTTGTA | | |
| 942420 | 5577 | 5596 | 479 | 498 | CAGTTGTTCCTC | 54 | 895 |
| | | | | | CAGTTCCG | | |
| 942426 | 5621 | 5640 | 523 | 542 | AGCTCCTTGGAC | 91 | 896 |
| | | | | | AGCCGTGC | | |
| 942432 | 5681 | 5700 | 583 | 602 | TGCACCAGGCGG | 81 | 897 |
| | | | | | CCGCACAC | | |
| 942438 | 5688 | 5707 | 590 | 609 | GCGGTACTGCAC | 72 | 45 |
| | | | | | CAGGCGGC | | |
| 942444 | 5694 | 5713 | 596 | 615 | CTCGCCGCGGTA | 75 | 898 |
| | | | | | CTGCACCA | | |
| 942450 | 5720 | 5739 | 622 | 641 | GTGCTCTGGCCG | 76 | 899 |
| | | | | | AGCATGGC | | |
| 942456 | 5727 | 5746 | 629 | 648 | CTCCTCGGTGCT | 64 | 900 |
| | | | | | CTGGCCGA | | |
| 942462 | 5749 | 5768 | 651 | 670 | GGGAGGCGAGGC | 114 | 901 |
| | | | | | GCACCCGC | | |
| 942468 | 5767 | 5786 | 669 | 688 | TACGCAGCTTGC | 65 | 902 |
| | | | | | GCAGGTGG | | |
| 942474 | 5774 | 5793 | 676 | 695 | AGCCGCTTACGC | 87 | 903 |
| | | | | | AGCTTGCG | | |
| 942480 | 5780 | 5799 | 682 | 701 | CGGAGGAGCCGC | 64 | 904 |
| | | | | | TTACGCAG | | |
| 942486 | 5786 | 5805 | 688 | 707 | GCATCGCGGAGG | 100 | 905 |
| | | | | | AGCCGCTT | | |
| 942492 | 5793 | 5812 | 695 | 714 | GTCATCGGCATC | 83 | 906 |
| | | | | | GCGGAGGA | | |
| 942503 | 5829 | 5848 | 731 | 750 | GGCCCCGGCCTG | 93 | 907 |
| | | | | | GTACACTG | | |
| 942509 | 5857 | 5876 | 759 | 778 | CGCTGAGGCCGC | 81 | 908 |
| | | | | | GCTCGGCG | | |
| 942515 | 5904 | 5923 | 806 | 825 | CCGCACGCGGCC | 63 | 909 |
| | | | | | CTGTTCCA | | |
| 942521 | 5911 | 5930 | 813 | 832 | TGGCGGCCCGCA | 80 | 910 |
| | | | | | CGCGGCCC | | |
| 942527 | 5917 | 5936 | 819 | 838 | CCACAGTGGCGG | 62 | 911 |
| | | | | | CCCGCACG | | |
| 942533 | 5944 | 5963 | 846 | 865 | CCTGTAGCGGCT | 72 | 912 |
| | | | | | GGCCGGCC | | |
| 942539 | 5952 | 5971 | 854 | 873 | GGCCCGCTCCT | 94 | 913 |
| | | | | | GTAGCGGCT | | |
| 942545 | 5994 | 6013 | 896 | 915 | CATCTCCTCCAT | 64 | 914 |
| | | | | | CCGCGCGC | | |
| 942551 | 6012 | 6031 | 914 | 933 | GTCGCGGGTCCG | 80 | 915 |
| | | | | | GCTGCCCA | | |
| 942557 | 6018 | 6037 | 920 | 939 | CAGGCGGTCGCG | 90 | 916 |
| | | | | | GGTCCGGC | | |
| 942563 | 6058 | 6077 | 960 | 979 | GCTTGGCGCGCA | 70 | 917 |
| | | | | | CCTCCGCC | | |
| 942569 | 6065 | 6084 | 967 | 986 | TCCTCCAGCTTG | 82 | 918 |
| | | | | | GCGCGCAC | | |
| 942575 | 6127 | 6146 | 1029 | 1048 | CGAACCAGCTCT | 70 | 64 |
| | | | | | TGAGGCGG | | |
| 942581 | 6230 | 6249 | 1132 | 1151 | CAGTGATTGTCG | 89 | 68 |
| | | | | | CTGGGCAC | | |
| 942586 | 6253 | 6272 | 1155 | 1174 | GCATGGCTGCAG | 6 | 919 |
| | | | | | GCTTCGGC | | |
| 942590 | 6302 | 6321 | 1204 | 1223 | TCCCGCTGCAGG | 54 | 920 |
| | | | | | CTGCGCGG | | |
| 942596 | 6351 | 6370 | 1253 | 1272 | ATTAAACTAGGG | 42 | 921 |
| | | | | | TCCACCCC | | |
| 942602 | 2836 | 2855 | 88 | 107 | ACTGCGCTTCTC | 102 | 922 |
| | | | | | ACCGGCTC | | |
| 942608 | 2842 | 2861 | 94 | 113 | CCCCCGACTGCG | 82 | 923 |
| | | | | | CTTCTCAC | | |
| 942614 | 2848 | 2867 | 100 | 119 | CCCGTGCCCCCG | 137 | 924 |
| | | | | | ACTGCGCT | | |
| 942620 | 3421 | 3440 | N/A | N/A | CTATTTAACTCC | 67 | 925 |
| | | | | | CTCCTGGT | | |
| 942626 | 3450 | 3469 | N/A | N/A | CATTTACCAAGC | 74 | 926 |
| | | | | | CGCCCCCA | | |
| 942632 | 3456 | 3475 | N/A | N/A | CCAGCACATTTA | 108 | 927 |
| | | | | | CCAAGCCG | | |
| 942638 | 3498 | 3517 | N/A | N/A | TAGCCTTCCAAG | 74 | 928 |
| | | | | | CCTTGTTG | | |
| 942644 | 2928 | 2947 | N/A | N/A | CCCGAGTAGCTC | 95 | 929 |
| | | | | | TCCTGAGA | | |
| 942650 | 2934 | 2953 | N/A | N/A | CCCGACCCCGAG | 71 | 930 |
| | | | | | TAGCTCTC | | |
| 942656 | 3031 | 3050 | N/A | N/A | CCCCACCTTCTA | 75 | 931 |
| | | | | | GCGGGTCG | | |
| 942662 | 3073 | 3092 | N/A | N/A | AGTCCTGCTATG | 97 | 932 |
| | | | | | GCTTACAT | | |
| 942668 | 3167 | 3186 | N/A | N/A | ACCGTGTCGCTG | 86 | 81 |
| | | | | | CCCCTGGC | | |
| 942674 | 3173 | 3192 | N/A | N/A | CTAGCTACCGTG | 93 | 933 |
| | | | | | TCGCTGCC | | |
| 942680 | 3179 | 3198 | N/A | N/A | CGACGGCTAGCT | 124 | 934 |
| | | | | | ACCGTGTC | | |
| 942686 | 3195 | 3214 | N/A | N/A | TTTAAAGTTCTC | 78 | 935 |
| | | | | | CAATCGAC | | |
| 942692 | 3292 | 3311 | N/A | N/A | AGTCCCAGTCTC | 73 | 936 |
| | | | | | GCATTCCT | | |
| 942698 | 3308 | 3327 | N/A | N/A | CCGCCGGTTCCA | 74 | 937 |
| | | | | | TCTCAGTC | | |
| 942704 | 3375 | 3394 | N/A | N/A | CGGCCTCCATAG | 108 | 938 |
| | | | | | AAAATTCC | | |
| 942710 | 3509 | 3528 | N/A | N/A | TCACCCCAGGTT | 85 | 939 |
| | | | | | AGCCTTCC | | |
| 942716 | 3559 | 3578 | N/A | N/A | CAACCGCCAGTG | 78 | 940 |
| | | | | | AGGACTCC | | |
| 942722 | 3674 | 3693 | N/A | N/A | GAACCGAGCAAG | 72 | 941 |
| | | | | | CCCCGCCC | | |
| 942728 | 4212 | 4231 | N/A | N/A | GTCTGGTATTCA | 102 | 942 |
| | | | | | CTATCTGC | | |
| 942734 | 4223 | 4242 | N/A | N/A | AGCTGCCCCGTG | 79 | 943 |
| | | | | | TCTGGTAT | | |
| 942740 | 4303 | 4322 | N/A | N/A | GCCCAGCGGAAA | 85 | 944 |
| | | | | | AGCATGTA | | |
| 942746 | 4694 | 4713 | N/A | N/A | CCAGGCATCTAG | 99 | 945 |
| | | | | | TACCTAGG | | |
| 942752 | 4702 | 4721 | N/A | N/A | GACCCCGTCCAG | 78 | 946 |
| | | | | | GCATCTAG | | |
| 942758 | 4712 | 4731 | N/A | N/A | GGGTCCTTCTGA | 121 | 947 |
| | | | | | CCCCGTCC | | |
| 942764 | 4975 | 4994 | N/A | N/A | CCGCCCACCAGG | 87† | 948 |
| | | | | | AGGGTCAA | | |
| 942770 | 4982 | 5001 | N/A | N/A | GGTATAGCCGCC | 120† | 949 |
| | | | | | CACCAGGA | | |
| 942776 | 5031 | 5050 | N/A | N/A | GCCCCCCAAGAC | 60† | 950 |
| | | | | | TTAGCGAC | | |
| 942782 | 5491 | 5510 | N/A | N/A | GTGTCGCACAGT | 110† | 951 |
| | | | | | GGGAGGCG | | |

TABLE 18
Reduction of APOE RNA by 5-10-5 MOE gapmers
with mixed PO/PS internucleoside linkages at
a concentration of 5000 nM in transgenic
primary mouse hepatocytes plated at 15,000
cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 689046 | 6364 | 6381 | 1266 | 1283 | GGTGAATCTTTA | 3 | 478 |
| | | | | | TTAAAC | | |
| 708021 | 6254 | 6273 | 1156 | 1175 | CGCATGGCTGCA | 5 | 70 |
| | | | | | GGCTTCGG | | |
| 708024 | 6371 | 6390 | 1273 | 1292 | GTGAAACTTGGT | 9 | 77 |
| | | | | | GAATCTTT | | |
| 708025 | 5565 | 5584 | 467 | 486 | CAGTTCCGATTT | 70 | 42 |
| | | | | | GTAGGCCT | | |
| 708029 | 4872 | 4891 | 354 | 373 | GCACCCAGCGCA | 6† | 33 |
| | | | | | GGTAATCC | | |
| 942336 | 2752 | 2771 | 4 | 23 | AATTATAGGGCT | 112 | 952 |
| | | | | | CCCCCTGT | | |
| 942342 | 2759 | 2778 | 11 | 30 | CTTGTCCAATTA | 142 | 953 |
| | | | | | TAGGGCTC | | |
| 942348 | 2769 | 2788 | 21 | 40 | GGATCCCAGACT | 105 | 954 |
| | | | | | TGTCCAAT | | |
| 942354 | 3635 | 3654 | 209 | 228 | GACCAGCAACGC | 51 | 955 |
| | | | | | AGCCCACA | | |
| 942360 | 3641 | 3660 | 215 | 234 | GAATGTGACCAG | 65 | 956 |
| | | | | | CAACGCAG | | |
| 942366 | 4774 | 4793 | 256 | 275 | GTCTCCACCGCT | 114 | 957 |
| | | | | | TGCTCCAC | | |
| 942372 | 4809 | 4828 | 291 | 310 | ACTCGGTCTGCT | 113 | 958 |
| | | | | | GGCGCAGC | | |
| 942378 | 4847 | 4866 | 329 | 348 | GCGACCCAGTGC | 61† | 959 |
| | | | | | CAGTTCCC | | |
| 942384 | 4854 | 4873 | 336 | 355 | CCCAAAAGCGAC | 17† | 30 |
| | | | | | CCAGTGCC | | |
| 942390 | 4860 | 4879 | 342 | 361 | GGTAATCCCAAA | 32† | 960 |
| | | | | | AGCGACCC | | |
| 942395 | 4866 | 4885 | 348 | 367 | AGCGCAGGTAAT | 42† | 961 |
| | | | | | CCCAAAAG | | |
| 942404 | 5559 | 5578 | 461 | 480 | CGATTTGTAGGC | 136 | 41 |
| | | | | | CTTCAACT | | |
| 942415 | 5571 | 5590 | 473 | 492 | TTCCTCCAGTTC | 82 | 962 |
| | | | | | CGATTTGT | | |
| 942421 | 5581 | 5600 | 483 | 502 | GGGTCAGTTGTT | 78 | 963 |
| | | | | | CCTCCAGT | | |
| 942427 | 5657 | 5676 | 559 | 578 | TCCATGTCCGCG | 55 | 964 |
| | | | | | CCCAGCCG | | |
| 942433 | 5683 | 5702 | 585 | 604 | ACTGCACCAGGC | 56 | 965 |
| | | | | | GGCCGCAC | | |
| 942439 | 5689 | 5708 | 591 | 610 | CGCGGTACTGCA | 84 | 966 |
| | | | | | CCAGGCGG | | |
| 942445 | 5695 | 5714 | 597 | 616 | CCTCGCCGCGGT | 64 | 967 |
| | | | | | ACTGCACC | | |
| 942451 | 5722 | 5741 | 624 | 643 | CGGTGCTCTGGC | 89 | 968 |
| | | | | | CGAGCATG | | |
| 942457 | 5738 | 5757 | 640 | 659 | CGCACCCGCAGC | 63 | 969 |
| | | | | | TCCTCGGT | | |
| 942463 | 5750 | 5769 | 652 | 671 | TGGGAGGCGAGG | 123 | 970 |
| | | | | | CGCACCCG | | |
| 942469 | 5768 | 5787 | 670 | 689 | TTACGCAGCTTG | 83 | 971 |
| | | | | | CGCAGGTG | | |
| 942475 | 5775 | 5794 | 677 | 696 | GAGCCGCTTACG | 100 | 49 |
| | | | | | CAGCTTGC | | |
| 942481 | 5781 | 5800 | 683 | 702 | GCGGAGGAGCCG | 77 | 972 |
| | | | | | CTTACGCA | | |
| 942487 | 5787 | 5806 | 689 | 708 | GGCATCGCGGAG | 83 | 973 |
| | | | | | GAGCCGCT | | |
| 942493 | 5794 | 5813 | 696 | 715 | GGTCATCGGCAT | 64 | 974 |
| | | | | | CGCGGAGG | | |
| 942498 | 5800 | 5819 | 702 | 721 | TCTGCAGGTCAT | 63 | 975 |
| | | | | | CGGCATCG | | |
| 942504 | 5830 | 5849 | 732 | 751 | GGGCCCCGGCCT | 64 | 976 |
| | | | | | GGTACACT | | |
| 942510 | 5858 | 5877 | 760 | 779 | GCGCTGAGGCCG | 127 | 977 |
| | | | | | CGCTCGGC | | |
| 942516 | 5905 | 5924 | 807 | 826 | CCCGCACGCGGC | 100 | 978 |
| | | | | | CCTGTTCC | | |
| 942522 | 5912 | 5931 | 814 | 833 | GTGGCGGCCCGC | 98 | 979 |
| | | | | | ACGCGGCC | | |
| 942528 | 5918 | 5937 | 820 | 839 | CCCACAGTGGCG | 70 | 980 |
| | | | | | GCCCGCAC | | |
| 942534 | 5945 | 5964 | 847 | 866 | TCCTGTAGCGGC | 115 | 981 |
| | | | | | TGGCCGGC | | |
| 942540 | 5953 | 5972 | 855 | 874 | GGGCCCGCTCCT | 79 | 982 |
| | | | | | GTAGCGGC | | |
| 942546 | 5995 | 6014 | 897 | 916 | CCATCTCCTCCA | 55 | 983 |
| | | | | | TCCGCGCG | | |
| 942552 | 6013 | 6032 | 915 | 934 | GGTCGCGGGTCC | 45 | 984 |
| | | | | | GGCTGCCC | | |
| 942558 | 6019 | 6038 | 921 | 940 | CCAGGCGGTCGC | 100 | 985 |
| | | | | | GGGTCCGG | | |
| 942564 | 6059 | 6078 | 961 | 980 | AGCTTGGCGCGC | 50 | 986 |
| | | | | | ACCTCCGC | | |
| 942570 | 6119 | 6138 | 1021 | 1040 | CTCTTGAGGCGG | 87 | 987 |
| | | | | | GCCTGGAA | | |
| 942576 | 6128 | 6147 | 1030 | 1049 | TCGAACCAGCTC | 76 | 988 |
| | | | | | TTGAGGCG | | |
| 942582 | 6231 | 6250 | 1133 | 1152 | TCAGTGATTGTC | 53 | 989 |
| | | | | | GCTGGGCA | | |
| 942591 | 6305 | 6324 | 1207 | 1226 | GTCTCCCGCTGC | 21 | 990 |
| | | | | | AGGCTGCG | | |
| 942597 | 6353 | 6372 | 1255 | 1274 | TTATTAAACTAG | 34 | 76 |
| | | | | | GGTCCACC | | |
| 942603 | 2837 | 2856 | 89 | 108 | GACTGCGCTTCT | 86 | 991 |
| | | | | | CACCGGCT | | |
| 942609 | 2843 | 2862 | 95 | 114 | GCCCCCGACTGC | 59 | 992 |
| | | | | | GCTTCTCA | | |
| 942615 | 2850 | 2869 | 102 | 121 | TCCCCGTGCCCC | 65 | 993 |
| | | | | | CGACTGCG | | |
| 942621 | 3442 | 3461 | N/A | N/A | AAGCCGCCCCCA | 55 | 994 |
| | | | | | ACCCATTC | | |
| 942627 | 3451 | 3470 | N/A | N/A | ACATTTACCAAG | 41 | 995 |
| | | | | | CCGCCCCC | | |
| 942633 | 3474 | 3493 | N/A | N/A | ATCTGCAACAGC | 151 | 996 |
| | | | | | CTAATCCC | | |
| 942639 | 3500 | 3519 | N/A | N/A | GTTAGCCTTCCA | 99 | 997 |
| | | | | | AGCCTTGT | | |
| 942645 | 2929 | 2948 | N/A | N/A | CCCCGAGTAGCT | 87 | 998 |
| | | | | | CTCCTGAG | | |
| 942651 | 2935 | 2954 | N/A | N/A | GCCCGACCCCGA | 56 | 999 |
| | | | | | GTAGCTCT | | |
| 942657 | 3032 | 3051 | N/A | N/A | ACCCCACCTTCT | 62 | 1000 |
| | | | | | AGCGGGTC | | |
| 942663 | 3075 | 3094 | N/A | N/A | GGAGTCCTGCTA | 113 | 1001 |
| | | | | | TGGCTTAC | | |
| 942669 | 3168 | 3187 | N/A | N/A | TACCGTGTCGCT | 88 | 1002 |
| | | | | | GCCCCTGG | | |
| 942675 | 3174 | 3193 | N/A | N/A | GCTAGCTACCGT | 59 | 1003 |
| | | | | | GTCGCTGC | | |
| 942681 | 3180 | 3199 | N/A | N/A | TCGACGGCTAGC | 108 | 1004 |
| | | | | | TACCGTGT | | |
| 942687 | 3228 | 3247 | N/A | N/A | CGTGTTCCATTT | 79 | 1005 |
| | | | | | ATGAGCTA | | |
| 942693 | 3293 | 3312 | N/A | N/A | CAGTCCCAGTCT | 101 | 1006 |
| | | | | | CGCATTCC | | |
| 942699 | 3309 | 3328 | N/A | N/A | ACCGCCGGTTCC | 58 | 1007 |
| | | | | | ATCTCAGT | | |
| 942705 | 3379 | 3398 | N/A | N/A | AGGTCGGCCTCC | 70 | 1008 |
| | | | | | ATAGAAAA | | |
| 942711 | 3514 | 3533 | N/A | N/A | CGGCCTCACCCC | 70 | 1009 |
| | | | | | AGGTTAGC | | |
| 942717 | 3562 | 3581 | N/A | N/A | AATCAACCGCCA | 73 | 1010 |
| | | | | | GTGAGGAC | | |
| 942723 | 3675 | 3694 | N/A | N/A | GGAACCGAGCAA | 102 | 1011 |
| | | | | | GCCCCGCC | | |
| 942729 | 4216 | 4235 | N/A | N/A | CCGTGTCTGGTA | 83 | 1012 |
| | | | | | TTCACTAT | | |
| 942735 | 4229 | 4248 | N/A | N/A | GATCACAGCTGC | 72 | 1013 |
| | | | | | CCCGTGTC | | |
| 942741 | 4305 | 4324 | N/A | N/A | GCGCCCAGCGGA | 72 | 1014 |
| | | | | | AAAGCATG | | |
| 942747 | 4696 | 4715 | N/A | N/A | GTCCAGGCATCT | 136 | 1015 |
| | | | | | AGTACCTA | | |
| 942753 | 4703 | 4722 | N/A | N/A | TGACCCCGTCCA | 73 | 1016 |
| | | | | | GGCATCTA | | |
| 942759 | 4714 | 4733 | N/A | N/A | CAGGGTCCTTCT | 67 | 1017 |
| | | | | | GACCCCGT | | |
| 942765 | 4977 | 4996 | N/A | N/A | AGCCGCCCACCA | 44† | 1018 |
| | | | | | GGAGGGTC | | |
| 942771 | 4983 | 5002 | N/A | N/A | AGGTATAGCCGC | 128† | 1019 |
| | | | | | CCACCAGG | | |
| 942777 | 5032 | 5051 | N/A | N/A | GGCCCCCCAAGA | 61† | 1020 |
| | | | | | CTTAGCGA | | |
| 942783 | 5516 | 5535 | N/A | N/A | GCCCTGCGGCCG | 105† | 1021 |
| | | | | | AGAGGGCG | | |

TABLE 19
Reduction of APOE RNA by 5-10-5 MOE gapmers
with mixed PO/PS internucleoside linkages at
a concentration of 5000 nM in transgenic
primary mouse hepatocytes plated at 15,000
cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 689046 | 6364 | 6381 | 1266 | 1283 | GGTGAATCTT | 6 | 478 |
| | | | | | TATTAAAC | | |
| 708024 | 6371 | 6390 | 1273 | 1292 | GTGAAACTTG | 21 | 77 |
| | | | | | GTGAATCTTT | | |
| 942337 | 2753 | 2772 | 5 | 24 | CAATTATAGG | 83 | 1022 |
| | | | | | GCTCCCCCTG | | |
| 942343 | 2760 | 2779 | 12 | 31 | ACTTGTCCAA | 94 | 1023 |
| | | | | | TTATAGGGCT | | |
| 942349 | 2783 | 2802 | 35 | 54 | TGAGTAGGAC | 99 | 1024 |
| | | | | | TCAAGGATCC | | |
| 942355 | 3636 | 3655 | 210 | 229 | TGACCAGCAA | 123 | 23 |
| | | | | | CGCAGCCCAC | | |
| 942361 | 3642 | 3661 | 216 | 235 | GGAATGTGAC | 126 | 24 |
| | | | | | CAGCAACGCA | | |
| 942367 | 4804 | 4823 | 286 | 305 | GTCTGCTGGC | 105 | 1025 |
| | | | | | GCAGCTCGGG | | |
| 942373 | 4810 | 4829 | 292 | 311 | CACTCGGTCT | 100 | 1026 |
| | | | | | GCTGGCGCAG | | |
| 942379 | 4848 | 4867 | 330 | 349 | AGCGACCCAG | 45† | 1027 |
| | | | | | TGCCAGTTCC | | |
| 942385 | 4855 | 4874 | 337 | 356 | TCCCAAAAGC | 25† | 1028 |
| | | | | | GACCCAGTGC | | |
| 942391 | 4861 | 4880 | 343 | 362 | AGGTAATCCC | 26† | 31 |
| | | | | | AAAAGCGACC | | |
| 942396 | 4867 | 4886 | 349 | 368 | CAGCGCAGGT | 33† | 1029 |
| | | | | | AATCCCAAAA | | |
| 942401 | 4873 | 4892 | 355 | 374 | TGCACCCAGC | 22† | 1030 |
| | | | | | GCAGGTAATC | | |
| 942405 | 5560 | 5579 | 462 | 481 | CCGATTTGTA | 124 | 1031 |
| | | | | | GGCCTTCAAC | | |
| 942410 | 5566 | 5585 | 468 | 487 | CCAGTTCCGA | 85 | 1032 |
| | | | | | TTTGTAGGCC | | |
| 942416 | 5572 | 5591 | 474 | 493 | GTTCCTCCAG | 73 | 1033 |
| | | | | | TTCCGATTTG | | |
| 942422 | 5616 | 5635 | 518 | 537 | CTTGGACAGC | 97 | 1034 |
| | | | | | CGTGCCCGCG | | |
| 942428 | 5660 | 5679 | 562 | 581 | TCCTCCATGT | 81 | 1035 |
| | | | | | CCGCGCCCAG | | |
| 942434 | 5684 | 5703 | 586 | 605 | TACTGCACCA | 92 | 1036 |
| | | | | | GGCGGCCGCA | | |
| 942440 | 5690 | 5709 | 592 | 611 | CCGCGGTACT | 87 | 1037 |
| | | | | | GCACCAGGCG | | |
| 942446 | 5696 | 5715 | 598 | 617 | ACCTCGCCGC | 95 | 1038 |
| | | | | | GGTACTGCAC | | |
| 942452 | 5723 | 5742 | 625 | 644 | TCGGTGCTCT | 90 | 1039 |
| | | | | | GGCCGAGCAT | | |
| 942458 | 5744 | 5763 | 646 | 665 | GCGAGGCGCA | 77 | 1040 |
| | | | | | CCCGCAGCTC | | |
| 942464 | 5759 | 5778 | 661 | 680 | TTGCGCAGGT | 144 | 1041 |
| | | | | | GGGAGGCGAG | | |
| 942470 | 5769 | 5788 | 671 | 690 | CTTACGCAGC | 135 | 1042 |
| | | | | | TTGCGCAGGT | | |
| 942476 | 5776 | 5795 | 678 | 697 | GGAGCCGCTT | 94 | 1043 |
| | | | | | ACGCAGCTTG | | |
| 942482 | 5782 | 5801 | 684 | 703 | CGCGGAGGAG | 77 | 1044 |
| | | | | | CCGCTTACGC | | |
| 942488 | 5789 | 5808 | 691 | 710 | TCGGCATCGC | 99 | 1045 |
| | | | | | GGAGGAGCCG | | |
| 942494 | 5795 | 5814 | 697 | 716 | AGGTCATCGG | 90 | 1046 |
| | | | | | CATCGCGGAG | | |
| 942499 | 5824 | 5843 | 726 | 745 | CGGCCTGGTA | 62 | 1047 |
| | | | | | CACTGCCAGG | | |
| 942505 | 5831 | 5850 | 733 | 752 | CGGGCCCCGG | 89 | 1048 |
| | | | | | CCTGGTACAC | | |
| 942511 | 5900 | 5919 | 802 | 821 | ACGCGGCCCT | 98 | 1049 |
| | | | | | GTTCCACCAG | | |
| 942517 | 5906 | 5925 | 808 | 827 | GCCCGCACGC | 68 | 1050 |
| | | | | | GGCCCTGTTC | | |
| 942523 | 5913 | 5932 | 815 | 834 | AGTGGCGGCC | 125 | 1051 |
| | | | | | CGCACGCGGC | | |
| 942529 | 5919 | 5938 | 821 | 840 | GCCCACAGTG | 91 | 55 |
| | | | | | GCGGCCCGCA | | |
| 942535 | 5946 | 5965 | 848 | 867 | CTCCTGTAGC | 92 | 1052 |
| | | | | | GGCTGGCCGG | | |
| 942541 | 5954 | 5973 | 856 | 875 | TGGGCCCGCT | 82 | 1053 |
| | | | | | CCTGTAGCGG | | |
| 942547 | 6008 | 6027 | 910 | 929 | CGGGTCCGGC | 76 | 1054 |
| | | | | | TGCCCATCTC | | |
| 942553 | 6014 | 6033 | 916 | 935 | CGGTCGCGGG | 92 | 1055 |
| | | | | | TCCGGCTGCC | | |
| 942559 | 6020 | 6039 | 922 | 941 | TCCAGGCGGT | 87 | 1056 |
| | | | | | CGCGGGTCCG | | |
| 942565 | 6060 | 6079 | 962 | 981 | CAGCTTGGCG | 82 | 1057 |
| | | | | | CGCACCTCCG | | |
| 942571 | 6120 | 6139 | 1022 | 1041 | GCTCTTGAGG | 110 | 1058 |
| | | | | | CGGGCCTGGA | | |
| 942577 | 6129 | 6148 | 1031 | 1050 | CTCGAACCAG | 101 | 1059 |
| | | | | | CTCTTGAGGC | | |
| 942583 | 6232 | 6251 | 1134 | 1153 | TTCAGTGATT | 78 | 1060 |
| | | | | | GTCGCTGGGC | | |
| 942587 | 6255 | 6274 | 1157 | 1176 | TCGCATGGCT | 10 | 1061 |
| | | | | | GCAGGCTTCG | | |
| 942592 | 6341 | 6360 | 1243 | 1262 | GGTCCACCCC | 80 | 1062 |
| | | | | | AGGAGGACGG | | |
| 942598 | 6372 | 6391 | 1274 | 1293 | CGTGAAACTT | 15 | 1063 |
| | | | | | GGTGAATCTT | | |
| 942604 | 2838 | 2857 | 90 | 109 | CGACTGCGCT | 64 | 1064 |
| | | | | | TCTCACCGGC | | |
| 942610 | 2844 | 2863 | 96 | 115 | TGCCCCCGAC | 68 | 1065 |
| | | | | | TGCGCTTCTC | | |
| 942616 | 2851 | 2870 | 103 | 122 | ATCCCCGTGC | 69 | 1066 |
| | | | | | CCCCGACTGC | | |
| 942622 | 3443 | 3462 | N/A | N/A | CAAGCCGCCC | 76 | 1067 |
| | | | | | CCAACCCATT | | |
| 942628 | 3452 | 3471 | N/A | N/A | CACATTTACC | 94 | 1068 |
| | | | | | AAGCCGCCCC | | |
| 942634 | 3490 | 3509 | N/A | N/A | CAAGCCTTGT | 104 | 1069 |
| | | | | | TGCATTATCT | | |
| 942640 | 3501 | 3520 | N/A | N/A | GGTTAGCCTT | 87 | 1070 |
| | | | | | CCAAGCCTTG | | |
| 942646 | 2930 | 2949 | N/A | N/A | ACCCCGAGTA | 126 | 1071 |
| | | | | | GCTCTCCTGA | | |
| 942652 | 2936 | 2955 | N/A | N/A | AGCCCGACCC | 85 | 1072 |
| | | | | | CGAGTAGCTC | | |
| 942658 | 3033 | 3052 | N/A | N/A | CACCCCACCT | 92 | 1073 |
| | | | | | TCTAGCGGGT | | |
| 942664 | 3076 | 3095 | N/A | N/A | TGGAGTCCTG | 90 | 1074 |
| | | | | | CTATGGCTTA | | |
| 942670 | 3169 | 3188 | N/A | N/A | CTACCGTGTC | 64 | 1075 |
| | | | | | GCTGCCCCTG | | |
| 942676 | 3175 | 3194 | N/A | N/A | GGCTAGCTAC | 73 | 1076 |
| | | | | | CGTGTCGCTG | | |
| 942682 | 3184 | 3203 | N/A | N/A | CCAATCGACG | 97 | 1077 |
| | | | | | GCTAGCTACC | | |
| 942688 | 3230 | 3249 | N/A | N/A | GCCGTGTTCC | 69 | 1078 |
| | | | | | ATTTATGAGC | | |
| 942694 | 3296 | 3315 | N/A | N/A | TCTCAGTCCC | 89 | 1079 |
| | | | | | AGTCTCGCAT | | |
| 942700 | 3310 | 3329 | N/A | N/A | CACCGCCGGT | 76 | 1080 |
| | | | | | TCCATCTCAG | | |
| 942706 | 3381 | 3400 | N/A | N/A | CCAGGTCGGC | 68 | 1081 |
| | | | | | CTCCATAGAA | | |
| 942712 | 3515 | 3534 | N/A | N/A | CCGGCCTCAC | 87 | 1082 |
| | | | | | CCCAGGTTAG | | |
| 942718 | 3564 | 3583 | N/A | N/A | TCAATCAACC | 102 | 1083 |
| | | | | | GCCAGTGAGG | | |
| 942724 | 3676 | 3695 | N/A | N/A | GGGAACCGAG | 67 | 1084 |
| | | | | | CAAGCCCCGC | | |
| 942730 | 4218 | 4237 | N/A | N/A | CCCCGTGTCT | 98 | 1085 |
| | | | | | GGTATTCACT | | |
| 942736 | 4230 | 4249 | N/A | N/A | AGATCACAGC | 117 | 1086 |
| | | | | | TGCCCCGTGT | | |
| 942742 | 4610 | 4629 | N/A | N/A | CGGTTTAATC | 91 | 1087 |
| | | | | | ACTTGGAAAG | | |
| 942748 | 4698 | 4717 | N/A | N/A | CCGTCCAGGC | 153 | 1088 |
| | | | | | ATCTAGTACC | | |
| 942754 | 4705 | 4724 | N/A | N/A | TCTGACCCCG | 95 | 1089 |
| | | | | | TCCAGGCATC | | |
| 942760 | 4715 | 4734 | N/A | N/A | TCAGGGTCCT | 87 | 1090 |
| | | | | | TCTGACCCCG | | |
| 942766 | 4978 | 4997 | N/A | N/A | TAGCCGCCCA | 87† | 1091 |
| | | | | | CCAGGAGGGT | | |
| 942772 | 4984 | 5003 | N/A | N/A | GAGGTATAGC | 91† | 1092 |
| | | | | | CGCCCACCAG | | |
| 942778 | 5033 | 5052 | N/A | N/A | AGGCCCCCCA | 65† | 1093 |
| | | | | | AGACTTAGCG | | |
| 942784 | 5517 | 5536 | N/A | N/A | CGCCCTGCGG | 98† | 1094 |
| | | | | | CCGAGAGGGC | | |

TABLE 20
Reduction of APOE RNA by 5-10-5 MOE gapmers
with mixed PO/PS internucleoside linkages at
a concentration of 5000 nM in transgenic
primary mouse hepatocytes plated at 15,000
cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 689046 | 6364 | 6381 | 1266 | 1283 | GGTGAATCTT | 9 | 478 |
| | | | | | TATTAAAC | | |
| 708024 | 6371 | 6390 | 1273 | 1292 | GTGAAACTTG | 24 | 77 |
| | | | | | GTGAATCTTT | | |
| 942338 | 2754 | 2773 | 6 | 25 | CCAATTATAG | 75 | 1095 |
| | | | | | GGCTCCCCCT | | |
| 942344 | 2761 | 2780 | 13 | 32 | GACTTGTCCA | 69 | 1096 |
| | | | | | ATTATAGGGC | | |
| 942350 | 2784 | 2803 | 36 | 55 | CTGAGTAGGA | 99 | 1097 |
| | | | | | CTCAAGGATC | | |
| 942356 | 3637 | 3656 | 211 | 230 | GTGACCAGCA | 103 | 1098 |
| | | | | | ACGCAGCCCA | | |
| 942362 | 3643 | 3662 | 217 | 236 | AGGAATGTGA | 71 | 1099 |
| | | | | | CCAGCAACGC | | |
| 942368 | 4805 | 4824 | 287 | 306 | GGTCTGCTGG | 82 | 1100 |
| | | | | | CGCAGCTCGG | | |
| 942374 | 4811 | 4830 | 293 | 312 | CCACTCGGTC | 70 | 1101 |
| | | | | | TGCTGGCGCA | | |
| 942380 | 4849 | 4868 | 331 | 350 | AAGCGACCCA | 43† | 1102 |
| | | | | | GTGCCAGTTC | | |
| 942386 | 4856 | 4875 | 338 | 357 | ATCCCAAAAG | 31† | 1103 |
| | | | | | CGACCCAGTG | | |
| 942392 | 4862 | 4881 | 344 | 363 | CAGGTAATCC | 39† | 1104 |
| | | | | | CAAAAGCGAC | | |
| 942397 | 4868 | 4887 | 350 | 369 | CCAGCGCAGG | 12† | 1105 |
| | | | | | TAATCCCAAA | | |
| 942402 | 4875 | 4894 | 357 | 376 | TCTGCACCCA | 22† | 34 |
| | | | | | GCGCAGGTAA | | |
| 942406 | 5561 | 5580 | 463 | 482 | TCCGATTTGT | 81 | 1106 |
| | | | | | AGGCCTTCAA | | |
| 942411 | 5567 | 5586 | 469 | 488 | TCCAGTTCCG | 95 | 1107 |
| | | | | | ATTTGTAGGC | | |
| 942417 | 5574 | 5593 | 476 | 495 | TTGTTCCTCC | 79 | 1108 |
| | | | | | AGTTCCGATT | | |
| 942423 | 5617 | 5636 | 519 | 538 | CCTTGGACAG | 59 | 1109 |
| | | | | | CCGTGCCCGC | | |
| 942429 | 5661 | 5680 | 563 | 582 | GTCCTCCATG | 87 | 1110 |
| | | | | | TCCGCGCCCA | | |
| 942435 | 5685 | 5704 | 587 | 606 | GTACTGCACC | 100 | 1111 |
| | | | | | AGGCGGCCGC | | |
| 942441 | 5691 | 5710 | 593 | 612 | GCCGCGGTAC | 79 | 46 |
| | | | | | TGCACCAGGC | | |
| 942447 | 5716 | 5735 | 618 | 637 | TCTGGCCGAG | 130 | 1112 |
| | | | | | CATGGCCTGC | | |
| 942453 | 5724 | 5743 | 626 | 645 | CTCGGTGCTC | 72 | 1113 |
| | | | | | TGGCCGAGCA | | |
| 942459 | 5746 | 5765 | 648 | 667 | AGGCGAGGCG | 81 | 1114 |
| | | | | | CACCCGCAGC | | |
| 942465 | 5760 | 5779 | 662 | 681 | CTTGCGCAGG | 86 | 1115 |
| | | | | | TGGGAGGCGA | | |
| 942471 | 5771 | 5790 | 673 | 692 | CGCTTACGCA | 87 | 1116 |
| | | | | | GCTTGCGCAG | | |
| 942477 | 5777 | 5796 | 679 | 698 | AGGAGCCGCT | 95 | 1117 |
| | | | | | TACGCAGCTT | | |
| 942483 | 5783 | 5802 | 685 | 704 | TCGCGGAGGA | 72 | 1118 |
| | | | | | GCCGCTTACG | | |
| 942489 | 5790 | 5809 | 692 | 711 | ATCGGCATCG | 118 | 1119 |
| | | | | | CGGAGGAGCC | | |
| 942495 | 5796 | 5815 | 698 | 717 | CAGGTCATCG | 76 | 1120 |
| | | | | | GCATCGCGGA | | |
| 942500 | 5825 | 5844 | 727 | 746 | CCGGCCTGGT | 97 | 52 |
| | | | | | ACACTGCCAG | | |
| 942506 | 5832 | 5851 | 734 | 753 | GCGGGCCCCG | 87 | 54 |
| | | | | | GCCTGGTACA | | |
| 942512 | 5901 | 5920 | 803 | 822 | CACGCGGCCC | 54 | 1121 |
| | | | | | TGTTCCACCA | | |
| 942518 | 5907 | 5926 | 809 | 828 | GGCCCGCACG | 87 | 1122 |
| | | | | | CGGCCCTGTT | | |
| 942524 | 5914 | 5933 | 816 | 835 | CAGTGGCGGC | 81 | 1123 |
| | | | | | CCGCACGCGG | | |
| 942530 | 5920 | 5939 | 822 | 841 | AGCCCACAGT | 122 | 1124 |
| | | | | | GGCGGCCCGC | | |
| 942536 | 5947 | 5966 | 849 | 868 | GCTCCTGTAG | 83 | 1125 |
| | | | | | CGGCTGGCCG | | |
| 942542 | 5955 | 5974 | 857 | 876 | CTGGGCCCGC | 65 | 1126 |
| | | | | | TCCTGTAGCG | | |
| 942548 | 6009 | 6028 | 911 | 930 | GCGGGTCCGG | 73 | 60 |
| | | | | | CTGCCCATCT | | |
| 942554 | 6015 | 6034 | 917 | 936 | GCGGTCGCGG | 83 | 1127 |
| | | | | | GTCCGGCTGC | | |
| 942560 | 6021 | 6040 | 923 | 942 | GTCCAGGCGG | 69 | 1128 |
| | | | | | TCGCGGGTCC | | |
| 942566 | 6062 | 6081 | 964 | 983 | TCCAGCTTGG | 68 | 61 |
| | | | | | CGCGCACCTC | | |
| 942572 | 6124 | 6143 | 1026 | 1045 | ACCAGCTCTT | 82 | 1129 |
| | | | | | GAGGCGGGCC | | |
| 942578 | 6130 | 6149 | 1032 | 1051 | GCTCGAACCA | 97 | 1130 |
| | | | | | GCTCTTGAGG | | |
| 942584 | 6233 | 6252 | 1135 | 1154 | GTTCAGTGAT | 71 | 1131 |
| | | | | | TGTCGCTGGG | | |
| 942588 | 6256 | 6275 | 1158 | 1177 | GTCGCATGGC | 9 | 1132 |
| | | | | | TGCAGGCTTC | | |
| 942593 | 6347 | 6366 | 1249 | 1268 | AACTAGGGTC | 52 | 75 |
| | | | | | CACCCCAGGA | | |
| 942599 | 2832 | 2851 | 84 | 103 | CGCTTCTCAC | 80 | 1133 |
| | | | | | CGGCTCCTGG | | |
| 942605 | 2839 | 2858 | 91 | 110 | CCGACTGCGC | 103 | 1134 |
| | | | | | TTCTCACCGG | | |
| 942611 | 2845 | 2864 | 97 | 116 | GTGCCCCCGA | 86 | 1135 |
| | | | | | CTGCGCTTCT | | |
| 942617 | 2853 | 2872 | 105 | 124 | TCATCCCCGT | 52 | 1136 |
| | | | | | GCCCCCGACT | | |
| 942623 | 3447 | 3466 | N/A | N/A | TTACCAAGCC | 96 | 1137 |
| | | | | | GCCCCCAACC | | |
| 942629 | 3453 | 3472 | N/A | N/A | GCACATTTAC | 65 | 1138 |
| | | | | | CAAGCCGCCC | | |
| 942635 | 3491 | 3510 | N/A | N/A | CCAAGCCTTG | 76 | 1139 |
| | | | | | TTGCATTATC | | |
| 942641 | 2923 | 2942 | N/A | N/A | GTAGCTCTCC | 117 | 1140 |
| | | | | | TGAGACTACC | | |
| 942647 | 2931 | 2950 | N/A | N/A | GACCCCGAGT | 98 | 1141 |
| | | | | | AGCTCTCCTG | | |
| 942653 | 2937 | 2956 | N/A | N/A | AAGCCCGACC | 75 | 1142 |
| | | | | | CCGAGTAGCT | | |
| 942659 | 3065 | 3084 | N/A | N/A | TATGGCTTAC | 96 | 1143 |
| | | | | | ATCCCAGTCC | | |
| 942665 | 3078 | 3097 | N/A | N/A | CGTGGAGTCC | 66 | 1144 |
| | | | | | TGCTATGGCT | | |
| 942671 | 3170 | 3189 | N/A | N/A | GCTACCGTGT | 73 | 1145 |
| | | | | | CGCTGCCCCT | | |
| 942677 | 3176 | 3195 | N/A | N/A | CGGCTAGCTA | 99 | 1146 |
| | | | | | CCGTGTCGCT | | |
| 942683 | 3187 | 3206 | N/A | N/A | TCTCCAATCG | 108 | 1147 |
| | | | | | ACGGCTAGCT | | |
| 942689 | 3231 | 3250 | N/A | N/A | CGCCGTGTTC | 68 | 1148 |
| | | | | | CATTTATGAG | | |
| 942695 | 3305 | 3324 | N/A | N/A | CCGGTTCCAT | 79 | 1149 |
| | | | | | CTCAGTCCCA | | |
| 942701 | 3311 | 3330 | N/A | N/A | CCACCGCCGG | 81 | 1150 |
| | | | | | TTCCATCTCA | | |
| 942707 | 3383 | 3402 | N/A | N/A | CCCCAGGTCG | 78 | 1151 |
| | | | | | GCCTCCATAG | | |
| 942713 | 3556 | 3575 | N/A | N/A | CCGCCAGTGA | 77 | 1152 |
| | | | | | GGACTCCTCC | | |
| 942719 | 3567 | 3586 | N/A | N/A | CTGTCAATCA | 88 | 1153 |
| | | | | | ACCGCCAGTG | | |
| 942725 | 3785 | 3804 | N/A | N/A | ATCGCTGTTC | 92 | 1154 |
| | | | | | AGAGCCAGGA | | |
| 942731 | 4219 | 4238 | N/A | N/A | GCCCCGTGTC | 73 | 1155 |
| | | | | | TGGTATTCAC | | |
| 942737 | 4233 | 4252 | N/A | N/A | TAAAGATCAC | 82 | 1156 |
| | | | | | AGCTGCCCCG | | |
| 942743 | 4612 | 4631 | N/A | N/A | GTCGGTTTAA | 73 | 1157 |
| | | | | | TCACTTGGAA | | |
| 942749 | 4699 | 4718 | N/A | N/A | CCCGTCCAGG | 74 | 1158 |
| | | | | | CATCTAGTAC | | |
| 942755 | 4706 | 4725 | N/A | N/A | TTCTGACCCC | 108 | 1159 |
| | | | | | GTCCAGGCAT | | |
| 942761 | 4733 | 4752 | N/A | N/A | GAACAAGTTC | 98 | 1160 |
| | | | | | AAGGTGGGTC | | |
| 942767 | 4979 | 4998 | N/A | N/A | ATAGCCGCCC | 75† | 1161 |
| | | | | | ACCAGGAGGG | | |
| 942773 | 4985 | 5004 | N/A | N/A | GGAGGTATAG | 87† | 1162 |
| | | | | | CCGCCCACCA | | |
| 942779 | 5152 | 5171 | N/A | N/A | CGAGTGTGAG | 87† | 1163 |
| | | | | | AGACTGAGTC | | |
| 942785 | 5518 | 5537 | N/A | N/A | GCGCCCTGCG | 75† | 1164 |
| | | | | | GCCGAGAGGG | | |

TABLE 21
Reduction of APOE RNA by 5-10-5 MOE gapmers
with mixed PO/PS internucleoside linkages at
a concentration of 4000 nM in Hep3B cells
plated at 20,000 cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 942584 | 6233 | 6252 | 1135 | 1154 | GTTCAGTGAT | 43 | 1131 |
| | | | | | TGTCGCTGGG | | |
| 942586 | 6253 | 6272 | 1155 | 1174 | GCATGGCTGC | 14 | 919 |
| | | | | | AGGCTTCGGC | | |
| 942597 | 6353 | 6372 | 1255 | 1274 | TTATTAAACT | 48 | 76 |
| | | | | | AGGGTCCACC | | |
| 1517142 | 3368 | 3387 | N/A | N/A | CATAGAAAAT | 76 | 1165 |
| | | | | | TCCATCTTCC | | |
| 1517148 | 3219 | 3238 | N/A | N/A | TTTATGAGCT | 60 | 1166 |
| | | | | | AATTCAGTCC | | |
| 1517167 | 4819 | 4838 | 301 | 320 | CCGCTCTGCC | 63 | 1167 |
| | | | | | ACTCGGTCTG | | |
| 1517173 | 4210 | 4229 | N/A | N/A | CTGGTATTCA | 82 | 1168 |
| | | | | | CTATCTGCCT | | |
| 1517183 | 3446 | 3465 | N/A | N/A | TACCAAGCCG | 62 | 1169 |
| | | | | | CCCCCAACCC | | |
| 1517210 | 3005 | 3024 | N/A | N/A | CTCTGCTGCC | 89 | 1170 |
| | | | | | CAGCCCTTCC | | |
| 1517234 | 4782 | 4801 | 264 | 283 | CCGGCTCTGT | 59 | 1171 |
| | | | | | CTCCACCGCT | | |
| 1517246 | 3062 | 3081 | N/A | N/A | GGCTTACATC | 68 | 1172 |
| | | | | | CCAGTCCAGC | | |
| 1517253 | 5044 | 5063 | N/A | N/A | AGCAGAGACC | 76† | 1173 |
| | | | | | CAGGCCCCCC | | |
| 1517272 | 3850 | 3869 | N/A | N/A | AACAACAAAA | 81 | 1174 |
| | | | | | CAACTTCTAA | | |
| 1517280 | 6173 | 6192 | 1075 | 1094 | TCCACCAGCC | 82 | 1175 |
| | | | | | CGGCCCACTG | | |
| 1517287 | 3629 | 3648 | 203 | 222 | CAACGCAGCC | 62 | 1176 |
| | | | | | CACAGAACCT | | |
| 1517293 | 3398 | 3417 | N/A | N/A | CTCTTATCTC | 67 | 1177 |
| | | | | | CCCATCCCCA | | |
| 1517308 | 3295 | 3314 | N/A | N/A | CTCAGTCCCA | 62 | 1178 |
| | | | | | GTCTCGCATT | | |
| 1517316 | 4613 | 4632 | N/A | N/A | AGTCGGTTTA | 73 | 1179 |
| | | | | | ATCACTTGGA | | |
| 1517317 | 3434 | 3453 | N/A | N/A | CCCAACCCAT | 79 | 1180 |
| | | | | | TCCCTATTTA | | |
| 1517318 | 3254 | 3273 | N/A | N/A | TAAGCTCCAA | 68 | 1181 |
| | | | | | CCTCACAGTT | | |
| 1517322 | 4594 | 4613 | N/A | N/A | AAAGCATTAT | 92 | 1182 |
| | | | | | GTATTTATAA | | |
| 1517333 | 6213 | 6232 | 1115 | 1134 | CACAGGGGCG | 88 | 1183 |
| | | | | | GCGCTGGTGC | | |
| 1517341 | 4766 | 4785 | 248 | 267 | CGCTTGCTCC | 106 | 1184 |
| | | | | | ACCTTGGCCT | | |
| 1517343 | 3466 | 3485 | N/A | N/A | CAGCCTAATC | 79 | 1185 |
| | | | | | CCAGCACATT | | |
| 1517345 | 4887 | 4906 | 369 | 388 | GCTCAGACAG | 38† | 1186 |
| | | | | | TGTCTGCACC | | |
| 1517347 | 3527 | 3546 | N/A | N/A | CCCGGCCCCA | 110 | 1187 |
| | | | | | ACCCGGCCTC | | |
| 1517356 | 3266 | 3285 | N/A | N/A | CCCTTCACAT | 84 | 1188 |
| | | | | | TCTAAGCTCC | | |
| 1517365 | 3617 | 3636 | 191 | 210 | CAGAACCTTC | 52 | 1189 |
| | | | | | ATCTTCCTGC | | |
| 1517378 | 3410 | 3429 | N/A | N/A | CCTCCTGGTC | 68 | 1190 |
| | | | | | TTCTCTTATC | | |
| 1517392 | 3341 | 3360 | N/A | N/A | GGGTTCAAAT | 72 | 1191 |
| | | | | | TCCATCCCCC | | |
| 1517396 | 6333 | 6352 | 1235 | 1254 | CCAGGAGGAC | 77 | 1192 |
| | | | | | GGCTGGGGCG | | |
| 1517427 | 6363 | 6382 | 1265 | 1284 | TGGTGAATCT | 18 | 1193 |
| | | | | | TTATTAAACT | | |
| 1517441 | 5009 | 5028 | N/A | N/A | GGGCAGAATG | 85† | 1194 |
| | | | | | AAACCTGGAC | | |
| 1517447 | 3513 | 3532 | N/A | N/A | GGCCTCACCC | 84 | 1195 |
| | | | | | CAGGTTAGCC | | |
| 1517456 | 3319 | 3338 | N/A | N/A | CCCCCTCCCC | 67 | 1196 |
| | | | | | ACCGCCGGTT | | |
| 1517459 | 3424 | 3443 | N/A | N/A | TCCCTATTTA | 90 | 1197 |
| | | | | | ACTCCCTCCT | | |
| 1517464 | 3667 | 3686 | N/A | N/A | GCAAGCCCCG | 71 | 1198 |
| | | | | | CCCCCATACC | | |
| 1517483 | 3105 | 3124 | N/A | N/A | GGTGCTCGAT | 82 | 1199 |
| | | | | | AAATGATAGT | | |
| 1517492 | 4540 | 4559 | N/A | N/A | AGCGGCCTGA | 94 | 1200 |
| | | | | | ACATGGTTCA | | |
| 1517493 | 3768 | 3787 | N/A | N/A | GGAAGCAGCA | 65 | 1201 |
| | | | | | CAGAAGCCTC | | |
| 1517510 | 3571 | 3590 | N/A | N/A | GAAACTGTCA | 80 | 1202 |
| | | | | | ATCAACCGCC | | |
| 1517512 | 2881 | 2900 | 133 | 152 | TCCCAGCTCT | 84 | 1203 |
| | | | | | TTCTAGAGGC | | |
| 1517531 | 4707 | 4726 | N/A | N/A | CTTCTGACCC | 81 | 1204 |
| | | | | | CGTCCAGGCA | | |
| 1517535 | 6263 | 6282 | 1165 | 1184 | GCGTGGGGTC | 23 | 1205 |
| | | | | | GCATGGCTGC | | |
| 1517548 | 3206 | 3225 | N/A | N/A | TCAGTCCTCA | 81 | 1206 |
| | | | | | TTTTAAAGTT | | |
| 1517555 | 6303 | 6322 | 1205 | 1224 | CTCCCGCTGC | 33 | 1207 |
| | | | | | AGGCTGCGCG | | |
| 1517556 | 3551 | 3570 | N/A | N/A | AGTGAGGACT | 76 | 1208 |
| | | | | | CCTCCCACCC | | |
| 1517558 | 6153 | 6172 | 1055 | 1074 | GCGCTGCATG | 55 | 66 |
| | | | | | TCTTCCACCA | | |
| 1517560 | 6273 | 6292 | 1175 | 1194 | GCACGGGGTG | 59 | 1209 |
| | | | | | GCGTGGGGTC | | |
| 1517561 | 3050 | 3069 | N/A | N/A | AGTCCAGCTG | 69 | 1210 |
| | | | | | CTCTCCCCAC | | |
| 1517581 | 5056 | 5075 | N/A | N/A | GAAGCTAGAA | 82† | 1211 |
| | | | | | CCAGCAGAGA | | |
| 1517601 | 2864 | 2883 | 116 | 135 | GGCCCCTGAG | 82 | 1212 |
| | | | | | CTCATCCCCG | | |
| 1517603 | 2988 | 3007 | N/A | N/A | TCCCAGGTCC | 91 | 1213 |
| | | | | | AGTCCCCTGC | | |
| 1517612 | 3193 | 3212 | N/A | N/A | TAAAGTTCTC | 65 | 1214 |
| | | | | | CAATCGACGG | | |
| 1517617 | 6133 | 6152 | 1035 | 1054 | GGGGCTCGAA | 81 | 1215 |
| | | | | | CCAGCTCTTG | | |
| 1517646 | 2962 | 2981 | N/A | N/A | CCTCACCCCC | 117 | 1216 |
| | | | | | GCTCCTCCTC | | |
| 1517648 | 6243 | 6262 | 1145 | 1164 | AGGCTTCGGC | 63 | 1217 |
| | | | | | GTTCAGTGAT | | |
| 1517660 | 3380 | 3399 | N/A | N/A | CAGGTCGGCC | 83 | 1218 |
| | | | | | TCCATAGAAA | | |
| 1517677 | 3283 | 3302 | N/A | N/A | CTCGCATTCC | 52 | 1219 |
| | | | | | TCATTCTCCC | | |
| 1517688 | 6193 | 6212 | 1095 | 1114 | CCACGGCAGC | 64 | 1220 |
| | | | | | CTGCACCTTC | | |
| 1517714 | 4194 | 4213 | N/A | N/A | GCCTGCAATG | 78 | 1221 |
| | | | | | CATTAGAAAC | | |
| 1517743 | 4242 | 4261 | N/A | N/A | GATGGAGAAT | 81 | 1222 |
| | | | | | AAAGATCACA | | |
| 1517751 | 3494 | 3513 | N/A | N/A | CTTCCAAGCC | 85 | 1223 |
| | | | | | TTGTTGCATT | | |
| 1517755 | 6313 | 6332 | 1215 | 1234 | GGGACAGGGT | 46 | 1224 |
| | | | | | CTCCCGCTGC | | |
| 1517766 | 6343 | 6362 | 1245 | 1264 | AGGGTCCACC | 87 | 74 |
| | | | | | CCAGGAGGAC | | |
| 1517773 | 3840 | 3859 | N/A | N/A | CAACTTCTAA | 91 | 1225 |
| | | | | | CTCCTATCTC | | |
| 1517790 | 6283 | 6302 | 1185 | 1204 | GAGGCAGGAG | 86 | 1226 |
| | | | | | GCACGGGGTG | | |
| 1517796 | 3658 | 3677 | N/A | N/A | GCCCCCATAC | 91 | 1227 |
| | | | | | CTGCCAGGAA | | |
| 1517814 | 4909 | 4928 | 391 | 410 | CTGAGCAGCT | 44† | 1228 |
| | | | | | CCTCCTGCAC | | |
| 1517817 | 6293 | 6312 | 1195 | 1214 | AGGCTGCGCG | 45 | 1229 |
| | | | | | GAGGCAGGAG | | |
| 1517834 | 2972 | 2991 | N/A | N/A | CTGCTGCTTG | 76 | 1230 |
| | | | | | CCTCACCCCC | | |
| 1517849 | 6374 | 6393 | 1276 | 1295 | TGCGTGAAAC | 19 | 1231 |
| | | | | | TTGGTGAATC | | |
| 1517853 | 6364 | 6383 | 1266 | 1285 | TTGGTGAATC | 22 | 1232 |
| | | | | | TTTATTAAAC | | |
| 1517860 | 2948 | 2967 | N/A | N/A | CTCCTCTCCC | 79 | 1233 |
| | | | | | CAAGCCCGAC | | |
| 1517894 | 6323 | 6342 | 1225 | 1244 | GGCTGGGGCG | 116 | 1234 |
| | | | | | GGGACAGGGT | | |
| 1517897 | 2919 | 2938 | N/A | N/A | CTCTCCTGAG | 63 | 1235 |
| | | | | | ACTACCTGGA | | |
| 1517902 | 4184 | 4203 | N/A | N/A | CATTAGAAAC | 104 | 1236 |
| | | | | | CTCTAACTCC | | |
| 1517905 | 3477 | 3496 | N/A | N/A | ATTATCTGCA | 75 | 1237 |
| | | | | | ACAGCCTAAT | | |

TABLE 22
Reduction of APOE RNA by 5-10-5 MOE gapmers
with mixed PO/PS internucleoside linkages at
a concentration of 4000 nM in Hep3B cells
plated at 20,000 cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 942582 | 6231 | 6250 | 1133 | 1152 | TCAGTGATTG | 61 | 989 |
| | | | | | TCGCTGGGCA | | |
| 942585 | 6252 | 6271 | 1154 | 1173 | CATGGCTGCA | 36 | 847 |
| | | | | | GGCTTCGGCG | | |
| 942590 | 6302 | 6321 | 1204 | 1223 | TCCCGCTGCA | 83 | 920 |
| | | | | | GGCTGCGCGG | | |
| 1517150 | 3409 | 3428 | N/A | N/A | CTCCTGGTCT | 71 | 1238 |
| | | | | | TCTCTTATCT | | |
| 1517180 | 6312 | 6331 | 1214 | 1233 | GGACAGGGTC | 26 | 1239 |
| | | | | | TCCCGCTGCA | | |
| 1517187 | 4193 | 4212 | N/A | N/A | CCTGCAATGC | 78 | 1240 |
| | | | | | ATTAGAAACC | | |
| 1517192 | 4208 | 4227 | N/A | N/A | GGTATTCACT | 77 | 1241 |
| | | | | | ATCTGCCTGC | | |
| 1517199 | 3253 | 3272 | N/A | N/A | AAGCTCCAAC | 82 | 1242 |
| | | | | | CTCACAGTTA | | |
| 1517200 | 3340 | 3359 | N/A | N/A | GGTTCAAATT | 75 | 1243 |
| | | | | | CCATCCCCCC | | |
| 1517201 | 3004 | 3023 | N/A | N/A | TCTGCTGCCC | 78 | 1244 |
| | | | | | AGCCCTTCCC | | |
| 1517227 | 3205 | 3224 | N/A | N/A | CAGTCCTCAT | 74 | 1245 |
| | | | | | TTTAAAGTTC | | |
| 1517232 | 5008 | 5027 | N/A | N/A | GGCAGAATGA | 72† | 1246 |
| | | | | | AACCTGGACC | | |
| 1517235 | 4697 | 4716 | N/A | N/A | CGTCCAGGCA | 67 | 1247 |
| | | | | | TCTAGTACCT | | |
| 1517236 | 3422 | 3441 | N/A | N/A | CCTATTTAAC | 74 | 1248 |
| | | | | | TCCCTCCTGG | | |
| 1517245 | 3378 | 3397 | N/A | N/A | GGTCGGCCTC | 50 | 1249 |
| | | | | | CATAGAAAAT | | |
| 1517252 | 3190 | 3209 | N/A | N/A | AGTTCTCCAA | 91 | 1250 |
| | | | | | TCGACGGCTA | | |
| 1517289 | 2961 | 2980 | N/A | N/A | CTCACCCCCG | 103 | 1251 |
| | | | | | CTCCTCCTCT | | |
| 1517290 | 6151 | 6170 | 1053 | 1072 | GCTGCATGTC | 71 | 1252 |
| | | | | | TTCCACCAGG | | |
| 1517294 | 6272 | 6291 | 1174 | 1193 | CACGGGGTGG | 84 | 1253 |
| | | | | | CGTGGGGTCG | | |
| 1517328 | 3061 | 3080 | N/A | N/A | GCTTACATCC | 72 | 1254 |
| | | | | | CAGTCCAGCT | | |
| 1517370 | N/A | N/A | 232 | 251 | GCCTGGCATC | 88 | 1255 |
| | | | | | CTGCCAGGAA | | |
| 1517371 | 2971 | 2990 | N/A | N/A | TGCTGCTTGC | 86 | 1256 |
| | | | | | CTCACCCCCG | | |
| 1517412 | 4240 | 4259 | N/A | N/A | TGGAGAATAA | 71 | 1257 |
| | | | | | AGATCACAGC | | |
| 1517426 | 4183 | 4202 | N/A | N/A | ATTAGAAACC | 90 | 1258 |
| | | | | | TCTAACTCCC | | |
| 1517427 | 6363 | 6382 | 1265 | 1284 | TGGTGAATCT | 14 | 1193 |
| | | | | | TTATTAAACT | | |
| 1517428 | 4765 | 4784 | 247 | 266 | GCTTGCTCCA | 164 | 1259 |
| | | | | | CCTTGGCCTG | | |
| 1517430 | 3849 | 3868 | N/A | N/A | ACAACAAAAC | 76 | 1260 |
| | | | | | AACTTCTAAC | | |
| 1517438 | 3616 | 3635 | 190 | 209 | AGAACCTTCA | 85 | 1261 |
| | | | | | TCTTCCTGCC | | |
| 1517450 | 3282 | 3301 | N/A | N/A | TCGCATTCCT | 69 | 1262 |
| | | | | | CATTCTCCCT | | |
| 1517460 | 3627 | 3646 | 201 | 220 | ACGCAGCCCA | 61 | 1263 |
| | | | | | CAGAACCTTC | | |
| 1517461 | 3666 | 3685 | N/A | N/A | CAAGCCCCGC | 66 | 1264 |
| | | | | | CCCCATACCT | | |
| 1517477 | 6262 | 6281 | 1164 | 1183 | CGTGGGGTCG | 32 | 1265 |
| | | | | | CATGGCTGCA | | |
| 1517485 | 3218 | 3237 | N/A | N/A | TTATGAGCTA | 63 | 1266 |
| | | | | | ATTCAGTCCT | | |
| 1517487 | 3839 | 3858 | N/A | N/A | AACTTCTAAC | 85 | 1267 |
| | | | | | TCCTATCTCA | | |
| 1517494 | 5042 | 5061 | N/A | N/A | CAGAGACCCA | 82† | 1268 |
| | | | | | GGCCCCCCAA | | |
| 1517498 | 6292 | 6311 | 1194 | 1213 | GGCTGCGCGG | 39 | 1269 |
| | | | | | AGGCAGGAGG | | |
| 1517504 | 4593 | 4612 | N/A | N/A | AAGCATTATG | 84 | 1270 |
| | | | | | TATTTATAAA | | |
| 1517506 | 6282 | 6301 | 1184 | 1203 | AGGCAGGAGG | 81 | 1271 |
| | | | | | CACGGGGTGG | | |
| 1517522 | 4611 | 4630 | N/A | N/A | TCGGTTTAAT | 108 | 1272 |
| | | | | | CACTTGGAAA | | |
| 1517542 | 3476 | 3495 | N/A | N/A | TTATCTGCAA | 76 | 1273 |
| | | | | | CAGCCTAATC | | |
| 1517554 | 5055 | 5074 | N/A | N/A | AAGCTAGAAC | 93† | 1274 |
| | | | | | CAGCAGAGAC | | |
| 1517557 | 4539 | 4558 | N/A | N/A | GCGGCCTGAA | 79 | 1275 |
| | | | | | CATGGTTCAC | | |
| 1517559 | 6342 | 6361 | 1244 | 1263 | GGGTCCACCC | 69 | 1276 |
| | | | | | CAGGAGGACG | | |
| 1517569 | 3294 | 3313 | N/A | N/A | TCAGTCCCAG | 88 | 1277 |
| | | | | | TCTCGCATTC | | |
| 1517583 | 2916 | 2935 | N/A | N/A | TCCTGAGACT | 86 | 1278 |
| | | | | | ACCTGGAGGC | | |
| 1517586 | 3526 | 3545 | N/A | N/A | CCGGCCCCAA | 100 | 1279 |
| | | | | | CCCGGCCTCA | | |
| 1517600 | 3512 | 3531 | N/A | N/A | GCCTCACCCC | 95 | 1280 |
| | | | | | AGGTTAGCCT | | |
| 1517605 | 3465 | 3484 | N/A | N/A | AGCCTAATCC | 81 | 1281 |
| | | | | | CAGCACATTT | | |
| 1517606 | 3570 | 3589 | N/A | N/A | AAACTGTCAA | 101 | 1282 |
| | | | | | TCAACCGCCA | | |
| 1517614 | 3264 | 3283 | N/A | N/A | CTTCACATTC | 74 | 1283 |
| | | | | | TAAGCTCCAA | | |
| 1517620 | 5102 | 5121 | N/A | N/A | GAGAGAATTC | 84† | 1284 |
| | | | | | CAGAGAGCTA | | |
| 1517626 | 6211 | 6230 | 1113 | 1132 | CAGGGGCGGC | 88 | 1285 |
| | | | | | GCTGGTGCCC | | |
| 1517631 | 6322 | 6341 | 1224 | 1243 | GCTGGGGCGG | 89 | 1286 |
| | | | | | GGACAGGGTC | | |
| 1517679 | 3367 | 3386 | N/A | N/A | ATAGAAAATT | 78 | 1287 |
| | | | | | CCATCTTCCT | | |
| 1517697 | 6352 | 6371 | 1254 | 1273 | TATTAAACTA | 49 | 1288 |
| | | | | | GGGTCCACCC | | |
| 1517698 | 6191 | 6210 | 1093 | 1112 | ACGGCAGCCT | 70 | 1289 |
| | | | | | GCACCTTCTC | | |
| 1517700 | 2947 | 2966 | N/A | N/A | TCCTCTCCCC | 76 | 1290 |
| | | | | | AAGCCCGACC | | |
| 1517710 | 3048 | 3067 | N/A | N/A | TCCAGCTGCT | 75 | 1291 |
| | | | | | CTCCCCACCC | | |
| 1517711 | 4908 | 4927 | 390 | 409 | TGAGCAGCTC | 41† | 1292 |
| | | | | | CTCCTGCACC | | |
| 1517716 | 2863 | 2882 | 115 | 134 | GCCCCTGAGC | 93 | 1293 |
| | | | | | TCATCCCCGT | | |
| 1517717 | 3550 | 3569 | N/A | N/A | GTGAGGACTC | 75 | 1294 |
| | | | | | CTCCCACCCC | | |
| 1517728 | 3493 | 3512 | N/A | N/A | TTCCAAGCCT | 78 | 1295 |
| | | | | | TGTTGCATTA | | |
| 1517738 | 3433 | 3452 | N/A | N/A | CCAACCCATT | 87 | 1296 |
| | | | | | CCCTATTTAA | | |
| 1517752 | 3103 | 3122 | N/A | N/A | TGCTCGATAA | 73 | 1297 |
| | | | | | ATGATAGTGA | | |
| 1517754 | 4781 | 4800 | 263 | 282 | CGGCTCTGTC | 61 | 1298 |
| | | | | | TCCACCGCTT | | |
| 1517756 | 2987 | 3006 | N/A | N/A | CCCAGGTCCA | 78 | 1299 |
| | | | | | GTCCCCTGCT | | |
| 1517770 | 6362 | 6381 | 1264 | 1283 | GGTGAATCTT | 8 | 1300 |
| | | | | | TATTAAACTA | | |
| 1517785 | 3318 | 3337 | N/A | N/A | CCCCTCCCCA | 80 | 1301 |
| | | | | | CCGCCGGTTC | | |
| 1517801 | 6171 | 6190 | 1073 | 1092 | CACCAGCCCG | 89 | 1302 |
| | | | | | GCCCACTGGC | | |
| 1517807 | 4881 | 4900 | 363 | 382 | ACAGTGTCTG | 19† | 1303 |
| | | | | | CACCCAGCGC | | |
| 1517831 | 3767 | 3786 | N/A | N/A | GAAGCAGCAC | 90 | 1304 |
| | | | | | AGAAGCCTCA | | |
| 1517837 | 6373 | 6392 | 1275 | 1294 | GCGTGAAACT | 25 | 1305 |
| | | | | | TGGTGAATCT | | |
| 1517856 | 3397 | 3416 | N/A | N/A | TCTTATCTCC | 69 | 1306 |
| | | | | | CCATCCCCAG | | |
| 1517865 | 4818 | 4837 | 300 | 319 | CGCTCTGCCA | 107 | 1307 |
| | | | | | CTCGGTCTGC | | |
| 1517866 | 3445 | 3464 | N/A | N/A | ACCAAGCCGC | 77 | 1308 |
| | | | | | CCCCAACCCA | | |
| 1517869 | 6332 | 6351 | 1234 | 1253 | CAGGAGGACG | 90 | 1309 |
| | | | | | GCTGGGGCGG | | |
| 1517870 | 2880 | 2899 | 132 | 151 | CCCAGCTCTT | 77 | 1310 |
| | | | | | TCTAGAGGCC | | |
| 1517882 | 6242 | 6261 | 1144 | 1163 | GGCTTCGGCG | 56 | 1311 |
| | | | | | TTCAGTGATT | | |

TABLE 23
Reduction of APOE RNA by 5-10-5 MOE gapmers
with mixed PO/PS internucleoside linkages
at a concentration of 4000 nM in Hep3B
cells plated at 20,000 cells per well

| | SEQ | SEQ | SEQ | SEQ | | | |
|---|---|---|---|---|---|---|---|
| | ID | ID | ID | ID | | | |
| | No: | No: | No: | No: | | | |
| Com- | 1 | 1 | 2 | 2 | | APOE | SEQ |
| pound | Start | Stop | Start | Stop | Sequence | (% | ID |
| Number | Site | Site | Site | Site | (5′ to 3′) | UTC) | NO |
| 942589 | 6301 | 6320 | 1203 | 1222 | CCCGCTGCAG | 51 | 848 |
| | | | | | GCTGCGCGGA | | |
| 942592 | 6341 | 6360 | 1243 | 1262 | GGTCCACCCC | 74 | 1062 |
| | | | | | AGGAGGACGG | | |
| 942596 | 6351 | 6370 | 1253 | 1272 | ATTAAACTAG | 54 | 921 |
| | | | | | GGTCCACCCC | | |
| 942598 | 6372 | 6391 | 1274 | 1293 | CGTGAAACTT | 30 | 1063 |
| | | | | | GGTGAATCTT | | |
| 1517149 | 6241 | 6260 | 1143 | 1162 | GCTTCGGCGT | 70 | 1312 |
| | | | | | TCAGTGATTG | | |
| 1517168 | 3444 | 3463 | N/A | N/A | CCAAGCCGCC | 87 | 1313 |
| | | | | | CCCAACCCAT | | |
| 1517169 | 3102 | 3121 | N/A | N/A | GCTCGATAAA | 91 | 1314 |
| | | | | | TGATAGTGAC | | |
| 1517170 | 6281 | 6300 | 1183 | 1202 | GGCAGGAGGC | 49 | 1315 |
| | | | | | ACGGGGTGGC | | |
| 1517178 | 3204 | 3223 | N/A | N/A | AGTCCTCATT | 78 | 1316 |
| | | | | | TTAAAGTTCT | | |
| 1517191 | 6251 | 6270 | 1153 | 1172 | ATGGCTGCAG | 49 | 1317 |
| | | | | | GCTTCGGCGT | | |
| 1517194 | 2985 | 3004 | N/A | N/A | CAGGTCCAGT | 89 | 1318 |
| | | | | | CCCCTGCTGC | | |
| 1517197 | 6149 | 6168 | 1051 | 1070 | TGCATGTCTT | 78 | 1319 |
| | | | | | CCACCAGGGG | | |
| 1517226 | 3281 | 3300 | N/A | N/A | CGCATTCCTC | 63 | 1320 |
| | | | | | ATTCTCCCTT | | |
| 1517229 | 5040 | 5059 | N/A | N/A | GAGACCCAGG | 98† | 1321 |
| | | | | | CCCCCCAAGA | | |
| 1517276 | 3408 | 3427 | N/A | N/A | TCCTGGTCTT | 84 | 1322 |
| | | | | | CTCTTATCTC | | |
| 1517291 | 3525 | 3544 | N/A | N/A | CGGCCCCAAC | 107 | 1323 |
| | | | | | CCGGCCTCAC | | |
| 1517304 | 4608 | 4627 | N/A | N/A | GTTTAATCAC | 77 | 1324 |
| | | | | | TTGGAAAGCA | | |
| 1517306 | 3549 | 3568 | N/A | N/A | TGAGGACTCC | 80 | 1325 |
| | | | | | TCCCACCCCC | | |
| 1517309 | 6331 | 6350 | 1233 | 1252 | AGGAGGACGG | 90 | 1326 |
| | | | | | CTGGGGCGGG | | |
| 1517311 | 4880 | 4899 | 362 | 381 | CAGTGTCTGC | 36† | 1327 |
| | | | | | ACCCAGCGCA | | |
| 1517336 | 3464 | 3483 | N/A | N/A | GCCTAATCCC | 86 | 1328 |
| | | | | | AGCACATTTA | | |
| 1517337 | 3047 | 3066 | N/A | N/A | CCAGCTGCTC | 74 | 1329 |
| | | | | | TCCCCACCCC | | |
| 1517379 | 3252 | 3271 | N/A | N/A | AGCTCCAACC | 73 | 1330 |
| | | | | | TCACAGTTAA | | |
| 1517388 | 6321 | 6340 | 1223 | 1242 | CTGGGGCGGG | 94 | 1331 |
| | | | | | GACAGGGTCT | | |
| 1517401 | 3366 | 3385 | N/A | N/A | TAGAAAATTC | 96 | 1332 |
| | | | | | CATCTTCCTC | | |
| 1517406 | 6169 | 6188 | 1071 | 1090 | CCAGCCCGGC | 79 | 1333 |
| | | | | | CCACTGGCGC | | |
| 1517427 | 6363 | 6382 | 1265 | 1284 | TGGTGAATCT | 25 | 1193 |
| | | | | | TTATTAAACT | | |
| 1517448 | 4192 | 4211 | N/A | N/A | CTGCAATGCA | 89 | 1334 |
| | | | | | TTAGAAACCT | | |
| 1517454 | 3420 | 3439 | N/A | N/A | TATTTAACTC | 91 | 1335 |
| | | | | | CCTCCTGGTC | | |
| 1517455 | 3003 | 3022 | N/A | N/A | CTGCTGCCCA | 74 | 1336 |
| | | | | | GCCCTTCCCA | | |
| 1517458 | 3838 | 3857 | N/A | N/A | ACTTCTAACT | 79 | 1337 |
| | | | | | CCTATCTCAA | | |
| 1517463 | 4665 | 4684 | N/A | N/A | AAGGTGCTCC | 92 | 1338 |
| | | | | | ACAAATGCTT | | |
| 1517472 | 3848 | 3867 | N/A | N/A | CAACAAAACA | 86 | 1339 |
| | | | | | ACTTCTAACT | | |
| 1517474 | 3487 | 3506 | N/A | N/A | GCCTTGTTGC | 96 | 1340 |
| | | | | | ATTATCTGCA | | |
| 1517486 | 4779 | 4798 | 261 | 280 | GCTCTGTCTC | 84 | 1341 |
| | | | | | CACCGCTTGC | | |
| 1517489 | 3263 | 3282 | N/A | N/A | TTCACATTCT | 70 | 1342 |
| | | | | | AAGCTCCAAC | | |
| 1517491 | 3291 | 3310 | N/A | N/A | GTCCCAGTCT | 71 | 1343 |
| | | | | | CGCATTCCTC | | |
| 1517495 | 4239 | 4258 | N/A | N/A | GGAGAATAAA | 63 | 1344 |
| | | | | | GATCACAGCT | | |
| 1517511 | 3339 | 3358 | N/A | N/A | GTTCAAATTC | 75 | 1345 |
| | | | | | CATCCCCCCA | | |
| 1517523 | 3626 | 3645 | 200 | 219 | CGCAGCCCAC | 80 | 1346 |
| | | | | | AGAACCTTCA | | |
| 1517528 | 4817 | 4836 | 299 | 318 | GCTCTGCCAC | 106 | 1347 |
| | | | | | TCGGTCTGCT | | |
| 1517538 | 2861 | 2880 | 113 | 132 | CCCTGAGCTC | 80 | 1348 |
| | | | | | ATCCCCGTGC | | |
| 1517546 | 4591 | 4610 | N/A | N/A | GCATTATGTA | 92 | 1349 |
| | | | | | TTTATAAACA | | |
| 1517566 | 3217 | 3236 | N/A | N/A | TATGAGCTAA | 87 | 1350 |
| | | | | | TTCAGTCCTC | | |
| 1517587 | 6189 | 6208 | 1091 | 1110 | GGCAGCCTGC | 67 | 1351 |
| | | | | | ACCTTCTCCA | | |
| 1517619 | 2960 | 2979 | N/A | N/A | TCACCCCCGC | 78 | 1352 |
| | | | | | TCCTCCTCTC | | |
| 1517628 | 3511 | 3530 | N/A | N/A | CCTCACCCCA | 92 | 1353 |
| | | | | | GGTTAGCCTT | | |
| 1517629 | 3317 | 3336 | N/A | N/A | CCCTCCCCAC | 99 | 1354 |
| | | | | | CGCCGGTTCC | | |
| 1517632 | 6261 | 6280 | 1163 | 1182 | GTGGGGTCGC | 41 | 1355 |
| | | | | | ATGGCTGCAG | | |
| 1517636 | 6291 | 6310 | 1193 | 1212 | GCTGCGCGGA | 32 | 1356 |
| | | | | | GGCAGGAGGC | | |
| 1517653 | 2946 | 2965 | N/A | N/A | CCTCTCCCCA | 91 | 1357 |
| | | | | | AGCCCGACCC | | |
| 1517657 | 4182 | 4201 | N/A | N/A | TTAGAAACCT | 83 | 1358 |
| | | | | | CTAACTCCCA | | |
| 1517672 | 5007 | 5026 | N/A | N/A | GCAGAATGAA | 73† | 1359 |
| | | | | | ACCTGGACCT | | |
| 1517673 | 3396 | 3415 | N/A | N/A | CTTATCTCCC | 90 | 1360 |
| | | | | | CATCCCCAGG | | |
| 1517682 | 3764 | 3783 | N/A | N/A | GCAGCACAGA | 66 | 1361 |
| | | | | | AGCCTCAGAA | | |
| 1517683 | 6229 | 6248 | 1131 | 1150 | AGTGATTGTC | 57 | 1362 |
| | | | | | GCTGGGCACA | | |
| 1517712 | 3377 | 3396 | N/A | N/A | GTCGGCCTCC | 93 | 1363 |
| | | | | | ATAGAAAATT | | |
| 1517713 | 6271 | 6290 | 1173 | 1192 | ACGGGGTGGC | 67 | 1364 |
| | | | | | GTGGGGTCGC | | |
| 1517721 | 4763 | 4782 | 245 | 264 | TTGCTCCACC | 96 | 1365 |
| | | | | | TTGGCCTGGC | | |
| 1517725 | 3569 | 3588 | N/A | N/A | AACTGTCAAT | 84 | 1366 |
| | | | | | CAACCGCCAG | | |
| 1517746 | 4907 | 4926 | 389 | 408 | GAGCAGCTCC | 28† | 1367 |
| | | | | | TCCTGCACCT | | |
| 1517765 | 3615 | 3634 | 189 | 208 | GAACCTTCAT | 74 | 1368 |
| | | | | | CTTCCTGCCT | | |
| 1517767 | 3475 | 3494 | N/A | N/A | TATCTGCAAC | 104 | 1369 |
| | | | | | AGCCTAATCC | | |
| 1517802 | 3059 | 3078 | N/A | N/A | TTACATCCCA | 73 | 1370 |
| | | | | | GTCCAGCTGC | | |
| 1517804 | 3665 | 3684 | N/A | N/A | AAGCCCCGCC | 82 | 1371 |
| | | | | | CCCATACCTG | | |
| 1517819 | 2879 | 2898 | 131 | 150 | CCAGCTCTTT | 75 | 1372 |
| | | | | | CTAGAGGCCC | | |
| 1517823 | 6311 | 6330 | 1213 | 1232 | GACAGGGTCT | 32 | 1373 |
| | | | | | CCCGCTGCAG | | |
| 1517828 | 4534 | 4553 | N/A | N/A | CTGAACATGG | 75 | 1374 |
| | | | | | TTCACTGCAA | | |
| 1517852 | 5054 | 5073 | N/A | N/A | AGCTAGAACC | 74† | 1375 |
| | | | | | AGCAGAGACC | | |
| 1517862 | 2970 | 2989 | N/A | N/A | GCTGCTTGCC | 79 | 1376 |
| | | | | | TCACCCCCGC | | |
| 1517884 | 2915 | 2934 | N/A | N/A | CCTGAGACTA | 87 | 1377 |
| | | | | | CCTGGAGGCC | | |
| 1517885 | 6361 | 6380 | 1263 | 1282 | GTGAATCTTT | 24 | 1378 |
| | | | | | ATTAAACTAG | | |
| 1517887 | 6209 | 6228 | 1111 | 1130 | GGGGCGGCGC | 89 | 1379 |
| | | | | | TGGTGCCCAC | | |
| 1517893 | 3657 | 3676 | N/A | N/A | CCCCCATACC | 78 | 1380 |
| | | | | | TGCCAGGAAT | | |
| 1517898 | 5097 | 5116 | N/A | N/A | AATTCCAGAG | 80† | 1381 |
| | | | | | AGCTAAAGCC | | |
| 1517903 | 3432 | 3451 | N/A | N/A | CAACCCATTC | 85 | 1382 |
| | | | | | CCTATTTAAC | | |
| 1517904 | 3165 | 3184 | N/A | N/A | CGTGTCGCTG | 84 | 1383 |
| | | | | | CCCCTGGCTC | | |
| 1517910 | 4207 | 4226 | N/A | N/A | GTATTCACTA | 78 | 1384 |
| | | | | | TCTGCCTGCA | | |

TABLE 24
Reduction of APOE RNA by 5-10-5 MOE gapmers with mixed PO/PS internucleoside
linkages at a concentration of 4000 nM in Hep3B cells plated at 20,000 cells
per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 708024 | 6371 | 6390 | 1273 | 1292 | GTGAAACTTGGTGAATCTTT | 25 | 77 |
| 942595 | 6350 | 6369 | 1252 | 1271 | TTAAACTAGGGTCCACCCCA | 50 | 849 |
| 1517155 | 4206 | 4225 | N/A | N/A | TATTCACTATCTGCCTGCAA | 73 | 1385 |
| 1517157 | 6300 | 6319 | 1202 | 1221 | CCGCTGCAGGCTGCGCGGAG | 89 | 1386 |
| 1517158 | 6270 | 6289 | 1172 | 1191 | CGGGGTGGCGTGGGGTCGCA | 49 | 1387 |
| 1517160 | 6147 | 6166 | 1049 | 1068 | CATGTCTTCCACCAGGGGCT | 85 | 1388 |
| 1517162 | 4800 | 4819 | 282 | 301 | GCTGGCGCAGCTCGGGCTCC | 85 | 1389 |
| 1517166 | 3613 | 3632 | 187 | 206 | ACCTTCATCTTCCTGCCTGT | 61 | 1390 |
| 1517171 | 3473 | 3492 | N/A | N/A | TCTGCAACAGCCTAATCCCA | 71 | 1391 |
| 1517172 | 3216 | 3235 | N/A | N/A | ATGAGCTAATTCAGTCCTCA | 75 | 1392 |
| 1517175 | 6280 | 6299 | 1182 | 1201 | GCAGGAGGCACGGGGTGGCG | 49 | 1393 |
| 1517212 | 5096 | 5115 | N/A | N/A | ATTCCAGAGAGCTAAAGCCA | 72† | 1394 |
| 1517230 | 6207 | 6226 | 1109 | 1128 | GGCGGCGCTGGTGCCCACGG | 67 | 1395 |
| 1517255 | 4762 | 4781 | 244 | 263 | TGCTCCACCTTGGCCTGGCA | 96 | 1396 |
| 1517257 | 3847 | 3866 | N/A | N/A | AACAAAACAACTTCTAACTC | 102 | 1397 |
| 1517263 | 4606 | 4625 | N/A | N/A | TTAATCACTTGGAAAGCATT | 64 | 1398 |
| 1517270 | 3164 | 3183 | N/A | N/A | GTGTCGCTGCCCCTGGCTCC | 71 | 1399 |
| 1517312 | 4191 | 4210 | N/A | N/A | TGCAATGCATTAGAAACCTC | 62 | 1400 |
| 1517315 | 4976 | 4995 | N/A | N/A | GCCGCCCACCAGGAGGGTCA | 83† | 85 |
| 1517323 | 3376 | 3395 | N/A | N/A | TCGGCCTCCATAGAAAATTC | 76 | 1401 |
| 1517325 | 3365 | 3384 | N/A | N/A | AGAAAATTCCATCTTCCTCT | 77 | 1402 |
| 1517348 | 2959 | 2978 | N/A | N/A | CACCCCCGCTCCTCCTCTCC | 80 | 1403 |
| 1517358 | 3290 | 3309 | N/A | N/A | TCCCAGTCTCGCATTCCTCA | 70 | 1404 |
| 1517363 | 3441 | 3460 | N/A | N/A | AGCCGCCCCCAACCCATTCC | 69 | 1405 |
| 1517367 | 6260 | 6279 | 1162 | 1181 | TGGGGTCGCATGGCTGCAGG | 35 | 1406 |
| 1517372 | 3203 | 3222 | N/A | N/A | GTCCTCATTTTAAAGTTCTC | 77 | 1407 |
| 1517375 | 3431 | 3450 | N/A | N/A | AACCCATTCCCTATTTAACT | 87 | 1408 |
| 1517383 | 6360 | 6379 | 1262 | 1281 | TGAATCTTTATTAAACTAGG | 16 | 1409 |
| 1517390 | 2984 | 3003 | N/A | N /A | AGGTCCAGTCCCCTGCTGCT | 75 | 1410 |
| 1517404 | 3250 | 3269 | N/A | N/A | CTCCAACCTCACAGTTAAGC | 79 | 1411 |
| 1517410 | 3664 | 3683 | N/A | N/A | AGCCCCGCCCCCATACCTGC | 91 | 1412 |
| 1517424 | 6240 | 6259 | 1142 | 1161 | CTTCGGCGTTCAGTGATTGT | 54 | 1413 |
| 1517427 | 6363 | 6382 | 1265 | 1284 | TGGTGAATCTTTATTAAACT | 28 | 1193 |
| 1517432 | 6330 | 6349 | 1232 | 1251 | GGAGGACGGCTGGGGCGGGG | 76 | 1414 |
| 1517434 | 6227 | 6246 | 1129 | 1148 | TGATTGTCGCTGGGCACAGG | 67 | 1415 |
| 1517440 | N/A | N/A | 231 | 250 | CCTGGCATCCTGCCAGGAAT | 90 | 1416 |
| 1517443 | 3568 | 3587 | N/A | N/A | ACTGTCAATCAACCGCCAGT | 98 | 1417 |
| 1517468 | 3507 | 3526 | N/A | N/A | ACCCCAGGTTAGCCTTCCAA | 85 | 1418 |
| 1517473 | 3101 | 3120 | N/A | N/A | CTCGATAAATGATAGTGACA | 53 | 1419 |
| 1517497 | 3625 | 3644 | 199 | 218 | GCAGCCCACAGAACCTTCAT | 58 | 21 |
| 1517513 | 3548 | 3567 | N/A | N/A | GAGGACTCCTCCCACCCCCA | 103 | 1420 |
| 1517529 | 4902 | 4921 | 384 | 403 | GCTCCTCCTGCACCTGCTCA | 14† | 1421 |
| 1517536 | 2945 | 2964 | N/A | N/A | CTCTCCCCAAGCCCGACCCC | 87 | 1422 |
| 1517539 | 3058 | 3077 | N/A | N/A | TACATCCCAGTCCAGCTGCT | 87 | 1423 |
| 1517549 | 6187 | 6206 | 1089 | 1108 | CAGCCTGCACCTTCTCCACC | 66 | 1424 |
| 1517574 | 6310 | 6329 | 1212 | 1231 | ACAGGGTCTCCCGCTGCAGG | 24 | 1425 |
| 1517577 | 3837 | 3856 | N/A | N/A | CTTCTAACTCCTATCTCAAG | 83 | 1426 |
| 1517592 | 3463 | 3482 | N/A | N/A | CCTAATCCCAGCACATTTAC | 89 | 1427 |
| 1517607 | 4590 | 4609 | N/A | N/A | CATTATGTATTTATAAACAG | 72 | 1428 |
| 1517616 | 3395 | 3414 | N/A | N/A | TTATCTCCCCATCCCCAGGT | 72 | 1429 |
| 1517621 | 5039 | 5058 | N/A | N/A | AGACCCAGGCCCCCCAAGAC | 78† | 1430 |
| 1517645 | 2999 | 3018 | N/A | N/A | TGCCCAGCCCTTCCCAGGTC | 90 | 1431 |
| 1517649 | 3407 | 3426 | N/A | N/A | CCTGGTCTTCTCTTATCTCC | 96 | 1432 |
| 1517654 | 2878 | 2897 | 130 | 149 | CAGCTCTTTCTAGAGGCCCC | 65 | 1433 |
| 1517684 | 3042 | 3061 | N/A | N/A | TGCTCTCCCCACCCCACCTT | 77 | 1434 |
| 1517693 | 2914 | 2933 | N/A | N/A | CTGAGACTACCTGGAGGCCA | 81 | 1435 |
| 1517702 | 4181 | 4200 | N/A | N/A | TAGAAACCTCTAACTCCCAG | 96 | 1436 |
| 1517705 | 3338 | 3357 | N/A | N/A | TTCAAATTCCATCCCCCCAC | 75 | 1437 |
| 1517706 | 6340 | 6359 | 1242 | 1261 | GTCCACCCCAGGAGGACGGC | 86 | 73 |
| 1517720 | 4237 | 4256 | N/A | N/A | AGAATAAAGATCACAGCTGC | 67 | 1438 |
| 1517730 | 4778 | 4797 | 260 | 279 | CTCTGTCTCCACCGCTTGCT | 89 | 1439 |
| 1517736 | 4874 | 4893 | 356 | 375 | CTGCACCCAGCGCAGGTAAT | 28† | 1440 |
| 1517740 | 6250 | 6269 | 1152 | 1171 | TGGCTGCAGGCTTCGGCGTT | 49 | 1441 |
| 1517744 | 6320 | 6339 | 1222 | 1241 | TGGGGCGGGGACAGGGTCTC | 77 | 1442 |
| 1517749 | 3316 | 3335 | N/A | N/A | CCTCCCCACCGCCGGTTCCA | 71 | 1443 |
| 1517750 | 6290 | 6309 | 1192 | 1211 | CTGCGCGGAGGCAGGAGGCA | 61 | 1444 |
| 1517762 | 3486 | 3505 | N/A | N/A | CCTTGTTGCATTATCTGCAA | 92 | 1445 |
| 1517763 | 2860 | 2879 | 112 | 131 | CCTGAGCTCATCCCCGTGCC | 65 | 1446 |
| 1517772 | 2969 | 2988 | N/A | N/A | CTGCTTGCCTCACCCCCGCT | 74 | 1447 |
| 1517775 | 3419 | 3438 | N/A | N/A | ATTTAACTCCCTCCTGGTCT | 73 | 1448 |
| 1517789 | 5053 | 5072 | N/A | N/A | GCTAGAACCAGCAGAGACCC | 69† | 1449 |
| 1517803 | 3274 | 3293 | N/A | N/A | CTCATTCTCCCTTCACATTC | 78 | 1450 |
| 1517840 | 3262 | 3281 | N/A | N/A | TCACATTCTAAGCTCCAACC | 86 | 1451 |
| 1517844 | 6167 | 6186 | 1069 | 1088 | AGCCCGGCCCACTGGCGCTG | 70 | 1452 |
| 1517851 | 4664 | 4683 | N/A | N/A | AGGTGCTCCACAAATGCTTC | 67 | 1453 |
| 1517883 | 4307 | 4326 | N/A | N/A | CCGCGCCCAGCGGAAAAGCA | 73 | 1454 |
| 1517900 | 3759 | 3778 | N/A | N/A | ACAGAAGCCTCAGAAGAGGG | 77 | 1455 |
| 1517909 | 3523 | 3542 | N/A | N/A | GCCCCAACCCGGCCTCACCC | 91 | 1456 |

TABLE 25
Reduction of APOE RNA by 5-10-5 MOE gapmers with mixed PO/PS internucleoside
linkages at a concentration of 4000 nM in Hep3B cells plated at 20,000 cells
per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 942594 | 6349 | 6368 | 1251 | 1270 | TAAACTAGGGTCCACCCCAG | 79 | 776 |
| 1517154 | 3202 | 3221 | N/A | N/A | TCCTCATTTTAAAGTTCTCC | 80 | 1457 |
| 1517156 | 5052 | 5071 | N/A | N/A | CTAGAACCAGCAGAGACCCA | 76† | 1458 |
| 1517164 | 6319 | 6338 | 1221 | 1240 | GGGGCGGGGACAGGGTCTCC | 124 | 1459 |
| 1517182 | 6299 | 6318 | 1201 | 1220 | CGCTGCAGGCTGCGCGGAGG | 52 | 1460 |
| 1517208 | 6269 | 6288 | 1171 | 1190 | GGGGTGGCGTGGGGTCGCAT | 26 | 1461 |
| 1517238 | 3430 | 3449 | N/A | N/A | ACCCATTCCCTATTTAACTC | 103 | 1462 |
| 1517249 | 3505 | 3524 | N/A | N/A | CCCAGGTTAGCCTTCCAAGC | 81 | 1463 |
| 1517250 | 3057 | 3076 | N/A | N/A | ACATCCCAGTCCAGCTGCTC | 90 | 1464 |
| 1517261 | 3289 | 3308 | N/A | N/A | CCCAGTCTCGCATTCCTCAT | 80 | 1465 |
| 1517274 | 6279 | 6298 | 1181 | 1200 | CAGGAGGCACGGGGTGGCGT | 59 | 1466 |
| 1517275 | 6289 | 6308 | 1191 | 1210 | TGCGCGGAGGCAGGAGGCAC | 76 | 1467 |
| 1517277 | 2859 | 2878 | 111 | 130 | CTGAGCTCATCCCCGTGCCC | 113 | 1468 |
| 1517281 | 6309 | 6328 | 1211 | 1230 | CAGGGTCTCCCGCTGCAGGC | 30 | 1469 |
| 1517288 | 3163 | 3182 | N/A | N/A | TGTCGCTGCCCCTGGCTCCC | 89 | 1470 |
| 1517301 | 3836 | 3855 | N/A | N/A | TTCTAACTCCTATCTCAAGG | 108 | 1471 |
| 1517307 | 4777 | 4796 | 259 | 278 | TCTGTCTCCACCGCTTGCTC | 115 | 1472 |
| 1517329 | 3394 | 3413 | N/A | N/A | TATCTCCCCATCCCCAGGTC | 106 | 1473 |
| 1517338 | 4944 | 4963 | N/A | N/A | GGGACACTCACCTCAGTTCC | 98† | 1474 |
| 1517339 | 3663 | 3682 | N/A | N/A | GCCCCGCCCCCATACCTGCC | 105 | 1475 |
| 1517344 | 3374 | 3393 | N/A | N/A | GGCCTCCATAGAAAATTCCA | 110 | 1476 |
| 1517346 | 3337 | 3356 | N/A | N/A | TCAAATTCCATCCCCCCACC | 94 | 1477 |
| 1517349 | 4302 | 4321 | N/A | N/A | CCCAGCGGAAAAGCATGTAT | 108 | 1478 |
| 1517368 | 3846 | 3865 | N/A | N/A | ACAAAACAACTTCTAACTCC | 123 | 1479 |
| 1517382 | 3418 | 3437 | N/A | N/A | TTTAACTCCCTCCTGGTCTT | 99 | 1480 |
| 1517386 | 3485 | 3504 | N/A | N/A | CTTGTTGCATTATCTGCAAC | 111 | 1481 |
| 1517408 | 3566 | 3585 | N/A | N/A | TGTCAATCAACCGCCAGTGA | 106 | 1482 |
| 1517411 | 2955 | 2974 | N/A | N/A | CCCGCTCCTCCTCTCCCCAA | 111 | 1483 |
| 1517417 | 2968 | 2987 | N/A | N/A | TGCTTGCCTCACCCCCGCTC | 110 | 1484 |
| 1517425 | 3611 | 3630 | 185 | 204 | CTTCATCTTCCTGCCTGTGA | 78 | 1485 |
| 1517427 | 6363 | 6382 | 1265 | 1284 | TGGTGAATCTTTATTAAACT | 18 | 1193 |
| 1517436 | 6145 | 6164 | 1047 | 1066 | TGTCTTCCACCAGGGGCTCG | 104 | 1486 |
| 1517449 | 5038 | 5057 | N/A | N/A | GACCCAGGCCCCCCAAGACT | 103† | 1487 |
| 1517452 | 4796 | 4815 | 278 | 297 | GCGCAGCTCGGGCTCCGGCT | 126 | 1488 |
| 1517462 | 3249 | 3268 | N/A | N/A | TCCAACCTCACAGTTAAGCG | 83 | 1489 |
| 1517471 | 2943 | 2962 | N/A | N/A | CTCCCCAAGCCCGACCCCGA | 92 | 1490 |
| 1517478 | 6165 | 6184 | 1067 | 1086 | CCCGGCCCACTGGCGCTGCA | 91 | 1491 |
| 1517503 | 3522 | 3541 | N/A | N/A | CCCCAACCCGGCCTCACCCC | 101 | 1492 |
| 1517508 | 6359 | 6378 | 1261 | 1280 | GAATCTTTATTAAACTAGGG | 18 | 1493 |
| 1517518 | 3462 | 3481 | N/A | N/A | CTAATCCCAGCACATTTACC | 83 | 1494 |
| 1517550 | 4180 | 4199 | N/A | N/A | AGAAACCTCTAACTCCCAGC | 77 | 1495 |
| 1517552 | N/A | N/A | 226 | 245 | CATCCTGCCAGGAATGTGAC | 102 | 26 |
| 1517564 | 4663 | 4682 | N/A | N/A | GGTGCTCCACAAATGCTTCT | 107 | 1496 |
| 1517568 | 4236 | 4255 | N/A | N/A | GAATAAAGATCACAGCTGCC | 93 | 1497 |
| 1517570 | 6185 | 6204 | 1087 | 1106 | GCCTGCACCTTCTCCACCAG | 79 | 1498 |
| 1517585 | 3406 | 3425 | N/A | N/A | CTGGTCTTCTCTTATCTCCC | 99 | 1499 |
| 1517591 | 3472 | 3491 | N/A | N/A | CTGCAACAGCCTAATCCCAG | 102 | 1500 |
| 1517611 | 2998 | 3017 | N/A | N/A | GCCCAGCCCTTCCCAGGTCC | 101 | 1501 |
| 1517624 | 2891 | 2910 | 143 | 162 | GTTCCCAGGGTCCCAGCTCT | 112 | 1502 |
| 1517638 | 3213 | 3232 | N/A | N/A | AGCTAATTCAGTCCTCATTT | 88 | 1503 |
| 1517640 | 2983 | 3002 | N/A | N/A | GGTCCAGTCCCCTGCTGCTT | 102 | 1504 |
| 1517671 | 5093 | 5112 | N/A | N/A | CCAGAGAGCTAAAGCCAGGA | 93† | 1505 |
| 1517708 | 3542 | 3561 | N/A | N/A | TCCTCCCACCCCCAGCCCGG | 110 | 1506 |
| 1517729 | 6249 | 6268 | 1151 | 1170 | GGCTGCAGGCTTCGGCGTTC | 31 | 1507 |
| 1517739 | 6339 | 6358 | 1241 | 1260 | TCCACCCCAGGAGGACGGCT | 99 | 1508 |
| 1517742 | 3364 | 3383 | N/A | N/A | GAAAATTCCATCTTCCTCTC | 77 | 1509 |
| 1517747 | 6205 | 6224 | 1107 | 1126 | CGGCGCTGGTGCCCACGGCA | 91 | 1510 |
| 1517764 | 3312 | 3331 | N/A | N/A | CCCACCGCCGGTTCCATCTC | 105 | 1511 |
| 1517768 | 6239 | 6258 | 1141 | 1160 | TTCGGCGTTCAGTGATTGTC | 65 | 1512 |
| 1517769 | 4601 | 4620 | N/A | N/A | CACTTGGAAAGCATTATGTA | 91 | 1513 |
| 1517777 | 3623 | 3642 | 197 | 216 | AGCCCACAGAACCTTCATCT | 72 | 1514 |
| 1517778 | 3673 | 3692 | N/A | N/A | AACCGAGCAAGCCCCGCCCC | 104 | 1515 |
| 1517795 | 6329 | 6348 | 1231 | 1250 | GAGGACGGCTGGGGCGGGGA | 109 | 1516 |
| 1517799 | 2872 | 2891 | 124 | 143 | TTTCTAGAGGCCCCTGAGCT | 101 | 1517 |
| 1517810 | 3037 | 3056 | N/A | N/A | TCCCCACCCCACCTTCTAGC | 101 | 1518 |
| 1517818 | 6225 | 6244 | 1127 | 1146 | ATTGTCGCTGGGCACAGGGG | 87 | 1519 |
| 1517833 | 3440 | 3459 | N/A | N/A | GCCGCCCCCAACCCATTCCC | 108 | 1520 |
| 1517838 | 3261 | 3280 | N/A | N/A | CACATTCTAAGCTCCAACCT | 87 | 1521 |
| 1517839 | 4190 | 4209 | N/A | N/A | GCAATGCATTAGAAACCTCT | 87 | 1522 |
| 1517843 | 4205 | 4224 | N/A | N/A | ATTCACTATCTGCCTGCAAT | 97 | 1523 |
| 1517850 | 3273 | 3292 | N/A | N/A | TCATTCTCCCTTCACATTCT | 90 | 1524 |
| 1517863 | 4900 | 4919 | 382 | 401 | TCCTCCTGCACCTGCTCAGA | 17† | 1525 |
| 1517874 | 6370 | 6389 | 1272 | 1291 | TGAAACTTGGTGAATCTTTA | 23 | 1526 |
| 1517877 | 6259 | 6278 | 1161 | 1180 | GGGGTCGCATGGCTGCAGGC | 64 | 1527 |
| 1517889 | 4842 | 4861 | 324 | 343 | CCAGTGCCAGTTCCCAGCGC | 135† | 1528 |
| 1517896 | 4760 | 4779 | 242 | 261 | CTCCACCTTGGCCTGGCATC | 120 | 1529 |
| 1517899 | 4587 | 4606 | N/A | N/A | TATGTATTTATAAACAGGGT | 110 | 1530 |
| 1517907 | 3077 | 3096 | N/A | N/A | GTGGAGTCCTGCTATGGCTT | 100 | 1531 |

TABLE 26
Reduction of APOE RNA by 5-10-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 1517176 | 4586 | 4605 | N/A | N/A | ATGTATTTATAAACAGGGTC | 94 | 1532 |
| 1517184 | 3260 | 3279 | N/A | N/A | ACATTCTAAGCTCCAACCTC | 98 | 1533 |
| 1517185 | 4235 | 4254 | N/A | N/A | AATAAAGATCACAGCTGCCC | 81 | 1534 |
| 1517186 | 6278 | 6297 | 1180 | 1199 | AGGAGGCACGGGGTGGCGTG | 104 | 1535 |
| 1517215 | 3504 | 3523 | N/A | N/A | CCAGGTTAGCCTTCCAAGCC | 91 | 1536 |
| 1517221 | 4600 | 4619 | N/A | N/A | ACTTGGAAAGCATTATGTAT | 97 | 1537 |
| 1517240 | 6223 | 6242 | 1125 | 1144 | TGTCGCTGGGCACAGGGGCG | 103 | 1538 |
| 1517259 | 3200 | 3219 | N/A | N/A | CTCATTTTAAAGTTCTCCAA | 89 | 1539 |
| 1517279 | 6298 | 6317 | 1200 | 1219 | GCTGCAGGCTGCGCGGAGGC | 52 | 1540 |
| 1517282 | 6258 | 6277 | 1160 | 1179 | GGGTCGCATGGCTGCAGGCT | 72 | 1541 |
| 1517299 | 6143 | 6162 | 1045 | 1064 | TCTTCCACCAGGGGCTCGAA | 107 | 1542 |
| 1517314 | 3212 | 3231 | N/A | N/A | GCTAATTCAGTCCTCATTTT | 99 | 1543 |
| 1517330 | 3672 | 3691 | N/A | N/A | ACCGAGCAAGCCCCGCCCCC | 75 | 1544 |
| 1517332 | 3373 | 3392 | N/A | N/A | GCCTCCATAGAAAATTCCAT | 107 | 1545 |
| 1517334 | 3835 | 3854 | N/A | N/A | TCTAACTCCTATCTCAAGGA | 93 | 1546 |
| 1517384 | 2858 | 2877 | 110 | 129 | TGAGCTCATCCCCGTGCCCC | 111 | 1547 |
| 1517387 | 5037 | 5056 | N/A | N/A | ACCCAGGCCCCCCAAGACTT | 99† | 1548 |
| 1517400 | 6348 | 6367 | 1250 | 1269 | AAACTAGGGTCCACCCCAGG | 90 | 1549 |
| 1517403 | 3610 | 3629 | 184 | 203 | TTCATCTTCCTGCCTGTGAT | 85 | 1550 |
| 1517413 | 2997 | 3016 | N/A | N/A | CCCAGCCCTTCCCAGGTCCA | 111 | 1551 |
| 1517415 | 3302 | 3321 | N/A | N/A | GTTCCATCTCAGTCCCAGTC | 95 | 1552 |
| 1517416 | 3393 | 3412 | N/A | N/A | ATCTCCCCATCCCCAGGTCG | 94 | 1553 |
| 1517427 | 6363 | 6382 | 1265 | 1284 | TGGTGAATCTTTATTAAACT | 44 | 1193 |
| 1517431 | 4826 | 4845 | 308 | 327 | GCGCTGGCCGCTCTGCCACT | 166 | 1554 |
| 1517433 | 6203 | 6222 | 1105 | 1124 | GCGCTGGTGCCCACGGCAGC | 100 | 1555 |
| 1517437 | 4899 | 4918 | 381 | 400 | CCTCCTGCACCTGCTCAGAC | 20† | 1556 |
| 1517439 | 4662 | 4681 | N/A | N/A | GTGCTCCACAAATGCTTCTT | 113 | 1557 |
| 1517457 | 2954 | 2973 | N/A | N/A | CCGCTCCTCCTCTCCCCAAG | 114 | 1558 |
| 1517475 | 2871 | 2890 | 123 | 142 | TTCTAGAGGCCCCTGAGCTC | 101 | 1559 |
| 1517490 | 3461 | 3480 | N/A | N/A | TAATCCCAGCACATTTACCA | 90 | 1560 |
| 1517496 | 3288 | 3307 | N/A | N/A | CCAGTCTCGCATTCCTCATT | 117 | 1561 |
| 1517507 | 6238 | 6257 | 1140 | 1159 | TCGGCGTTCAGTGATTGTCG | 92 | 1562 |
| 1517517 | 3158 | 3177 | N/A | N/A | CTGCCCCTGGCTCCCCAGTT | 102 | 1563 |
| 1517519 | 6369 | 6388 | 1271 | 1290 | GAAACTTGGTGAATCTTTAT | 30 | 1564 |
| 1517540 | 3417 | 3436 | N/A | N/A | TTAACTCCCTCCTGGTCTTC | 111 | 1565 |
| 1517541 | 3845 | 3864 | N/A | N/A | CAAAACAACTTCTAACTCCT | 92 | 1566 |
| 1517553 | 3336 | 3355 | N/A | N/A | CAAATTCCATCCCCCCACCC | 98 | 1567 |
| 1517580 | 4179 | 4198 | N/A | N/A | GAAACCTCTAACTCCCAGCC | 93 | 1568 |
| 1517593 | 6163 | 6182 | 1065 | 1084 | CGGCCCACTGGCGCTGCATG | 94 | 1569 |
| 1517602 | 3363 | 3382 | N/A | N/A | AAAATTCCATCTTCCTCTCC | 91 | 1570 |
| 1517608 | 6288 | 6307 | 1190 | 1209 | GCGCGGAGGCAGGAGGCACG | 70 | 1571 |
| 1517622 | 6308 | 6327 | 1210 | 1229 | AGGGTCTCCCGCTGCAGGCT | 52 | 1572 |
| 1517623 | 4201 | 4220 | N/A | N/A | ACTATCTGCCTGCAATGCAT | 84 | 1573 |
| 1517627 | 4776 | 4795 | 258 | 277 | CTGTCTCCACCGCTTGCTCC | 121 | 1574 |
| 1517647 | 2941 | 2960 | N/A | N/A | CCCCAAGCCCGACCCCGAGT | 96 | 1575 |
| 1517652 | 6358 | 6377 | 1260 | 1279 | AATCTTTATTAAACTAGGGT | 41 | 1576 |
| 1517658 | 4790 | 4809 | 272 | 291 | CTCGGGCTCCGGCTCTGTCT | 119 | 1577 |
| 1517668 | 3429 | 3448 | N/A | N/A | CCCATTCCCTATTTAACTCC | 94 | 1578 |
| 1517676 | 6183 | 6202 | 1085 | 1104 | CTGCACCTTCTCCACCAGCC | 86 | 1579 |
| 1517689 | 3560 | 3579 | N/A | N/A | TCAACCGCCAGTGAGGACTC | 100 | 1580 |
| 1517703 | 3056 | 3075 | N/A | N/A | CATCCCAGTCCAGCTGCTCT | 100 | 1581 |
| 1517707 | 3036 | 3055 | N/A | N/A | CCCCACCCCACCTTCTAGCG | 77 | 1582 |
| 1517709 | 2980 | 2999 | N/A | N/A | CCAGTCCCCTGCTGCTTGCC | 99 | 1583 |
| 1517715 | 6338 | 6357 | 1240 | 1259 | CCACCCCAGGAGGACGGCTG | 91 | 1584 |
| 1517723 | 3229 | 3248 | N/A | N/A | CCGTGTTCCATTTATGAGCT | 83 | 1585 |
| 1517727 | 3405 | 3424 | N/A | N/A | TGGTCTTCTCTTATCTCCCC | 101 | 1586 |
| 1517748 | 4189 | 4208 | N/A | N/A | CAATGCATTAGAAACCTCTA | 86 | 1587 |
| 1517784 | 2967 | 2986 | N/A | N/A | GCTTGCCTCACCCCCGCTCC | 95 | 1588 |
| 1517788 | 2889 | 2908 | 141 | 160 | TCCCAGGGTCCCAGCTCTTT | 114 | 1589 |
| 1517793 | 6248 | 6267 | 1150 | 1169 | GCTGCAGGCTTCGGCGTTCA | 53 | 1590 |
| 1517805 | 4754 | 4773 | N/A | N/A | CTTGGCCTGGCATCCTGTGT | 98 | 1591 |
| 1517808 | 3662 | 3681 | N/A | N/A | CCCCGCCCCCATACCTGCCA | 113 | 1592 |
| 1517815 | 3622 | 3641 | 196 | 215 | GCCCACAGAACCTTCATCTT | 88 | 1593 |
| 1517822 | 3534 | 3553 | N/A | N/A | CCCCCAGCCCGGCCCCAACC | 145 | 1594 |
| 1517835 | 3484 | 3503 | N/A | N/A | TTGTTGCATTATCTGCAACA | 99 | 1595 |
| 1517836 | 3521 | 3540 | N/A | N/A | CCCAACCCGGCCTCACCCCA | 89 | 1596 |
| 1517847 | 5049 | 5068 | N/A | N/A | GAACCAGCAGAGACCCAGGC | 102† | 1597 |
| 1517854 | 3072 | 3091 | N/A | N/A | GTCCTGCTATGGCTTACATC | 101 | 1598 |
| 1517858 | 4301 | 4320 | N/A | N/A | CCAGCGGAAAAGCATGTATT | 100 | 1599 |
| 1517861 | 6268 | 6287 | 1170 | 1189 | GGGTGGCGTGGGGTCGCATG | 77 | 1600 |
| 1517867 | 3471 | 3490 | N/A | N/A | TGCAACAGCCTAATCCCAGC | 108 | 1601 |
| 1517875 | 3651 | 3670 | N/A | N/A | TACCTGCCAGGAATGTGACC | 107 | 1602 |
| 1517878 | 6318 | 6337 | 1220 | 1239 | GGGCGGGGACAGGGTCTCCC | 67 | 1603 |
| 1517886 | 4943 | 4962 | N/A | N/A | GGACACTCACCTCAGTTCCT | 125† | 1604 |
| 1517890 | 3272 | 3291 | N/A | N/A | CATTCTCCCTTCACATTCTA | 110 | 1605 |
| 1517892 | 3439 | 3458 | N/A | N/A | CCGCCCCCAACCCATTCCCT | 133 | 1606 |
| 1517901 | 6328 | 6347 | 1230 | 1249 | AGGACGGCTGGGGCGGGGAC | 150 | 1607 |
| 1517911 | 5091 | 5110 | N/A | N/A | AGAGAGCTAAAGCCAGGAGT | 115† | 1608 |

TABLE 27
Reduction of APOE RNA by 5-10-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 708022 | 6257 | 6276 | 1159 | 1178 | GGTCGCATGGCTGCAGGCTT | 36 | 71 |
| 942593 | 6347 | 6366 | 1249 | 1268 | AACTAGGGTCCACCCCAGGA | 45 | 75 |
| 1517144 | 4200 | 4219 | N/A | N/A | CTATCTGCCTGCAATGCATT | 75 | 1609 |
| 1517147 | 2940 | 2959 | N/A | N/A | CCCAAGCCCGACCCCGAGTA | 108 | 1610 |
| 1517193 | 6267 | 6286 | 1169 | 1188 | GGTGGCGTGGGGTCGCATGG | 54 | 1611 |
| 1517203 | 2888 | 2907 | 140 | 159 | CCCAGGGTCCCAGCTCTTTC | 65 | 78 |
| 1517206 | 4272 | 4291 | N/A | N/A | TGTGTGCCCCAGGCAGGGCT | 112 | 83 |
| 1517217 | 3519 | 3538 | N/A | N/A | CAACCCGGCCTCACCCCAGG | 103 | 1612 |
| 1517218 | 2953 | 2972 | N/A | N/A | CGCTCCTCCTCTCCCCAAGC | 92 | 1613 |
| 1517223 | 3391 | 3410 | N/A | N/A | CTCCCCATCCCCAGGTCGGC | 82 | 1614 |
| 1517224 | 3259 | 3278 | N/A | N/A | CATTCTAAGCTCCAACCTCA | 80 | 1615 |
| 1517243 | N/A | N/A | 225 | 244 | ATCCTGCCAGGAATGTGACC | 97 | 1616 |
| 1517265 | 3362 | 3381 | N/A | N/A | AAATTCCATCTTCCTCTCCC | 95 | 1617 |
| 1517267 | 2996 | 3015 | N/A | N/A | CCAGCCCTTCCCAGGTCCAG | 83 | 1618 |
| 1517268 | 5090 | 5109 | N/A | N/A | GAGAGCTAAAGCCAGGAGTC | 83† | 1619 |
| 1517269 | 4898 | 4917 | 380 | 399 | CTCCTGCACCTGCTCAGACA | 15† | 1620 |
| 1517271 | 4234 | 4253 | N/A | N/A | ATAAAGATCACAGCTGCCCC | 90 | 1621 |
| 1517283 | 3301 | 3320 | N/A | N/A | TTCCATCTCAGTCCCAGTCT | 74 | 1622 |
| 1517302 | 2857 | 2876 | 109 | 128 | GAGCTCATCCCCGTGCCCCC | 79 | 1623 |
| 1517303 | 4941 | 4960 | N/A | N/A | ACACTCACCTCAGTTCCTGG | 94† | 1624 |
| 1517319 | 5048 | 5067 | N/A | N/A | AACCAGCAGAGACCCAGGCC | 101† | 1625 |
| 1517340 | 4789 | 4808 | 271 | 290 | TCGGGCTCCGGCTCTGTCTC | 91 | 1626 |
| 1517359 | 6247 | 6266 | 1149 | 1168 | CTGCAGGCTTCGGCGTTCAG | 63 | 1627 |
| 1517364 | 6287 | 6306 | 1189 | 1208 | CGCGGAGGCAGGAGGCACGG | 61 | 1628 |
| 1517374 | 3671 | 3690 | N/A | N/A | CCGAGCAAGCCCCGCCCCCA | 77 | 1629 |
| 1517381 | 3211 | 3230 | N/A | N/A | CTAATTCAGTCCTCATTTTA | 85 | 1630 |
| 1517389 | 4717 | 4736 | N/A | N/A | GGTCAGGGTCCTTCTGACCC | 104 | 1631 |
| 1517394 | 3482 | 3501 | N/A | N/A | GTTGCATTATCTGCAACAGC | 86 | 1632 |
| 1517407 | 6237 | 6256 | 1139 | 1158 | CGGCGTTCAGTGATTGTCGC | 66 | 1633 |
| 1517409 | 2870 | 2889 | 122 | 141 | TCTAGAGGCCCCTGAGCTCA | 102 | 1634 |
| 1517427 | 6363 | 6382 | 1265 | 1284 | TGGTGAATCTTTATTAAACT | 20 | 1193 |
| 1517429 | 3225 | 3244 | N/A | N/A | GTTCCATTTATGAGCTAATT | 78 | 1635 |
| 1517444 | 4824 | 4843 | 306 | 325 | GCTGGCCGCTCTGCCACTCG | 96 | 1636 |
| 1517451 | 4188 | 4207 | N/A | N/A | AATGCATTAGAAACCTCTAA | 92 | 1637 |
| 1517453 | 3199 | 3218 | N/A | N/A | TCATTTTAAAGTTCTCCAAT | 77 | 1638 |
| 1517480 | 3428 | 3447 | N/A | N/A | CCATTCCCTATTTAACTCCC | 82 | 1639 |
| 1517499 | 3503 | 3522 | N/A | N/A | CAGGTTAGCCTTCCAAGCCT | 84 | 1640 |
| 1517502 | 3035 | 3054 | N/A | N/A | CCCACCCCACCTTCTAGCGG | 77 | 1641 |
| 1517520 | 3834 | 3853 | N/A | N/A | CTAACTCCTATCTCAAGGAT | 94 | 1642 |
| 1517534 | 3844 | 3863 | N/A | N/A | AAAACAACTTCTAACTCCTA | 81 | 1643 |
| 1517547 | 3460 | 3479 | N/A | N/A | AATCCCAGCACATTTACCAA | 89 | 1644 |
| 1517551 | 6141 | 6160 | 1043 | 1062 | TTCCACCAGGGGCTCGAACC | 95 | 1645 |
| 1517567 | 4599 | 4618 | N/A | N/A | CTTGGAAAGCATTATGTATT | 87 | 1646 |
| 1517575 | 4775 | 4794 | 257 | 276 | TGTCTCCACCGCTTGCTCCA | 109 | 1647 |
| 1517576 | 3157 | 3176 | N/A | N/A | TGCCCCTGGCTCCCCAGTTA | 103 | 1648 |
| 1517590 | 3287 | 3306 | N/A | N/A | CAGTCTCGCATTCCTCATTC | 86 | 1649 |
| 1517596 | 6337 | 6356 | 1239 | 1258 | CACCCCAGGAGGACGGCTGG | 105 | 1650 |
| 1517597 | 4585 | 4604 | N/A | N/A | TGTATTTATAAACAGGGTCT | 83 | 1651 |
| 1517599 | 3438 | 3457 | N/A | N/A | CGCCCCCAACCCATTCCCTA | 85 | 1652 |
| 1517604 | 6307 | 6326 | 1209 | 1228 | GGGTCTCCCGCTGCAGGCTG | 29 | 1653 |
| 1517610 | 6201 | 6220 | 1103 | 1122 | GCTGGTGCCCACGGCAGCCT | 94 | 1654 |
| 1517639 | 3532 | 3551 | N/A | N/A | CCCAGCCCGGCCCCAACCCG | 85 | 1655 |
| 1517641 | 4661 | 4680 | N/A | N/A | TGCTCCACAAATGCTTCTTT | 86 | 1656 |
| 1517643 | 3416 | 3435 | N/A | N/A | TAACTCCCTCCTGGTCTTCT | 91 | 1657 |
| 1517650 | 3555 | 3574 | N/A | N/A | CGCCAGTGAGGACTCCTCCC | 97 | 1658 |
| 1517651 | 3621 | 3640 | 195 | 214 | CCCACAGAACCTTCATCTTC | 37 | 1659 |
| 1517656 | 6327 | 6346 | 1229 | 1248 | GGACGGCTGGGGCGGGGACA | 97 | 1660 |
| 1517661 | 3271 | 3290 | N/A | N/A | ATTCTCCCTTCACATTCTAA | 86 | 1661 |
| 1517663 | 3372 | 3391 | N/A | N/A | CCTCCATAGAAAATTCCATC | 59 | 1662 |
| 1517674 | 3055 | 3074 | N/A | N/A | ATCCCAGTCCAGCTGCTCTC | 90 | 1663 |
| 1517680 | 4178 | 4197 | N/A | N/A | AAACCTCTAACTCCCAGCCA | 81 | 1664 |
| 1517690 | 6317 | 6336 | 1219 | 1238 | GGCGGGGACAGGGTCTCCCG | 64 | 1665 |
| 1517692 | 6221 | 6240 | 1123 | 1142 | TCGCTGGGCACAGGGGCGGC | 82 | 1666 |
| 1517699 | 2966 | 2985 | N/A | N/A | CTTGCCTCACCCCCGCTCCT | 88 | 1667 |
| 1517726 | 6161 | 6180 | 1063 | 1082 | GCCCACTGGCGCTGCATGTC | 56 | 1668 |
| 1517732 | 3070 | 3089 | N/A | N/A | CCTGCTATGGCTTACATCCC | 84 | 1669 |
| 1517753 | 5036 | 5055 | N/A | N/A | CCCAGGCCCCCCAAGACTTA | 99† | 1670 |
| 1517776 | 3470 | 3489 | N/A | N/A | GCAACAGCCTAATCCCAGCA | 82 | 1671 |
| 1517780 | N/A | N/A | 236 | 255 | CTTGGCCTGGCATCCTGCCA | 103 | 1672 |
| 1517791 | 3609 | 3628 | 183 | 202 | TCATCTTCCTGCCTGTGATT | 78 | 1673 |
| 1517812 | 6297 | 6316 | 1199 | 1218 | CTGCAGGCTGCGCGGAGGCA | 41 | 1674 |
| 1517821 | 6277 | 6296 | 1179 | 1198 | GGAGGCACGGGGTGGCGTGG | 92 | 1675 |
| 1517826 | 6181 | 6200 | 1083 | 1102 | GCACCTTCTCCACCAGCCCG | 93 | 1676 |
| 1517832 | 3332 | 3351 | N/A | N/A | TTCCATCCCCCCACCCCCTC | 85 | 1677 |
| 1517841 | 6357 | 6376 | 1259 | 1278 | ATCTTTATTAAACTAGGGTC | 24 | 1678 |
| 1517842 | 6368 | 6387 | 1270 | 1289 | AAACTTGGTGAATCTTTATT | 32 | 1679 |
| 1517846 | 2979 | 2998 | N/A | N/A | CAGTCCCCTGCTGCTTGCCT | 89 | 1680 |
| 1517857 | 3404 | 3423 | N/A | N/A | GGTCTTCTCTTATCTCCCCA | 92 | 1681 |

TABLE 28
Reduction of APOE RNA by 5-10-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 942588 | 6256 | 6275 | 1158 | 1177 | GTCGCATGGCTGCAGGCTTC | 23 | 1132 |
| 1517141 | 4939 | 4958 | N/A | N/A | ACTCACCTCAGTTCCTGGGT | 92† | 1682 |
| 1517146 | 6236 | 6255 | 1138 | 1157 | GGCGTTCAGTGATTGTCGCT | 73 | 1683 |
| 1517153 | 3286 | 3305 | N/A | N/A | AGTCTCGCATTCCTCATTCT | 79 | 1684 |
| 1517159 | 4598 | 4617 | N/A | N/A | TTGGAAAGCATTATGTATTT | 115 | 1685 |
| 1517181 | 3481 | 3500 | N/A | N/A | TTGCATTATCTGCAACAGCC | 80 | 1686 |
| 1517189 | 3403 | 3422 | N/A | N/A | GTCTTCTCTTATCTCCCCAT | 87 | 1687 |
| 1517190 | 2965 | 2984 | N/A | N/A | TTGCCTCACCCCCGCTCCTC | 102 | 1688 |
| 1517198 | 3620 | 3639 | 194 | 213 | CCACAGAACCTTCATCTTCC | 65 | 1689 |
| 1517202 | 6286 | 6305 | 1188 | 1207 | GCGGAGGCAGGAGGCACGGG | 73 | 1690 |
| 1517204 | 3661 | 3680 | N/A | N/A | CCCGCCCCCATACCTGCCAG | 104 | 1691 |
| 1517205 | 3518 | 3537 | N/A | N/A | AACCCGGCCTCACCCCAGGT | 87 | 1692 |
| 1517213 | 6199 | 6218 | 1101 | 1120 | TGGTGCCCACGGCAGCCTGC | 99 | 1693 |
| 1517216 | 3069 | 3088 | N/A | N/A | CTGCTATGGCTTACATCCCA | 82 | 1694 |
| 1517220 | 6367 | 6386 | 1269 | 1288 | AACTTGGTGAATCTTTATTA | 23 | 1695 |
| 1517225 | 3502 | 3521 | N/A | N/A | AGGTTAGCCTTCCAAGCCTT | 93 | 1696 |
| 1517228 | 4187 | 4206 | N/A | N/A | ATGCATTAGAAACCTCTAAC | 92 | 1697 |
| 1517231 | 3198 | 3217 | N/A | N/A | CATTTTAAAGTTCTCCAATC | 78 | 1698 |
| 1517237 | 5079 | 5098 | N/A | N/A | CCAGGAGTCAGAAATGGGAA | 74† | 1699 |
| 1517241 | 3650 | 3669 | N/A | N/A | ACCTGCCAGGAATGTGACCA | 83 | 1700 |
| 1517254 | 6356 | 6375 | 1258 | 1277 | TCTTTATTAAACTAGGGTCC | 31 | 1701 |
| 1517256 | 4822 | 4841 | 304 | 323 | TGGCCGCTCTGCCACTCGGT | 72 | 1702 |
| 1517273 | 2978 | 2997 | N/A | N/A | AGTCCCCTGCTGCTTGCCTC | 92 | 1703 |
| 1517285 | 4177 | 4196 | N/A | N/A | AACCTCTAACTCCCAGCCAG | 78 | 1704 |
| 1517292 | 3269 | 3288 | N/A | N/A | TCTCCCTTCACATTCTAAGC | 102 | 1705 |
| 1517296 | 5035 | 5054 | N/A | N/A | CCAGGCCCCCCAAGACTTAG | 91† | 1706 |
| 1517320 | 2887 | 2906 | 139 | 158 | CCAGGGTCCCAGCTCTTTCT | 95 | 1707 |
| 1517326 | 4894 | 4913 | 376 | 395 | TGCACCTGCTCAGACAGTGT | 49† | 1708 |
| 1517331 | 4786 | 4805 | 268 | 287 | GGCTCCGGCTCTGTCTCCAC | 67 | 1709 |
| 1517351 | 2993 | 3012 | N/A | N/A | GCCCTTCCCAGGTCCAGTCC | 79 | 1710 |
| 1517355 | 3210 | 3229 | N/A | N/A | TAATTCAGTCCTCATTTTAA | 88 | 1711 |
| 1517360 | 5047 | 5066 | N/A | N/A | ACCAGCAGAGACCCAGGCCC | 100† | 1712 |
| 1517377 | 6296 | 6315 | 1198 | 1217 | TGCAGGCTGCGCGGAGGCAG | 35 | 1713 |
| 1517397 | 3554 | 3573 | N/A | N/A | GCCAGTGAGGACTCCTCCCA | 98 | 1714 |
| 1517419 | 6139 | 6158 | 1041 | 1060 | CCACCAGGGGCTCGAACCAG | 84 | 1715 |
| 1517420 | 4584 | 4603 | N/A | N/A | GTATTTATAAACAGGGTCTT | 87 | 1716 |
| 1517427 | 6363 | 6382 | 1265 | 1284 | TGGTGAATCTTTATTAAACT | 20 | 1193 |
| 1517465 | 6326 | 6345 | 1228 | 1247 | GACGGCTGGGGCGGGGACAG | 89 | 1717 |
| 1517466 | 3414 | 3433 | N/A | N/A | ACTCCCTCCTGGTCTTCTCT | 106 | 1718 |
| 1517470 | 3670 | 3689 | N/A | N/A | CGAGCAAGCCCCGCCCCCAT | 113 | 1719 |
| 1517476 | 6159 | 6178 | 1061 | 1080 | CCACTGGCGCTGCATGTCTT | 69 | 1720 |
| 1517509 | 4245 | 4264 | N/A | N/A | GGTGATGGAGAATAAAGATC | 93 | 1721 |
| 1517515 | 6219 | 6238 | 1121 | 1140 | GCTGGGCACAGGGGCGGCGC | 100 | 1722 |
| 1517527 | 3053 | 3072 | N/A | N/A | CCCAGTCCAGCTGCTCTCCC | 97 | 1723 |
| 1517530 | 3459 | 3478 | N/A | N/A | ATCCCAGCACATTTACCAAG | 90 | 1724 |
| 1517544 | 3361 | 3380 | N/A | N/A | AATTCCATCTTCCTCTCCCG | 98 | 1725 |
| 1517565 | 6276 | 6295 | 1178 | 1197 | GAGGCACGGGGTGGCGTGGG | 57 | 1726 |
| 1517572 | 3258 | 3277 | N/A | N/A | ATTCTAAGCTCCAACCTCAC | 84 | 1727 |
| 1517579 | 4716 | 4735 | N/A | N/A | GTCAGGGTCCTTCTGACCCC | 117 | 1728 |
| 1517589 | 3322 | 3341 | N/A | N/A | CCACCCCCTCCCCACCGCCG | 95 | 1729 |
| 1517594 | 3778 | 3797 | N/A | N/A | TTCAGAGCCAGGAAGCAGCA | 100 | 1730 |
| 1517633 | 6246 | 6265 | 1148 | 1167 | TGCAGGCTTCGGCGTTCAGT | 67 | 1731 |
| 1517634 | 6316 | 6335 | 1218 | 1237 | GCGGGGACAGGGTCTCCCGC | 105 | 1732 |
| 1517664 | 4228 | 4247 | N/A | N/A | ATCACAGCTGCCCCGTGTCT | 99 | 1733 |
| 1517681 | 3605 | 3624 | 179 | 198 | CTTCCTGCCTGTGATTGGCC | 79 | 1734 |
| 1517686 | 6346 | 6365 | 1248 | 1267 | ACTAGGGTCCACCCCAGGAG | 60 | 1735 |
| 1517694 | 2854 | 2873 | 106 | 125 | CTCATCCCCGTGCCCCCGAC | 84 | 1736 |
| 1517695 | 3390 | 3409 | N/A | N/A | TCCCCATCCCCAGGTCGGCC | 100 | 1737 |
| 1517704 | 2925 | 2944 | N/A | N/A | GAGTAGCTCTCCTGAGACTA | 89 | 1738 |
| 1517735 | 3156 | 3175 | N/A | N/A | GCCCCTGGCTCCCCAGTTAT | 91 | 1739 |
| 1517741 | 2869 | 2888 | 121 | 140 | CTAGAGGCCCCTGAGCTCAT | 84 | 1740 |
| 1517781 | 3530 | 3549 | N/A | N/A | CAGCCCGGCCCCAACCCGGC | 108 | 1741 |
| 1517792 | 4197 | 4216 | N/A | N/A | TCTGCCTGCAATGCATTAGA | 94 | 1742 |
| 1517794 | 4772 | 4791 | 254 | 273 | CTCCACCGCTTGCTCCACCT | 75 | 1743 |
| 1517797 | 6266 | 6285 | 1168 | 1187 | GTGGCGTGGGGTCGCATGGC | 47 | 1744 |
| 1517800 | 4656 | 4675 | N/A | N/A | CACAAATGCTTCTTTGGAGC | 96 | 1745 |
| 1517806 | 6306 | 6325 | 1208 | 1227 | GGTCTCCCGCTGCAGGCTGC | 41 | 1746 |
| 1517809 | 3300 | 3319 | N/A | N/A | TCCATCTCAGTCCCAGTCTC | 99 | 1747 |
| 1517824 | 3371 | 3390 | N/A | N/A | CTCCATAGAAAATTCCATCT | 75 | 1748 |
| 1517830 | 3843 | 3862 | N/A | N/A | AAACAACTTCTAACTCCTAT | 94 | 1749 |
| 1517845 | 3224 | 3243 | N/A | N/A | TTCCATTTATGAGCTAATTC | 91 | 1750 |
| 1517855 | 3034 | 3053 | N/A | N/A | CCACCCCACCTTCTAGCGGG | 88 | 1751 |
| 1517864 | 3437 | 3456 | N/A | N/A | GCCCCCAACCCATTCCCTAT | 91 | 1752 |
| 1517868 | 3427 | 3446 | N/A | N/A | CATTCCCTATTTAACTCCCT | 98 | 1753 |
| 1517876 | 2952 | 2971 | N/A | N/A | GCTCCTCCTCTCCCCAAGCC | 65 | 1754 |
| 1517880 | 6336 | 6355 | 1238 | 1257 | ACCCCAGGAGGACGGCTGGG | 94 | 72 |
| 1517888 | 3469 | 3488 | N/A | N/A | CAACAGCCTAATCCCAGCAC | 84 | 1755 |
| 1517895 | 6179 | 6198 | 1081 | 1100 | ACCTTCTCCACCAGCCCGGC | 78 | 1756 |

TABLE 29
Reduction of APOE RNA by 5-10-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 942587 | 6255 | 6274 | 1157 | 1176 | TCGCATGGCTGCAGGCTTCG | 30 | 1061 |
| 942591 | 6305 | 6324 | 1207 | 1226 | GTCTCCCGCTGCAGGCTGCG | 48 | 990 |
| 1517143 | 6335 | 6354 | 1237 | 1256 | CCCCAGGAGGACGGCTGGGG | 87 | 1757 |
| 1517161 | 4196 | 4215 | N/A | N/A | CTGCCTGCAATGCATTAGAA | 81 | 1758 |
| 1517163 | 2867 | 2886 | 119 | 138 | AGAGGCCCCTGAGCTCATCC | 97 | 1759 |
| 1517177 | 6235 | 6254 | 1137 | 1156 | GCGTTCAGTGATTGTCGCTG | 63 | 1760 |
| 1517196 | 3553 | 3572 | N/A | N/A | CCAGTGAGGACTCCTCCCAC | 83 | 1761 |
| 1517214 | 4244 | 4263 | N/A | N/A | GTGATGGAGAATAAAGATCA | 81 | 1762 |
| 1517219 | 3257 | 3276 | N/A | N/A | TTCTAAGCTCCAACCTCACA | 87 | 1763 |
| 1517233 | 4186 | 4205 | N/A | N/A | TGCATTAGAAACCTCTAACT | 85 | 1764 |
| 1517239 | 2974 | 2993 | N/A | N/A | CCCTGCTGCTTGCCTCACCC | 81 | 1765 |
| 1517242 | 3052 | 3071 | N/A | N/A | CCAGTCCAGCTGCTCTCCCC | 85 | 1766 |
| 1517244 | 2950 | 2969 | N/A | N/A | TCCTCCTCTCCCCAAGCCCG | 99 | 1767 |
| 1517247 | 3529 | 3548 | N/A | N/A | AGCCCGGCCCCAACCCGGCC | 101 | 1768 |
| 1517251 | 3660 | 3679 | N/A | N/A | CCGCCCCCATACCTGCCAGG | 92 | 1769 |
| 1517258 | 3632 | 3651 | 206 | 225 | CAGCAACGCAGCCCACAGAA | 86 | 1770 |
| 1517262 | 3436 | 3455 | N/A | N/A | CCCCCAACCCATTCCCTATT | 124 | 1771 |
| 1517266 | 2849 | 2868 | 101 | 120 | CCCCGTGCCCCCGACTGCGC | 96 | 1772 |
| 1517305 | 3426 | 3445 | N/A | N/A | ATTCCCTATTTAACTCCCTC | 86 | 1773 |
| 1517310 | 4597 | 4616 | N/A | N/A | TGGAAAGCATTATGTATTTA | 85 | 1774 |
| 1517321 | 3669 | 3688 | N/A | N/A | GAGCAAGCCCCGCCCCCATA | 79 | 1775 |
| 1517324 | 3499 | 3518 | N/A | N/A | TTAGCCTTCCAAGCCTTGTT | 87 | 1776 |
| 1517335 | 4911 | 4930 | 393 | 412 | AGCTGAGCAGCTCCTCCTGC | 30† | 1777 |
| 1517342 | 4175 | 4194 | N/A | N/A | CCTCTAACTCCCAGCCAGGT | 103 | 1778 |
| 1517352 | 4713 | 4732 | N/A | N/A | AGGGTCCTTCTGACCCCGTC | 90 | 1779 |
| 1517354 | 6325 | 6344 | 1227 | 1246 | ACGGCTGGGGCGGGGACAGG | 108 | 1780 |
| 1517357 | 3298 | 3317 | N/A | N/A | CATCTCAGTCCCAGTCTCGC | 82 | 1781 |
| 1517366 | 3468 | 3487 | N/A | N/A | AACAGCCTAATCCCAGCACA | 78 | 1782 |
| 1517373 | 3517 | 3536 | N/A | N/A | ACCCGGCCTCACCCCAGGTT | 97 | 1783 |
| 1517380 | 6345 | 6364 | 1247 | 1266 | CTAGGGTCCACCCCAGGAGG | 71 | 1784 |
| 1517395 | 4655 | 4674 | N/A | N/A | ACAAATGCTTCTTTGGAGCC | 93 | 1785 |
| 1517399 | 2924 | 2943 | N/A | N/A | AGTAGCTCTCCTGAGACTAC | 106 | 1786 |
| 1517402 | 3360 | 3379 | N/A | N/A | ATTCCATCTTCCTCTCCCGG | 74 | 1787 |
| 1517405 | 3480 | 3499 | N/A | N/A | TGCATTATCTGCAACAGCCT | 80 | 1788 |
| 1517418 | 3321 | 3340 | N/A | N/A | CACCCCCTCCCCACCGCCGG | 130 | 1789 |
| 1517422 | 2964 | 2983 | N/A | N/A | TGCCTCACCCCCGCTCCTCC | 110 | 1790 |
| 1517427 | 6363 | 6382 | 1265 | 1284 | TGGTGAATCTTTATTAAACT | 26 | 1193 |
| 1517442 | 5060 | 5079 | N/A | N/A | AGAGGAAGCTAGAACCAGCA | 81† | 1791 |
| 1517445 | 6355 | 6374 | 1257 | 1276 | CTTTATTAAACTAGGGTCCA | 30 | 1792 |
| 1517446 | 6137 | 6156 | 1039 | 1058 | ACCAGGGGCTCGAACCAGCT | 76 | 1793 |
| 1517469 | 3008 | 3027 | N/A | N/A | CGTCTCTGCTGCCCAGCCCT | 95 | 1794 |
| 1517484 | 5034 | 5053 | N/A | N/A | CAGGCCCCCCAAGACTTAGC | 102† | 1795 |
| 1517505 | 4785 | 4804 | 267 | 286 | GCTCCGGCTCTGTCTCCACC | 66 | 1796 |
| 1517516 | 6197 | 6216 | 1099 | 1118 | GTGCCCACGGCAGCCTGCAC | 78 | 67 |
| 1517526 | 4821 | 4840 | 303 | 322 | GGCCGCTCTGCCACTCGGTC | 102 | 1797 |
| 1517532 | 6265 | 6284 | 1167 | 1186 | TGGCGTGGGGTCGCATGGCT | 27 | 1798 |
| 1517562 | 3153 | 3172 | N/A | N/A | CCTGGCTCCCCAGTTATGGA | 107 | 1799 |
| 1517563 | 6315 | 6334 | 1217 | 1236 | CGGGGACAGGGTCTCCCGCT | 92 | 1800 |
| 1517571 | 3066 | 3085 | N/A | N/A | CTATGGCTTACATCCCAGTC | 77 | 1801 |
| 1517582 | 3285 | 3304 | N/A | N/A | GTCTCGCATTCCTCATTCTC | 80 | 1802 |
| 1517598 | 4542 | 4561 | N/A | N/A | GCAGCGGCCTGAACATGGTT | 107 | 1803 |
| 1517615 | 6157 | 6176 | 1059 | 1078 | ACTGGCGCTGCATGTCTTCC | 74 | 1804 |
| 1517630 | 3370 | 3389 | N/A | N/A | TCCATAGAAAATTCCATCTT | 77 | 1805 |
| 1517635 | 3402 | 3421 | N/A | N/A | TCTTCTCTTATCTCCCCATC | 86 | 1806 |
| 1517642 | 3777 | 3796 | N/A | N/A | TCAGAGCCAGGAAGCAGCAC | 111 | 1807 |
| 1517644 | 3223 | 3242 | N/A | N/A | TCCATTTATGAGCTAATTCA | 69 | 1808 |
| 1517655 | 5046 | 5065 | N/A | N/A | CCAGCAGAGACCCAGGCCCC | 87† | 1809 |
| 1517662 | 3619 | 3638 | 193 | 212 | CACAGAACCTTCATCTTCCT | 74 | 1810 |
| 1517665 | 6275 | 6294 | 1177 | 1196 | AGGCACGGGGTGGCGTGGGG | 77 | 1811 |
| 1517666 | 2886 | 2905 | 138 | 157 | CAGGGTCCCAGCTCTTTCTA | 82 | 1812 |
| 1517669 | 6177 | 6196 | 1079 | 1098 | CTTCTCCACCAGCCCGGCCC | 92 | 1813 |
| 1517670 | 3209 | 3228 | N/A | N/A | AATTCAGTCCTCATTTTAAA | 89 | 1814 |
| 1517718 | 3268 | 3287 | N/A | N/A | CTCCCTTCACATTCTAAGCT | 93 | 1815 |
| 1517733 | 3196 | 3215 | N/A | N/A | TTTTAAAGTTCTCCAATCGA | 71 | 1816 |
| 1517734 | 3842 | 3861 | N/A | N/A | AACAACTTCTAACTCCTATC | 73 | 1817 |
| 1517757 | 3412 | 3431 | N/A | N/A | TCCCTCCTGGTCTTCTCTTA | 82 | 1818 |
| 1517774 | 4214 | 4233 | N/A | N/A | GTGTCTGGTATTCACTATCT | 96 | 1819 |
| 1517786 | 3604 | 3623 | 178 | 197 | TTCCTGCCTGTGATTGGCCA | 73 | 1820 |
| 1517787 | 4893 | 4912 | 375 | 394 | GCACCTGCTCAGACAGTGTC | 52† | 37 |
| 1517813 | 6285 | 6304 | 1187 | 1206 | CGGAGGCAGGAGGCACGGGG | 95 | 1821 |
| 1517825 | 6217 | 6236 | 1119 | 1138 | TGGGCACAGGGGCGGCGCTG | 91 | 1822 |
| 1517827 | 4771 | 4790 | 253 | 272 | TCCACCGCTTGCTCCACCTT | 86 | 1823 |
| 1517848 | 6245 | 6264 | 1147 | 1166 | GCAGGCTTCGGCGTTCAGTG | 50 | 1824 |
| 1517871 | 6295 | 6314 | 1197 | 1216 | GCAGGCTGCGCGGAGGCAGG | 45 | 1825 |
| 1517873 | 3389 | 3408 | N/A | N/A | CCCCATCCCCAGGTCGGCCT | 95 | 1826 |
| 1517891 | 6366 | 6385 | 1268 | 1287 | ACTTGGTGAATCTTTATTAA | 32 | 1827 |
| 1517906 | 2992 | 3011 | N/A | N/A | CCCTTCCCAGGTCCAGTCCC | 96 | 1828 |
| 1517908 | 3458 | 3477 | N/A | N/A | TCCCAGCACATTTACCAAGC | 87 | 1829 |

TABLE 30
Reduction of APOE RNA by 5-10-5 MOE gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 708021 | 6254 | 6273 | 1156 | 1175 | CGCATGGCTGCAGGCTTCGG | 19 | 70 |
| 708027 | 6234 | 6253 | 1136 | 1155 | CGTTCAGTGATTGTCGCTGG | 58 | 69 |
| 1517151 | 3479 | 3498 | N/A | N/A | GCATTATCTGCAACAGCCTA | 90 | 1830 |
| 1517152 | 3388 | 3407 | N/A | N/A | CCCATCCCCAGGTCGGCCTC | 109 | 1831 |
| 1517165 | 3284 | 3303 | N/A | N/A | TCTCGCATTCCTCATTCTCC | 78 | 1832 |
| 1517174 | 2963 | 2982 | N/A | N/A | GCCTCACCCCCGCTCCTCCT | 90 | 1833 |
| 1517179 | 5058 | 5077 | N/A | N/A | AGGAAGCTAGAACCAGCAGA | 98† | 1834 |
| 1517188 | 6344 | 6363 | 1246 | 1265 | TAGGGTCCACCCCAGGAGGA | 93 | 1835 |
| 1517195 | 4195 | 4214 | N/A | N/A | TGCCTGCAATGCATTAGAAA | 96 | 1836 |
| 1517209 | 6195 | 6214 | 1097 | 1116 | GCCCACGGCAGCCTGCACCT | 104 | 1837 |
| 1517211 | 4820 | 4839 | 302 | 321 | GCCGCTCTGCCACTCGGTCT | 93 | 1838 |
| 1517222 | 3064 | 3083 | N/A | N/A | ATGGCTTACATCCCAGTCCA | 93 | 1839 |
| 1517248 | 4767 | 4786 | 249 | 268 | CCGCTTGCTCCACCTTGGCC | 88 | 1840 |
| 1517264 | 3552 | 3571 | N/A | N/A | CAGTGAGGACTCCTCCCACC | 100 | 1841 |
| 1517278 | 2866 | 2885 | 118 | 137 | GAGGCCCCTGAGCTCATCCC | 93 | 1842 |
| 1517286 | 3359 | 3378 | N/A | N/A | TTCCATCTTCCTCTCCCGGG | 80 | 1843 |
| 1517295 | 6244 | 6263 | 1146 | 1165 | CAGGCTTCGGCGTTCAGTGA | 62 | 1844 |
| 1517297 | 2884 | 2903 | 136 | 155 | GGGTCCCAGCTCTTTCTAGA | 109 | 1845 |
| 1517298 | 4708 | 4727 | N/A | N/A | CCTTCTGACCCCGTCCAGGC | 92 | 1846 |
| 1517300 | 3399 | 3418 | N/A | N/A | TCTCTTATCTCCCCATCCCC | 85 | 1847 |
| 1517327 | 3496 | 3515 | N/A | N/A | GCCTTCCAAGCCTTGTTGCA | 89 | 1848 |
| 1517350 | 3841 | 3860 | N/A | N/A | ACAACTTCTAACTCCTATCT | 87 | 1849 |
| 1517353 | 3411 | 3430 | N/A | N/A | CCCTCCTGGTCTTCTCTTAT | 94 | 1850 |
| 1517361 | 6294 | 6313 | 1196 | 1215 | CAGGCTGCGCGGAGGCAGGA | 54 | 1851 |
| 1517362 | 4783 | 4802 | 265 | 284 | TCCGGCTCTGTCTCCACCGC | 81 | 1852 |
| 1517369 | 3320 | 3339 | N/A | N/A | ACCCCCTCCCCACCGCCGGT | 89 | 1853 |
| 1517385 | 4910 | 4929 | 392 | 411 | GCTGAGCAGCTCCTCCTGCA | 37† | 1854 |
| 1517391 | 3207 | 3226 | N/A | N/A | TTCAGTCCTCATTTTAAAGT | 82 | 1855 |
| 1517393 | 6274 | 6293 | 1176 | 1195 | GGCACGGGGTGGCGTGGGGT | 73 | 1856 |
| 1517414 | 6175 | 6194 | 1077 | 1096 | TCTCCACCAGCCCGGCCCAC | 116 | 1857 |
| 1517423 | 3194 | 3213 | N/A | N/A | TTAAAGTTCTCCAATCGACG | 77 | 1858 |
| 1517427 | 6363 | 6382 | 1265 | 1284 | TGGTGAATCTTTATTAAACT | 24 | 1193 |
| 1517435 | 3267 | 3286 | N/A | N/A | TCCCTTCACATTCTAAGCTC | 99 | 1859 |
| 1517479 | 3425 | 3444 | N/A | N/A | TTCCCTATTTAACTCCCTCC | 86 | 1860 |
| 1517481 | 2949 | 2968 | N/A | N/A | CCTCCTCTCCCCAAGCCCGA | 87 | 1861 |
| 1517514 | 3770 | 3789 | N/A | N/A | CAGGAAGCAGCACAGAAGCC | 86 | 1862 |
| 1517521 | 2835 | 2854 | 87 | 106 | CTGCGCTTCTCACCGGCTCC | 109 | 1863 |
| 1517524 | 3668 | 3687 | N/A | N/A | AGCAAGCCCCGCCCCCATAC | 86 | 1864 |
| 1517525 | 3255 | 3274 | N/A | N/A | CTAAGCTCCAACCTCACAGT | 101 | 1865 |
| 1517533 | 4892 | 4911 | 374 | 393 | CACCTGCTCAGACAGTGTCT | 49† | 1866 |
| 1517543 | 3631 | 3650 | 205 | 224 | AGCAACGCAGCCCACAGAAC | 79 | 1867 |
| 1517545 | 6324 | 6343 | 1226 | 1245 | CGGCTGGGGCGGGGACAGGG | 86 | 1868 |
| 1517573 | 4185 | 4204 | N/A | N/A | GCATTAGAAACCTCTAACTC | 93 | 1869 |
| 1517578 | 6365 | 6384 | 1267 | 1286 | CTTGGTGAATCTTTATTAAA | 28 | 1870 |
| 1517584 | 5028 | 5047 | N/A | N/A | CCCCAAGACTTAGCGACAGG | 85† | 1871 |
| 1517588 | 3435 | 3454 | N/A | N/A | CCCCAACCCATTCCCTATTT | 88 | 1872 |
| 1517595 | 6135 | 6154 | 1037 | 1056 | CAGGGGCTCGAACCAGCTCT | 96 | 1873 |
| 1517609 | 3220 | 3239 | N/A | N/A | ATTTATGAGCTAATTCAGTC | 103 | 1874 |
| 1517613 | 3051 | 3070 | N/A | N/A | CAGTCCAGCTGCTCTCCCCA | 86 | 1875 |
| 1517625 | 6314 | 6333 | 1216 | 1235 | GGGGACAGGGTCTCCCGCTG | 79 | 1876 |
| 1517637 | 4595 | 4614 | N/A | N/A | GAAAGCATTATGTATTTATA | 88 | 1877 |
| 1517659 | 3006 | 3025 | N/A | N/A | TCTCTGCTGCCCAGCCCTTC | 100 | 1878 |
| 1517667 | 6304 | 6323 | 1206 | 1225 | TCTCCCGCTGCAGGCTGCGC | 52 | 1879 |
| 1517675 | 2991 | 3010 | N/A | N/A | CCTTCCCAGGTCCAGTCCCC | 92 | 1880 |
| 1517685 | 3659 | 3678 | N/A | N/A | CGCCCCCATACCTGCCAGGA | 99 | 1881 |
| 1517691 | 4173 | 4192 | N/A | N/A | TCTAACTCCCAGCCAGGTGC | 90 | 1882 |
| 1517696 | 6334 | 6353 | 1236 | 1255 | CCCAGGAGGACGGCTGGGGC | 105 | 1883 |
| 1517719 | 2973 | 2992 | N/A | N/A | CCTGCTGCTTGCCTCACCCC | 75 | 1884 |
| 1517722 | 6264 | 6283 | 1166 | 1185 | GGCGTGGGGTCGCATGGCTG | 41 | 1885 |
| 1517724 | 6354 | 6373 | 1256 | 1275 | TTTATTAAACTAGGGTCCAC | 56 | 1886 |
| 1517731 | 3574 | 3593 | N/A | N/A | GGAGAAACTGTCAATCAACC | 90 | 1887 |
| 1517737 | 6155 | 6174 | 1057 | 1076 | TGGCGCTGCATGTCTTCCAC | 68 | 1888 |
| 1517758 | 3457 | 3476 | N/A | N/A | CCCAGCACATTTACCAAGCC | 80 | 1889 |
| 1517759 | 3528 | 3547 | N/A | N/A | GCCCGGCCCCAACCCGGCCT | 96 | 1890 |
| 1517761 | 4541 | 4560 | N/A | N/A | CAGCGGCCTGAACATGGTTC | 92 | 1891 |
| 1517771 | 4653 | 4672 | N/A | N/A | AAATGCTTCTTTGGAGCCAT | 88 | 1892 |
| 1517779 | 2922 | 2941 | N/A | N/A | TAGCTCTCCTGAGACTACCT | 89 | 1893 |
| 1517782 | 6215 | 6234 | 1117 | 1136 | GGCACAGGGGCGGCGCTGGT | 93 | 1894 |
| 1517783 | 3369 | 3388 | N/A | N/A | CCATAGAAAATTCCATCTTC | 92 | 1895 |
| 1517798 | 3516 | 3535 | N/A | N/A | CCCGGCCTCACCCCAGGTTA | 118 | 1896 |
| 1517811 | 3297 | 3316 | N/A | N/A | ATCTCAGTCCCAGTCTCGCA | 82 | 1897 |
| 1517816 | 3150 | 3169 | N/A | N/A | GGCTCCCCAGTTATGGAGAT | 93 | 1898 |
| 1517820 | 4211 | 4230 | N/A | N/A | TCTGGTATTCACTATCTGCC | 79 | 1899 |
| 1517829 | 6284 | 6303 | 1186 | 1205 | GGAGGCAGGAGGCACGGGGT | 81 | 1900 |
| 1517859 | 5045 | 5064 | N/A | N/A | CAGCAGAGACCCAGGCCCCC | 100† | 1901 |
| 1517872 | 3467 | 3486 | N/A | N/A | ACAGCCTAATCCCAGCACAT | 83 | 1902 |
| 1517879 | 3618 | 3637 | 192 | 211 | ACAGAACCTTCATCTTCCTG | 84 | 1903 |
| 1517881 | 4243 | 4262 | N/A | N/A | TGATGGAGAATAAAGATCAC | 100 | 1904 |

### Example 4: Effect of 3-10-3 cEt Mixed Backbone Modified Oligonucleotides on Human APOE RNA In Vitro, Single Dose

Modified oligonucleotides complementary to human APOE nucleic acid were designed and tested for their single dose effects on APOE RNA in vitro. The modified oligonucleotides were tested in a series of experiments that had the same culture conditions.

The modified oligonucleotides in the tables below are 3-10-3 cEt gapmers with mixed PO/PS internucleoside linkages. The gapmers are 16 nucleosides in length, wherein the central gap segment consists often 2′-β-D-deoxynucleosides, and wherein the 5′ and 3′ wing segments each consist of three cEt modified nucleosides. The sugar motif for the gapmers is (from 5′ to 3′): kkkddddddddddkkk; wherein each ‘d’ represents a 2′-β-D-deoxyribosyl sugar moiety, and each ‘k’ represents a cEt sugar moiety. The internucleoside linkage motif for the gapmers is (from 5′ to 3′): soossssssssssos; wherein each ‘o’ represents a phosphodiester internucleoside linkage and each ‘s’ represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

Cultured Hep3B cells were treated with modified oligonucleotide at a concentration of 4000 nM by electroporation at a density of 20,000 cells per well. After a treatment period of approximately 24 hours, total RNA was isolated from the cells and APOE RNA levels were measured by quantitative real-time RTPCR. APOE RNA levels were measured by human primer-probe set RTS3073 (described herein in Example 1). APOE RNA levels were normalized to total RNA content, as measured by RIBOGREEN®. Results are presented in the tables below as percent APOE RNA relative to untreated control cells (% UTC). Each table represents results from an individual assay plate. The values marked with an “†” indicate that the modified oligonucleotide is complementary to the amplicon region of the primer probe set. Additional assays may be used to measure the potency and efficacy of the modified oligonucleotides complementary to the amplicon region.

TABLE 31
Reduction of APOE RNA by 3-10-3 cEt gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 1335585 | 4190 | 4205 | N/A | N/A | TGCATTAGAAACCTCT | 76 | 1905 |
| 1335661 | 6353 | 6368 | 1255 | 1270 | TAAACTAGGGTCCACC | 13 | 1906 |
| 1516543 | 6329 | 6344 | 1231 | 1246 | ACGGCTGGGGCGGGGA | 70 | 1907 |
| 1516550 | 5064 | 5079 | N/A | N/A | AGAGGAAGCTAGAACC | 89† | 1908 |
| 1516555 | 6265 | 6280 | 1167 | 1182 | GTGGGGTCGCATGGCT | 15 | 1909 |
| 1516556 | 6132 | 6147 | 1034 | 1049 | TCGAACCAGCTCTTGA | 72 | 1910 |
| 1516597 | 3340 | 3355 | N/A | N/A | CAAATTCCATCCCCCC | 60 | 1911 |
| 1516600 | 5910 | 5925 | 812 | 827 | GCCCGCACGCGGCCCT | 63 | 1912 |
| 1516604 | 2853 | 2868 | 105 | 120 | CCCCGTGCCCCCGACT | 106 | 1913 |
| 1516614 | 6313 | 6328 | 1215 | 1230 | CAGGGTCTCCCGCTGC | 37 | 1914 |
| 1516625 | 6249 | 6264 | 1151 | 1166 | GCAGGCTTCGGCGTTC | 33 | 1915 |
| 1516649 | 6289 | 6304 | 1191 | 1206 | CGGAGGCAGGAGGCAC | 74 | 1916 |
| 1516653 | 3656 | 3671 | N/A | N/A | ATACCTGCCAGGAATG | 104 | 1917 |
| 1516654 | 5036 | 5051 | N/A | N/A | GGCCCCCCAAGACTTA | 102† | 1918 |
| 1516660 | 2939 | 2954 | N/A | N/A | GCCCGACCCCGAGTAG | 143 | 1919 |
| 1516663 | 2915 | 2930 | N/A | N/A | AGACTACCTGGAGGCC | 81 | 1920 |
| 1516672 | 6361 | 6376 | 1263 | 1278 | ATCTTTATTAAACTAG | 13 | 1921 |
| 1516678 | 5718 | 5733 | 620 | 635 | TGGCCGAGCATGGCCT | 98 | 1922 |
| 1516687 | 4616 | 4631 | N/A | N/A | GTCGGTTTAATCACTT | 71 | 1923 |
| 1516693 | 2874 | 2889 | 126 | 141 | TCTAGAGGCCCCTGAG | 104 | 1924 |
| 1516728 | 4706 | 4721 | N/A | N/A | GACCCCGTCCAGGCAT | 85 | 1925 |
| 1516731 | 3253 | 3268 | N/A | N/A | TCCAACCTCACAGTTA | 79 | 1926 |
| 1516735 | 5770 | 5785 | 672 | 687 | ACGCAGCTTGCGCAGG | 57 | 1927 |
| 1516740 | 6378 | 6393 | 1280 | 1295 | TGCGTGAAACTTGGTG | 24 | 1928 |
| 1516747 | 3505 | 3520 | N/A | N/A | GGTTAGCCTTCCAAGC | 71 | 1929 |
| 1516781 | 3431 | 3446 | N/A | N/A | CATTCCCTATTTAACT | 86 | 1930 |
| 1516800 | 5857 | 5872 | 759 | 774 | GAGGCCGCGCTCGGCG | 88 | 1931 |
| 1516806 | 4540 | 4555 | N/A | N/A | GCCTGAACATGGTTCA | 83 | 1932 |
| 1516808 | 6273 | 6288 | 1175 | 1190 | GGGGTGGCGTGGGGTC | 66 | 1933 |
| 1516818 | 3452 | 3467 | N/A | N/A | TTTACCAAGCCGCCCC | 95 | 1934 |
| 1516825 | 3833 | 3848 | N/A | N/A | TCCTATCTCAAGGATG | 97 | 1935 |
| 1516836 | 4231 | 4246 | N/A | N/A | TCACAGCTGCCCCGTG | 82 | 1936 |
| 1516838 | 5195 | 5210 | N/A | N/A | ATATAAAAGAGCTAGG | 87† | 1937 |
| 1516862 | 6257 | 6272 | 1159 | 1174 | GCATGGCTGCAGGCTT | 10 | 1938 |
| 1516863 | 2931 | 2946 | N/A | N/A | CCGAGTAGCTCTCCTG | 90 | 1939 |
| 1516864 | 5785 | 5800 | 687 | 702 | GCGGAGGAGCCGCTTA | 86 | 1940 |
| 1516873 | 5957 | 5972 | 859 | 874 | GGGCCCGCTCCTGTAG | 89 | 1941 |
| 1516893 | 3573 | 3588 | N/A | N/A | AACTGTCAATCAACCG | 84 | 1942 |
| 1516899 | 3522 | 3537 | N/A | N/A | AACCCGGCCTCACCCC | 94 | 1943 |
| 1516902 | 3073 | 3088 | N/A | N/A | CTGCTATGGCTTACAT | 71 | 1944 |
| 1516903 | 3153 | 3168 | N/A | N/A | GCTCCCCAGTTATGGA | 106 | 1945 |
| 1516911 | 6345 | 6360 | 1247 | 1262 | GGTCCACCCCAGGAGG | 61 | 1946 |
| 1516912 | 3419 | 3434 | N/A | N/A | AACTCCCTCCTGGTCT | 81 | 1947 |
| 1516914 | 3311 | 3326 | N/A | N/A | CGCCGGTTCCATCTCA | 82 | 1948 |
| 1516923 | 2948 | 2963 | N/A | N/A | TCTCCCCAAGCCCGAC | 107 | 1949 |
| 1516932 | 6370 | 6385 | 1272 | 1287 | ACTTGGTGAATCTTTA | 23 | 1950 |
| 1516933 | 3791 | 3806 | N/A | N/A | AAATCGCTGTTCAGAG | 85 | 1951 |
| 1516943 | 6321 | 6336 | 1223 | 1238 | GGCGGGGACAGGGTCT | 42 | 1952 |
| 1516952 | 3463 | 3478 | N/A | N/A | ATCCCAGCACATTTAC | 57 | 1953 |
| 1516960 | 6281 | 6296 | 1183 | 1198 | GGAGGCACGGGGTGGC | 48 | 1954 |
| 1516973 | 6305 | 6320 | 1207 | 1222 | CCCGCTGCAGGCTGCG | 31 | 1955 |
| 1516975 | 3485 | 3500 | N/A | N/A | TTGCATTATCTGCAAC | 87 | 1956 |
| 1516976 | 4800 | 4815 | 282 | 297 | GCGCAGCTCGGGCTCC | 132 | 1957 |
| 1516979 | 4987 | 5002 | N/A | N/A | AGGTATAGCCGCCCAC | 87† | 1958 |
| 1516988 | 3063 | 3078 | N/A | N/A | TTACATCCCAGTCCAG | 70 | 1959 |
| 1516990 | 4126 | 4141 | N/A | N/A | GGAGATCGAAACGGGC | 85 | 1960 |
| 1517001 | 5645 | 5660 | 547 | 562 | GCCGGGCCTGCGCCGC | 57 | 1961 |
| 1517009 | 5987 | 6002 | 889 | 904 | TCCGCGCGCGCAGCCG | 63 | 1962 |
| 1517015 | 3634 | 3649 | 208 | 223 | GCAACGCAGCCCACAG | 59 | 1963 |
| 1517017 | 5692 | 5707 | 594 | 609 | GCGGTACTGCACCAGG | 75 | 1964 |
| 1517020 | 4978 | 4993 | N/A | N/A | CGCCCACCAGGAGGGT | 87† | 1965 |
| 1517029 | 6233 | 6248 | 1135 | 1150 | AGTGATTGTCGCTGGG | 22 | 1966 |
| 1517037 | 4856 | 4871 | 338 | 353 | CAAAAGCGACCCAGTG | 62† | 1967 |
| 1517052 | 6110 | 6125 | 1012 | 1027 | CCTGGAAGGCCTCGGC | 70 | 1968 |
| 1517066 | 5012 | 5027 | N/A | N/A | GGCAGAATGAAACCTG | 92† | 1969 |
| 1517068 | 3444 | 3459 | N/A | N/A | GCCGCCCCCAACCCAT | 89 | 1970 |
| 1517074 | 5832 | 5847 | 734 | 749 | GCCCCGGCCTGGTACA | 100 | 1971 |
| 1517075 | 5570 | 5585 | 472 | 487 | CCAGTTCCGATTTGTA | 58 | 1972 |
| 1517078 | 3226 | 3241 | N/A | N/A | CCATTTATGAGCTAAT | 70 | 1973 |
| 1517079 | 3184 | 3199 | N/A | N/A | TCGACGGCTAGCTACC | 71 | 1974 |
| 1517081 | 4864 | 4879 | 346 | 361 | GGTAATCCCAAAAGCG | 58† | 1975 |
| 1517101 | 3532 | 3547 | N/A | N/A | GCCCGGCCCCAACCCG | 84 | 1976 |
| 1517104 | 3668 | 3683 | N/A | N/A | AGCCCCGCCCCCATAC | 94 | 1977 |
| 1517105 | 6337 | 6352 | 1239 | 1254 | CCAGGAGGACGGCTGG | 78 | 1978 |
| 1517110 | 3197 | 3212 | N/A | N/A | TAAAGTTCTCCAATCG | 70 | 1979 |
| 1517119 | 6241 | 6256 | 1143 | 1158 | CGGCGTTCAGTGATTG | 39 | 1980 |
| 1517123 | 6297 | 6312 | 1199 | 1214 | AGGCTGCGCGGAGGCA | 15 | 1981 |
| 1517130 | 6365 | 6380 | 1267 | 1282 | GTGAATCTTTATTAAA | 20 | 1982 |

TABLE 32
Reduction of APOE RNA by 3-10-3 cEt gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 1335562 | 3196 | 3211 | N/A | N/A | AAAGTTCTCCAATCGA | 65 | 1983 |
| 1335576 | 3428 | 3443 | N/A | N/A | TCCCTATTTAACTCCC | 96 | 1984 |
| 1335671 | 5011 | 5026 | N/A | N/A | GCAGAATGAAACCTGG | 80† | 1985 |
| 1335675 | 4189 | 4204 | N/A | N/A | GCATTAGAAACCTCTA | 83 | 1986 |
| 1335681 | 3846 | 3861 | N/A | N/A | AACAACTTCTAACTCC | 69 | 1987 |
| 1516527 | 4225 | 4240 | N/A | N/A | CTGCCCCGTGTCTGGT | 88 | 1988 |
| 1516528 | 4977 | 4992 | N/A | N/A | GCCCACCAGGAGGGTC | 92† | 1989 |
| 1516532 | 6288 | 6303 | 1190 | 1205 | GGAGGCAGGAGGCACG | 60 | 1990 |
| 1516539 | 6256 | 6271 | 1158 | 1173 | CATGGCTGCAGGCTTC | 14 | 1991 |
| 1516548 | 4855 | 4870 | 337 | 352 | AAAAGCGACCCAGTGC | 61† | 1992 |
| 1516558 | 3310 | 3325 | N/A | N/A | GCCGGTTCCATCTCAG | 76 | 1993 |
| 1516573 | 4986 | 5001 | N/A | N/A | GGTATAGCCGCCCACC | 81† | 1994 |
| 1516578 | 3462 | 3477 | N/A | N/A | TCCCAGCACATTTACC | 82 | 1995 |
| 1516589 | 6320 | 6335 | 1222 | 1237 | GCGGGGACAGGGTCTC | 28 | 1996 |
| 1516607 | 6240 | 6255 | 1142 | 1157 | GGCGTTCAGTGATTGT | 14 | 1997 |
| 1516609 | 3790 | 3805 | N/A | N/A | AATCGCTGTTCAGAGC | 74 | 1998 |
| 1516621 | 5747 | 5762 | 649 | 664 | CGAGGCGCACCCGCAG | 84 | 1999 |
| 1516624 | 5782 | 5797 | 684 | 699 | GAGGAGCCGCTTACGC | 72 | 2000 |
| 1516627 | 5943 | 5958 | 845 | 860 | AGCGGCTGGCCGGCCA | 80 | 2001 |
| 1516655 | 5061 | 5076 | N/A | N/A | GGAAGCTAGAACCAGC | 67† | 2002 |
| 1516673 | 4310 | 4325 | N/A | N/A | CGCGCCCAGCGGAAAA | 94 | 2003 |
| 1516697 | 6344 | 6359 | 1246 | 1261 | GTCCACCCCAGGAGGA | 84 | 2004 |
| 1516700 | 3521 | 3536 | N/A | N/A | ACCCGGCCTCACCCCA | 107 | 2005 |
| 1516723 | 3037 | 3052 | N/A | N/A | CACCCCACCTTCTAGC | 101 | 2006 |
| 1516734 | 2930 | 2945 | N/A | N/A | CGAGTAGCTCTCCTGA | 82 | 2007 |
| 1516745 | 2914 | 2929 | N/A | N/A | GACTACCTGGAGGCCA | 76 | 2008 |
| 1516748 | 3443 | 3458 | N/A | N/A | CCGCCCCCAACCCATT | 96 | 2009 |
| 1516765 | 5559 | 5574 | 461 | 476 | TTGTAGGCCTTCAACT | 66 | 2010 |
| 1516772 | 6108 | 6123 | 1010 | 1025 | TGGAAGGCCTCGGCCT | 86 | 2011 |
| 1516778 | 6360 | 6375 | 1262 | 1277 | TCTTTATTAAACTAGG | 4 | 2012 |
| 1516779 | 5831 | 5846 | 733 | 748 | CCCCGGCCTGGTACAC | 84 | 2013 |
| 1516791 | 3451 | 3466 | N/A | N/A | TTACCAAGCCGCCCCC | 96 | 2014 |
| 1516796 | 6194 | 6209 | 1096 | 1111 | CGGCAGCCTGCACCTT | 66 | 2015 |
| 1516824 | 3655 | 3670 | N/A | N/A | TACCTGCCAGGAATGT | 79 | 2016 |
| 1516841 | 2849 | 2864 | 101 | 116 | GTGCCCCCGACTGCGC | 99 | 2017 |
| 1516847 | 3531 | 3546 | N/A | N/A | CCCGGCCCCAACCCGG | 96 | 2018 |
| 1516848 | 3183 | 3198 | N/A | N/A | CGACGGCTAGCTACCG | 65 | 2019 |
| 1516854 | 5841 | 5856 | 743 | 758 | CCCTCGCGGGCCCCGG | 84 | 2020 |
| 1516858 | 3664 | 3679 | N/A | N/A | CCGCCCCCATACCTGC | 95 | 2021 |
| 1516867 | 3152 | 3167 | N/A | N/A | CTCCCCAGTTATGGAG | 89 | 2022 |
| 1516876 | 3832 | 3847 | N/A | N/A | CCTATCTCAAGGATGG | 85 | 2023 |
| 1516881 | 6264 | 6279 | 1166 | 1181 | TGGGGTCGCATGGCTG | 21 | 2024 |
| 1516886 | 6296 | 6311 | 1198 | 1213 | GGCTGCGCGGAGGCAG | 18 | 2025 |
| 1516890 | 6312 | 6327 | 1214 | 1229 | AGGGTCTCCCGCTGCA | 13 | 2026 |
| 1516892 | 3072 | 3087 | N/A | N/A | TGCTATGGCTTACATC | 78 | 2027 |
| 1516896 | 3570 | 3585 | N/A | N/A | TGTCAATCAACCGCCA | 77 | 2028 |
| 1516915 | 3502 | 3517 | N/A | N/A | TAGCCTTCCAAGCCTT | 85 | 2029 |
| 1516917 | 3481 | 3496 | N/A | N/A | ATTATCTGCAACAGCC | 67 | 2030 |
| 1516922 | 4863 | 4878 | 345 | 360 | GTAATCCCAAAAGCGA | 40† | 2031 |
| 1516925 | 5986 | 6001 | 888 | 903 | CCGCGCGCGCAGCCGC | 56 | 2032 |
| 1516929 | 3633 | 3648 | 207 | 222 | CAACGCAGCCCACAGA | 57 | 2033 |
| 1516930 | 5194 | 5209 | N/A | N/A | TATAAAAGAGCTAGGG | 74† | 2034 |
| 1516934 | 6131 | 6146 | 1033 | 1048 | CGAACCAGCTCTTGAG | 53 | 2035 |
| 1516939 | 2938 | 2953 | N/A | N/A | CCCGACCCCGAGTAGC | 72 | 2036 |
| 1516947 | 5637 | 5652 | 539 | 554 | TGCGCCGCCTGCAGCT | 84 | 2037 |
| 1516962 | 5909 | 5924 | 811 | 826 | CCCGCACGCGGCCCTG | 68 | 2038 |
| 1516968 | 6248 | 6263 | 1150 | 1165 | CAGGCTTCGGCGTTCA | 41 | 2039 |
| 1516972 | 3319 | 3334 | N/A | N/A | CTCCCCACCGCCGGTT | 88 | 2040 |
| 1516992 | 6328 | 6343 | 1230 | 1245 | CGGCTGGGGCGGGGAC | 89 | 2041 |
| 1516994 | 5717 | 5732 | 619 | 634 | GGCCGAGCATGGCCTG | 98 | 2042 |
| 1517000 | 3393 | 3408 | N/A | N/A | CCCCATCCCCAGGTCG | 98 | 2043 |
| 1517010 | 4776 | 4791 | 258 | 273 | CTCCACCGCTTGCTCC | 145 | 2044 |
| 1517016 | 6336 | 6351 | 1238 | 1253 | CAGGAGGACGGCTGGG | 40 | 2045 |
| 1517024 | 6377 | 6392 | 1279 | 1294 | GCGTGAAACTTGGTGA | 21 | 2046 |
| 1517027 | 2947 | 2962 | N/A | N/A | CTCCCCAAGCCCGACC | 104 | 2047 |
| 1517040 | 6272 | 6287 | 1174 | 1189 | GGGTGGCGTGGGGTCG | 32 | 2048 |
| 1517050 | 6304 | 6319 | 1206 | 1221 | CCGCTGCAGGCTGCGC | 46 | 2049 |
| 1517059 | 3252 | 3267 | N/A | N/A | CCAACCTCACAGTTAA | 70 | 2050 |
| 1517063 | 6369 | 6384 | 1271 | 1286 | CTTGGTGAATCTTTAT | 24 | 2051 |
| 1517073 | 2873 | 2888 | 125 | 140 | CTAGAGGCCCCTGAGC | 82 | 2052 |
| 1517083 | 5035 | 5050 | N/A | N/A | GCCCCCCAAGACTTAG | 101† | 2053 |
| 1517088 | 5691 | 5706 | 593 | 608 | CGGTACTGCACCAGGC | 74 | 2054 |
| 1517100 | 6280 | 6295 | 1182 | 1197 | GAGGCACGGGGTGGCG | 41 | 2055 |
| 1517116 | 4615 | 4630 | N/A | N/A | TCGGTTTAATCACTTG | 63 | 2056 |
| 1517126 | 4705 | 4720 | N/A | N/A | ACCCCGTCCAGGCATC | 106 | 2057 |
| 1517129 | 3222 | 3237 | N/A | N/A | TTATGAGCTAATTCAG | 63 | 2058 |
| 1517130 | 6365 | 6380 | 1267 | 1282 | GTGAATCTTTATTAAA | 17 | 1982 |
| 1517131 | 6352 | 6367 | 1254 | 1269 | AAACTAGGGTCCACCC | 18 | 2059 |

TABLE 33
Reduction of APOE RNA by 3-10-3 cEt gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 1335568 | 4214 | 4229 | N/A | N/A | CTGGTATTCACTATCT | 71 | 2060 |
| 1335581 | 4614 | 4629 | N/A | N/A | CGGTTTAATCACTTGG | 58 | 2061 |
| 1516525 | 5779 | 5794 | 681 | 696 | GAGCCGCTTACGCAGC | 90 | 2062 |
| 1516534 | 3530 | 3545 | N/A | N/A | CCGGCCCCAACCCGGC | 95 | 2063 |
| 1516536 | 4704 | 4719 | N/A | N/A | CCCCGTCCAGGCATCT | 105 | 2064 |
| 1516552 | 6351 | 6366 | 1253 | 1268 | AACTAGGGTCCACCCC | 37 | 2065 |
| 1516559 | 6255 | 6270 | 1157 | 1172 | ATGGCTGCAGGCTTCG | 23 | 2066 |
| 1516561 | 6319 | 6334 | 1221 | 1236 | CGGGGACAGGGTCTCC | 63 | 2067 |
| 1516576 | 3317 | 3332 | N/A | N/A | CCCCACCGCCGGTTCC | 103 | 2068 |
| 1516593 | 2937 | 2952 | N/A | N/A | CCGACCCCGAGTAGCT | 105 | 2069 |
| 1516594 | 3190 | 3205 | N/A | N/A | CTCCAATCGACGGCTA | 72 | 2070 |
| 1516602 | 5908 | 5923 | 810 | 825 | CCGCACGCGGCCCTGT | 81 | 2071 |
| 1516638 | 2867 | 2882 | 119 | 134 | GCCCCTGAGCTCATCC | 78 | 2072 |
| 1516641 | 3501 | 3516 | N/A | N/A | AGCCTTCCAAGCCTTG | 70 | 2073 |
| 1516658 | 6359 | 6374 | 1261 | 1276 | CTTTATTAAACTAGGG | 6 | 2074 |
| 1516668 | 6327 | 6342 | 1229 | 1244 | GGCTGGGGCGGGGACA | 77 | 2075 |
| 1516670 | 3842 | 3857 | N/A | N/A | ACTTCTAACTCCTATC | 74 | 2076 |
| 1516674 | 4985 | 5000 | N/A | N/A | GTATAGCCGCCCACCA | 88† | 2077 |
| 1516675 | 6106 | 6121 | 1008 | 1023 | GAAGGCCTCGGCCTGC | 90 | 2078 |
| 1516677 | 5984 | 5999 | 886 | 901 | GCGCGCGCAGCCGCTC | 82 | 2079 |
| 1516684 | 3035 | 3050 | N/A | N/A | CCCCACCTTCTAGCGG | 84 | 2080 |
| 1516686 | 5009 | 5024 | N/A | N/A | AGAATGAAACCTGGAC | 90† | 2081 |
| 1516703 | 4187 | 4202 | N/A | N/A | ATTAGAAACCTCTAAC | 81 | 2082 |
| 1516707 | 6343 | 6358 | 1245 | 1260 | TCCACCCCAGGAGGAC | 86 | 2083 |
| 1516708 | 4774 | 4789 | 256 | 271 | CCACCGCTTGCTCCAC | 89 | 2084 |
| 1516710 | 5165 | 5180 | N/A | N/A | GAGCCAGGACGAGTGT | 98† | 2085 |
| 1516713 | 3142 | 3157 | N/A | N/A | ATGGAGATCTGAGGAC | 83 | 2086 |
| 1516718 | 4975 | 4990 | N/A | N/A | CCACCAGGAGGGTCAA | 82† | 2087 |
| 1516726 | 6376 | 6391 | 1278 | 1293 | CGTGAAACTTGGTGAA | 33 | 2088 |
| 1516755 | 3427 | 3442 | N/A | N/A | CCCTATTTAACTCCCT | 82 | 2089 |
| 1516763 | 4854 | 4869 | 336 | 351 | AAAGCGACCCAGTGCC | 32† | 2090 |
| 1516775 | 3831 | 3846 | N/A | N/A | CTATCTCAAGGATGGG | 77 | 2091 |
| 1516782 | 6303 | 6318 | 1205 | 1220 | CGCTGCAGGCTGCGCG | 81 | 2092 |
| 1516788 | 4862 | 4877 | 344 | 359 | TAATCCCAAAAGCGAC | 55† | 2093 |
| 1516792 | 6279 | 6294 | 1181 | 1196 | AGGCACGGGGTGGCGT | 56 | 2094 |
| 1516794 | 6239 | 6254 | 1141 | 1156 | GCGTTCAGTGATTGTC | 19 | 2095 |
| 1516797 | 5829 | 5844 | 731 | 746 | CCGGCCTGGTACACTG | 95 | 2096 |
| 1516803 | 3251 | 3266 | N/A | N/A | CAACCTCACAGTTAAG | 80 | 2097 |
| 1516809 | 6247 | 6262 | 1149 | 1164 | AGGCTTCGGCGTTCAG | 38 | 2098 |
| 1516814 | 4309 | 4324 | N/A | N/A | GCGCCCAGCGGAAAAG | 97 | 2099 |
| 1516815 | 6287 | 6302 | 1189 | 1204 | GAGGCAGGAGGCACGG | 80 | 2100 |
| 1516837 | 3569 | 3584 | N/A | N/A | GTCAATCAACCGCCAG | 79 | 2101 |
| 1516850 | 3071 | 3086 | N/A | N/A | GCTATGGCTTACATCC | 87 | 2102 |
| 1516860 | 3680 | 3695 | N/A | N/A | GGGAACCGAGCAAGCC | 99 | 2103 |
| 1516865 | 5556 | 5571 | 458 | 473 | TAGGCCTTCAACTCCT | 88 | 2104 |
| 1516884 | 6172 | 6187 | 1074 | 1089 | CAGCCCGGCCCACTGG | 81 | 2105 |
| 1516900 | 3480 | 3495 | N/A | N/A | TTATCTGCAACAGCCT | 79 | 2106 |
| 1516907 | 5716 | 5731 | 618 | 633 | GCCGAGCATGGCCTGC | 86 | 2107 |
| 1516908 | 5840 | 5855 | 742 | 757 | CCTCGCGGGCCCCGGC | 100 | 2108 |
| 1516935 | 3182 | 3197 | N/A | N/A | GACGGCTAGCTACCGT | 88 | 2109 |
| 1516936 | 6311 | 6326 | 1213 | 1228 | GGGTCTCCCGCTGCAG | 26 | 2110 |
| 1516938 | 5688 | 5703 | 590 | 605 | TACTGCACCAGGCGGC | 66 | 2111 |
| 1516948 | 6271 | 6286 | 1173 | 1188 | GGTGGCGTGGGGTCGC | 34 | 2112 |
| 1516951 | 3645 | 3660 | 219 | 234 | GAATGTGACCAGCAAC | 54 | 2113 |
| 1516955 | 5746 | 5761 | 648 | 663 | GAGGCGCACCCGCAGC | 99 | 2114 |
| 1516961 | 5636 | 5651 | 538 | 553 | GCGCCGCCTGCAGCTC | 75 | 2115 |
| 1516963 | 3450 | 3465 | N/A | N/A | TACCAAGCCGCCCCCA | 90 | 2116 |
| 1516964 | 2913 | 2928 | N/A | N/A | ACTACCTGGAGGCCAG | 99 | 2117 |
| 1516974 | 6368 | 6383 | 1270 | 1285 | TTGGTGAATCTTTATT | 25 | 2118 |
| 1516977 | 5059 | 5074 | N/A | N/A | AAGCTAGAACCAGCAG | 81† | 2119 |
| 1516978 | 5934 | 5949 | 836 | 851 | CCGGCCAGGGAGCCCA | 85 | 2120 |
| 1516981 | 3515 | 3530 | N/A | N/A | CCTCACCCCAGGTTAG | 83 | 2121 |
| 1516987 | 6130 | 6145 | 1032 | 1047 | GAACCAGCTCTTGAGG | 59 | 2122 |
| 1516991 | 3221 | 3236 | N/A | N/A | TATGAGCTAATTCAGT | 71 | 2123 |
| 1517005 | 3309 | 3324 | N/A | N/A | CCGGTTCCATCTCAGT | 82 | 2124 |
| 1517014 | 6295 | 6310 | 1197 | 1212 | GCTGCGCGGAGGCAGG | 76 | 2125 |
| 1517021 | 2848 | 2863 | 100 | 115 | TGCCCCCGACTGCGCT | 92 | 2126 |
| 1517031 | 6263 | 6278 | 1165 | 1180 | GGGGTCGCATGGCTGC | 59 | 2127 |
| 1517033 | 3458 | 3473 | N/A | N/A | AGCACATTTACCAAGC | 87 | 2128 |
| 1517036 | 2929 | 2944 | N/A | N/A | GAGTAGCTCTCCTGAG | 79 | 2129 |
| 1517038 | 6335 | 6350 | 1237 | 1252 | AGGAGGACGGCTGGGG | 87 | 2130 |
| 1517046 | 5034 | 5049 | N/A | N/A | CCCCCCAAGACTTAGC | 83† | 2131 |
| 1517054 | 3663 | 3678 | N/A | N/A | CGCCCCCATACCTGCC | 85 | 2132 |
| 1517057 | 3442 | 3457 | N/A | N/A | CGCCCCCAACCCATTC | 86 | 2133 |
| 1517065 | 2946 | 2961 | N/A | N/A | TCCCCAAGCCCGACCC | 79 | 2134 |
| 1517069 | 3632 | 3647 | 206 | 221 | AACGCAGCCCACAGAA | 67 | 2135 |
| 1517109 | 3391 | 3406 | N/A | N/A | CCATCCCCAGGTCGGC | 97 | 2136 |
| 1517130 | 6365 | 6380 | 1267 | 1282 | GTGAATCTTTATTAAA | 29 | 1982 |

TABLE 34
Reduction of APOE RNA by 3-10-3 cEt gapmers with mixed PO/PS internucleoside linkages
at a concentration of 4000 nM in Hep3B cells plated at 20,000 cells per well

| | SEQ ID | SEQ ID | SEQ ID | SEQ ID | | | |
|---|---|---|---|---|---|---|---|
| | No: 1 | No: 1 | No: 2 | No: 2 | | APOE | |
| Compound | Start | Stop | Start | Stop | | (% | SEQ ID |
| Number | Site | Site | Site | Site | Sequence (5′ to 3′) | UTC) | NO |
| 1516551 | 2847 | 2862 | 99 | 114 | GCCCCCGACTGCGCTT | 92 | 2137 |
| 1516560 | 2864 | 2879 | 116 | 131 | CCTGAGCTCATCCCCG | 99 | 2138 |
| 1516563 | 3640 | 3655 | 214 | 229 | TGACCAGCAACGCAGC | 50 | 2139 |
| 1516575 | 6318 | 6333 | 1220 | 1235 | GGGGACAGGGTCTCCC | 75 | 2140 |
| 1516579 | 3476 | 3491 | N/A | N/A | CTGCAACAGCCTAATC | 89 | 2141 |
| 1516581 | 5686 | 5701 | 588 | 603 | CTGCACCAGGCGGCCG | 126 | 2142 |
| 1516588 | 2944 | 2959 | N/A | N/A | CCCAAGCCCGACCCCG | 92 | 2143 |
| 1516595 | 5704 | 5719 | 606 | 621 | CTGCACCTCGCCGCGG | 94 | 2144 |
| 1516596 | 4185 | 4200 | N/A | N/A | TAGAAACCTCTAACTC | 88 | 2145 |
| 1516606 | 3189 | 3204 | N/A | N/A | TCCAATCGACGGCTAG | 77 | 2146 |
| 1516616 | 5778 | 5793 | 680 | 695 | AGCCGCTTACGCAGCT | 99 | 2147 |
| 1516618 | 4613 | 4628 | N/A | N/A | GGTTTAATCACTTGGA | 74 | 2148 |
| 1516623 | 4896 | 4911 | 378 | 393 | CACCTGCTCAGACAGT | 24† | 2149 |
| 1516632 | 5057 | 5072 | N/A | N/A | GCTAGAACCAGCAGAG | 88† | 2150 |
| 1516644 | 6171 | 6186 | 1073 | 1088 | AGCCCGGCCCACTGGC | 87 | 2151 |
| 1516645 | 3631 | 3646 | 205 | 220 | ACGCAGCCCACAGAAC | 74 | 2152 |
| 1516646 | 6238 | 6253 | 1140 | 1155 | CGTTCAGTGATTGTCG | 21 | 2153 |
| 1516656 | 6254 | 6269 | 1156 | 1171 | TGGCTGCAGGCTTCGG | 17 | 2154 |
| 1516662 | 4984 | 4999 | N/A | N/A | TATAGCCGCCCACCAG | 90† | 2155 |
| 1516667 | 5008 | 5023 | N/A | N/A | GAATGAAACCTGGACC | 100† | 2156 |
| 1516680 | 3294 | 3309 | N/A | N/A | TCCCAGTCTCGCATTC | 85 | 2157 |
| 1516681 | 6367 | 6382 | 1269 | 1284 | TGGTGAATCTTTATTA | 26 | 2158 |
| 1516682 | 5033 | 5048 | N/A | N/A | CCCCCAAGACTTAGCG | 100† | 2159 |
| 1516688 | 6278 | 6293 | 1180 | 1195 | GGCACGGGGTGGCGTG | 38 | 2160 |
| 1516696 | 6105 | 6120 | 1007 | 1022 | AAGGCCTCGGCCTGCA | 84 | 2161 |
| 1516704 | 3457 | 3472 | N/A | N/A | GCACATTTACCAAGCC | 89 | 2162 |
| 1516706 | 3426 | 3441 | N/A | N/A | CCTATTTAACTCCCTC | 75 | 2163 |
| 1516709 | 4861 | 4876 | 343 | 358 | AATCCCAAAAGCGACC | 25† | 2164 |
| 1516716 | 3437 | 3452 | N/A | N/A | CCAACCCATTCCCTAT | 112 | 2165 |
| 1516730 | 5824 | 5839 | 726 | 741 | CTGGTACACTGCCAGG | 89 | 2166 |
| 1516736 | 6294 | 6309 | 1196 | 1211 | CTGCGCGGAGGCAGGA | 64 | 2167 |
| 1516742 | 3498 | 3513 | N/A | N/A | CTTCCAAGCCTTGTTG | 94 | 2168 |
| 1516743 | 6375 | 6390 | 1277 | 1292 | GTGAAACTTGGTGAAT | 35 | 2169 |
| 1516744 | 4853 | 4868 | 335 | 350 | AAGCGACCCAGTGCCA | 33† | 2170 |
| 1516746 | 6326 | 6341 | 1228 | 1243 | GCTGGGGCGGGGACAG | 84 | 2171 |
| 1516749 | 3567 | 3582 | N/A | N/A | CAATCAACCGCCAGTG | 98 | 2172 |
| 1516756 | 3529 | 3544 | N/A | N/A | CGGCCCCAACCCGGCC | 83 | 2173 |
| 1516761 | 3679 | 3694 | N/A | N/A | GGAACCGAGCAAGCCC | 86 | 2174 |
| 1516762 | 6334 | 6349 | 1236 | 1251 | GGAGGACGGCTGGGGC | 82 | 2175 |
| 1516774 | 6286 | 6301 | 1188 | 1203 | AGGCAGGAGGCACGGG | 29 | 2176 |
| 1516783 | 3809 | 3824 | N/A | N/A | CGAGGCCCAGAGAGCG | 79 | 2177 |
| 1516785 | 5547 | 5562 | 449 | 464 | AACTCCTTCATGGTCT | 51 | 2178 |
| 1516790 | 3316 | 3331 | N/A | N/A | CCCACCGCCGGTTCCA | 84 | 2179 |
| 1516805 | 5983 | 5998 | 885 | 900 | CGCGCGCAGCCGCTCG | 75 | 2180 |
| 1516807 | 6342 | 6357 | 1244 | 1259 | CCACCCCAGGAGGACG | 64 | 2181 |
| 1516812 | 3250 | 3265 | N/A | N/A | AACCTCACAGTTAAGC | 76 | 2182 |
| 1516822 | 3514 | 3529 | N/A | N/A | CTCACCCCAGGTTAGC | 85 | 2183 |
| 1516827 | 6129 | 6144 | 1031 | 1046 | AACCAGCTCTTGAGGC | 73 | 2184 |
| 1516869 | 2879 | 2894 | 131 | 146 | CTCTTTCTAGAGGCCC | 74 | 2185 |
| 1516888 | 3839 | 3854 | N/A | N/A | TCTAACTCCTATCTCA | 79 | 2186 |
| 1516913 | 5624 | 5639 | 526 | 541 | GCTCCTTGGACAGCCG | 87 | 2187 |
| 1516924 | 3662 | 3677 | N/A | N/A | GCCCCCATACCTGCCA | 93 | 2188 |
| 1516928 | 3449 | 3464 | N/A | N/A | ACCAAGCCGCCCCCAA | 91 | 2189 |
| 1516950 | 6270 | 6285 | 1172 | 1187 | GTGGCGTGGGGTCGCA | 29 | 2190 |
| 1516953 | 5933 | 5948 | 835 | 850 | CGGCCAGGGAGCCCAC | 75 | 2191 |
| 1516956 | 5745 | 5760 | 647 | 662 | AGGCGCACCCGCAGCT | 76 | 2192 |
| 1516957 | 2995 | 3010 | N/A | N/A | CCTTCCCAGGTCCAGT | 91 | 2193 |
| 1516958 | 3220 | 3235 | N/A | N/A | ATGAGCTAATTCAGTC | 61 | 2194 |
| 1516965 | 6302 | 6317 | 1204 | 1219 | GCTGCAGGCTGCGCGG | 43 | 2195 |
| 1516966 | 5839 | 5854 | 741 | 756 | CTCGCGGGCCCCGGCC | 70 | 2196 |
| 1516969 | 3069 | 3084 | N/A | N/A | TATGGCTTACATCCCA | 80 | 2197 |
| 1516984 | 3181 | 3196 | N/A | N/A | ACGGCTAGCTACCGTG | 91 | 2198 |
| 1516996 | 3137 | 3152 | N/A | N/A | GATCTGAGGACACTGG | 86 | 2199 |
| 1517006 | 6350 | 6365 | 1252 | 1267 | ACTAGGGTCCACCCCA | 49 | 2200 |
| 1517019 | 4213 | 4228 | N/A | N/A | TGGTATTCACTATCTG | 69 | 2201 |
| 1517025 | 3389 | 3404 | N/A | N/A | ATCCCCAGGTCGGCCT | 86 | 2202 |
| 1517042 | 4741 | 4756 | N/A | N/A | TGTGGAACAAGTTCAA | 87 | 2203 |
| 1517056 | 5907 | 5922 | 809 | 824 | CGCACGCGGCCCTGTT | 64 | 2204 |
| 1517061 | 4305 | 4320 | N/A | N/A | CCAGCGGAAAAGCATG | 101 | 2205 |
| 1517062 | 6246 | 6261 | 1148 | 1163 | GGCTTCGGCGTTCAGT | 34 | 2206 |
| 1517077 | 2923 | 2938 | N/A | N/A | CTCTCCTGAGACTACC | 84 | 2207 |
| 1517082 | 6358 | 6373 | 1260 | 1275 | TTTATTAAACTAGGGT | 10 | 2208 |
| 1517085 | 6262 | 6277 | 1164 | 1179 | GGGTCGCATGGCTGCA | 18 | 2209 |
| 1517099 | 2936 | 2951 | N/A | N/A | CGACCCCGAGTAGCTC | 79 | 2210 |
| 1517107 | 6310 | 6325 | 1212 | 1227 | GGTCTCCCGCTGCAGG | 31 | 2211 |
| 1517124 | 5102 | 5117 | N/A | N/A | GAATTCCAGAGAGCTA | 90† | 2212 |
| 1517130 | 6365 | 6380 | 1267 | 1282 | GTGAATCTTTATTAAA | 32 | 1982 |
| 1517135 | 4700 | 4715 | N/A | N/A | GTCCAGGCATCTAGTA | 75 | 2213 |

TABLE 35
Reduction of APOE RNA by 3-10-3 cEt gapmers
with mixed PO/PS internucleoside linkages at a
concentration of 4000 nM in Hep3B cells
plated at 20,000 cells per well

| | SEQ | SEQ | SEQ | SEQ | | | |
|---|---|---|---|---|---|---|---|
| | ID No: | ID No: | ID No: | ID No: | | | |
| | 1 | 1 | 2 | 2 | | APOE | |
| Compound | Start | Stop | Start | Stop | Sequence | (% | SEQ |
| Number | Site | Site | Site | Site | (5′ to 3′) | UTC) | ID NO |
| 1335571 | 3068 | 3083 | N/A | N/A | ATGGCTTACATCCCAG | 99 | 2214 |
| 1335663 | 4184 | 4199 | N/A | N/A | AGAAACCTCTAACTCC | 50 | 2215 |
| 1335673 | 3678 | 3693 | N/A | N/A | GAACCGAGCAAGCCCC | 86 | 2216 |
| 1335678 | 3425 | 3440 | N/A | N/A | CTATTTAACTCCCTCC | 82 | 2217 |
| 1335679 | 3105 | 3120 | N/A | N/A | CTCGATAAATGATAGT | 75 | 2218 |
| 1516529 | 4696 | 4711 | N/A | N/A | AGGCATCTAGTACCTA | 78 | 2219 |
| 1516537 | 4860 | 4875 | 342 | 357 | ATCCCAAAAGCGACCC | 15† | 2220 |
| 1516538 | 5545 | 5560 | 447 | 462 | CTCCTTCATGGTCTCG | 47 | 2221 |
| 1516545 | 5837 | 5852 | 739 | 754 | CGCGGGCCCCGGCCTG | 75 | 2222 |
| 1516553 | 6357 | 6372 | 1259 | 1274 | TTATTAAACTAGGGTC | 16 | 2223 |
| 1516567 | 6055 | 6070 | 957 | 972 | GCGCACCTCCGCCACC | 66 | 2224 |
| 1516568 | 6116 | 6131 | 1018 | 1033 | GGCGGGCCTGGAAGGC | 74 | 2225 |
| 1516570 | 6253 | 6268 | 1155 | 1170 | GGCTGCAGGCTTCGGC | 18 | 2226 |
| 1516571 | 5006 | 5021 | N/A | N/A | ATGAAACCTGGACCTG | 88† | 2227 |
| 1516591 | 3639 | 3654 | 213 | 228 | GACCAGCAACGCAGCC | 67 | 2228 |
| 1516598 | 3626 | 3641 | 200 | 215 | GCCCACAGAACCTTCA | 68 | 2229 |
| 1516603 | 6277 | 6292 | 1179 | 1194 | GCACGGGGTGGCGTGG | 39 | 2230 |
| 1516611 | 3513 | 3528 | N/A | N/A | TCACCCCAGGTTAGCC | 111 | 2231 |
| 1516613 | 6366 | 6381 | 1268 | 1283 | GGTGAATCTTTATTAA | 6 | 2232 |
| 1516615 | 3456 | 3471 | N/A | N/A | CACATTTACCAAGCCG | 65 | 2233 |
| 1516629 | 2921 | 2936 | N/A | N/A | CTCCTGAGACTACCTG | 77 | 2234 |
| 1516630 | 5623 | 5638 | 525 | 540 | CTCCTTGGACAGCCGT | 44 | 2235 |
| 1516633 | 4211 | 4226 | N/A | N/A | GTATTCACTATCTGCC | 85 | 2236 |
| 1516657 | 5777 | 5792 | 679 | 694 | GCCGCTTACGCAGCTT | 91 | 2237 |
| 1516661 | 3475 | 3490 | N/A | N/A | TGCAACAGCCTAATCC | 80 | 2238 |
| 1516666 | 4869 | 4884 | 351 | 366 | GCGCAGGTAATCCCAA | 29† | 2239 |
| 1516692 | 4852 | 4867 | 334 | 349 | AGCGACCCAGTGCCAG | 30† | 2240 |
| 1516750 | 6237 | 6252 | 1139 | 1154 | GTTCAGTGATTGTCGC | 20 | 2241 |
| 1516757 | 3180 | 3195 | N/A | N/A | CGGCTAGCTACCGTGT | 70 | 2242 |
| 1516769 | 2878 | 2893 | 130 | 145 | TCTTTCTAGAGGCCCC | 85 | 2243 |
| 1516770 | 5925 | 5940 | 827 | 842 | GAGCCCACAGTGGCGG | 96 | 2244 |
| 1516777 | 3838 | 3853 | N/A | N/A | CTAACTCCTATCTCAA | 65 | 2245 |
| 1516780 | 5055 | 5070 | N/A | N/A | TAGAACCAGCAGAGAC | 95† | 2246 |
| 1516784 | 6309 | 6324 | 1211 | 1226 | GTCTCCCGCTGCAGGC | 31 | 2247 |
| 1516793 | 3377 | 3392 | N/A | N/A | GCCTCCATAGAAAATT | 109 | 2248 |
| 1516799 | 4983 | 4998 | N/A | N/A | ATAGCCGCCCACCAGG | 82† | 2249 |
| 1516802 | 6349 | 6364 | 1251 | 1266 | CTAGGGTCCACCCCAG | 60 | 2250 |
| 1516810 | 3315 | 3330 | N/A | N/A | CCACCGCCGGTTCCAT | 96 | 2251 |
| 1516811 | 6170 | 6185 | 1072 | 1087 | GCCCGGCCCACTGGCG | 44 | 2252 |
| 1516819 | 6317 | 6332 | 1219 | 1234 | GGGACAGGGTCTCCCG | 72 | 2253 |
| 1516828 | 6269 | 6284 | 1171 | 1186 | TGGCGTGGGGTCGCAT | 28 | 2254 |
| 1516834 | 6374 | 6389 | 1276 | 1291 | TGAAACTTGGTGAATC | 22 | 2255 |
| 1516842 | 5032 | 5047 | N/A | N/A | CCCCAAGACTTAGCGA | 91† | 2256 |
| 1516844 | 6341 | 6356 | 1243 | 1258 | CACCCCAGGAGGACGG | 70 | 2257 |
| 1516845 | 3248 | 3263 | N/A | N/A | CCTCACAGTTAAGCGC | 69 | 2258 |
| 1516866 | 6261 | 6276 | 1163 | 1178 | GGTCGCATGGCTGCAG | 11 | 2259 |
| 1516875 | 2845 | 2860 | 97 | 112 | CCCCGACTGCGCTTCT | 87 | 2260 |
| 1516882 | 3188 | 3203 | N/A | N/A | CCAATCGACGGCTAGC | 96 | 2261 |
| 1516885 | 2935 | 2950 | N/A | N/A | GACCCCGAGTAGCTCT | 79 | 2262 |
| 1516904 | 3436 | 3451 | N/A | N/A | CAACCCATTCCCTATT | 72 | 2263 |
| 1516906 | 5097 | 5112 | N/A | N/A | CCAGAGAGCTAAAGCC | 77† | 2264 |
| 1516940 | 6285 | 6300 | 1187 | 1202 | GGCAGGAGGCACGGGG | 46 | 2265 |
| 1516946 | 3263 | 3278 | N/A | N/A | CATTCTAAGCTCCAAC | 74 | 2266 |
| 1516949 | 6333 | 6348 | 1235 | 1250 | GAGGACGGCTGGGGCG | 50 | 2267 |
| 1516954 | 3526 | 3541 | N/A | N/A | CCCCAACCCGGCCTCA | 84 | 2268 |
| 1516959 | 3795 | 3810 | N/A | N/A | CGTCAAATCGCTGTTC | 71 | 2269 |
| 1516982 | 5823 | 5838 | 725 | 740 | TGGTACACTGCCAGGC | 46 | 2270 |
| 1516983 | 5906 | 5921 | 808 | 823 | GCACGCGGCCCTGTTC | 56 | 2271 |
| 1516985 | 6245 | 6260 | 1147 | 1162 | GCTTCGGCGTTCAGTG | 32 | 2272 |
| 1516993 | 5743 | 5758 | 645 | 660 | GCGCACCCGCAGCTCC | 90 | 2273 |
| 1517002 | 4548 | 4563 | N/A | N/A | GTGCAGCGGCCTGAAC | 93 | 2274 |
| 1517022 | 4304 | 4319 | N/A | N/A | CAGCGGAAAAGCATGT | 97 | 2275 |
| 1517034 | 6325 | 6340 | 1227 | 1242 | CTGGGGCGGGGACAGG | 93 | 2276 |
| 1517043 | 4739 | 4754 | N/A | N/A | TGGAACAAGTTCAAGG | 103 | 2277 |
| 1517044 | 3566 | 3581 | N/A | N/A | AATCAACCGCCAGTGA | 80 | 2278 |
| 1517047 | 5697 | 5712 | 599 | 614 | TCGCCGCGGTACTGCA | 94 | 2279 |
| 1517053 | 2994 | 3009 | N/A | N/A | CTTCCCAGGTCCAGTC | 70 | 2280 |
| 1517060 | 6293 | 6308 | 1195 | 1210 | TGCGCGGAGGCAGGAG | 38 | 2281 |
| 1517084 | 2859 | 2874 | 111 | 126 | GCTCATCCCCGTGCCC | 65 | 2282 |
| 1517094 | 3448 | 3463 | N/A | N/A | CCAAGCCGCCCCCAAC | 77 | 2283 |
| 1517095 | 6301 | 6316 | 1203 | 1218 | CTGCAGGCTGCGCGGA | 63 | 2284 |
| 1517106 | 3660 | 3675 | N/A | N/A | CCCCATACCTGCCAGG | 93 | 2285 |
| 1517112 | 2943 | 2958 | N/A | N/A | CCAAGCCCGACCCCGA | 88 | 2286 |
| 1517118 | 3497 | 3512 | N/A | N/A | TTCCAAGCCTTGTTGC | 80 | 2287 |
| 1517130 | 6365 | 6380 | 1267 | 1282 | GTGAATCTTTATTAAA | 19 | 1982 |
| 1517134 | 3218 | 3233 | N/A | N/A | GAGCTAATTCAGTCCT | 68 | 2288 |
| 1517137 | 5685 | 5700 | 587 | 602 | TGCACCAGGCGGCCGC | 96 | 2289 |
| 1517138 | 5982 | 5997 | 884 | 899 | GCGCGCAGCCGCTCGC | 84 | 2290 |

TABLE 36
Reduction of APOE RNA by 3-10-3 cEt gapmers
with mixed PO/PS internucleoside linkages at a
concentration of 4000 nM in Hep3B cells plated
at 20,000 cells per well

| | SEQ | SEQ | SEQ | SEQ | | | |
|---|---|---|---|---|---|---|---|
| | ID No: | ID No: | ID No: | ID No: | | | |
| | 1 | 1 | 2 | 2 | | APOE | |
| Compound | Start | Stop | Start | Stop | Sequence | (% | SEQ |
| Number | Site | Site | Site | Site | (5′ to 3′) | UTC) | ID NO |
| 1335572 | 3474 | 3489 | N/A | N/A | GCAACAGCCTAATCCC | 65 | 2291 |
| 1335676 | 3455 | 3470 | N/A | N/A | ACATTTACCAAGCCGC | 74 | 2292 |
| 1516531 | 2942 | 2957 | N/A | N/A | CAAGCCCGACCCCGAG | 93 | 2293 |
| 1516535 | 3447 | 3462 | N/A | N/A | CAAGCCGCCCCCAACC | 79 | 2294 |
| 1516540 | 6340 | 6355 | 1242 | 1257 | ACCCCAGGAGGACGGC | 65 | 2295 |
| 1516541 | 5522 | 5537 | N/A | N/A | GCGCCCTGCGGCCGAG | 102† | 2296 |
| 1516542 | 3837 | 3852 | N/A | N/A | TAACTCCTATCTCAAG | 129 | 2297 |
| 1516547 | 5662 | 5677 | 564 | 579 | CTCCATGTCCGCGCCC | 46 | 2298 |
| 1516564 | 3565 | 3580 | N/A | N/A | ATCAACCGCCAGTGAG | 89 | 2299 |
| 1516569 | 4694 | 4709 | N/A | N/A | GCATCTAGTACCTAGG | 72 | 2300 |
| 1516574 | 4738 | 4753 | N/A | N/A | GGAACAAGTTCAAGGT | 94 | 2301 |
| 1516587 | 5835 | 5850 | 737 | 752 | CGGGCCCCGGCCTGGT | 91 | 2302 |
| 1516592 | 3578 | 3593 | N/A | N/A | GGAGAAACTGTCAATC | 101 | 2303 |
| 1516620 | 6356 | 6371 | 1258 | 1273 | TATTAAACTAGGGTCC | 28 | 2304 |
| 1516642 | 5742 | 5757 | 644 | 659 | CGCACCCGCAGCTCCT | 62 | 2305 |
| 1516659 | 3659 | 3674 | N/A | N/A | CCCATACCTGCCAGGA | 95 | 2306 |
| 1516671 | 5914 | 5929 | 816 | 831 | GGCGGCCCGCACGCGG | 105 | 2307 |
| 1516679 | 3314 | 3329 | N/A | N/A | CACCGCCGGTTCCATC | 83 | 2308 |
| 1516690 | 2877 | 2892 | 129 | 144 | CTTTCTAGAGGCCCCT | 87 | 2309 |
| 1516691 | 3217 | 3232 | N/A | N/A | AGCTAATTCAGTCCTC | 73 | 2310 |
| 1516694 | 3677 | 3692 | N/A | N/A | AACCGAGCAAGCCCCG | 100 | 2311 |
| 1516695 | 6114 | 6129 | 1016 | 1031 | CGGGCCTGGAAGGCCT | 94 | 2312 |
| 1516712 | 6292 | 6307 | 1194 | 1209 | GCGCGGAGGCAGGAGG | 74 | 2313 |
| 1516715 | 6236 | 6251 | 1138 | 1153 | TTCAGTGATTGTCGCT | 28 | 2314 |
| 1516717 | 3512 | 3527 | N/A | N/A | CACCCCAGGTTAGCCT | 93 | 2315 |
| 1516721 | 5980 | 5995 | 882 | 897 | GCGCAGCCGCTCGCCC | 76 | 2316 |
| 1516732 | 4982 | 4997 | N/A | N/A | TAGCCGCCCACCAGGA | 86† | 2317 |
| 1516737 | 6252 | 6267 | 1154 | 1169 | GCTGCAGGCTTCGGCG | 66 | 2318 |
| 1516741 | 3376 | 3391 | N/A | N/A | CCTCCATAGAAAATTC | 90 | 2319 |
| 1516753 | 5094 | 5109 | N/A | N/A | GAGAGCTAAAGCCAGG | 81† | 2320 |
| 1516758 | 6373 | 6388 | 1275 | 1290 | GAAACTTGGTGAATCT | 20 | 2321 |
| 1516760 | 5005 | 5020 | N/A | N/A | TGAAACCTGGACCTGG | 79† | 2322 |
| 1516768 | 5776 | 5791 | 678 | 693 | CCGCTTACGCAGCTTG | 64 | 2323 |
| 1516771 | 6260 | 6275 | 1162 | 1177 | GTCGCATGGCTGCAGG | 20 | 2324 |
| 1516787 | 3496 | 3511 | N/A | N/A | TCCAAGCCTTGTTGCA | 80 | 2325 |
| 1516789 | 4194 | 4209 | N/A | N/A | GCAATGCATTAGAAAC | 75 | 2326 |
| 1516817 | 3525 | 3540 | N/A | N/A | CCCAACCCGGCCTCAC | 126 | 2327 |
| 1516823 | 5905 | 5920 | 807 | 822 | CACGCGGCCCTGTTCC | 77 | 2328 |
| 1516829 | 2992 | 3007 | N/A | N/A | TCCCAGGTCCAGTCCC | 104 | 2329 |
| 1516830 | 6015 | 6030 | 917 | 932 | TCGCGGGTCCGGCTGC | 79 | 2330 |
| 1516832 | 6284 | 6299 | 1186 | 1201 | GCAGGAGGCACGGGGT | 29 | 2331 |
| 1516840 | 2919 | 2934 | N/A | N/A | CCTGAGACTACCTGGA | 77 | 2332 |
| 1516843 | 3067 | 3082 | N/A | N/A | TGGCTTACATCCCAGT | 83 | 2333 |
| 1516846 | 4545 | 4560 | N/A | N/A | CAGCGGCCTGAACATG | 92 | 2334 |
| 1516851 | 6332 | 6347 | 1234 | 1249 | AGGACGGCTGGGGCGG | 39 | 2335 |
| 1516857 | 2856 | 2871 | 108 | 123 | CATCCCCGTGCCCCCG | 84 | 2336 |
| 1516861 | 2844 | 2859 | 96 | 111 | CCCGACTGCGCTTCTC | 91 | 2337 |
| 1516871 | 4859 | 4874 | 341 | 356 | TCCCAAAAGCGACCCA | 14† | 2338 |
| 1516880 | 5695 | 5710 | 597 | 612 | GCCGCGGTACTGCACC | 60 | 2339 |
| 1516887 | 6316 | 6331 | 1218 | 1233 | GGACAGGGTCTCCCGC | 43 | 2340 |
| 1516909 | 5031 | 5046 | N/A | N/A | CCCAAGACTTAGCGAC | 80† | 2341 |
| 1516916 | 3422 | 3437 | N/A | N/A | TTTAACTCCCTCCTGG | 66 | 2342 |
| 1516921 | 6364 | 6379 | 1266 | 1281 | TGAATCTTTATTAAAC | 38 | 2343 |
| 1516931 | 6308 | 6323 | 1210 | 1225 | TCTCCCGCTGCAGGCT | 17 | 2344 |
| 1516937 | 4868 | 4883 | 350 | 365 | CGCAGGTAATCCCAAA | 9† | 2345 |
| 1516942 | 3104 | 3119 | N/A | N/A | TCGATAAATGATAGTG | 80 | 2346 |
| 1516970 | 3637 | 3652 | 211 | 226 | CCAGCAACGCAGCCCA | 58 | 2347 |
| 1516986 | 6300 | 6315 | 1202 | 1217 | TGCAGGCTGCGCGGAG | 31 | 2348 |
| 1517018 | 3794 | 3809 | N/A | N/A | GTCAAATCGCTGTTCA | 78 | 2349 |
| 1517030 | 5799 | 5814 | 701 | 716 | AGGTCATCGGCATCGC | 47 | 2350 |
| 1517049 | 6244 | 6259 | 1146 | 1161 | CTTCGGCGTTCAGTGA | 43 | 2351 |
| 1517058 | 6324 | 6339 | 1226 | 1241 | TGGGGCGGGGACAGGG | 48 | 2352 |
| 1517064 | 5053 | 5068 | N/A | N/A | GAACCAGCAGAGACCC | 48† | 2353 |
| 1517072 | 3187 | 3202 | N/A | N/A | CAATCGACGGCTAGCT | 76 | 2354 |
| 1517080 | 4302 | 4317 | N/A | N/A | GCGGAAAAGCATGTAT | 82 | 2355 |
| 1517098 | 3236 | 3251 | N/A | N/A | GCGCCGTGTTCCATTT | 90 | 2356 |
| 1517102 | 3179 | 3194 | N/A | N/A | GGCTAGCTACCGTGTC | 86 | 2357 |
| 1517108 | 6268 | 6283 | 1170 | 1185 | GGCGTGGGGTCGCATG | 32 | 2358 |
| 1517113 | 5619 | 5634 | 521 | 536 | TTGGACAGCCGTGCCC | 80 | 2359 |
| 1517117 | 3262 | 3277 | N/A | N/A | ATTCTAAGCTCCAACC | 71 | 2360 |
| 1517120 | 6348 | 6363 | 1250 | 1265 | TAGGGTCCACCCCAGG | 35 | 2361 |
| 1517121 | 6276 | 6291 | 1178 | 1193 | CACGGGGTGGCGTGGG | 47 | 2362 |
| 1517122 | 2934 | 2949 | N/A | N/A | ACCCCGAGTAGCTCTC | 65 | 2363 |
| 1517128 | 4177 | 4192 | N/A | N/A | TCTAACTCCCAGCCAG | 109 | 2364 |
| 1517130 | 6365 | 6380 | 1267 | 1282 | GTGAATCTTTATTAAA | 20 | 1982 |
| 1517132 | 6169 | 6184 | 1071 | 1086 | CCCGGCCCACTGGCGC | 98 | 2365 |
| 1517136 | 4851 | 4866 | 333 | 348 | GCGACCCAGTGCCAGT | 27† | 2366 |
| 1517140 | 3435 | 3450 | N/A | N/A | AACCCATTCCCTATTT | 108 | 2367 |

TABLE 37
Reduction of APOE RNA by 3-10-3 cEt gapmers
with mixed PO/PS internucleoside linkages at a
concentration of 4000 nM in Hep3B cells plated
at 20,000 cells per well

| | SEQ | SEQ | SEQ | SEQ | | | |
|---|---|---|---|---|---|---|---|
| | ID No: | ID No: | ID No: | ID No: | | | |
| | 1 | 1 | 2 | 2 | | APOE | |
| Compound | Start | Stop | Start | Stop | Sequence | (% | SEQ |
| Number | Site | Site | Site | Site | (5′ to 3′) | UTC) | ID NO |
| 1335667 | 3575 | 3590 | N/A | N/A | GAAACTGTCAATCAAC | 113 | 2368 |
| 1335670 | 3793 | 3808 | N/A | N/A | TCAAATCGCTGTTCAG | 91 | 2369 |
| 1335672 | 6355 | 6370 | 1257 | 1272 | ATTAAACTAGGGTCCA | 24 | 2370 |
| 1516524 | 4737 | 4752 | N/A | N/A | GAACAAGTTCAAGGTG | 77 | 2371 |
| 1516544 | 3313 | 3328 | N/A | N/A | ACCGCCGGTTCCATCT | 53 | 2372 |
| 1516562 | 5775 | 5790 | 677 | 692 | CGCTTACGCAGCTTGC | 93 | 2373 |
| 1516572 | 5834 | 5849 | 736 | 751 | GGGCCCCGGCCTGGTA | 107 | 2374 |
| 1516577 | 3216 | 3231 | N/A | N/A | GCTAATTCAGTCCTCA | 74 | 2375 |
| 1516583 | 5091 | 5106 | N/A | N/A | AGCTAAAGCCAGGAGT | 90† | 2376 |
| 1516590 | 2933 | 2948 | N/A | N/A | CCCCGAGTAGCTCTCC | 94 | 2377 |
| 1516601 | 6275 | 6290 | 1177 | 1192 | ACGGGGTGGCGTGGGG | 79 | 2378 |
| 1516608 | 5798 | 5813 | 700 | 715 | GGTCATCGGCATCGCG | 50 | 2379 |
| 1516617 | 6363 | 6378 | 1265 | 1280 | GAATCTTTATTAAACT | 12 | 2380 |
| 1516619 | 2831 | 2846 | 83 | 98 | CTCACCGGCTCCTGGG | 86 | 2381 |
| 1516622 | 3564 | 3579 | N/A | N/A | TCAACCGCCAGTGAGG | 93 | 2382 |
| 1516631 | 3473 | 3488 | N/A | N/A | CAACAGCCTAATCCCA | 83 | 2383 |
| 1516634 | 5992 | 6007 | 894 | 909 | CTCCATCCGCGCGCGC | 94 | 2384 |
| 1516635 | 4866 | 4881 | 348 | 363 | CAGGTAATCCCAAAAG | 63† | 2385 |
| 1516636 | 5977 | 5992 | 879 | 894 | CAGCCGCTCGCCCCAG | 102 | 2386 |
| 1516637 | 6134 | 6149 | 1036 | 1051 | GCTCGAACCAGCTCTT | 66 | 2387 |
| 1516639 | 5740 | 5755 | 642 | 657 | CACCCGCAGCTCCTCG | 89 | 2388 |
| 1516640 | 6331 | 6346 | 1233 | 1248 | GGACGGCTGGGGCGGG | 78 | 2389 |
| 1516643 | 5656 | 5671 | 558 | 573 | GTCCGCGCCCAGCCGG | 80 | 2390 |
| 1516650 | 3454 | 3469 | N/A | N/A | CATTTACCAAGCCGCC | 80 | 2391 |
| 1516651 | 3658 | 3673 | N/A | N/A | CCATACCTGCCAGGAA | 80 | 2392 |
| 1516652 | 6307 | 6322 | 1209 | 1224 | CTCCCGCTGCAGGCTG | 26 | 2393 |
| 1516664 | 3494 | 3509 | N/A | N/A | CAAGCCTTGTTGCATT | 79 | 2394 |
| 1516683 | 3177 | 3192 | N/A | N/A | CTAGCTACCGTGTCGC | 92 | 2395 |
| 1516685 | 6323 | 6338 | 1225 | 1240 | GGGGCGGGGACAGGGT | 114 | 2396 |
| 1516689 | 3360 | 3375 | N/A | N/A | CATCTTCCTCTCCCGG | 86 | 2397 |
| 1516698 | 2855 | 2870 | 107 | 122 | ATCCCCGTGCCCCCGA | 108 | 2398 |
| 1516711 | 2965 | 2980 | N/A | N/A | CTCACCCCCGCTCCTC | 89 | 2399 |
| 1516719 | 3421 | 3436 | N/A | N/A | TTAACTCCCTCCTGGT | 111 | 2400 |
| 1516720 | 3186 | 3201 | N/A | N/A | AATCGACGGCTAGCTA | 88 | 2401 |
| 1516722 | 3836 | 3851 | N/A | N/A | AACTCCTATCTCAAGG | 96 | 2402 |
| 1516724 | 6283 | 6298 | 1185 | 1200 | CAGGAGGCACGGGGTG | 65 | 2403 |
| 1516725 | 3228 | 3243 | N/A | N/A | TTCCATTTATGAGCTA | 70 | 2404 |
| 1516727 | 5913 | 5928 | 815 | 830 | GCGGCCCGCACGCGGC | 102 | 2405 |
| 1516733 | 5617 | 5632 | 519 | 534 | GGACAGCCGTGCCCGC | 80 | 2406 |
| 1516739 | 5694 | 5709 | 596 | 611 | CCGCGGTACTGCACCA | 105 | 2407 |
| 1516752 | 5904 | 5919 | 806 | 821 | ACGCGGCCCTGTTCCA | 93 | 2408 |
| 1516766 | 3524 | 3539 | N/A | N/A | CCAACCCGGCCTCACC | 92 | 2409 |
| 1516776 | 3676 | 3691 | N/A | N/A | ACCGAGCAAGCCCCGC | 132 | 2410 |
| 1516798 | 3434 | 3449 | N/A | N/A | ACCCATTCCCTATTTA | 117 | 2411 |
| 1516801 | 3103 | 3118 | N/A | N/A | CGATAAATGATAGTGA | 77 | 2412 |
| 1516816 | 4858 | 4873 | 340 | 355 | CCCAAAAGCGACCCAG | 14† | 2413 |
| 1516820 | 4301 | 4316 | N/A | N/A | CGGAAAAGCATGTATT | 88 | 2414 |
| 1516821 | 5515 | 5530 | N/A | N/A | GCGGCCGAGAGGGCGG | 109† | 2415 |
| 1516833 | 4193 | 4208 | N/A | N/A | CAATGCATTAGAAACC | 88 | 2416 |
| 1516839 | 6259 | 6274 | 1161 | 1176 | TCGCATGGCTGCAGGC | 10 | 2417 |
| 1516849 | 3446 | 3461 | N/A | N/A | AAGCCGCCCCCAACCC | 96 | 2418 |
| 1516852 | 3065 | 3080 | N/A | N/A | GCTTACATCCCAGTCC | 128 | 2419 |
| 1516853 | 6291 | 6306 | 1193 | 1208 | CGCGGAGGCAGGAGGC | 53 | 2420 |
| 1516855 | 4980 | 4995 | N/A | N/A | GCCGCCCACCAGGAGG | 98† | 2421 |
| 1516856 | 4989 | 5004 | N/A | N/A | GGAGGTATAGCCGCCC | 97† | 2422 |
| 1516859 | 2917 | 2932 | N/A | N/A | TGAGACTACCTGGAGG | 95 | 2423 |
| 1516872 | 5029 | 5044 | N/A | N/A | CAAGACTTAGCGACAG | 71† | 2424 |
| 1516878 | 3636 | 3651 | 210 | 225 | CAGCAACGCAGCCCAC | 68 | 2425 |
| 1516894 | 6339 | 6354 | 1241 | 1256 | CCCCAGGAGGACGGCT | 92 | 2426 |
| 1516910 | 3508 | 3523 | N/A | N/A | CCAGGTTAGCCTTCCA | 92 | 2427 |
| 1516918 | 6235 | 6250 | 1137 | 1152 | TCAGTGATTGTCGCTG | 70 | 2428 |
| 1516919 | 6347 | 6362 | 1249 | 1264 | AGGGTCCACCCCAGGA | 82 | 2429 |
| 1516920 | 2941 | 2956 | N/A | N/A | AAGCCCGACCCCGAGT | 110 | 2430 |
| 1516927 | 6112 | 6127 | 1014 | 1029 | GGCCTGGAAGGCCTCG | 92 | 2431 |
| 1516944 | 6315 | 6330 | 1217 | 1232 | GACAGGGTCTCCCGCT | 28 | 2432 |
| 1516971 | 6251 | 6266 | 1153 | 1168 | CTGCAGGCTTCGGCGT | 78 | 2433 |
| 1516995 | 5038 | 5053 | N/A | N/A | CAGGCCCCCCAAGACT | 104† | 2434 |
| 1516999 | 4543 | 4558 | N/A | N/A | GCGGCCTGAACATGGT | 94 | 2435 |
| 1517003 | 6372 | 6387 | 1274 | 1289 | AAACTTGGTGAATCTT | 26 | 2436 |
| 1517004 | 3261 | 3276 | N/A | N/A | TTCTAAGCTCCAACCT | 89 | 2437 |
| 1517011 | 6299 | 6314 | 1201 | 1216 | GCAGGCTGCGCGGAGG | 38 | 2438 |
| 1517013 | 4175 | 4190 | N/A | N/A | TAACTCCCAGCCAGGT | 121 | 2439 |
| 1517026 | 6267 | 6282 | 1169 | 1184 | GCGTGGGGTCGCATGG | 15 | 2440 |
| 1517041 | 4693 | 4708 | N/A | N/A | CATCTAGTACCTAGGG | 88 | 2441 |
| 1517097 | 6243 | 6258 | 1145 | 1160 | TTCGGCGTTCAGTGAT | 57 | 2442 |
| 1517115 | 4850 | 4865 | 332 | 347 | CGACCCAGTGCCAGTT | 98† | 2443 |
| 1517125 | 2876 | 2891 | 128 | 143 | TTTCTAGAGGCCCCTG | 75 | 2444 |
| 1517130 | 6365 | 6380 | 1267 | 1282 | GTGAATCTTTATTAAA | 34 | 1982 |

TABLE 38
Reduction of APOE RNA by 3-10-3 cEt gapmers
with mixed PO/PS internucleoside linkages at a
concentration of 4000 nM in Hep3B cells plated
at 20,000 cells per well

| | SEQ | SEQ | SEQ | SEQ | | | |
|---|---|---|---|---|---|---|---|
| | ID No: | ID No: | ID No: | ID No: | | | |
| | 1 | 1 | 2 | 2 | | APOE | |
| Compound | Start | Stop | Start | Stop | Sequence | (% | SEQ |
| Number | Site | Site | Site | Site | (5′ to 3′) | UTC) | ID NO |
| 1335665 | 5028 | 5043 | N/A | N/A | AAGACTTAGCGACAGG | 89† | 2445 |
| 1335683 | 4191 | 4206 | N/A | N/A | ATGCATTAGAAACCTC | 95 | 2446 |
| 1516526 | 6322 | 6337 | 1224 | 1239 | GGGCGGGGACAGGGTC | 61 | 2447 |
| 1516530 | 3492 | 3507 | N/A | N/A | AGCCTTGTTGCATTAT | 95 | 2448 |
| 1516533 | 6371 | 6386 | 1273 | 1288 | AACTTGGTGAATCTTT | 45 | 2449 |
| 1516546 | 4673 | 4688 | N/A | N/A | ACAGAAGGTGCTCCAC | 110 | 2450 |
| 1516549 | 6362 | 6377 | 1264 | 1279 | AATCTTTATTAAACTA | 43 | 2451 |
| 1516554 | 3258 | 3273 | N/A | N/A | TAAGCTCCAACCTCAC | 76 | 2452 |
| 1516557 | 5572 | 5587 | 474 | 489 | CTCCAGTTCCGATTTG | 70 | 2453 |
| 1516565 | 5976 | 5991 | 878 | 893 | AGCCGCTCGCCCCAGG | 118 | 2454 |
| 1516566 | 3792 | 3807 | N/A | N/A | CAAATCGCTGTTCAGA | 102 | 2455 |
| 1516580 | 5037 | 5052 | N/A | N/A | AGGCCCCCCAAGACTT | 111† | 2456 |
| 1516584 | 3657 | 3672 | N/A | N/A | CATACCTGCCAGGAAT | 108 | 2457 |
| 1516585 | 5693 | 5708 | 595 | 610 | CGCGGTACTGCACCAG | 94 | 2458 |
| 1516586 | 4128 | 4143 | N/A | N/A | TGGGAGATCGAAACGG | 90 | 2459 |
| 1516605 | 3574 | 3589 | N/A | N/A | AAACTGTCAATCAACC | 113 | 2460 |
| 1516626 | 6234 | 6249 | 1136 | 1151 | CAGTGATTGTCGCTGG | 54 | 2461 |
| 1516628 | 6266 | 6281 | 1168 | 1183 | CGTGGGGTCGCATGGC | 18 | 2462 |
| 1516647 | 2916 | 2931 | N/A | N/A | GAGACTACCTGGAGGC | 108 | 2463 |
| 1516648 | 5833 | 5848 | 735 | 750 | GGCCCCGGCCTGGTAC | 116 | 2464 |
| 1516665 | 5990 | 6005 | 892 | 907 | CCATCCGCGCGCGCAG | 65 | 2465 |
| 1516669 | 3835 | 3850 | N/A | N/A | ACTCCTATCTCAAGGA | 104 | 2466 |
| 1516676 | 3176 | 3191 | N/A | N/A | TAGCTACCGTGTCGCT | 99 | 2467 |
| 1516699 | 6133 | 6148 | 1035 | 1050 | CTCGAACCAGCTCTTG | 92 | 2468 |
| 1516701 | 6250 | 6265 | 1152 | 1167 | TGCAGGCTTCGGCGTT | 51 | 2469 |
| 1516702 | 6242 | 6257 | 1144 | 1159 | TCGGCGTTCAGTGATT | 72 | 2470 |
| 1516705 | 3312 | 3327 | N/A | N/A | CCGCCGGTTCCATCTC | 87 | 2471 |
| 1516714 | 3635 | 3650 | 209 | 224 | AGCAACGCAGCCCACA | 71 | 2472 |
| 1516738 | 6314 | 6329 | 1216 | 1231 | ACAGGGTCTCCCGCTG | 70 | 2473 |
| 1516751 | 6330 | 6345 | 1232 | 1247 | GACGGCTGGGGCGGGG | 119 | 2474 |
| 1516754 | 2805 | 2820 | 57 | 72 | CTTCACCTCCGCTGGG | 94 | 2475 |
| 1516764 | 3472 | 3487 | N/A | N/A | AACAGCCTAATCCCAG | 40 | 2476 |
| 1516767 | 3432 | 3447 | N/A | N/A | CCATTCCCTATTTAAC | 117 | 2477 |
| 1516804 | 3445 | 3460 | N/A | N/A | AGCCGCCCCCAACCCA | 108 | 2478 |
| 1516813 | 5912 | 5927 | 814 | 829 | CGGCCCGCACGCGGCC | 111 | 2479 |
| 1516826 | 3341 | 3356 | N/A | N/A | TCAAATTCCATCCCCC | 85 | 2480 |
| 1516831 | 6306 | 6321 | 1208 | 1223 | TCCCGCTGCAGGCTGC | 56 | 2481 |
| 1516835 | 2854 | 2869 | 106 | 121 | TCCCCGTGCCCCCGAC | 111 | 2482 |
| 1516868 | 6338 | 6353 | 1240 | 1255 | CCCAGGAGGACGGCTG | 89 | 2483 |
| 1516870 | 3420 | 3435 | N/A | N/A | TAACTCCCTCCTGGTC | 101 | 2484 |
| 1516874 | 3507 | 3522 | N/A | N/A | CAGGTTAGCCTTCCAA | 106 | 2485 |
| 1516877 | 6258 | 6273 | 1160 | 1175 | CGCATGGCTGCAGGCT | 35 | 2486 |
| 1516879 | 5739 | 5754 | 641 | 656 | ACCCGCAGCTCCTCGG | 104 | 2487 |
| 1516883 | 4233 | 4248 | N/A | N/A | GATCACAGCTGCCCCG | 112 | 2488 |
| 1516889 | 4979 | 4994 | N/A | N/A | CCGCCCACCAGGAGGG | 104† | 2489 |
| 1516891 | 6354 | 6369 | 1256 | 1271 | TTAAACTAGGGTCCAC | 48 | 2490 |
| 1516895 | 5090 | 5105 | N/A | N/A | GCTAAAGCCAGGAGTC | 107† | 2491 |
| 1516898 | 3453 | 3468 | N/A | N/A | ATTTACCAAGCCGCCC | 115 | 2492 |
| 1516901 | 6298 | 6313 | 1200 | 1215 | CAGGCTGCGCGGAGGC | 46 | 2493 |
| 1516905 | 3097 | 3112 | N/A | N/A | ATGATAGTGACAACTC | 79 | 2494 |
| 1516926 | 2875 | 2890 | 127 | 142 | TTCTAGAGGCCCCTGA | 104 | 2495 |
| 1516941 | 4988 | 5003 | N/A | N/A | GAGGTATAGCCGCCCA | 119† | 2496 |
| 1516945 | 6274 | 6289 | 1176 | 1191 | CGGGGTGGCGTGGGGT | 122 | 2497 |
| 1516967 | 3185 | 3200 | N/A | N/A | ATCGACGGCTAGCTAC | 108 | 2498 |
| 1516980 | 6282 | 6297 | 1184 | 1199 | AGGAGGCACGGGGTGG | 78 | 2499 |
| 1516997 | 5344 | 5359 | N/A | N/A | GCAGAGACGAAGAAGG | 89† | 2500 |
| 1517007 | 2949 | 2964 | N/A | N/A | CTCTCCCCAAGCCCGA | 86 | 2501 |
| 1517008 | 3557 | 3572 | N/A | N/A | CCAGTGAGGACTCCTC | 123 | 2502 |
| 1517012 | 5787 | 5802 | 689 | 704 | TCGCGGAGGAGCCGCT | 99 | 2503 |
| 1517028 | 5773 | 5788 | 675 | 690 | CTTACGCAGCTTGCGC | 99 | 2504 |
| 1517032 | 6111 | 6126 | 1013 | 1028 | GCCTGGAAGGCCTCGG | 104 | 2505 |
| 1517035 | 2940 | 2955 | N/A | N/A | AGCCCGACCCCGAGTA | 104 | 2506 |
| 1517045 | 2932 | 2947 | N/A | N/A | CCCGAGTAGCTCTCCT | 108 | 2507 |
| 1517051 | 3674 | 3689 | N/A | N/A | CGAGCAAGCCCCGCCC | 131 | 2508 |
| 1517055 | 5648 | 5663 | 550 | 565 | CCAGCCGGGCCTGCGC | 90 | 2509 |
| 1517067 | 4857 | 4872 | 339 | 354 | CCAAAAGCGACCCAGT | 23† | 2510 |
| 1517070 | 6290 | 6305 | 1192 | 1207 | GCGGAGGCAGGAGGCA | 81 | 2511 |
| 1517071 | 6346 | 6361 | 1248 | 1263 | GGGTCCACCCCAGGAG | 107 | 2512 |
| 1517087 | 5903 | 5918 | 805 | 820 | CGCGGCCCTGTTCCAC | 58 | 2513 |
| 1517089 | 3198 | 3213 | N/A | N/A | TTAAAGTTCTCCAATC | 92 | 2514 |
| 1517090 | 3064 | 3079 | N/A | N/A | CTTACATCCCAGTCCA | 97 | 2515 |
| 1517092 | 3523 | 3538 | N/A | N/A | CAACCCGGCCTCACCC | 129 | 2516 |
| 1517093 | 4842 | 4857 | 324 | 339 | TGCCAGTTCCCAGCGC | 177 | 2517 |
| 1517096 | 4865 | 4880 | 347 | 362 | AGGTAATCCCAAAAGC | 87† | 2518 |
| 1517103 | 3227 | 3242 | N/A | N/A | TCCATTTATGAGCTAA | 112 | 2519 |
| 1517127 | 4542 | 4557 | N/A | N/A | CGGCCTGAACATGGTT | 114 | 2520 |
| 1517130 | 6365 | 6380 | 1267 | 1282 | GTGAATCTTTATTAAA | 34 | 1982 |
| 1517139 | 4707 | 4722 | N/A | N/A | TGACCCCGTCCAGGCA | 110 | 2521 |

### Example 5: Dose-Dependent Inhibition of Human APOE in HepG2 Cells by Modified Oligonucleotides

Modified oligonucleotides selected from Examples 1-4 above were tested at various doses in HepG2 cells. Cultured HepG2 cells at a density of 10,000 cells per well were treated by Lipofectin with various concentrations of modified oligonucleotide as specified in the tables below. After a treatment period of approximately 24 hours, total RNA was isolated from the cells and APOE RNA levels were measured by quantitative real-time RTPCR. Human APOE primer-probe set RTS3073 (described herein in Example 1) was used to measure RNA levels as described above. APOE RNA levels were normalized to total RNA content, as measured by RIBOGREEN®. Results are presented in the tables below as percent APOE RNA, relative to untreated control cells (% UTC). Modified oligonucleotides marked with an “†” indicate that the modified oligonucleotide is complementary to the amplicon region of the primer probe set. Additional assays may be used to measure the potency and efficacy of the modified oligonucleotides complementary to the amplicon region.

The half maximal inhibitory concentration (IC₅₀) of each modified oligonucleotide was calculated using a linear regression on a log/linear plot of the data in Excel and is also presented in the tables below.

TABLE 39
Dose-dependent reduction of human APOE RNA
in HepG2 cells by modified oligonucleotides

| | APOE RNA (% UTC) | | | | | | |
|---|---|---|---|---|---|---|---|
| Compound | 6.25 | 12.5 | 25 | 50 | 100 | 200 | IC₅₀ |
| No. | nM | nM | nM | nM | nM | nM | (μM) |
| 426010† | 130 | 117 | 108 | 81 | 48 | 38 | 0.14 |
| 426011† | 124 | 113 | 96 | 71 | 29 | 24 | 0.08 |
| 426013† | 84 | 76 | 52 | 24 | 7 | 5 | 0.03 |
| 426017† | 100 | 98 | 66 | 38 | 14 | 12 | 0.04 |
| 426020 | 142 | 123 | 114 | 92 | 55 | 54 | >0.2 |
| 426028 | 128 | 117 | 98 | 69 | 47 | 39 | 0.12 |
| 426047 | 124 | 121 | 120 | 108 | 60 | 53 | >0.2 |
| 426048 | 107 | 104 | 70 | 57 | 50 | 32 | 0.09 |
| 426049 | 121 | 119 | 109 | 80 | 59 | 40 | 0.16 |
| 426055 | 120 | 112 | 105 | 86 | 83 | 60 | >0.2 |

### Example 6: Dose-Dependent Inhibition of Human APOE in Hep3B Cells by Modified Oligonucleotides

Modified oligonucleotides selected from Examples 1˜4 above were tested at various doses in Hep3B cells. Cultured Hep3B cells at a density of 20,000 cells per well were treated by electroporation with various concentrations of modified oligonucleotide as specified in the tables below. After a treatment period of approximately 24 hours, total RNA was isolated from the cells and APOE RNA levels were measured by quantitative real-time RTPCR. Human APOE primer-probe set RTS3073 (described herein in Example 1) was used to measure RNA levels as described above. APOE RNA levels were normalized to total RNA content, as measured by RIBOGREEN®. Reduction of APOE RNA is presented in the tables below as percent APOE RNA, relative to untreated control cells (% UTC). Modified oligonucleotides marked with an “f” indicate that the modified oligonucleotide is complementary to the amplicon region of the primer probe set. Additional assays may be used to measure the potency and efficacy of the modified oligonucleotides complementary to the amplicon region.

The half maximal inhibitory concentration (IC₅₀) of each modified oligonucleotide was calculated using a linear regression on a log/linear plot of the data in Excel and is also presented in the tables below.

TABLE 40
Dose-dependent reduction of human APOE RNA
in Hep3B cells by modified oligonucleotides

| Compound | APOE RNA (% UTC) | | | | | IC₅₀ |
|---|---|---|---|---|---|---|
| No. | 62 nM | 185 nM | 556 nM | 1667 nM | 5000 nM | (μM) |
| 426048 | 116 | 85 | 57 | 38 | 24 | 1.10 |
| 688999 | 137 | 131 | 151 | 100 | 79 | >5 |
| 689003 | 115 | 109 | 92 | 69 | 48 | 5.68 |
| 689009 | 117 | 136 | 126 | 78 | 72 | >5 |
| 689013 | 112 | 63 | 54 | 64 | 18 | 1.13 |
| 689014 | 97 | 122 | 67 | 44 | 19 | 1.40 |
| 689015 | 99 | 50 | 32 | 30 | 12 | 0.41 |
| 689016 | 131 | 75 | 54 | 58 | 38 | 1.72 |
| 689017 | 145 | 98 | 82 | 71 | 26 | 2.29 |
| 689023 | 132 | 172 | 116 | 84 | 53 | >5 |
| 689027 | 115 | 99 | 100 | 65 | 66 | >5 |
| 689028 | 123 | 96 | 102 | 68 | 29 | 3.11 |
| 689029 | 136 | 114 | 99 | 75 | 56 | >5 |
| 689040 | 135 | 121 | 93 | 103 | 74 | >5 |
| 689042 | 124 | 133 | 110 | 93 | 66 | >5 |

TABLE 41
Dose-dependent reduction of human APOE RNA
in Hep3B cells by modified oligonucleotides

| Compound | APOE RNA (% UTC) | | | | IC₅₀ |
|---|---|---|---|---|---|
| No. | 185 nM | 556 nM | 1667 nM | 5000 nM | (μM) |
| 426048 | 71 | 72 | 41 | 15 | 0.94 |
| 688711 | 184 | 226 | 167 | 132 | >5 |
| 688848 | 77 | 94 | 97 | 73 | >5 |
| 688886 | 82 | 103 | 108 | 66 | >5 |
| 689015 | 55 | 31 | 15 | 9 | 0.19 |
| 689016 | 71 | 62 | 50 | 22 | 1.06 |
| 689029 | 73 | 71 | 64 | 39 | 3.22 |
| 689043 | 65 | 57 | 38 | 12 | 0.63 |
| 689044 | 135 | 101 | 53 | 31 | 2.42 |
| 689046 | 58 | 32 | 26 | 15 | 0.24 |
| 689047 | 92 | 91 | 72 | 50 | >5 |
| 689048 | 53 | 31 | 25 | 13 | 0.17 |
| 689049 | 99 | 76 | 35 | 32 | 1.55 |
| 689050 | 75 | 69 | 18 | 15 | 0.73 |
| 689051 | 61 | 39 | 29 | 14 | 0.34 |
| 689090† | 92 | 116 | 110 | 92 | >5 |
| 689102† | 100 | 138 | 98 | 85 | >5 |
| 689110 | 95 | 96 | 78 | 83 | >5 |
| 689118 | 67 | 49 | 29 | 8 | 0.50 |

TABLE 42
Dose-dependent reduction of human APOE RNA
in Hep3B cells by modified oligonucleotides

| Compound | APOE RNA (% UTC) | | | | IC₅₀ |
|---|---|---|---|---|---|
| No. | 78.125 nM | 312.5 nM | 1250 nM | 5000 nM | (μM) |
| 1516537† | 46 | 31 | 19 | 11 | <0.08 |
| 1516553 | 63 | 46 | 17 | 19 | 0.19 |
| 1516570 | 71 | 56 | 33 | 23 | 0.43 |
| 1516613 | 35 | 21 | 8 | 3 | <0.08 |
| 1516617 | 84 | 54 | 30 | 16 | 0.49 |
| 1516620 | 76 | 59 | 33 | 15 | 0.46 |
| 1516715 | 74 | 58 | 29 | 11 | 0.41 |
| 1516750 | 72 | 62 | 33 | 11 | 0.44 |
| 1516758 | 44 | 26 | 25 | 26 | <0.08 |
| 1516771 | 82 | 46 | 32 | 17 | 0.43 |
| 1516828 | 64 | 43 | 20 | 15 | 0.19 |
| 1516834 | 51 | 32 | 26 | 30 | <0.08 |
| 1516839 | 56 | 41 | 17 | 14 | 0.12 |
| 1516866 | 59 | 42 | 18 | 10 | 0.15 |
| 1516871† | 29 | 16 | 10 | 7 | <0.08 |
| 1516931 | 101 | 58 | 29 | 21 | 0.71 |
| 1516937† | 40 | 18 | 6 | 5 | <0.08 |
| 1517136† | 69 | 51 | 34 | 24 | 0.37 |
| 1517427 | 95 | 50 | 25 | 27 | 0.60 |

TABLE 43
Dose-dependent reduction of human APOE RNA
in Hep3B cells by modified oligonucleotides

| Compound | APOE RNA (% UTC) | | | | IC₅₀ |
|---|---|---|---|---|---|
| No. | 78.125 nM | 312.5 nM | 1250 nM | 5000 nM | (μM) |
| 689043 | 76 | 55 | 35 | 18 | 0.47 |
| 708021 | 84 | 50 | 28 | 14 | 0.45 |
| 1335661 | 78 | 65 | 34 | 14 | 0.55 |
| 1335672 | 82 | 56 | 25 | 12 | 0.44 |
| 1516555 | 73 | 40 | 33 | 15 | 0.29 |
| 1516628 | 71 | 46 | 29 | 11 | 0.29 |
| 1516652 | 70 | 57 | 41 | 17 | 0.47 |
| 1516672 | 72 | 53 | 35 | 25 | 0.45 |
| 1516816† | 24 | 13 | 11 | 5 | <0.08 |
| 1516862 | 62 | 43 | 19 | 11 | 0.17 |
| 1516944 | 70 | 51 | 36 | 13 | 0.36 |
| 1517003 | 44 | 38 | 25 | 25 | <0.08 |
| 1517026 | 33 | 19 | 10 | 5 | <0.08 |
| 1517067† | 54 | 35 | 21 | 10 | 0.10 |
| 1517123 | 96 | 73 | 44 | 26 | 1.10 |
| 1517427 | 75 | 60 | 32 | 10 | 0.44 |
| 1517437† | 28 | 13 | 4 | 4 | <0.08 |
| 1517532 | 66 | 37 | 25 | 11 | 0.19 |
| 1517578 | 79 | 62 | 52 | 15 | 0.72 |

TABLE 44
Dose-dependent reduction of human APOE RNA
in Hep3B cells by modified oligonucleotides

| Compound | APOE RNA (% UTC) | | | | IC₅₀ |
|---|---|---|---|---|---|
| No. | 78.125 nM | 312.5 nM | 1250 nM | 5000 nM | (μM) |
| 1516539 | 86 | 65 | 36 | 19 | 0.68 |
| 1516559 | 81 | 55 | 31 | 21 | 0.51 |
| 1516607 | 96 | 67 | 38 | 22 | 0.87 |
| 1516658 | 50 | 31 | 14 | 11 | <0.08 |
| 1516740 | 31 | 20 | 17 | 27 | <0.08 |
| 1516778 | 51 | 26 | 18 | 5 | <0.08 |
| 1516794 | 91 | 62 | 33 | 22 | 0.70 |
| 1516881 | 82 | 38 | 45 | 24 | 0.50 |
| 1516886 | 99 | 78 | 45 | 28 | 1.28 |
| 1516890 | 56 | 58 | 26 | 11 | 0.23 |
| 1516932 | 45 | 30 | 23 | 23 | <0.08 |
| 1516936 | 75 | 57 | 32 | 16 | 0.44 |
| 1516974 | 66 | 46 | 23 | 30 | 0.25 |
| 1517024 | 27 | 25 | 23 | 23 | <0.08 |
| 1517029 | 72 | 57 | 30 | 23 | 0.43 |
| 1517063 | 72 | 39 | 28 | 27 | 0.28 |
| 1517130 | 94 | 76 | 46 | 34 | 1.43 |
| 1517131 | 88 | 60 | 36 | 23 | 0.70 |
| 1517427 | 89 | 66 | 36 | 29 | 0.87 |

TABLE 45
Dose-dependent reduction of human APOE RNA
in Hep3B cells by modified oligonucleotides

| Compound | APOE RNA (% UTC) | | | | IC₅₀ |
|---|---|---|---|---|---|
| No. | 78.125 nM | 312.5 nM | 1250 nM | 5000 nM | (μM) |
| 942586 | 78 | 52 | 25 | 12 | 0.38 |
| 1516589 | 80 | 79 | 51 | 33 | 1.53 |
| 1516824 | 89 | 94 | 92 | 93 | >5 |
| 1516892 | 87 | 95 | 87 | 88 | >5 |
| 1517016 | 96 | 89 | 71 | 51 | >5 |
| 1517130 | 89 | 73 | 46 | 23 | 1.00 |
| 1517180 | 91 | 75 | 53 | 31 | 1.47 |
| 1517383 | 71 | 45 | 21 | 9 | 0.26 |
| 1517427 | 85 | 57 | 29 | 15 | 0.52 |
| 1517529† | 33 | 17 | 13 | 9 | <0.08 |
| 1517535 | 80 | 53 | 29 | 21 | 0.46 |
| 1517574 | 93 | 73 | 48 | 30 | 1.29 |
| 1517746† | 84 | 57 | 32 | 18 | 0.54 |
| 1517770 | 56 | 26 | 18 | 5 | 0.08 |
| 1517807† | 63 | 30 | 20 | 14 | 0.12 |
| 1517837 | 52 | 32 | 29 | 25 | <0.08 |
| 1517849 | 66 | 49 | 32 | 27 | 0.33 |
| 1517853 | 73 | 56 | 28 | 16 | 0.39 |
| 1517885 | 79 | 58 | 33 | 18 | 0.52 |

TABLE 46
Dose-dependent reduction of human APOE RNA
in Hep3B cells by modified oligonucleotides

| Compound | APOE RNA (% UTC) | | | | IC₅₀ |
|---|---|---|---|---|---|
| No. | 78.125 nM | 312.5 nM | 1250 nM | 5000 nM | (μM) |
| 708024 | 46 | 25 | 26 | 20 | <0.08 |
| 942588 | 82 | 57 | 31 | 25 | 0.57 |
| 1516623† | 74 | 44 | 20 | 9 | 0.27 |
| 1516646 | 87 | 65 | 37 | 23 | 0.76 |
| 1516656 | 73 | 46 | 16 | 11 | 0.27 |
| 1516681 | 77 | 42 | 27 | 24 | 0.35 |
| 1516709† | 68 | 50 | 28 | 9 | 0.29 |
| 1517082 | 66 | 32 | 16 | 9 | 0.15 |
| 1517085 | 77 | 46 | 17 | 9 | 0.30 |
| 1517130 | 88 | 66 | 45 | 22 | 0.87 |
| 1517208 | 100 | 79 | 75 | 45 | 4.89 |
| 1517220 | 80 | 47 | 30 | 17 | 0.41 |
| 1517269† | 48 | 32 | 20 | 12 | <0.08 |
| 1517427 | 81 | 52 | 27 | 13 | 0.42 |
| 1517508 | 67 | 50 | 25 | 15 | 0.28 |
| 1517736† | 77 | 73 | 53 | 25 | 1.07 |
| 1517841 | 86 | 72 | 64 | 34 | 2.02 |
| 1517863† | 39 | 24 | 20 | 14 | <0.08 |
| 1517874 | 81 | 51 | 29 | 29 | 0.52 |

### Example 7: Dose-Dependent Inhibition of Human APOE in Hep3B Cells by Modified Oligonucleotides

Modified oligonucleotides selected from Examples 1˜4 above were tested at various doses in Hep3B cells. Cultured Hep3B cells at a density of 20,000 cells per well were treated by electroporation with various concentrations of modified oligonucleotide as specified in the tables below. After a treatment period of approximately 24 hours, total RNA was isolated from the cells and APOE RNA levels were measured by quantitative real-time RTPCR. Human APOE primer-probe set RTS4652 (forward sequence CGCCTGGACGAGGTGAAG, designated herein as SEQ ID NO: 13; reverse sequence CCACTGGCGCTGCATGT, designated herein as SEQ ID NO: 14; probe sequence TTCCAGGCCCGCCTCAAGAGC, designated herein as SEQ ID NO: 15) was used to measure RNA levels as described above. APOE RNA levels were normalized to total RNA content, as measured by RIBOGREEN®. Reduction of APOE RNA is presented in the tables below as percent APOE RNA, relative to untreated control cells (% UTC). Modified oligonucleotides marked with an “†” indicate that the modified oligonucleotide is complementary to the amplicon region of the primer probe set. Additional assays may be used to measure the potency and efficacy of the modified oligonucleotides complementary to the amplicon region.

The half maximal inhibitory concentration (IC₅₀) of each modified oligonucleotide was calculated using a linear regression on a log/linear plot of the data in Excel and is also presented in the tables below.

TABLE 47
Dose-dependent reduction of human APOE RNA
in Hep3B cells by modified oligonucleotides

| Compound | APOE RNA (% UTC) | | | | IC₅₀ |
|---|---|---|---|---|---|
| No. | 185 nM | 556 nM | 1667 nM | 5000 nM | (μM) |
| 426048 | 95 | 67 | 37 | 16 | 1.13 |
| 688739† | 100 | 108 | 112 | 67 | >5 |
| 688740† | 94 | 76 | 86 | 55 | >5 |
| 688742† | 65 | 112 | 70 | 77 | >5 |
| 688746† | 114 | 109 | 76 | 71 | >5 |
| 688750† | 104 | 80 | 122 | 108 | >5 |
| 688753† | 94 | 81 | 83 | 62 | >5 |
| 688758† | 70 | 76 | 72 | 56 | >5 |
| 688759† | 85 | 125 | 70 | 74 | >5 |
| 688761† | 91 | 95 | 88 | 93 | >5 |
| 688763† | 101 | 90 | 109 | 101 | >5 |
| 688769† | 80 | 97 | 87 | 71 | >5 |
| 688770† | 69 | 73 | 81 | 53 | >5 |
| 688776 | 117 | 89 | 87 | 85 | >5 |
| 688783 | 72 | 62 | 53 | 35 | 1.62 |
| 688787 | 107 | 119 | 73 | 57 | >5 |
| 688802 | 74 | 30 | 46 | 20 | 0.55 |
| 689014 | 102 | 85 | 54 | 43 | 2.86 |
| 689016 | 84 | 50 | 70 | 26 | 1.68 |

TABLE 48
Dose-dependent reduction of human APOE RNA
in Hep3B cells by modified oligonucleotides

| Compound | APOE RNA (% UTC) | | | | IC₅₀ |
|---|---|---|---|---|---|
| No. | 185 nM | 556 nM | 1667 nM | 5000 nM | (μM) |
| 426048 | 65 | 74 | 47 | 56 | >5 |
| 688738† | 81 | 76 | 85 | 87 | >5 |
| 688748† | 94 | 139 | 123 | 114 | >5 |
| 688749† | 109 | 88 | 100 | 58 | >5 |
| 688752† | 108 | 131 | 147 | 95 | >5 |
| 688756† | 70 | 73 | 88 | 55 | >5 |
| 688762† | 100 | 71 | 71 | 51 | >5 |
| 688771† | 97 | 95 | 101 | 89 | >5 |
| 688774† | 135 | 106 | 112 | 97 | >5 |
| 688775† | 137 | 112 | 201 | 104 | >5 |
| 688777 | 112 | 99 | 113 | 97 | >5 |
| 688778 | 116 | 75 | 78 | 58 | >5 |
| 688780 | 99 | 76 | 94 | 66 | >5 |
| 688800 | 125 | 108 | 92 | 92 | >5 |
| 688801 | 128 | 92 | 94 | 95 | >5 |
| 688806 | 76 | 152 | 116 | 75 | >5 |
| 688811 | 88 | 164 | 62 | 140 | >5 |
| 689014 | 83 | 81 | 98 | 56 | >5 |
| 689016 | 95 | 84 | 102 | 49 | >5 |

### Example 8: Dose-Dependent Inhibition of Human APOE in Transgenic Primary Mouse Hepatocytes by Modified Oligonucleotides

Modified oligonucleotides selected from Examples 1-4 above were tested at various doses in transgenic primary mouse hepatocytes. An ApoE4 transgenic mouse model (model #1549) was obtained from Taconic Biosciences. Cultured transgenic primary mouse hepatocytes at a density of 15,000 cells per well were treated by free uptake with various concentrations of modified oligonucleotide as specified in the tables below. After a treatment period of approximately 24 hours, total RNA was isolated from the cells and APOE RNA levels were measured by quantitative real-time RTPCR. Human APOE primer-probe set RTS3073 (described herein in Example 1) was used to measure RNA levels as described above. APOE RNA levels were normalized to total RNA content, as measured by RIBOGREEN®. Reduction of APOE RNA is presented in the tables below as percent APOE RNA, relative to untreated control cells (% UTC). Modified oligonucleotides marked with an “†” indicate that the modified oligonucleotide is complementary to the amplicon region of the primer probe set. Additional assays may be used to measure the potency and efficacy of the modified oligonucleotides complementary to the amplicon region.

The half maximal inhibitory concentration (IC₅₀) of each modified oligonucleotide was calculated using a linear regression on a log/linear plot of the data in Excel and is also presented in the tables below.

TABLE 49
Dose-dependent reduction of human APOE RNA in transgenic
primary mouse hepatocytes by modified oligonucleotides

| | APOE RNA (% UTC) | | | | |
|---|---|---|---|---|---|
| Compound | 222 | 667 | 2000 | 6000 | IC₅₀ |
| No. | nM | nM | nM | nM | (μM) |
| 689046 | 6 | 7 | 4 | 5 | <0.2 |
| 942358 | 45 | 53 | 57 | 58 | 0.54 |
| 942363 | 77 | 87 | 81 | 118 | >6 |
| 942472 | 59 | 79 | 92 | 104 | >6 |
| 942490 | 78 | 73 | 86 | 107 | >6 |
| 942496 | 63 | 81 | 81 | 94 | >6 |
| 942497 | 85 | 73 | 67 | 81 | >6 |
| 942514 | 76 | 75 | 71 | 61 | >6 |
| 942585 | 24 | 24 | 22 | 36 | <0.2 |
| 942591 | 23 | 25 | 24 | 18 | <0.2 |
| 942594 | 45 | 46 | 53 | 50 | 2.28 |
| 942595 | 43 | 45 | 36 | 38 | <0.2 |
| 942597 | 56 | 52 | 42 | 49 | 1.01 |
| 942618 | 99 | 83 | 76 | 111 | >6 |
| 942619 | 97 | 92 | 75 | 63 | >6 |
| 942627 | 144 | 107 | 86 | 63 | >6 |
| 942649 | 76 | 73 | 64 | 68 | >6 |
| 942765† | 131 | 86 | 79 | 95 | >6 |
| 942774† | 75 | 75 | 80 | 92 | >6 |

TABLE 50
Dose-dependent reduction of human APOE RNA in transgenic
primary mouse hepatocytes by modified oligonucleotides

| | APOE RNA (% UTC) | | | | |
|---|---|---|---|---|---|
| Compound | 222 | 667 | 2000 | 6000 | IC₅₀ |
| No. | nM | nM | nM | nM | (μM) |
| 689046 | 9 | 6 | 5 | 5 | <0.2 |
| 942354 | 57 | 58 | 72 | 103 | 0.23 |
| 942423 | 76 | 68 | 68 | 62 | >6 |
| 942499 | 90 | 94 | 92 | 106 | >6 |
| 942512 | 78 | 93 | 75 | 90 | >6 |
| 942542 | 135 | 113 | 102 | 142 | >6 |
| 942552 | 94 | 85 | 79 | 110 | >6 |
| 942564 | 67 | 89 | 83 | 109 | >6 |
| 942587 | 5 | 6 | 5 | 6 | <0.2 |
| 942588 | 4 | 5 | 4 | 4 | <0.2 |
| 942593 | 60 | 33 | 35 | 35 | 0.28 |
| 942598 | 15 | 13 | 14 | 17 | <0.2 |
| 942604 | 83 | 85 | 100 | 127 | >6 |
| 942617 | 82 | 87 | 72 | 69 | >6 |
| 942629 | 101 | 98 | 106 | 103 | >6 |
| 942665 | 120 | 120 | 87 | 110 | >6 |
| 942670 | 101 | 136 | 141 | 183 | >6 |
| 942724 | 96 | 74 | 86 | 80 | >6 |
| 942778† | 108 | 107 | 99 | 102 | >6 |

### Example 9: Design of RNAi Compounds with Antisense RNAi Oligonucleotides Complementary to a Human APOE Nucleic Acid

RNAi compounds comprising antisense RNAi oligonucleotides complementary to a human APOE nucleic acid and sense RNAi oligonucleotides complementary to the antisense RNAi oligonucleotides were designed as follows.

The RNAi compounds in the tables below consist of an antisense RNAi oligonucleotide and a sense RNAi oligonucleotide. The antisense RNAi oligonucleotide in each case is 23 nucleosides in length; has a sugar motif (from 5′ to 3′) of: yfyfyfyfyfyfyfyfyfyfyyy; wherein “y” represents a 2′40-methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and ‘s’ represents a phosphorothioate internucleoside linkage. The sense RNAi oligonucleotide in each case is 21 nucleosides in length; has a sugar motif (from 5′ to 3′) of: fyfyfyfyfyfyfyfyfyfyf; wherein “y” represents a 2′-O-methylribosyl sugar, and the “f” represents a 2′-fluororibosyl sugar; and a linkage motif (from 5′ to 3′) of: ssooooooooooooooooss; wherein ‘o’ represents a phosphodiester internucleoside linkage and ‘s’ represents a phosphorothioate internucleoside linkage. Each antisense RNAi oligonucleotides is complementary to the target nucleic acid (APOE), and each sense RNAi oligonucleotides is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

“Start site” indicates the 5′-most nucleoside to which the antisense RNAi oligonucleotides is complementary in the human gene sequence. “Stop site” indicates the 3′-most nucleoside to which the antisense RNAi oligonucleotide is complementary in the human gene sequence. Each modified antisense RNAi oligonucleoside listed in the tables below is 100% complementary to SEQ ID NO: 2 (described herein above). In cases where the antisense RNAi oligonucleotide is not 100% complementary to SEQ ID NO: 2, the antisense RNAi oligonucleotide is mapped to SEQ ID NO: 3 as shown in Table 52 below. Any mismatches of the antisense RNAi oligonucleotide to SEQ ID NO:3 are indicated as below.

TABLE 51
RNAi compounds targeting human APOE SEQ ID NO: 2

| | | | | SEQ ID | SEQ ID | | | |
|---|---|---|---|---|---|---|---|---|
| | Anti- | | | NO: 2 | NO: 2 | | | |
| | sense | | | Anti- | Anti- | Sense | | |
| | RNAi | Antisense | SEQ | sense | sense | RNAi | Sense | SEQ |
| Compoud | Oligo | Sequence | ID | Start | Stop | oligo | Sequence | ID |
| Number | ID | (5′ to 3′) | NO | Site | Site | ID | (5′ to 3′) | NO |
| 1518137 | 1518154 | UCACCUCCGCUG | 2552 | 48 | 70 | 1518148 | ACUCAGCCCCA | 2743 |
| | | GGGCUGAGUAG | | | | | GCGGAGGUGA | |
| 1518139 | 1518153 | AUGUGACCAGCA | 2553 | 210 | 232 | 1518145 | GGGCUGCGUUG | 2744 |
| | | ACGCAGCCCAC | | | | | CUGGUCACAU | |
| 1518140 | 1518152 | CACAGAACCUUC | 2554 | 190 | 212 | 1518147 | CAGGAAGAUGA | 2745 |
| | | AUCUUCCUGCC | | | | | AGGUUCUGUG | |
| 1518141 | 1518150 | CUCCUGGGGAAG | 2555 | 68 | 90 | 1518146 | AAGGACGUCCU | 2746 |
| | | GACGUCCUUCA | | | | | UCCCCAGGAG | |
| 1518142 | 1518151 | GGCCUGGCAUCC | 2556 | 230 | 252 | 1518144 | UUCCUGGCAGG | 2747 |
| | | UGCCAGGAAUG | | | | | AUGCCAGGCC | |
| 1518155 | 1518171 | UCCACCGCUUGC | 2557 | 250 | 272 | 1518164 | CAAGGUGGAGC | 2748 |
| | | UCCACCUUGGC | | | | | AAGCGGUGGA | |
| 1518156 | 1518169 | CACCCAGCGCAG | 2558 | 350 | 372 | 1518161 | UGGGAUUACCU | 2749 |
| | | GUAAUCCCAAA | | | | | GCGCUGGGUG | |
| 1518157 | 1518168 | AAAAGCGACCCA | 2559 | 330 | 352 | 1518162 | AACUGGCACUG | 2750 |
| | | GUGCCAGUUCC | | | | | GGUCGCUUUU | |
| 1518158 | 1518172 | UCCCAGCGCUGG | 2560 | 310 | 332 | 1518165 | GCAGAGCGGCC | 2751 |
| | | CCGCUCUGCCA | | | | | AGCGCUGGGA | |
| 1518159 | 1518167 | CCACUCGGUCUG | 2561 | 290 | 312 | 1518163 | CUGCGCCAGCA | 2752 |
| | | CUGGCGCAGCU | | | | | GACCGAGUGG | |
| 1518160 | 1518170 | GCUCGGGCUCCG | 2562 | 270 | 292 | 1518166 | AGACAGAGCCG | 2753 |
| | | GCUCUGUCUCC | | | | | GAGCCCGAGC | |
| 1518173 | 1518183 | ACCUGCUCAGAC | 2563 | 370 | 392 | 1518181 | GCAGACACUGU | 2754 |
| | | AGUGUCUGCAC | | | | | CUGAGCAGGU | |
| 1518174 | 1518185 | AGGCCUUCAACU | 2564 | 450 | 472 | 1518180 | CCAUGAAGGAG | 2755 |
| | | CCUUCAUGGUC | | | | | UUGAAGGCCU | |
| 1518175 | 1518187 | GUCUCGUCCAUC | 2565 | 430 | 452 | 1518178 | GAGGGCGCUGA | 2756 |
| | | AGCGCCCUCAG | | | | | UGGACGAGAC | |
| 1518176 | 1518190 | AGCUGAGCAGCU | 2566 | 390 | 412 | 1518186 | UGCAGGAGGAG | 2757 |
| | | CCUCCUGCACC | | | | | CUGCUCAGCU | |
| 1518177 | 1518188 | CAGUUCCUGGGU | 2567 | 410 | 432 | 1518182 | UCCCAGGUCAC | 2758 |
| | | GACCUGGGAGC | | | | | CCAGGAACUG | |
| 1518179 | 1518189 | UUCCUCCAGUUC | 2568 | 470 | 492 | 1518184 | UACAAAUCGGA | 2759 |
| | | CGAUUUGUAGG | | | | | ACUGGAGGAA | |
| 1518191 | 1518204 | UCCGCCACCGGG | 2569 | 490 | 512 | 1518200 | ACAACUGACCC | 2760 |
| | | GUCAGUUGUUC | | | | | CGGUGGCGGA | |
| 1518192 | 1518208 | GCCGCGGUACUG | 2570 | 590 | 612 | 1518198 | CGCCUGGUGCA | 2761 |
| | | CACCAGGCGGC | | | | | GUACCGCGGC | |
| 1518193 | 1518207 | GGCCGCACACGU | 2571 | 570 | 592 | 1518199 | ACAUGGAGGAC | 2762 |
| | | CCUCCAUGUCC | | | | | GUGUGCGGCC | |
| 1518194 | 1518203 | UCCGCGCCCAGC | 2572 | 550 | 572 | 1518197 | GCAGGCCCGGC | 2763 |
| | | CGGGCCUGCGC | | | | | UGGGCGCGGA | |
| 1518195 | 1518205 | CGCCGCCUGCAG | 2573 | 530 | 552 | 1518202 | UCCAAGGAGCU | 2764 |
| | | CUCCUUGGACA | | | | | GCAGGCGGCG | |
| 1518196 | 1518206 | ACAGCCGUGCCC | 2574 | 510 | 532 | 1518201 | AGGAGACGCGG | 2765 |
| | | GCGUCUCCUCC | | | | | GCACGGCUGU | |
| 1518209 | 1518221 | CCGAGCAUGGCC | 2575 | 610 | 632 | 1518218 | CGAGGUGCAGG | 2766 |
| | | UGCACCUCGCC | | | | | CCAUGCUCGG | |
| 1518210 | 1518222 | UGCCAGGCGCUU | 2576 | 710 | 732 | 1518217 | GACCUGCAGAA | 2767 |
| | | CUGCAGGUCAU | | | | | GCGCCUGGCA | |
| 1518211 | 1518226 | CAUCGGCAUCGC | 2577 | 690 | 712 | 1518216 | GGCUCCUCCGC | 2768 |
| | | GGAGGAGCCGC | | | | | GAUGCCGAUG | |
| 1518212 | 1518223 | CGCUUACGCAGC | 2578 | 670 | 692 | 1518215 | CCUGCGCAAGC | 2769 |
| | | UUGCGCAGGUG | | | | | UGCGUAAGCG | |
| 1518213 | 1518224 | GUGGGAGGCGAG | 2579 | 650 | 672 | 1518220 | CGGGUGCGCCU | 2770 |
| | | GCGCACCCGCA | | | | | CGCCUCCCAC | |
| 1518214 | 1518225 | GCAGCUCCUCGG | 2580 | 630 | 652 | 1518219 | GCCAGAGCACC | 2771 |
| | | UGCUCUGGCCG | | | | | GAGGAGCUGC | |
| 1518227 | 1518240 | CGGGCCCCGGCC | 2581 | 730 | 752 | 1518234 | AGUGUACCAGG | 2772 |
| | | UGGUACACUGC | | | | | CCGGGGCCCG | |
| 1518228 | 1518241 | UGGCGGCCCGCA | 2582 | 810 | 832 | 1518233 | AGGGCCGCGUG | 2773 |
| | | CGCGGCCCUGU | | | | | CGGGCCGCCA | |
| 1518229 | 1518239 | UGUUCCACCAGG | 2583 | 790 | 812 | 1518235 | CCUGGGGCCCC | 2774 |
| | | GGCCCCAGGCG | | | | | UGGUGGAACA | |
| 1518230 | 1518242 | GCGCUCGCGGAU | 2584 | 770 | 792 | 1518237 | CUCAGCGCCAU | 2775 |
| | | GGCGCUGAGGC | | | | | CCGCGAGCGC | |
| 1518231 | 1518243 | GGCCGCGCUCGG | 2585 | 750 | 772 | 1518236 | GCGAGGGCGCC | 2776 |
| | | CGCCCUCGCGG | | | | | GAGCGCGGCC | |
| 1518232 | 1518244 | GCCGGCCAGGGA | 2586 | 830 | 852 | 1518238 | ACUGUGGGCUC | 2777 |
| | | GCCCACAGUGG | | | | | CCUGGCCGGC | |
| 1518245 | 1518257 | GCCCGCUCCUGU | 2587 | 850 | 872 | 1518253 | CCAGCCGCUAC | 2778 |
| | | AGCGGCUGGCC | | | | | AGGAGCGGGC | |
| 1518246 | 1518261 | GCGCACCUCCGC | 2588 | 950 | 972 | 1518254 | GAGCAGGUGGC | 2779 |
| | | CACCUGCUCCU | | | | | GGAGGUGCGC | |
| 1518247 | 1518259 | CCUUCACCUCGU | 2589 | 930 | 952 | 1518252 | ACCGCCUGGAC | 2780 |
| | | CCAGGCGGUCG | | | | | GAGGUGAAGG | |
| 1518248 | 1518258 | CUCCUCCAUCCG | 2590 | 890 | 912 | 1518251 | CUGCGCGCGCG | 2781 |
| | | CGCGCGCAGCC | | | | | GAUGGAGGAG | |
| 1518249 | 1518260 | GCCGCUCGCCCC | 2591 | 870 | 892 | 1518255 | CCCAGGCCUGG | 2782 |
| | | AGGCCUGGGCC | | | | | GGCGAGCGGC | |
| 1518250 | 1518262 | UCGCGGGUCCGG | 2592 | 910 | 932 | 1518256 | GAUGGGCAGCC | 2783 |
| | | CUGCCCAUCUC | | | | | GGACCCGCGA | |
| 1518263 | 1518279 | GCCUGCUCCUCC | 2593 | 970 | 992 | 1518271 | CGCCAAGCUGG | 2784 |
| | | AGCUUGGCGCG | | | | | AGGAGCAGGC | |
| 1518264 | 1518276 | GCUGCAUGUCUU | 2594 | 1050 | 1072 | 1518270 | CCCUGGUGGAA | 2785 |
| | | CCACCAGGGGC | | | | | GACAUGCAGC | |
| 1518265 | 1518278 | CACCAGCCCGGC | 2595 | 1070 | 1092 | 1518273 | CGCCAGUGGGC | 2786 |
| | | CCACUGGCGCU | | | | | CGGGCUGGUG | |
| 1518266 | 1518275 | GGCUCGAACCAG | 2596 | 1030 | 1052 | 1518269 | CCUCAAGAGCU | 2787 |
| | | CUCUUGAGGCG | | | | | GGUUCGAGCC | |
| 1518267 | 1518280 | CCUGCAGGCGUA | 2597 | 990 | 1012 | 1518272 | CCCAGCAGAUA | 2788 |
| | | UCUGCUGGGCC | | | | | CGCCUGCAGG | |
| 1518268 | 1518277 | GCGGGCCUGGAA | 2598 | 1010 | 1032 | 1518274 | GCCGAGGCCUU | 2789 |
| | | GGCCUCGGCCU | | | | | CCAGGCCCGC | |
| 1518281 | 1518293 | ACGGCAGCCUGC | 2599 | 1090 | 1112 | 1518288 | GGAGAAGGUGC | 2790 |
| | | ACCUUCUCCAC | | | | | AGGCUGCCGU | |
| 1518282 | 1518295 | CGGCGUUCAGUG | 2600 | 1136 | 1158 | 1518289 | AGCGACAAUCA | 2791 |
| | | AUUGUCGCUGG | | | | | CUGAACGCCG | |
| 1518283 | 1518294 | GGCGUUCAGUGA | 2601 | 1135 | 1157 | 1518287 | CAGCGACAAUC | 2792 |
| | | UUGUCGCUGGG | | | | | ACUGAACGCC | |
| 1518284 | 1518297 | UCAGUGAUUGUC | 2602 | 1130 | 1152 | 1518291 | GUGCCCAGCGA | 2793 |
| | | GCUGGGCACAG | | | | | CAAUCACUGA | |
| 1518285 | 1518296 | CAGGGGCGGCGC | 2603 | 1110 | 1132 | 1518290 | UGGGCACCAGC | 2794 |
| | | UGGUGCCCACG | | | | | GCCGCCCCUG | |
| 1518286 | 1518298 | UCGGCGUUCAGU | 2604 | 1137 | 1159 | 1518292 | GCGACAAUCAC | 2795 |
| | | GAUUGUCGCUG | | | | | UGAACGCCGA | |
| 1518299 | 1518312 | UUCGGCGUUCAG | 2605 | 1138 | 1160 | 1518305 | CGACAAUCACU | 2796 |
| | | UGAUUGUCGCU | | | | | GAACGCCGAA | |
| 1518300 | 1518311 | AGGCUUCGGCGU | 2606 | 1142 | 1164 | 1518306 | AAUCACUGAAC | 2797 |
| | | UCAGUGAUUGU | | | | | GCCGAAGCCU | |
| 1518301 | 1518313 | GGCUUCGGCGUU | 2607 | 1141 | 1163 | 1518307 | CAAUCACUGAA | 2798 |
| | | CAGUGAUUGUC | | | | | CGCCGAAGCC | |
| 1518302 | 1518314 | GCUUCGGCGUUC | 2608 | 1140 | 1162 | 1518308 | ACAAUCACUGA | 2799 |
| | | AGUGAUUGUCG | | | | | ACGCCGAAGC | |
| 1518303 | 1518316 | CUUCGGCGUUCA | 2609 | 1139 | 1161 | 1518309 | GACAAUCACUG | 2800 |
| | | GUGAUUGUCGC | | | | | AACGCCGAAG | |
| 1518304 | 1518315 | CAGGCUUCGGCG | 2610 | 1143 | 1165 | 1518310 | AUCACUGAACG | 2801 |
| | | UUCAGUGAUUG | | | | | CCGAAGCCUG | |
| 1518317 | 1518329 | UGCAGGCUUCGG | 2611 | 1145 | 1167 | 1518323 | CACUGAACGCC | 2802 |
| | | CGUUCAGUGAU | | | | | GAAGCCUGCA | |
| 1518318 | 1518331 | UGGCUGCAGGCU | 2612 | 1149 | 1171 | 1518326 | GAACGCCGAAG | 2803 |
| | | UCGGCGUUCAG | | | | | CCUGCAGCCA | |
| 1518319 | 1518334 | GCAGGCUUCGGC | 2613 | 1144 | 1166 | 1518324 | UCACUGAACGC | 2804 |
| | | GUUCAGUGAUU | | | | | CGAAGCCUGC | |
| 1518320 | 1518332 | GGCUGCAGGCUU | 2614 | 1148 | 1170 | 1518325 | UGAACGCCGAA | 2805 |
| | | CGGCGUUCAGU | | | | | GCCUGCAGCC | |
| 1518321 | 1518333 | GCUGCAGGCUUC | 2615 | 1147 | 1169 | 1518328 | CUGAACGCCGA | 2806 |
| | | GGCGUUCAGUG | | | | | AGCCUGCAGC | |
| 1518322 | 1518330 | CUGCAGGCUUCG | 2616 | 1146 | 1168 | 1518327 | ACUGAACGCCG | 2807 |
| | | GCGUUCAGUGA | | | | | AAGCCUGCAG | |
| 1518335 | 1518347 | AUGGCUGCAGGC | 2617 | 1150 | 1172 | 1518342 | AACGCCGAAGC | 2808 |
| | | UUCGGCGUUCA | | | | | CUGCAGCCAU | |
| 1518336 | 1518348 | UCGCAUGGCUGC | 2618 | 1154 | 1176 | 1518341 | CCGAAGCCUGC | 2809 |
| | | AGGCUUCGGCG | | | | | AGCCAUGCGA | |
| 1518337 | 1518351 | CGCAUGGCUGCA | 2619 | 1153 | 1175 | 1518346 | GCCGAAGCCUG | 2810 |
| | | GGCUUCGGCGU | | | | | CAGCCAUGCG | |
| 1518338 | 1518350 | GCAUGGCUGCAG | 2620 | 1152 | 1174 | 1518344 | CGCCGAAGCCU | 2811 |
| | | GCUUCGGCGUU | | | | | GCAGCCAUGC | |
| 1518339 | 1518352 | CAUGGCUGCAGG | 2621 | 1151 | 1173 | 1518343 | ACGCCGAAGCC | 2812 |
| | | CUUCGGCGUUC | | | | | UGCAGCCAUG | |
| 1518340 | 1518349 | GUCGCAUGGCUG | 2622 | 1155 | 1177 | 1518345 | CGAAGCCUGCA | 2813 |
| | | CAGGCUUCGGC | | | | | GCCAUGCGAC | |
| 1518353 | 1518355 | GGUCGCAUGGCU | 2623 | 1156 | 1178 | 1518354 | GAAGCCUGCAG | 2814 |
| | | GCAGGCUUCGG | | | | | CCAUGCGACC | |
| 1518356 | 1518369 | GGGUCGCAUGGC | 2624 | 1157 | 1179 | 1518364 | AAGCCUGCAGC | 2815 |
| | | UGCAGGCUUCG | | | | | CAUGCGACCC | |
| 1518357 | 1518367 | GGGGUCGCAUGG | 2625 | 1158 | 1180 | 1518362 | AGCCUGCAGCC | 2816 |
| | | CUGCAGGCUUC | | | | | AUGCGACCCC | |
| 1518358 | 1518366 | CGUGGGGUCGCA | 2626 | 1161 | 1183 | 1518361 | CUGCAGCCAUG | 2817 |
| | | UGGCUGCAGGC | | | | | CGACCCCACG | |
| 1518359 | 1518370 | GUGGGGUCGCAU | 2627 | 1160 | 1182 | 1518363 | CCUGCAGCCAU | 2818 |
| | | GGCUGCAGGCU | | | | | GCGACCCCAC | |
| 1518360 | 1518368 | UGGGGUCGCAUG | 2628 | 1159 | 1181 | 1518365 | GCCUGCAGCCA | 2819 |
| | | GCUGCAGGCUU | | | | | UGCGACCCCA | |
| 1518371 | 1518385 | UGGCGUGGGGUC | 2629 | 1164 | 1186 | 1518380 | CAGCCAUGCGA | 2820 |
| | | GCAUGGCUGCA | | | | | CCCCACGCCA | |
| 1518372 | 1518388 | GGGUGGCGUGGG | 2630 | 1167 | 1189 | 1518379 | CCAUGCGACCCC | 2821 |
| | | GUCGCAUGGCU | | | | | ACGCCACCC | |
| 1518373 | 1518386 | GGUGGCGUGGGG | 2631 | 1166 | 1188 | 1518381 | GCCAUGCGACC | 2822 |
| | | UCGCAUGGCUG | | | | | CCACGCCACC | |
| 1518374 | 1518387 | GGCGUGGGGUCG | 2632 | 1163 | 1185 | 1518377 | GCAGCCAUGCG | 2823 |
| | | CAUGGCUGCAG | | | | | ACCCCACGCC | |
| 1518375 | 1518384 | GUGGCGUGGGGU | 2633 | 1165 | 1187 | 1518382 | AGCCAUGCGAC | 2824 |
| | | CGCAUGGCUGC | | | | | CCCACGCCAC | |
| 1518376 | 1518383 | GCGUGGGGUCGC | 2634 | 1162 | 1184 | 1518378 | UGCAGCCAUGC | 2825 |
| | | AUGGCUGCAGG | | | | | GACCCCACGC | |
| 1518389 | 1518406 | CGGGGUGGCGUG | 2635 | 1169 | 1191 | 1518400 | AUGCGACCCCA | 2826 |
| | | GGGUCGCAUGG | | | | | CGCCACCCCG | |
| 1518390 | 1518404 | GGCACGGGGUGG | 2636 | 1173 | 1195 | 1518397 | GACCCCACGCCA | 2827 |
| | | CGUGGGGUCGC | | | | | CCCCGUGCC | |
| 1518391 | 1518402 | GCACGGGGUGGC | 2637 | 1172 | 1194 | 1518399 | CGACCCCACGCC | 2828 |
| | | GUGGGGUCGCA | | | | | ACCCCGUGC | |
| 1518392 | 1518403 | CACGGGGUGGCG | 2638 | 1171 | 1193 | 1518398 | GCGACCCCACGC | 2829 |
| | | UGGGGUCGCAU | | | | | CACCCCGUG | |
| 1518393 | 1518405 | GGGGUGGCGUGG | 2639 | 1168 | 1190 | 1518396 | CAUGCGACCCC | 2830 |
| | | GGUCGCAUGGC | | | | | ACGCCACCCC | |
| 1518394 | 1518401 | ACGGGGUGGCGU | 2640 | 1170 | 1192 | 1518395 | UGCGACCCCAC | 2831 |
| | | GGGGUCGCAUG | | | | | GCCACCCCGU | |
| 1518407 | 1518419 | GAGGCACGGGGU | 2641 | 1175 | 1197 | 1518416 | CCCCACGCCACC | 2832 |
| | | GGCGUGGGGUC | | | | | CCGUGCCUC | |
| 1518408 | 1518423 | GCAGGAGGCACG | 2642 | 1179 | 1201 | 1518415 | ACGCCACCCCGU | 2833 |
| | | GGGUGGCGUGG | | | | | GCCUCCUGC | |
| 1518409 | 1518424 | CAGGAGGCACGG | 2643 | 1178 | 1200 | 1518413 | CACGCCACCCCG | 2834 |
| | | GGUGGCGUGGG | | | | | UGCCUCCUG | |
| 1518410 | 1518421 | GGAGGCACGGGG | 2644 | 1176 | 1198 | 1518414 | CCCACGCCACCC | 2835 |
| | | UGGCGUGGGGU | | | | | CGUGCCUCC | |
| 1518411 | 1518422 | AGGAGGCACGGG | 2645 | 1177 | 1199 | 1518418 | CCACGCCACCCC | 2836 |
| | | GUGGCGUGGGG | | | | | GUGCCUCCU | |
| 1518412 | 1518420 | AGGCACGGGGUG | 2646 | 1174 | 1196 | 1518417 | ACCCCACGCCAC | 2837 |
| | | GCGUGGGGUCG | | | | | CCCGUGCCU | |
| 1518425 | 1518440 | GGCAGGAGGCAC | 2647 | 1180 | 1202 | 1518432 | CGCCACCCCGUG | 2838 |
| | | GGGGUGGCGUG | | | | | CCUCCUGCC | |
| 1518426 | 1518441 | GCGGAGGCAGGA | 2648 | 1185 | 1207 | 1518433 | CCCCGUGCCUCC | 2839 |
| | | GGCACGGGGUG | | | | | UGCCUCCGC | |
| 1518427 | 1518442 | GGAGGCAGGAGG | 2649 | 1183 | 1205 | 1518436 | CACCCCGUGCCU | 2840 |
| | | CACGGGGUGGC | | | | | CCUGCCUCC | |
| 1518428 | 1518438 | CGGAGGCAGGAG | 2650 | 1184 | 1206 | 1518435 | ACCCCGUGCCUC | 2841 |
| | | GCACGGGGUGG | | | | | CUGCCUCCG | |
| 1518429 | 1518437 | GAGGCAGGAGGC | 2651 | 1182 | 1204 | 1518434 | CCACCCCGUGCC | 2842 |
| | | ACGGGGUGGCG | | | | | UCCUGCCUC | |
| 1518430 | 1518439 | AGGCAGGAGGCA | 2652 | 1181 | 1203 | 1518431 | GCCACCCCGUGC | 2843 |
| | | CGGGGUGGCGU | | | | | CUCCUGCCU | |
| 1518443 | 1518456 | CGCGGAGGCAGG | 2653 | 1186 | 1208 | 1518450 | CCCGUGCCUCCU | 2844 |
| | | AGGCACGGGGU | | | | | GCCUCCGCG | |
| 1518444 | 1518460 | GGCUGCGCGGAG | 2654 | 1191 | 1213 | 1518451 | GCCUCCUGCCUC | 2845 |
| | | GCAGGAGGCAC | | | | | CGCGCAGCC | |
| 1518445 | 1518455 | GCUGCGCGGAGG | 2655 | 1190 | 1212 | 1518453 | UGCCUCCUGCC | 2846 |
| | | CAGGAGGCACG | | | | | UCCGCGCAGC | |
| 1518446 | 1518458 | UGCGCGGAGGCA | 2656 | 1188 | 1210 | 1518454 | CGUGCCUCCUG | 2847 |
| | | GGAGGCACGGG | | | | | CCUCCGCGCA | |
| 1518447 | 1518459 | GCGCGGAGGCAG | 2657 | 1187 | 1209 | 1518449 | CCGUGCCUCCU | 2848 |
| | | GAGGCACGGGG | | | | | GCCUCCGCGC | |
| 1518448 | 1518457 | CUGCGCGGAGGC | 2658 | 1189 | 1211 | 1518452 | GUGCCUCCUGC | 2849 |
| | | AGGAGGCACGG | | | | | CUCCGCGCAG | |
| 1518461 | 1518477 | CAGGCUGCGCGG | 2659 | 1193 | 1215 | 1518467 | CUCCUGCCUCCG | 2850 |
| | | AGGCAGGAGGC | | | | | CGCAGCCUG | |
| 1518462 | 1518476 | CGCUGCAGGCUG | 2660 | 1198 | 1220 | 1518469 | GCCUCCGCGCA | 2851 |
| | | CGCGGAGGCAG | | | | | GCCUGCAGCG | |
| 1518463 | 1518474 | CUGCAGGCUGCG | 2661 | 1196 | 1218 | 1518472 | CUGCCUCCGCGC | 2852 |
| | | CGGAGGCAGGA | | | | | AGCCUGCAG | |
| 1518464 | 1518473 | UGCAGGCUGCGC | 2662 | 1195 | 1217 | 1518471 | CCUGCCUCCGCG | 2853 |
| | | GGAGGCAGGAG | | | | | CAGCCUGCA | |
| 1518465 | 1518478 | AGGCUGCGCGGA | 2663 | 1192 | 1214 | 1518470 | CCUCCUGCCUCC | 2854 |
| | | GGCAGGAGGCA | | | | | GCGCAGCCU | |
| 1518466 | 1518475 | GCAGGCUGCGCG | 2664 | 1194 | 1216 | 1518468 | UCCUGCCUCCGC | 2855 |
| | | GAGGCAGGAGG | | | | | GCAGCCUGC | |
| 1518479 | 1518492 | CCGCUGCAGGCU | 2665 | 1199 | 1221 | 1518489 | CCUCCGCGCAGC | 2856 |
| | | GCGCGGAGGCA | | | | | CUGCAGCGG | |
| 1518480 | 1518493 | UCUCCCGCUGCA | 2666 | 1203 | 1225 | 1518486 | CGCGCAGCCUG | 2857 |
| | | GGCUGCGCGGA | | | | | CAGCGGGAGA | |
| 1518481 | 1518491 | CUCCCGCUGCAG | 2667 | 1202 | 1224 | 1518485 | CCGCGCAGCCU | 2858 |
| | | GCUGCGCGGAG | | | | | GCAGCGGGAG | |
| 1518482 | 1518495 | CCCGCUGCAGGC | 2668 | 1200 | 1222 | 1518488 | CUCCGCGCAGCC | 2859 |
| | | UGCGCGGAGGC | | | | | UGCAGCGGG | |
| 1518483 | 1518496 | UCCCGCUGCAGG | 2669 | 1201 | 1223 | 1518487 | UCCGCGCAGCC | 2860 |
| | | CUGCGCGGAGG | | | | | UGCAGCGGGA | |
| 1518484 | 1518494 | GCUGCAGGCUGC | 2670 | 1197 | 1219 | 1518490 | UGCCUCCGCGC | 2861 |
| | | GCGGAGGCAGG | | | | | AGCCUGCAGC | |
| 1518497 | 1518509 | GUCUCCCGCUGC | 2671 | 1204 | 1226 | 1518501 | GCGCAGCCUGC | 2862 |
| | | AGGCUGCGCGG | | | | | AGCGGGAGAC | |
| 1518498 | 1518514 | GGUCUCCCGCUG | 2672 | 1205 | 1227 | 1518506 | CGCAGCCUGCA | 2863 |
| | | CAGGCUGCGCG | | | | | GCGGGAGACC | |
| 1518499 | 1518511 | ACAGGGUCUCCC | 2673 | 1209 | 1231 | 1518504 | GCCUGCAGCGG | 2864 |
| | | GCUGCAGGCUG | | | | | GAGACCCUGU | |
| 1518500 | 1518512 | AGGGUCUCCCGC | 2674 | 1207 | 1229 | 1518508 | CAGCCUGCAGC | 2865 |
| | | UGCAGGCUGCG | | | | | GGGAGACCCU | |
| 1518502 | 1518513 | GGGUCUCCCGCU | 2675 | 1206 | 1228 | 1518505 | GCAGCCUGCAG | 2866 |
| | | GCAGGCUGCGC | | | | | CGGGAGACCC | |
| 1518503 | 1518510 | CAGGGUCUCCCG | 2676 | 1208 | 1230 | 1518507 | AGCCUGCAGCG | 2867 |
| | | CUGCAGGCUGC | | | | | GGAGACCCUG | |
| 1518515 | 1518530 | GACAGGGUCUCC | 2677 | 1210 | 1232 | 1518525 | CCUGCAGCGGG | 2868 |
| | | CGCUGCAGGCU | | | | | AGACCCUGUC | |
| 1518516 | 1518531 | GCGGGGACAGGG | 2678 | 1215 | 1237 | 1518521 | AGCGGGAGACC | 2869 |
| | | UCUCCCGCUGC | | | | | CUGUCCCCGC | |
| 1518517 | 1518529 | CGGGGACAGGGU | 2679 | 1214 | 1236 | 1518522 | CAGCGGGAGAC | 2870 |
| | | CUCCCGCUGCA | | | | | CCUGUCCCCG | |
| 1518518 | 1518528 | GGGGACAGGGUC | 2680 | 1213 | 1235 | 1518524 | GCAGCGGGAGA | 2871 |
| | | UCCCGCUGCAG | | | | | CCCUGUCCCC | |
| 1518519 | 1518532 | GGACAGGGUCUC | 2681 | 1211 | 1233 | 1518526 | CUGCAGCGGGA | 2872 |
| | | CCGCUGCAGGC | | | | | GACCCUGUCC | |
| 1518520 | 1518527 | GGGACAGGGUCU | 2682 | 1212 | 1234 | 1518523 | UGCAGCGGGAG | 2873 |
| | | CCCGCUGCAGG | | | | | ACCCUGUCCC | |
| 1518533 | 1518550 | GGCGGGGACAGG | 2683 | 1216 | 1238 | 1518540 | GCGGGAGACCC | 2874 |
| | | GUCUCCCGCUG | | | | | UGUCCCCGCC | |
| 1518534 | 1518545 | GCUGGGGCGGGG | 2684 | 1221 | 1243 | 1518539 | AGACCCUGUCC | 2875 |
| | | ACAGGGUCUCC | | | | | CCGCCCCAGC | |
| 1518535 | 1518549 | CUGGGGCGGGGA | 2685 | 1220 | 1242 | 1518541 | GAGACCCUGUC | 2876 |
| | | CAGGGUCUCCC | | | | | CCCGCCCCAG | |
| 1518536 | 1518546 | UGGGGCGGGGAC | 2686 | 1219 | 1241 | 1518542 | GGAGACCCUGU | 2877 |
| | | AGGGUCUCCCG | | | | | CCCCGCCCCA | |
| 1518537 | 1518548 | GGGCGGGGACAG | 2687 | 1217 | 1239 | 1518544 | CGGGAGACCCU | 2878 |
| | | GGUCUCCCGCU | | | | | GUCCCCGCCC | |
| 1518538 | 1518547 | GGGGCGGGGACA | 2688 | 1218 | 1240 | 1518543 | GGGAGACCCUG | 2879 |
| | | GGGUCUCCCGC | | | | | UCCCCGCCCC | |
| 1518551 | 1518563 | GGACGGCUGGGG | 2689 | 1226 | 1248 | 1518558 | CUGUCCCCGCCC | 2880 |
| | | CGGGGACAGGG | | | | | CAGCCGUCC | |
| 1518552 | 1518564 | GGCUGGGGCGGG | 2690 | 1222 | 1244 | 1518557 | GACCCUGUCCCC | 2881 |
| | | GACAGGGUCUC | | | | | GCCCCAGCC | |
| 1518553 | 1518565 | GACGGCUGGGGC | 2691 | 1225 | 1247 | 1518559 | CCUGUCCCCGCC | 2882 |
| | | GGGGACAGGGU | | | | | CCAGCCGUC | |
| 1518554 | 1518567 | ACGGCUGGGGCG | 2692 | 1224 | 1246 | 1518561 | CCCUGUCCCCGC | 2883 |
| | | GGGACAGGGUC | | | | | CCCAGCCGU | |
| 1518555 | 1518566 | CGGCUGGGGCGG | 2693 | 1223 | 1245 | 1518560 | ACCCUGUCCCCG | 2884 |
| | | GGACAGGGUCU | | | | | CCCCAGCCG | |
| 1518556 | 1518568 | AGGACGGCUGGG | 2694 | 1227 | 1249 | 1518562 | UGUCCCCGCCCC | 2885 |
| | | GCGGGGACAGG | | | | | AGCCGUCCU | |
| 1518569 | 1518582 | GAGGACGGCUGG | 2695 | 1228 | 1250 | 1518579 | GUCCCCGCCCCA | 2886 |
| | | GGCGGGGACAG | | | | | GCCGUCCUC | |
| 1518570 | 1518586 | CCAGGAGGACGG | 2696 | 1232 | 1254 | 1518578 | CCGCCCCAGCCG | 2887 |
| | | CUGGGGCGGGG | | | | | UCCUCCUGG | |
| 1518571 | 1518585 | CCCAGGAGGACG | 2697 | 1233 | 1255 | 1518576 | CGCCCCAGCCGU | 2888 |
| | | GCUGGGGCGGG | | | | | CCUCCUGGG | |
| 1518572 | 1518581 | CAGGAGGACGGC | 2698 | 1231 | 1253 | 1518575 | CCCGCCCCAGCC | 2889 |
| | | UGGGGCGGGGA | | | | | GUCCUCCUG | |
| 1518573 | 1518583 | AGGAGGACGGCU | 2699 | 1230 | 1252 | 1518577 | CCCCGCCCCAGC | 2890 |
| | | GGGGCGGGGAC | | | | | CGUCCUCCU | |
| 1518574 | 1518584 | GGAGGACGGCUG | 2700 | 1229 | 1251 | 1518580 | UCCCCGCCCCAG | 2891 |
| | | GGGCGGGGACA | | | | | CCGUCCUCC | |
| 1518587 | 1518600 | CCCCAGGAGGAC | 2701 | 1234 | 1256 | 1518596 | GCCCCAGCCGUC | 2892 |
| | | GGCUGGGGCGG | | | | | CUCCUGGGG | |
| 1518588 | 1518599 | GUCCACCCCAGG | 2702 | 1239 | 1261 | 1518597 | AGCCGUCCUCC | 2893 |
| | | AGGACGGCUGG | | | | | UGGGGUGGAC | |
| 1518589 | 1518602 | UCCACCCCAGGA | 2703 | 1238 | 1260 | 1518593 | CAGCCGUCCUCC | 2894 |
| | | GGACGGCUGGG | | | | | UGGGGUGGA | |
| 1518590 | 1518603 | CCACCCCAGGAG | 2704 | 1237 | 1259 | 1518595 | CCAGCCGUCCUC | 2895 |
| | | GACGGCUGGGG | | | | | CUGGGGUGG | |
| 1518591 | 1518604 | CACCCCAGGAGG | 2705 | 1236 | 1258 | 1518598 | CCCAGCCGUCCU | 2896 |
| | | ACGGCUGGGGC | | | | | CCUGGGGUG | |
| 1518592 | 1518601 | ACCCCAGGAGGA | 2706 | 1235 | 1257 | 1518594 | CCCCAGCCGUCC | 2897 |
| | | CGGCUGGGGCG | | | | | UCCUGGGGU | |
| 1518605 | 1518617 | GGUCCACCCCAG | 2707 | 1240 | 1262 | 1518612 | GCCGUCCUCCU | 2898 |
| | | GAGGACGGCUG | | | | | GGGGUGGACC | |
| 1518606 | 1518618 | UAGGGUCCACCC | 2708 | 1243 | 1265 | 1518611 | GUCCUCCUGGG | 2899 |
| | | CAGGAGGACGG | | | | | GUGGACCCUA | |
| 1518607 | 1518620 | GGGUCCACCCCA | 2709 | 1241 | 1263 | 1518615 | CCGUCCUCCUG | 2900 |
| | | GGAGGACGGCU | | | | | GGGUGGACCC | |
| 1518608 | 1518621 | AGGGUCCACCCC | 2710 | 1242 | 1264 | 1518614 | CGUCCUCCUGG | 2901 |
| | | AGGAGGACGGC | | | | | GGUGGACCCU | |
| 1518609 | 1518619 | CUAGGGUCCACC | 2711 | 1244 | 1266 | 1518613 | UCCUCCUGGGG | 2902 |
| | | CCAGGAGGACG | | | | | UGGACCCUAG | |
| 1518610 | 1518622 | ACUAGGGUCCAC | 2712 | 1245 | 1267 | 1518616 | CCUCCUGGGGU | 2903 |
| | | CCCAGGAGGAC | | | | | GGACCCUAGU | |
| 1518623 | 1518637 | AACUAGGGUCCA | 2713 | 1246 | 1268 | 1518629 | CUCCUGGGGUG | 2904 |
| | | CCCCAGGAGGA | | | | | GACCCUAGUU | |
| 1518624 | 1518640 | AUUAAACUAGGG | 2714 | 1250 | 1272 | 1518632 | UGGGGUGGACC | 2905 |
| | | UCCACCCCAGG | | | | | CUAGUUUAAU | |
| 1518625 | 1518639 | UUAAACUAGGGU | 2715 | 1249 | 1271 | 1518634 | CUGGGGUGGAC | 2906 |
| | | CCACCCCAGGA | | | | | CCUAGUUUAA | |
| 1518626 | 1518635 | UAUUAAACUAGG | 2716 | 1251 | 1273 | 1518633 | GGGGUGGACCC | 2907 |
| | | GUCCACCCCAG | | | | | UAGUUUAAUA | |
| 1518627 | 1518636 | AAACUAGGGUCC | 2717 | 1247 | 1269 | 1518630 | UCCUGGGGUGG | 2908 |
| | | ACCCCAGGAGG | | | | | ACCCUAGUUU | |
| 1518628 | 1518638 | UAAACUAGGGUC | 2718 | 1248 | 1270 | 1518631 | CCUGGGGUGGA | 2909 |
| | | CACCCCAGGAG | | | | | CCCUAGUUUA | |
| 1518641 | 1518656 | CUUUAUUAAACU | 2719 | 1254 | 1276 | 1518651 | GUGGACCCUAG | 2910 |
| | | AGGGUCCACCC | | | | | UUUAAUAAAG | |
| 1518642 | 1518654 | AAUCUUUAUUAA | 2720 | 1257 | 1279 | 1518649 | GACCCUAGUUU | 2911 |
| | | ACUAGGGUCCA | | | | | AAUAAAGAUU | |
| 1518643 | 1518655 | AUCUUUAUUAAA | 2721 | 1256 | 1278 | 1518647 | GGACCCUAGUU | 2912 |
| | | CUAGGGUCCAC | | | | | UAAUAAAGAU | |
| 1518644 | 1518653 | UCUUUAUUAAAC | 2722 | 1255 | 1277 | 1518648 | UGGACCCUAGU | 2913 |
| | | UAGGGUCCACC | | | | | UUAAUAAAGA | |
| 1518645 | 1518658 | UUUAUUAAACUA | 2723 | 1253 | 1275 | 1518650 | GGUGGACCCUA | 2914 |
| | | GGGUCCACCCC | | | | | GUUUAAUAAA | |
| 1518646 | 1518657 | UUAUUAAACUAG | 2724 | 1252 | 1274 | 1518652 | GGGUGGACCCU | 2915 |
| | | GGUCCACCCCA | | | | | AGUUUAAUAA | |
| 1518659 | 1518671 | GAAUCUUUAUUA | 2725 | 1258 | 1280 | 1518665 | ACCCUAGUUUA | 2916 |
| | | AACUAGGGUCC | | | | | AUAAAGAUUC | |
| 1518660 | 1518672 | UGGUGAAUCUUU | 2726 | 1262 | 1284 | 1518666 | UAGUUUAAUAA | 2917 |
| | | AUUAAACUAGG | | | | | AGAUUCACCA | |
| 1518661 | 1518673 | GGUGAAUCUUUA | 2727 | 1261 | 1283 | 1518667 | CUAGUUUAAUA | 2918 |
| | | UUAAACUAGGG | | | | | AAGAUUCACC | |
| 1518662 | 1518674 | GUGAAUCUUUAU | 2728 | 1260 | 1282 | 1518668 | CCUAGUUUAAU | 2919 |
| | | UAAACUAGGGU | | | | | AAAGAUUCAC | |
| 1518663 | 1518676 | UGAAUCUUUAUU | 2729 | 1259 | 1281 | 1518669 | CCCUAGUUUAA | 2920 |
| | | AAACUAGGGUC | | | | | UAAAGAUUCA | |
| 1518664 | 1518675 | UUGGUGAAUCUU | 2730 | 1263 | 1285 | 1518670 | AGUUUAAUAAA | 2921 |
| | | UAUUAAACUAG | | | | | GAUUCACCAA | |
| 1518677 | 1518692 | CUUGGUGAAUCU | 2731 | 1264 | 1286 | 1518686 | GUUUAAUAAAG | 2922 |
| | | UUAUUAAACUA | | | | | AUUCACCAAG | |
| 1518678 | 1518689 | UGAAACUUGGUG | 2732 | 1269 | 1291 | 1518684 | AUAAAGAUUCA | 2923 |
| | | AAUCUUUAUUA | | | | | CCAAGUUUCA | |
| 1518679 | 1518690 | GAAACUUGGUGA | 2733 | 1268 | 1290 | 1518688 | AAUAAAGAUUC | 2924 |
| | | AUCUUUAUUAA | | | | | ACCAAGUUUC | |
| 1518680 | 1518693 | AAACUUGGUGAA | 2734 | 1267 | 1289 | 1518683 | UAAUAAAGAUU | 2925 |
| | | UCUUUAUUAAA | | | | | CACCAAGUUU | |
| 1518681 | 1518691 | AACUUGGUGAAU | 2735 | 1266 | 1288 | 1518685 | UUAAUAAAGAU | 2926 |
| | | CUUUAUUAAAC | | | | | UCACCAAGUU | |
| 1518682 | 1518694 | ACUUGGUGAAUC | 2736 | 1265 | 1287 | 1518687 | UUUAAUAAAGA | 2927 |
| | | UUUAUUAAACU | | | | | UUCACCAAGU | |
| 1518695 | 1518705 | CGUGAAACUUGG | 2737 | 1271 | 1293 | 1518700 | AAAGAUUCACC | 2928 |
| | | UGAAUCUUUAU | | | | | AAGUUUCACG | |
| 1518697 | 1518706 | GCGUGAAACUUG | 2738 | 1272 | 1294 | 1518701 | AAGAUUCACCA | 2929 |
| | | GUGAAUCUUUA | | | | | AGUUUCACGC | |
| 1518698 | 1518707 | UGCGUGAAACUU | 2739 | 1273 | 1295 | 1518702 | AGAUUCACCAA | 2930 |
| | | GGUGAAUCUUU | | | | | GUUUCACGCA | |
| 1518699 | 1518709 | GUGAAACUUGGU | 2740 | 1270 | 1292 | 1518703 | UAAAGAUUCAC | 2931 |
| | | GAAUCUUUAUU | | | | | CAAGUUUCAC | |

TABLE 52
RNAi compounds targeting human APOE SEQ ID NO: 3

| | | | | SEQ ID | SEQ ID | Sense | | |
|---|---|---|---|---|---|---|---|---|
| | Antisense | Antisense | SEQ | NO: 3 | NO: 3 | RNAi | Sense | SEQ |
| Compound | RNAi | Sequence | ID | Antisense | Antisense | Oligo | Sequence | ID |
| Number | Oligo ID | (5′ to 3′) | NO | Start Site | Stop Site | ID | (5′ to 3′) | NO |
| 1518138 | 1518149 | GCCUGUGAUUG | 2741 | 40 | 62 | 1518143 | GCCGACUGGCC | 2932 |
| | | GCCAGUCGGCU | | | | | AAUCACAGGC | |
| | | C | | | | | | |
| 1518696 | 1518708* | GGCGGCCGCGC | 2742 | 438 | 460 | 1518704 | UGGAGGACGU | 2933 |
| | | ACGUCCUCCAU | | | | | GCGCGGCCGCC | |
| | | G | | | | | | |

*Compound No. 1518708 has a single mismatch to SEQ ID NO: 3 located at position 10 (from 5′ to 3′) of the antisense strand.

### Example 10: Effect of RNAi Compounds on Human APOE RNA In Vitro, Single Dose

Double-stranded RNAi compounds described above were tested in a series of experiments under the same culture conditions. The results for each experiment are presented in separate tables below.

Cultured Hep3B cells at a density of 20,000 cells per well were transfected using RNAiMAX with 20 nM of double stranded RNAi. After a treatment period of approximately 24 hours, RNA was isolated from the cells and APOE RNA levels were measured by quantitative real-time RTPCR. Human primer probe set RTS3073 (described herein in Example 1 above) was used to measure RNA levels. APOE RNA levels were normalized to total RNA content, as measured by RIBOGREEN®. Results are presented as percent APOE RNA relative to the amount in untreated control cells (% UTC). The values marked with an “†” indicate that the modified oligonucleotide is complementary to the amplicon region of the primer probe set. Additional assays may be used to measure the potency and efficacy of the modified oligonucleotides complementary to the amplicon region. N.D. in the tables below refers to instances where the value was Not Defined.

TABLE 53
Reduction of APOE RNA by RNAi

| Compound Number | APOE (% UTC) |
|---|---|
| 1518137 | 107 |
| 1518138 | 87 |
| 1518139 | 7 |
| 1518140 | 7 |
| 1518141 | 81 |
| 1518142 | 132 |
| 1518155 | 43 |
| 1518156 | 76† |
| 1518157 | 52† |
| 1518158 | 104 |
| 1518159 | 25 |
| 1518160 | 89 |
| 1518173 | 59† |
| 1518174 | 12 |
| 1518175 | 42† |
| 1518176 | 88† |
| 1518177 | 81† |
| 1518179 | 97 |
| 1518191 | 90 |
| 1518192 | 81 |
| 1518193 | 111 |
| 1518194 | 103 |
| 1518195 | 35 |
| 1518196 | 84 |
| 1518209 | 76 |
| 1518210 | 23 |
| 1518211 | 96 |
| 1518212 | 43 |
| 1518213 | 83 |
| 1518214 | 91 |
| 1518227 | 88 |
| 1518228 | 77 |
| 1518229 | 89 |
| 1518230 | 71 |
| 1518231 | 104 |
| 1518232 | 75 |
| 1518245 | 74 |
| 1518246 | 66 |
| 1518247 | 88 |
| 1518248 | 84 |
| 1518249 | 84 |
| 1518250 | 86 |
| 1518263 | 75 |
| 1518264 | 62 |
| 1518265 | 92 |
| 1518266 | 7 |
| 1518267 | 80 |
| 1518268 | 74 |
| 1518281 | 57 |
| 1518282 | 5 |
| 1518283 | 21 |
| 1518284 | 57 |
| 1518285 | 72 |
| 1518286 | 23 |
| 1518299 | 23 |
| 1518300 | 55 |
| 1518301 | 38 |
| 1518302 | 41 |
| 1518303 | 39 |
| 1518304 | 10 |
| 1518317 | 43 |
| 1518318 | 72 |
| 1518319 | 14 |
| 1518320 | 71 |
| 1518321 | 41 |
| 1518322 | 60 |
| 1518335 | 5 |
| 1518336 | 38 |
| 1518337 | 17 |
| 1518338 | 42 |
| 1518339 | 9 |
| 1518340 | 16 |
| 1518353 | 76 |
| 1518356 | 61 |
| 1518357 | 11 |
| 1518358 | 64 |
| 1518359 | 53 |
| 1518360 | 40 |
| 1518372 | 68 |
| 1518376 | 68 |

TABLE 54
Reduction of APOE RNA by RNAi

| Compound Number | APOE (% UTC) |
|---|---|
| 1518425 | 106 |
| 1518426 | 111 |
| 1518427 | 107 |
| 1518428 | 118 |
| 1518429 | 109 |
| 1518430 | 110 |
| 1518443 | 107 |
| 1518444 | 112 |
| 1518445 | 103 |
| 1518446 | 100 |
| 1518447 | 113 |
| 1518448 | 87 |
| 1518461 | 88 |
| 1518462 | 91 |
| 1518463 | 97 |
| 1518464 | 101 |
| 1518465 | 83 |
| 1518466 | 101 |
| 1518479 | 87 |
| 1518480 | N.D. |
| 1518481 | N.D. |
| 1518482 | 68 |
| 1518483 | 44 |
| 1518484 | 82 |
| 1518497 | 67 |
| 1518498 | N.D. |
| 1518499 | 61 |
| 1518500 | 79 |
| 1518502 | 82 |
| 1518503 | 15 |
| 1518515 | 55 |
| 1518516 | 53 |
| 1518517 | N.D. |
| 1518518 | 67 |
| 1518519 | 11 |
| 1518520 | 68 |
| 1518533 | 86 |
| 1518534 | 69 |
| 1518535 | 79 |
| 1518536 | 89 |
| 1518537 | 72 |
| 1518538 | 94 |
| 1518551 | 84 |
| 1518552 | N.D. |
| 1518553 | N.D. |
| 1518554 | 76 |
| 1518555 | 74 |
| 1518556 | 61 |
| 1518569 | 60 |
| 1518570 | 65 |
| 1518571 | 67 |
| 1518572 | 82 |
| 1518573 | N.D. |
| 1518574 | 73 |
| 1518587 | 66 |
| 1518588 | N.D. |
| 1518589 | 82 |
| 1518590 | 66 |
| 1518591 | 74 |
| 1518592 | 71 |
| 1518605 | 60 |
| 1518606 | N.D. |
| 1518607 | 70 |
| 1518608 | 67 |
| 1518609 | 57 |
| 1518610 | 71 |
| 1518623 | 65 |
| 1518624 | 50 |
| 1518625 | 67 |
| 1518626 | 6 |
| 1518627 | 21 |
| 1518628 | 17 |
| 1518641 | 61 |
| 1518642 | 2 |
| 1518643 | 2 |
| 1518644 | 2 |
| 1518645 | 46 |
| 1518646 | 24 |
| 1518659 | 2 |
| 1518663 | 2 |

TABLE 55
Reduction of APOE RNA by RNAi

| Compound Number | APOE (% UTC) |
|---|---|
| 1518371 | 85 |
| 1518373 | 69 |
| 1518374 | 91 |
| 1518375 | 96 |
| 1518389 | 102 |
| 1518390 | 93 |
| 1518391 | 100 |
| 1518392 | 104 |
| 1518393 | 98 |
| 1518394 | 96 |
| 1518407 | 89 |
| 1518408 | 96 |
| 1518409 | 92 |
| 1518410 | 100 |
| 1518411 | 94 |
| 1518412 | 82 |
| 1518660 | 6 |
| 1518661 | 6 |
| 1518662 | N.D. |
| 1518664 | 8 |
| 1518677 | 6 |
| 1518678 | 5 |
| 1518679 | 6 |
| 1518680 | 7 |
| 1518681 | 3 |
| 1518682 | 4 |
| 1518695 | 12 |
| 1518696 | 98 |
| 1518697 | 7 |
| 1518698 | 61 |
| 1518699 | 7 |

### Example 11: Design of Modified Oligonucleotides Complementary to Human APOE Nucleic Acid

Modified oligonucleotides complementary to a human APOE nucleic acid were designed, as described in the tables below. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

The modified oligonucleotides in Table 56 are 5-10-5 MOE gapmers. The gapmers are 20 nucleosides in length, wherein the central gap segment consists of ten 2′-β-D-deoxynucleosides, and the 5′ and 3′ wing segments each consists of five 2′-MOE modified nucleosides. The sugar motif for the gapmers is (from 5′ to 3′): eeeeeddddddddddeeeee; wherein each ‘d’ represents a 2′-β-D-deoxyribosyl sugar moiety, and each ‘e’ represents a 2′-MOE modified sugar moiety. The gapmers have an internucleoside linkage motif of (from 5′ to 3′): sooosssssssssssooss; wherein each “s” represents a phosphorothioate internucleoside linkage, and each “o” represents a phosphodiester internucleoside linkage. Each cytosine residue is a 5-methyl cytosine.

TABLE 56
5-10-5 MOE gapmers with mixed PO/PS
internucleoside linkages complementary to human APOE

| | | SEQ | SEQ | SEQ | SEQ | |
|---|---|---|---|---|---|---|
| | | ID No: | ID No: | ID No: | ID No: | |
| Compound | | 1 Start | 1 Stop | 2 Start | 2 Stop | SEQ |
| Number | Sequence (5′ to 3′) | Site | Site | Site | Site | ID NO |
| 1401842 | CCTCATTTTAAAGTTCTCCA | 3201 | 3220 | N/A | N/A | 2522 |
| 1401843 | CCTCATTCTCCCTTCACATT | 3275 | 3294 | N/A | N/A | 2523 |
| 1401845 | GGGACACCCAGTAGGTGCTC | 3118 | 3137 | N/A | N/A | 2524 |
| 1401846 | TTCCTCATTCTCCCTTCACA | 3277 | 3296 | N/A | N/A | 2525 |
| 1401848 | GAGCTAATTCAGTCCTCATT | 3214 | 3233 | N/A | N/A | 2526 |
| 1401852 | TCCTCATTCTCCCTTCACAT | 3276 | 3295 | N/A | N/A | 2527 |
| 1401853 | CTCCAATCGACGGCTAGCTA | 3186 | 3205 | N/A | N/A | 2528 |
| 1401857 | TGAGCTAATTCAGTCCTCAT | 3215 | 3234 | N/A | N/A | 2529 |
| 1401861 | CATTCCTCATTCTCCCTTCA | 3279 | 3298 | N/A | N/A | 2530 |
| 1401862 | GCATTCCTCATTCTCCCTTC | 3280 | 3299 | N/A | N/A | 2531 |

The modified oligonucleotides in Table 57 are 5-10-5 MOE gapmers. The gapmers are 20 nucleosides in length, wherein the central gap segment consists of ten 2′-β-D-deoxynucleosides, and the 5′ and 3′ wing segments each consists of five 2′-MOE modified nucleosides. The sugar motif for the gapmers is (from 5′ to 3′): eeeeeddddddddddeeeee; wherein each ‘d’ represents a 2′-β-D-deoxyribosyl sugar moiety, and each ‘e’ represents a 2′-MOE modified sugar moiety. The gapmers have an internucleoside linkage motif of (from 5′ to 3′): soooossssssssssooss; wherein each “s” represents a phosphorothioate internucleoside linkage, and each “o” represents a phosphodiester internucleoside linkage. Each cytosine residue is a 5-methyl cytosine.

TABLE 57
5-10-5 MOE gapmers with mixed PO/PS
internucleoside linkages complementary to human APOE

| | | SEQ | SEQ | SEQ | SEQ | |
|---|---|---|---|---|---|---|
| | | ID No: | ID No: | ID No: | ID No: | |
| Compound | | 1 Start | 1 Stop | 2 Start | 2 Stop | SEQ |
| Number | Sequence (5′ to 3′) | Site | Site | Site | Site | ID NO |
| 1419518 | ATGCATTAGAAACCTCTAAC | 4187 | 4206 | N/A | N/A | 1697 |
| 1419519 | GTTGCATTATCTGCAACAGC | 3482 | 3501 | N/A | N/A | 1632 |
| 1419520 | CCTGCTATGGCTTACATCCC | 3070 | 3089 | N/A | N/A | 1669 |
| 1419521 | CTATGGCTTACATCCCAGTC | 3066 | 3085 | N/A | N/A | 1801 |
| 1419522 | TCTGGTATTCACTATCTGCC | 4211 | 4230 | N/A | N/A | 1899 |
| 1419523 | CCCTTCACATTCTAAGCTCC | 3266 | 3285 | N/A | N/A | 1188 |
| 1419524 | CTCATTTTAAAGTTCTCCAA | 3200 | 3219 | N/A | N/A | 1539 |
| 1419525 | CTCGATAAATGATAGTGACA | 3101 | 3120 | N/A | N/A | 1419 |
| 1419526 | GCATTATCTGCAACAGCCTA | 3479 | 3498 | N/A | N/A | 1830 |
| 1419527 | TCATTTTAAAGTTCTCCAAT | 3199 | 3218 | N/A | N/A | 1638 |
| 1419528 | TATGAGCTAATTCAGTCCTC | 3217 | 3236 | N/A | N/A | 1350 |
| 1419529 | CACATTCTAAGCTCCAACCT | 3261 | 3280 | N/A | N/A | 1521 |
| 1419530 | CCTCCATAGAAAATTCCATC | 3372 | 3391 | N/A | N/A | 1662 |
| 1419531 | TGCTCCACAAATGCTTCTTT | 4661 | 4680 | N/A | N/A | 1656 |
| 1419532 | CCTCATTTTAAAGTTCTCCA | 3201 | 3220 | N/A | N/A | 2522 |
| 1419533 | CAGCACATTTACCAAGCCGC | 3455 | 3474 | N/A | N/A | 855 |
| 1419534 | TCCTCATTTTAAAGTTCTCC | 3202 | 3221 | N/A | N/A | 1457 |
| 1419535 | TTCCATTTATGAGCTAATTC | 3224 | 3243 | N/A | N/A | 1750 |

The modified oligonucleotides in Table 58 are 5-10-5 MOE gapmers. The gapmers are 20 nucleosides in length, wherein the central gap segment consists of ten 2′-β-D-deoxynucleosides, and the 5′ and 3′ wing segments each consists of five 2′-MOE modified nucleosides. The sugar motif for the gapmers is (from 5′ to 3′): eeeeeddddddddddeeeee; wherein each represents a 2′-β-D-deoxyribosyl sugar moiety, and each ‘e’ represents a 2′-MOE modified sugar moiety. The gapmers have an internucleoside linkage motif of (from 5′ to 3′): sossssssssssssssoss; wherein each “s” represents a phosphorothioate internucleoside linkage, and each “o” represents a phosphodiester internucleoside linkage. Each cytosine residue is a 5-methyl cytosine.

TABLE 58
5-10-5 MOE gapmers with mixed PO/PS
internucleoside linkages complementary to human APOE

| | | SEQ | SEQ | SEQ | SEQ | |
|---|---|---|---|---|---|---|
| | | ID No: | ID No: | ID No: | ID No: | |
| Compound | | 1 Start | 1 Stop | 2 Start | 2 Stop | SEQ |
| Number | Sequence (5′ to 3′) | Site | Site | Site | Site | ID NO |
| 1449189 | CAGCACATTTACCAAGCCGC | 3455 | 3474 | N/A | N/A | 855 |
| 1449190 | CCTCATTTTAAAGTTCTCCA | 3201 | 3220 | N/A | N/A | 2522 |
| 1449191 | TATGAGCTAATTCAGTCCTC | 3217 | 3236 | N/A | N/A | 1350 |
| 1449192 | TTCCATTTATGAGCTAATTC | 3224 | 3243 | N/A | N/A | 1750 |
| 1449193 | TGGTGAATCTTTATTAAACT | 6363 | 6382 | 1265 | 1284 | 1193 |

The modified oligonucleotides in Table 59 are 3-10-3 cEt gapmers. The gapmers are 16 nucleosides in length, wherein the central gap segment consists of ten 2′-β-D-deoxynucleosides, and wherein the 5′ and 3′ wing segments each consist of three cEt modified nucleosides. The sugar motif for the gapmers is (from 5′ to 3′): kkkddddddddddkkk; wherein each represents a 2′-β-D-deoxyribosyl sugar moiety, and each 1′ represents a cEt sugar moiety. The gapmers have an internucleoside linkage motif of (from 5′ to 3′): soossssssssssos; wherein each “s” represents a phosphorothioate internucleoside linkage and each “o” represents a phosphodiester internucleoside linkage. Each cytosine residue is a 5-methyl cytosine.

TABLE 59
3-10-3 cEt gapmers with
mixed PO/PS internucleoside
linkages complementary to human APOE

| | | SEQ | SEQ | SEQ | SEQ | |
|---|---|---|---|---|---|---|
| | | ID | ID | ID | ID | |
| | | No: 1 | No: 1 | No: 2 | No: 2 | SEQ |
| Compound | Sequence | Start | Stop | Start | Stop | ID |
| Number | (5′ to 3′) | Site | Site | Site | Site | NO |
| 1335664 | GAAGCTAGAA | 5060 | 5075 | N/A | N/A | 2532 |
| | CCAGCA | | | | | |
| 1335669 | GTTTAATCAC | 4612 | 4627 | N/A | N/A | 2533 |
| | TTGGAA | | | | | |
| 1335682 | CCAATTATAG | 2758 | 2773 | 101 | 25 | 2534 |
| | GGCTCC | | | | | |

The modified oligonucleotides in Table 60 are 17 nucleosides in length. The sugar motif for each of the gapmers is (from 5′ to 3′): eeeeddddddddkkeee; wherein each ‘e’ represents a 2′-MOE modified sugar moiety, each represents a 2′-β-D-deoxyribosyl sugar moiety, and each 1′ represents a cEt sugar moiety. The gapmers each have an internucleoside linkage motif of (from 5′ to 3′): soosssssssssooss; wherein each “s” represents a phosphorothioate internucleoside linkage and each “o” represents a phosphodiester internucleoside linkage. Each cytosine residue is a 5-methyl cytosine.

TABLE 60
Modified oligonucleotides with mixed PO/PS
internucleoside linkages complementary to human APOE

| | | SEQ ID | SEQ ID | SEQ ID | SEQ ID | |
|---|---|---|---|---|---|---|
| | | No: 1 | No: 1 | No: 2 | No: 2 | |
| Compound | Sequence | Start | Stop | Start | Stop | SEQ |
| Number | (5′ to 3′) | Site | Site | Site | Site | ID NO |
| 1449171 | GTGAATCTTTATTAAAC | 6364 | 6380 | 1266 | 1282 | 2535 |
| 1449172 | CCTCATTTTAAAGTTCT | 3204 | 3220 | N/A | N/A | 2536 |
| 1449173 | CACATTTACCAAGCCGC | 3455 | 3471 | N/A | N/A | 2537 |
| 1449174 | GCACATTTACCAAGCCG | 3456 | 3472 | N/A | N/A | 2538 |
| 1449176 | CAGCACATTTACCAAGC | 3458 | 3474 | N/A | N/A | 2539 |
| 1449177 | CATTTATGAGCTAATTC | 3224 | 3240 | N/A | N/A | 2540 |
| 1449178 | CCATTTATGAGCTAATT | 3225 | 3241 | N/A | N/A | 2541 |
| 1449179 | TCCATTTATGAGCTAAT | 3226 | 3242 | N/A | N/A | 2542 |
| 1449180 | TTCCATTTATGAGCTAA | 3227 | 3243 | N/A | N/A | 2543 |
| 1449181 | GGTGAATCTTTATTAAA | 6365 | 6381 | 1267 | 1283 | 2544 |
| 1449182 | GAGCTAATTCAGTCCTC | 3217 | 3233 | N/A | N/A | 2545 |
| 1449183 | TGAGCTAATTCAGTCCT | 3218 | 3234 | N/A | N/A | 2546 |
| 1449184 | ATGAGCTAATTCAGTCC | 3219 | 3235 | N/A | N/A | 2547 |
| 1449185 | TATGAGCTAATTCAGTC | 3220 | 3236 | N/A | N/A | 2548 |
| 1449186 | CATTTTAAAGTTCTCCA | 3201 | 3217 | N/A | N/A | 2549 |
| 1449187 | TCATTTTAAAGTTCTCC | 3202 | 3218 | N/A | N/A | 2550 |
| 1449188 | CTCATTTTAAAGTTCTC | 3203 | 3219 | N/A | N/A | 2551 |

### Example 12: Activity of Modified Oligonucleotides Complementary to Human APOE in Transgenic Mice

ApoE4 transgenic mice (model #1549) were obtained from Taconic Biosciences. APOE transgenic mice were divided into groups of 4 mice each. Each mouse received a single intracerebroventricular (ICV) bolus of 100 μg, or 300 μg of modified oligonucleotide, as indicated in the tables below. A group of 4 mice received a single ICV bolus with PBS as a negative control, and the PCR values were normalized to this group.

Two weeks post treatment, mice were sacrificed and RNA was extracted from cortical brain tissue, and/or spinal cord for quantitative real-time RTPCR analysis of RNA expression of APOE using primer probe set RTS3073 (described herein above). Results are presented as percent change of RNA, relative to PBS control, normalized to mouse cyclophilin A (% control). Mouse cyclophilin A was amplified using primer probe set m_cyclo24 (forward sequence TCGCCGCTTGCTGCA, designated herein as SEQ ID NO: 16; reverse sequence ATCGGCCGTGATGTCGA, designated herein as SEQ ID NO: 17; probe sequence CCATGGTCAACCCCACCGTGTTC, designated herein as SEQ ID NO: 18). Data indicated as “n.d.” (no data) means that no data is available for that tissue for that compound.

As shown in the table below, treatment with modified oligonucleotides resulted in reduction of APOE RNA in comparison to the PBS control.

TABLE 61
Reduction of human APOE RNA in transgenic mice, 100 μg dose

| | APOE RNA |
|---|---|
| Compound No. | (% control) - CORTEX |
| PBS | 100 |
| 1335661 | 82 |
| 1335663 | 97 |
| 1335664 | 88 |
| 1335669 | 79 |
| 1335672 | 73 |
| 1335675 | 75 |
| 1335678 | 90 |
| 1335679 | 83 |
| 1335681 | 81 |
| 1335682 | 86 |
| 1335683 | 79 |

TABLE 62
Reduction of human APOE RNA in transgenic mice, 300 μg dose

| | APOE RNA |
|---|---|
| Compound No. | (% control) - CORTEX |
| PBS | 100 |
| 942665 | 92 |
| 942683 | 74 |
| 1335661 | 82 |
| 1335663 | 65 |
| 1335664 | 77 |
| 1335675 | 71 |
| 1335683 | 86 |
| 1401842 | 89 |
| 1401843 | 90 |
| 1401845 | 91 |
| 1401846 | 96 |
| 1401848 | 80 |
| 1401852 | 82 |
| 1401853 | 80 |
| 1401857 | 91 |
| 1401861 | 88 |
| 1401862 | 71 |

TABLE 63
Reduction of human APOE RNA in transgenic mice, 300 μg dose

| | APOE RNA |
|---|---|
| Compound No. | (% control) - CORTEX |
| PBS | 100 |
| 1419518 | 83 |
| 1419519 | 121 |
| 1419520 | 78 |
| 1419521 | 88 |
| 1419522 | 96 |
| 1419523 | 75 |
| 1419524 | 90 |
| 1419525 | 86 |
| 1419526 | 89 |
| 1419527 | 96 |
| 1419528 | 56 |
| 1419529 | 104 |
| 1419530 | 87 |
| 1419531 | 92 |
| 1419532 | 62 |
| 1419533 | 63 |
| 1419534 | 80 |
| 1419535 | 58 |

TABLE 64
Reduction of human APOE RNA in transgenic mice, 300 μg dose

| | |
|---|---|
| Compound | APOE RNA (% control) |

| No. | CORTEX | SPINAL CORD |
|---|---|---|
| PBS | 100 | 100 |
| 1449171 | 56 | 44 |
| 1449172 | 114 | 89 |
| 1449173 | 69 | 70 |
| 1449174 | 57 | 71 |
| 1449176 | 78 | 80 |
| 1449177 | 69 | 87 |
| 1449178 | 46 | 70 |
| 1449179 | 59 | 70 |
| 1449180 | 56 | 77 |
| 1449181 | 36 | 48 |
| 1449182 | 55 | 64 |
| 1449183 | 49 | 72 |
| 1449184 | 54 | 66 |
| 1449185 | 62 | 82 |
| 1449186 | 62 | 68 |
| 1449187 | 41 | 60 |
| 1449188 | 49 | 58 |
| 1449189 | 62 | 71 |
| 1449190 | 42 | 52 |
| 1449191 | 70 | 64 |
| 1449192 | 67 | 80 |
| 1449193 | 44 | 55 |

### Example 13: Activity of Modified Oligonucleotides Complementary to Human APOE in Knock-In Mice

APOE knock-in mice used in this study express the full-length human APOE gene knocked into the mouse locus. Humanization of APOE gene was done via CRISPR/Cas-9-mediated gene editing, allowing for generation of a model with constitutive expression of human APOE gene. Targeting strategy was based on NCBI transcripts NM_009696.4 (mouse) and NM_000041.4 (human). Mouse genomic sequence from exon 1 to exon 4 (including the 5′ and 3′ UTRs) was replaced with the human counterpart from 141 bp upstream of exon 1 to 28 bp downstream of exon 4. A plasmid allowing expression of Cas9 mRNA, specific gRNA, and the puromycin resistance cassette, and a plasmid containing the homology regions of the mouse APOE gene, and the replaced human region were co-transfected into the Taconic Biosciences C57BL/6N Tac ES cell line. Homologous recombination clones were isolated using positive puromycin selection, and humanized allele was obtained after Cas9-mediated gene editing. C57BL/6NTac-Apoeem7250_A-C03(APOE) Tac mice were used in these experiments, and are herein called APOE knock-in mice.

APOE knock-in mice were divided into groups of 2 mice each. Each mouse received a single ICV bolus of 300 μg of modified oligonucleotide. A group of 4 mice received a single ICV bolus with PBS as a negative control.

Two weeks post treatment, mice were sacrificed and RNA was extracted from cortical brain tissue, and spinal cord for quantitative real-time RTPCR analysis of RNA expression of APOE using primer probe set RTS3073 (described herein above). Results are presented as percent change of RNA, relative to PBS control, normalized to mouse cyclophilin A (% control). Mouse cyclophilin A was amplified using primer probe set m_cyclo24 (designated herein above). The values marked with a “t” indicate that the modified oligonucleotide is complementary to the amplicon region of the primer-probe set. In such cases, human primer-probe set RTS36365 (forward sequence CAGCGGAGGTGAAGGAC, designated herein as SEQ ID NO: 7; reverse sequence CAACGCAGCCCACAGAA, designated herein as SEQ ID NO: 8; probe sequence TCATCTTCCTGCCTGTGATTGGCC, designated herein as SEQ ID NO: 9) was used to confirm RNA expression of APOE.

As shown in the table below, treatment with modified oligonucleotides resulted in reduction of APOE RNA in comparison to the PBS control.

TABLE 65
Reduction of human APOE RNA in knock-in mice

| | APOE RNA (% control) | | APOE RNA (% control) | |
|---|---|---|---|---|
| | RTS3073 | | RTS36365 | |
| Compound | SPINAL | | SPINAL | |
| No. | CORD | CORTEX | CORD | CORTEX |
| PBS | 100 | 100 | 100 | 100 |
| 688711 | 64 | 78 | 64 | 78 |
| 688730 | 96 | 102 | 109 | 111 |
| 688775 | 103† | 92† | 111 | 98 |
| 688817 | 69 | 80 | 83 | 98 |
| 689046 | 45 | 52 | 48 | 55 |
| 729709 | 101 | 82 | 111 | 87 |
| 942364 | 80 | 92 | 90 | 99 |
| 942391 | 98† | 94† | 115 | 99 |
| 942393 | 99† | 96† | 114 | 110 |
| 942593 | 96 | 81 | 97 | 100 |
| 942728 | 78 | 78 | 89 | 91 |
| 1419528 | 83 | 82 | 90† | 93† |
| 1419533 | 79 | 89 | 81† | 97† |
| 1419535 | 77 | 91 | 81† | 93† |
| 1449190 | 67 | 73 | 70† | 81† |
| 1449193 | 61 | 59 | 72 | 71 |
| 1517154 | 80 | 72 | 77† | 82† |
| 1517173 | 89 | 90 | 89 | 95 |
| 1517222 | 90 | 87 | 89† | 85† |
| 1517226 | 80 | 70 | 84† | 69† |
| 1517228 | 94 | 86 | 95 | 90 |
| 1517234 | 106 | 87 | 101 | 93 |
| 1517246 | 78 | 78 | 75† | 79† |
| 1517248 | 89 | 93 | 87 | 91 |
| 1517254 | 52 | 57 | 53 | 56 |
| 1517281 | 71 | 81 | 77 | 84 |
| 1517311 | 76† | 100† | 72 | 104 |
| 1517316 | 90 | 98 | 94 | 107 |
| 1517356 | 94 | 91 | 95† | 96† |
| 1517372 | 115 | 96 | 116† | 101† |
| 1517383 | 81 | 69 | 73 | 72 |
| 1517394 | 129 | 103 | 117† | 111† |
| 1517508 | 77 | 71 | 63 | 73 |
| 1517529 | 226† | 97† | 175 | 108 |
| 1517561 | 85 | 96 | 87† | 102† |
| 1517564 | 108 | 95 | 108 | 103 |
| 1517641 | 107 | 99 | 101 | 102 |
| 1517651 | 100 | 99 | 96† | 105† |
| 1517652 | 90 | 79 | 78 | 87 |
| 1517663 | 103 | 105 | 96† | 141† |
| 1517732 | 87 | 89 | 102† | 100† |
| 1517770 | 33 | 35 | 40 | 39 |
| 1517853 | 65 | 53 | 70 | 65 |
| 1517891 | 65 | 49 | 77 | 66 |

TABLE 66
Reduction of human APOE RNA in knock-in mice

| | |
|---|---|
| Compound | APOE RNA (% control) |

| No. | SPINAL CORD | CORTEX |
|---|---|---|
| PBS | 100 | 100 |
| 689046 | 43.7‡ | 57‡ |
| 942594 | 83 | 107 |
| 942596 | 100 | 101 |
| 942684 | 85 | 85 |
| 1401848 | 79 | 87 |
| 1517718 | 91 | 85 |
| 1517841 | 78 | 73 |
| 1517885 | 63 | 58 |

‡Fewer than 2 samples available

TABLE 67
Reduction of human APOE RNA in knock-in mice

| | |
|---|---|
| Compound | APOE RNA (% control) |

| No. | SPINAL CORD | CORTEX |
|---|---|---|
| PBS | 100 | 100 |
| 1449193 | 65 | 71 |
| 1517770 | 28 | 35 |

### Example 14: Design of Modified Oligonucleotides Complementary to Human APOE Nucleic Acid

Modified oligonucleotides complementary to a human APOE nucleic acid were designed, as described in the tables below. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. ‘N/A’ indicates that the modified oligonucleotide is not 100% complementary to that particular target nucleic acid sequence.

The modified oligonucleotides in Table 56 are 6-10-4 MOE gapmers. The gapmers are 20 nucleosides in length. The sugar motif for the gapmers is (from 5′ to 3′): eeeeeeddddddddddeeee; wherein each ‘d’ represents a 2′-β-D-deoxyribosyl sugar moiety, and each ‘e’ represents a 2′-MOE modified sugar moiety. The gapmers have an internucleoside linkage motif of (from 5′ to 3′): sooooossssssssssoss; wherein each “s” represents a phosphorothioate internucleoside linkage, and each “o” represents a phosphodiester internucleoside linkage. Each cytosine residue is a 5-methyl cytosine.

TABLE 68
6-10-4 MOE gapmers with mixed PO/PS
internucleoside linkages complementary to human APOE

| | SEQ | SEQ | SEQ | SEQ | | |
|---|---|---|---|---|---|---|
| | ID No: | ID No: | ID No: | ID No: | | |
| Compound | 1 Start | 1 Stop | 2 Start | 2 Stop | | SEQ |
| Number | Site | Site | Site | Site | Sequence (5′ to 3′) | ID NO |
| 1601915 | 4761 | 4780 | 243 | 262 | GCTCCACCTTGGCCTGGCAT | 2934 |
| 1601916 | 3430 | 3449 | N/A | N/A | ACCCATTCCCTATTTAACTC | 1462 |
| 1601918 | 3070 | 3089 | N/A | N/A | CCTGCTATGGCTTACATCCC | 1669 |
| 1601924 | 3266 | 3285 | N/A | N/A | CCCTTCACATTCTAAGCTCC | 1188 |
| 1601926 | 3179 | 3198 | N/A | N/A | CGACGGCTAGCTACCGTGTC | 934 |
| 1601928 | 3284 | 3303 | N/A | N/A | TCTCGCATTCCTCATTCTCC | 1832 |
| 1601929 | 2970 | 2989 | N/A | N/A | GCTGCTTGCCTCACCCCCGC | 1376 |
| 1601931 | 4779 | 4798 | 261 | 280 | GCTCTGTCTCCACCGCTTGC | 1341 |
| 1601932 | 3405 | 3424 | N/A | N/A | TGGTCTTCTCTTATCTCCCC | 1586 |
| 1601934 | 3269 | 3288 | N/A | N/A | TCTCCCTTCACATTCTAAGC | 1705 |
| 1601937 | 3061 | 3080 | N/A | N/A | GCTTACATCCCAGTCCAGCT | 1254 |
| 1601938 | 2973 | 2992 | N/A | N/A | CCTGCTGCTTGCCTCACCCC | 1884 |
| 1601940 | 3297 | 3316 | N/A | N/A | ATCTCAGTCCCAGTCTCGCA | 1897 |
| 1601914 | 4613 | 4632 | N/A | N/A | AGTCGGTTTAATCACTTGGA | 1179 |
| 1601919 | 3424 | 3443 | N/A | N/A | TCCCTATTTAACTCCCTCCT | 1197 |
| 1601935 | 3283 | 3302 | N/A | N/A | CTCGCATTCCTCATTCTCCC | 1219 |
| 1601936 | 3408 | 3427 | N/A | N/A | TCCTGGTCTTCTCTTATCTC | 1322 |
| 1601939 | 3217 | 3236 | N/A | N/A | TATGAGCTAATTCAGTCCTC | 1350 |
| 1601945 | 3220 | 3239 | N/A | N/A | ATTTATGAGCTAATTCAGTC | 1874 |
| 1601951 | 4212 | 4231 | N/A | N/A | GTCTGGTATTCACTATCTGC | 942 |
| 1601952 | 3414 | 3433 | N/A | N/A | ACTCCCTCCTGGTCTTCTCT | 1718 |
| 1601954 | 3403 | 3422 | N/A | N/A | GTCTTCTCTTATCTCCCCAT | 1687 |
| 1601960 | 4206 | 4225 | N/A | N/A | TATTCACTATCTGCCTGCAA | 1385 |

### Example 15: Design of RNAi Compounds with Antisense RNAi Oligonucleotides Complementary to a Human APOE Nucleic Acid

RNAi compounds comprising antisense RNAi oligonucleotides complementary to a human APOE nucleic acid and sense RNAi oligonucleotides complementary to the antisense RNAi oligonucleotides were designed as follows.

The RNAi compounds in the tables below consist of an antisense RNAi oligonucleotide and a sense RNAi oligonucleotide. The antisense RNAi oligonucleotide in each case is 23 nucleosides in length; has a sugar motif (from 5′ to 3′) of: efyyyfyyyyyyyfyfyyyyyyy; wherein ‘e’ represents a 2′-MOE modified sugar moiety, each “y” represents a 2′-O-methylribosyl sugar, and each “f” represents a 2′-fluororibosyl sugar; and an internucleoside linkage motif (from 5′ to 3′) of: ssooooooooooooooooooss; wherein ‘o’ represents a phosphodiester internucleoside linkage and ‘s’ represents a phosphorothioate internucleoside linkage. Each antisense RNAi oligonucleotide described in the table below has a 5′-(E)-Vinylphosphonate on the 5′ end of the compound.

The sense RNAi oligonucleotide in each case is 21 nucleosides in length; has a sugar motif (from 5′ to 3′) of: yyyyyyfyfffyyyyyyyyyy; wherein each “y” represents a 2′-O-methylribosyl sugar, and each “f” represents a 2′-fluororibosyl sugar; and an internucleoside linkage motif (from 5′ to 3′) of: ssooooooooooooooooss; wherein ‘o’ represents a phosphodiester internucleoside linkage and ‘s’ represents a phosphorothioate internucleoside linkage. Each antisense RNAi oligonucleotides is complementary to the target nucleic acid (APOE), and each sense RNAi oligonucleotides is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Further, the RNAi sense oligonucleotides are conjugated to a [3nC7-C16] moiety at the 3′ end. “[3nC7-C16]” represents a palmitate moiety linked to a 3′-C7 amino modifier, as shown below, which is attached to the 3′-nucleoside via a phosphodiester linkage.

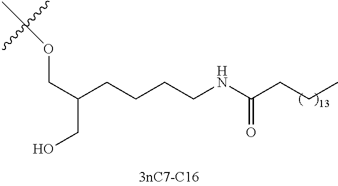

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified antisense RNAi oligonucleoside listed in the tables below is complementary to SEQ ID NO: 2 (described herein above), with a single mismatch to SEQ ID NO: 2 (described herein above) located at position 1 on 5′ end of the antisense strand. The non-complementary nucleobase is marked in the Antisense Sequence column in

[symbol]

TABLE 69A
RNAi compounds targeting human APOE

| | | | | SEQ ID | SEQ ID |
|---|---|---|---|---|---|
| | Anti- | | | NO: 2 | NO: 2 |
| | sense | | | Anti- | Anti- |
| | RNAi | | SEQ | sense | sense |
| Compound | Oligo | Antisense | ID | Start | Stop |
| Number | ID | Sequence (5′ to 3′) | NO | Site | Site |
| 1642901 | 1628239 | [symbol] AUCUUUAUUAAACUAGGGUCCA | 2935 | 1257 | 1278 |
| 1644690 | 1628956 | [symbol] AACUUGGUGAAUCUUUAUUAAA | 2936 | 1267 | 1288 |
| 1644691 | 1628970 | [symbol] GACAGGGUCUCCCGCUGCAGGC | 2937 | 1211 | 1232 |
| 1644692 | 1628972 | [symbol] AGGGUCUCCCGCUGCAGGCUGC | 2938 | 1208 | 1229 |
| 1644693 | 1628974 | [symbol] GCUCGAACCAGCUCUUGAGGCG | 2939 | 1030 | 1051 |

TABLE 69B
RNAi compounds targeting human APOE

| | Sense | | SEQ |
|---|---|---|---|
| Compound | RNAi | Sense | ID |
| Number | oligo ID | Sequence (5′ to 3′) | NO |
| 1642901 | 1628318 | GACCCUAGUUUAAUAAAGAUA | 2940 |
| 1644690 | 1640442 | UAAUAAAGAUUCACCAAGUUA | 2941 |
| 1644691 | 1640443 | CUGCAGCGGGAGACCCUGUCA | 2942 |
| 1644692 | 1640444 | AGCCUGCAGCGGGAGACCCUA | 2943 |
| 1644693 | 1640445 | CCUCAAGAGCUGGUUCGAGCA | 2944 |

### Example 16: Activity of Modified Oligonucleotides and RNAi Compounds that Target Human APOE in Knock-In Mice

ApoE4 knock-in mice (C57BL/6NTac-Apoeem7250_A-C03(APOE)Tac) were obtained from Taconic Biosciences. APOE knock-in mice were divided into groups of 2 mice each. Each mouse received a single ICV bolus of 300 μg of modified oligonucleotide or a single ICV bolus of 300 μg of RNAi compound. A group of 4 mice received a single ICV bolus with PBS as a negative control.

Two weeks post treatment, mice were sacrificed and RNA was extracted from cortical brain tissue, and spinal cord for quantitative real-time RTPCR analysis of RNA expression of APOE using primer probe set RTS3073 (described herein above). Results are presented as percent change of RNA, relative to PBS control, normalized to mouse cyclophilin A (% control). Mouse cyclophilin A was amplified using primer probe set m_cyclo24 (designated herein above).

As shown in the table below, treatment with modified oligonucleotides resulted in reduction of APOE RNA in comparison to the PBS control.

TABLE 70
Reduction of human APOE RNA in knock-in mice

| | |
|---|---|
| Compound | APOE RNA (% control) |

| No. | SPINAL CORD | CORTEX |
|---|---|---|
| 1601915 | 75 | 93 |
| 1601916 | 110 | 95 |
| 1601918 | 90 | 92 |
| 1601924 | 114 | 100 |
| 1601926 | 117 | 91 |
| 1601928 | 103 | 96 |
| 1601929 | 118 | 100 |
| 1601931 | 78 | 80 |
| 1601932 | 82 | 89 |
| 1601934 | 118 | 98 |
| 1601937 | 87 | 79 |
| 1601938 | 201 | 166 |
| 1601940 | 95 | 70 |

TABLE 71
Reduction of human APOE RNA in knock-in mice

| | |
|---|---|
| Compound | APOE RNA (% control) |

| No. | SPINAL CORD | CORTEX |
|---|---|---|
| PBS | 100 | 100 |
| 1601914 | 74 | 79 |
| 1601919 | 87 | 89 |
| 1601935 | 53 | 69 |
| 1601936 | 88 | 92 |
| 1601939 | 85 | 91 |
| 1601945 | 104 | 102 |
| 1601951 | 58 | 82 |
| 1601952 | 100 | 98 |
| 1601954 | 81 | 78 |
| 1601960 | 82 | 67 |
| 1642901 | 4 | 13 |
| 1644690 | 3‡ | 5‡ |
| 1644691 | 15 | 62 |
| 1644692 | 17 | 47 |

‡Fewer than 2 samples available

TABLE 72
Reduction of human APOE RNA in knock-in mice

| | |
|---|---|
| Compound | APOE RNA (% control) |

| No. | SPINAL CORD | CORTEX |
|---|---|---|
| 1644693 | 12 | 76 |
| 1644690 | 5 | 28 |

1. An oligomeric compound comprising a modified oligonucleotide consisting of 12 to 30 linked nucleosides wherein the nucleobase sequence of the modified oligonucleotide is at least 80% complementary to an equal length portion of an APOE RNA, and wherein the modified oligonucleotide comprises at least one modification selected from a modified sugar and a modified internucleoside linkage.

2. An oligomeric compound comprising a modified oligonucleotide consisting of 12 to 30 linked nucleosides, wherein the nucleobase sequence of the modified oligonucleotide comprises at least 12, at least 13, at least 14, at least 15, at least 16, at least 17, at least 18, at least 19, or at least 20 nucleobases of any of SEQ ID NOS: 20-2551 or 2934; wherein the modified oligonucleotide comprises at least one modification selected from a modified sugar and a modified internucleoside linkage.

3. An oligomeric compound comprising a modified oligonucleotide consisting of 12 to 30 linked nucleosides, wherein the nucleobase sequence of the modified oligonucleotide comprises at least 12, at least 13, at least 14, at least 15, at least 16, at least 17, at least 18, at least 19, at least 20, at least 21, at least 22, or at least 23 nucleobases of any of SEQ ID NOS: 2552-2742 or 2935-2944; wherein the modified oligonucleotide comprises at least one modification selected from a modified sugar and a modified internucleoside linkage.

4. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:
an equal length portion of nucleobases 1155-1178 of SEQ ID NO: 2;
an equal length portion of nucleobases 1207-1230 of SEQ ID NO: 2; or
an equal length portion of nucleobases 1259-1295 of SEQ ID NO: 2;
wherein the modified oligonucleotide comprises at least one modification selected from a modified sugar and a modified internucleoside linkage.

5. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:
an equal length portion of nucleobases 1135-1166 of SEQ ID NO: 2; or
an equal length portion of nucleobases 1255-1294 of SEQ ID NO: 2;
wherein the modified oligonucleotide comprises at least one modification selected from a modified sugar and a modified internucleoside linkage.

6. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of an equal length portion of nucleobases 1255-1295 of SEQ ID NO: 2, wherein the modified oligonucleotide comprises at least one modification selected from a modified sugar and a modified internucleoside linkage.

7. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of any of the nucleobase sequences of:
SEQ ID NOS: 70, 71, 169, 170, 447, 448, 543, 552, 553, 919, 1061, 1132, 1938, 1991, 2066, 2154, 2226, 2259, 2324, 2417, or 2486;
SEQ ID NOS: 460, 461, 462, 563, 564, 565, 566, 990, 1469, 1572, 1653, 1746, 1914, 1955, 2026, 2110, 2211, 2247, 2344, 2393, or 2481; or
SEQ ID NOS: 77, 475, 476, 477, 478, 479, 480, 481, 482, 483, 578, 579, 580, 581, 582, 622, 623, 624, 625, 626, 627, 1063, 1193, 1231, 1232, 1300, 1305, 1378, 1409, 1493, 1526, 1564, 1576, 1678, 1679, 1695, 1827, 1870, 1921, 1928, 1950, 1982, 2012, 2046, 2051, 2074, 2088, 2118, 2158, 2169, 2208, 2223, 2232, 2255, 2321, 2343, 2380, 2436, 2449, or 2451.

8. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of any of the nucleobase sequences of:
SEQ ID NOS: 2600, 2601, 2604, 2605, 2606, 2607, 2608, 2609, 2610, or 2613; or
SEQ ID NOS: 2720, 2721, 2722, 2726, 2727, 2729, 2730, 2731, 2732, 2733, 2734, 2735, 2736, 2737, 2738, or 2740.

9. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22 or at least 23 contiguous nucleobases of any of the nucleobase sequences of SEQ ID NOs: 76, 77, 473, 474, 475, 476, 477, 478, 479, 480, 481, 482, 483, 576, 578, 579, 580, 581, 582, 622, 623, 624, 625, 626, 627, 1063, 1193, 1231, 1232, 1300, 1305, 1378, 1409, 1493, 1526, 1564, 1576, 1678, 1679, 1695, 1701, 1792, 1827, 1870, 1886, 1906, 1921, 1928, 1950, 1982, 2012, 2046, 2051, 2074, 2088, 2118, 2158, 2169, 2208, 2223, 2232, 2255, 2321, 2343, 2370, 2380, 2436, 2449, 2490, 2451, 2720, 2721, 2722, 2725, 2726, 2727, 2729, 2730, 2731, 2732, 2733, 2734, 2735, 2736, 2737, 2738, 2740, 2935 or 2936.

10. The oligomeric compound of any of claims 1-9, wherein the nucleobase sequence of the modified oligonucleotide is at least 80%, 85%, 90%, 95%, or 100% complementary to any of the nucleobase sequences of SEQ ID NOs: 1-6 when measured across the entire nucleobase sequence of the modified oligonucleotide.

11. The oligomeric compound of any of claims 1-10, wherein at least one nucleoside of the modified oligonucleotide is a modified nucleoside.

12. The oligomeric compound of claim 11, wherein at least one modified nucleoside of the modified oligonucleotide comprises a modified sugar moiety.

13. The oligomeric compound of claim 12, wherein the modified sugar moiety comprises a bicyclic sugar moiety.

14. The oligomeric compound of claim 13, wherein the bicyclic sugar moiety comprises a 2′-4′ bridge selected from —O—CH₂—; and —O—CH(CH₃)—.

15. The oligomeric compound of any of claims 11-14, wherein at least one modified nucleoside of the modified oligonucleotide comprises a non-bicyclic modified sugar moiety.

16. The oligomeric compound of claim 15, wherein at least one modified nucleoside of the modified oligonucleotide comprises a bicyclic sugar moiety having a 2′-4′ bridge and at least one nucleoside comprising a non-bicyclic modified sugar moiety.

17. The oligomeric compound of claim 15 or 16, wherein the non-bicyclic modified sugar moiety is a 2′-O(CH₂)₂—OCH₃ ribosyl modified sugar moiety, a 2′-OMe modified sugar moiety, or a 2′-F modified sugar moiety.

18. The oligomeric compound of any of claims 1-17, wherein the modified oligonucleotide comprises at least one modified nucleoside comprising a sugar surrogate.

19. The oligomeric compound of claim 18, wherein at least one modified nucleoside of the modified oligonucleotide comprises a sugar surrogate selected from morpholino and PNA.

20. The oligomeric compound of any of claims 1-19, wherein the modified oligonucleotide comprises at least one modified internucleoside linkage.

21. The oligomeric compound of claim 20, wherein each internucleoside linkage of the modified oligonucleotide is a modified internucleoside linkage.

22. The oligomeric compound of claim 20 or 21, wherein at least one internucleoside linkage is a phosphorothioate internucleoside linkage.

23. The oligomeric compound of claim 20 or 22, wherein the modified oligonucleotide comprises at least one phosphodiester internucleoside linkage.

24. The oligomeric compound of any of claim 20, 22, or 23, wherein each internucleoside linkage is independently selected from a phosphodiester internucleoside linkage or a phosphorothioate internucleoside linkage.

25. The oligomeric compound of any of claim 20 or 22-24, wherein at least 4, at least 5, at least 6, at least 7, at least 8, at least 9, at least 10, at least 11, at least 12, at least 13, at least 14, at least 15, at least 16, at least 17, at least 18, or at least 19 internucleoside linkages of the modified oligonucleotide are phosphorothioate internucleoside linkages.

26. The oligomeric compound of any of claims 20-25, wherein each internucleoside linkage is a phosphorothioate internucleoside linkage.

27. The oligomeric compound of any of claim 20 or 22-26, wherein the internucleoside linkage motif of the modified oligonucleotide is selected from: 5′-sssssssssssssssssss-3′, 5′-soossssssssssooss-3′, 5′-sooosssssssssssooss-3′, 5′-soossssssssssos-3′, 5′-ssooooooooooooooooooss-3′, 5′-ssooooooooooooooooss-3′, 5′-soooossssssssssooss-3′, 5′-sossssssssssssssoss-3′, 5′-soosssssssssooss-3′, and 5′-sooooossssssssssoss-3′; wherein each ‘o’ represents a phosphodiester internucleoside linkage and each ‘s’ represents a phosphorothioate internucleoside linkage.

28. The oligomeric compound of any of claims 1-27, wherein the modified oligonucleotide comprises a modified nucleobase.

29. The oligomeric compound of claim 28, wherein the modified nucleobase is a 5-methyl cytosine.

30. The oligomeric compound of any of claims 1-29, wherein the oligomeric compound comprises a modified oligonucleotide consisting of 12-22, 12-20, 14-18, 14-20, 15-17, 15-25, 16-20, 16-18, 18-22, 18-25, 18-20, 20-25, or 21-23 linked nucleosides, or a pharmaceutically acceptable salt thereof

31. The oligomeric compound of claim 30, which is a pharmaceutically acceptable salt comprising one or more cations selected from sodium, potassium, calcium, and magnesium.

32. The oligomeric compound of any of claims 1-31, wherein the modified oligonucleotide consists of 16 or 18 linked nucleosides.

33. The oligomeric compound of any of claims 1-31, wherein the modified oligonucleotide consists of 20 linked nucleosides.

34. The oligomeric compound of any of claims 1-31, wherein the modified oligonucleotide consists of 21 linked nucleosides.

35. The oligomeric compound of any of claims 1-31, wherein the modified oligonucleotide consists of 23 linked nucleosides.

36. The oligomeric compound of any of claims 1-35, wherein the oligomeric compound is an RNase H compound.

37. The oligomeric compound of claim 36, wherein the modified oligonucleotide is a gapmer.

38. The oligomeric compound of any of claims 1-37, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprising a 2′-substituted sugar moiety.

39. The oligomeric compound of any of claim 1-34 or 36-37, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

40. The oligomeric compound of claim 39, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises either a cEt modified sugar moiety or a 2′-O(CH₂)₂—OCH₃ ribosyl modified sugar moiety, and each of the central region nucleosides comprises a 2′-β-D-deoxyribosyl sugar moiety.

41. The oligomeric compound of claim 39 or claim 40, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises a 2′-O(CH₂)₂—OCH₃ ribosyl modified sugar moiety, and each of the central region nucleosides comprises a 2′-β-D-deoxyribosyl sugar moiety.

42. The oligomeric compound of any of claims 1-35, wherein the oligomeric compound is an RNAi agent.

43. The oligomeric compound of any of claims 1-42, wherein the oligomeric compound comprises an antisense RNAi oligonucleotide comprising a targeting region comprising at least 15 contiguous nucleobases, wherein the targeting region is at least 90% complementary to an equal-length portion of an APOE RNA.

44. The oligomeric compound of claim 43, wherein the targeting region of the antisense RNAi oligonucleotide is at least 95% complementary or is 100% complementary to the equal length portion of an APOE RNA.

45. The oligomeric compound of any of claims 43-44, wherein the targeting region of the antisense RNAi oligonucleotide comprises at least 19, 20, 21, or 25 contiguous nucleobases.

46. The oligomeric compound of any of claims 43-45, wherein the APOE RNA has the nucleobase sequence of any of SEQ ID NOs: 1-6.

47. The oligomeric compound of any of claims 43-46, wherein at least one nucleoside of the antisense RNAi oligonucleotide comprises a modified sugar moiety selected from: 2′-F, 2′-O(CH₂)₂—OCH₃, 2′-NMA, LNA, and cEt; or a sugar surrogate selected from GNA, and UNA.

48. The oligomeric compound of any of claims 43-47, wherein each nucleoside of the antisense RNAi oligonucleotide comprises a modified sugar moiety or a sugar surrogate.

49. The oligomeric compound of any of claims 43-48, wherein at least 80%, at least 90%, or 100% of the nucleosides of the antisense RNAi oligonucleotide comprises a modified sugar moiety selected from 2′-F and 2′-OMe.

50. The oligomeric compound of any of claims 43-49, comprising a stabilized phosphate group attached to the 5′ position of the 5′-most nucleoside of the antisense RNAi oligonucleotide.

51. The oligomeric compound of claim 50, wherein the stabilized phosphate group comprises a cyclopropyl phosphonate or an (E)-vinyl phosphonate.

52. The oligomeric compound of any of claims 1-51, wherein the oligomeric compound is a single-stranded oligomeric compound.

53. The oligomeric compound of any of claims 1-52, consisting of the modified oligonucleotide or the RNAi antisense oligonucleotide.

54. The oligomeric compound of any of claims 1-53, comprising a conjugate group comprising a conjugate moiety and a conjugate linker.

55. The oligomeric compound of claim 54, wherein the conjugate linker consists of a single bond.

56. The oligomeric compound of claim 54, wherein the conjugate linker is cleavable.

57. The oligomeric compound of claim 54, wherein the conjugate linker comprises 1-3 linker-nucleosides.

58. The oligomeric compound of any of claims 54-57, wherein the conjugate group is attached to the 5′-end of the modified oligonucleotide or the antisense RNAi oligonucleotide.

59. The oligomeric compound of any of claims 54-57, wherein the conjugate group is attached to the 3′-end of the modified oligonucleotide or the antisense RNAi oligonucleotide.

60. The oligomeric compound of any of claims 1-59, comprising a terminal group.

61. The oligomeric compound of any of claim 1-56 or 58-60, wherein the oligomeric compound does not comprise linker-nucleosides.

62. An oligomeric duplex, comprising a first oligomeric compound comprising an antisense RNAi oligonucleotide of any of claims 43-61 and a second oligomeric compound comprising a sense RNAi oligonucleotide consisting of 17 to 30 linked nucleosides, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide.

63. The oligomeric duplex of claim 62, wherein the sense RNAi oligonucleotide consists of 18-25, 20-25, or 21-23 linked nucleosides.

64. The oligomeric duplex of claim 62, wherein the sense RNAi oligonucleotide consists of 21 or 23 linked nucleosides.

65. The oligomeric duplex of any of claims 62-64, wherein 1-4 3′-most nucleosides of the antisense or the sense RNAi oligonucleotide are overhanging nucleosides.

66. The oligomeric duplex of any of claims 62-65, wherein 1-4 5′-most nucleosides of the antisense or sense RNAi oligonucleotide are overhanging nucleosides.

67. The oligomeric duplex of any of claims 62-64, wherein the duplex is blunt ended at the 3′-end of the antisense RNAi oligonucleotide.

68. The oligomeric duplex of any of claims 62-64, wherein the duplex is blunt ended at the 5′-end of the antisense RNAi oligonucleotide.

69. The oligomeric duplex of any of claims 62-68, wherein at least one nucleoside of the sense RNAi oligonucleotide comprises a modified sugar moiety selected from: 2′-F, 2′-OMe, LNA, cEt, or a sugar surrogate selected from GNA, and UNA.

70. The oligomeric duplex of claim 69, wherein each nucleoside of the sense RNAi oligonucleotide comprises a modified sugar moiety or a sugar surrogate.

71. The oligomeric duplex of claim 70, wherein at least 80%, at least 90%, or 100% of the nucleosides of the sense RNAi oligonucleotide comprises a modified sugar moiety selected from 2′-F and 2′-OMe.

72. The oligomeric duplex of any of claims 62-71, wherein at least one nucleoside of the sense RNAi oligonucleotide comprises a modified nucleobase.

73. The oligomeric duplex of any of claims 62-72, wherein at least one internucleoside linkage of the sense RNAi oligonucleotide is a modified internucleoside linkage.

74. The oligomeric duplex of claim 73, wherein at least one internucleoside linkage of the sense RNAi oligonucleotide is a phosphorothioate internucleoside linkage.

75. The oligomeric duplex of any of claims 62-74, wherein the oligomeric duplex comprises 1-5 abasic sugar moieties attached to one or both ends of the antisense or sense RNA oligonucleotide.

76. The oligomeric duplex of any of claims 62-75, consisting of the antisense RNAi oligonucleotide and the sense RNAi oligonucleotide.

77. The oligomeric duplex of any of claims 62-75, wherein the second oligomeric compound comprises a conjugate group comprising a conjugate moiety and a conjugate linker.

78. The oligomeric duplex of claim 77, wherein the conjugate linker consists of a single bond.

79. The oligomeric duplex of claim 78, wherein the conjugate linker is cleavable.

80. The oligomeric duplex of claim 78, wherein the conjugate linker comprises 1-3 linker-nucleosides.

81. The oligomeric duplex of any of claims 78-80, wherein the conjugate group is attached to the 5′-end of the sense RNAi oligonucleotide.

82. The oligomeric duplex of any of claims 78-80, wherein the conjugate group is attached to the 3′-end of the sense RNAi oligonucleotide.

83. The oligomeric duplex of any of claims 78-80, wherein the conjugate group is attached via the 2′ position of a ribosyl sugar moiety at an internal position of the sense RNAi oligonucleotide.

84. The oligomeric compound of any of claims 54-59 or the oligomeric duplex of any of claims 77-83, wherein at least one conjugate group comprises a C₁₆ alkyl group.

85. The oligomeric duplex of claim 62, wherein the second oligomeric compound comprises a terminal group.

86. An antisense agent comprising an antisense compound, wherein the antisense compound is the oligomeric compound of any of claims 1-61.

87. The antisense agent of claim 86, wherein the antisense agent is the oligomeric duplex of any of claims 62-85.

88. The antisense agent of claim 86 or claim 87, wherein the antisense agent is:
i) an RNase H agent capable of reducing the amount of APOE nucleic acid through the activation of RNase H; or
ii) an RNAi agent capable of reducing the amount of APOE nucleic acid through the activation of RISC/Ago2.

89. The antisense agent of any of claims 86-88, wherein the antisense agent comprises a conjugate group, wherein the conjugate group comprises a cell-targeting moiety.

90. A chirally enriched population of oligomeric compounds of any of claims 1-61, wherein the population is enriched for modified oligonucleotides comprising at least one particular phosphorothioate internucleoside linkage having a particular stereochemical configuration.

91. The chirally enriched population of claim 90, wherein the population is enriched for modified oligonucleotides comprising at least one particular phosphorothioate internucleoside linkage having the (Sp) or (Rp) configuration.

92. The chirally enriched population of claim 90, wherein the population is enriched for modified oligonucleotides having a particular, independently selected stereochemical configuration at each phosphorothioate internucleoside linkage.

93. The chirally enriched population of claim 90, wherein the population is enriched for modified oligonucleotides having the (Rp) configuration at one particular phosphorothioate internucleoside linkage and the (Sp) configuration at each of the remaining phosphorothioate internucleoside linkages.

94. The chirally enriched population of claim 90, wherein the population is enriched for modified oligonucleotides having at least 3 contiguous phosphorothioate internucleoside linkages in the Sp, Sp, and Rp configurations, in the 5′ to 3′ direction.

95. A population of oligomeric compounds of any of claims 1-61, wherein all of the phosphorothioate internucleoside linkages of the modified oligonucleotide are stereorandom.

96. A pharmaceutical composition comprising an oligomeric compound of any of claims 1-61, an oligomeric duplex of any of claims 62-85, an antisense agent of any of claims 86-89, or a population of any of claims 90-95, and a pharmaceutically acceptable carrier or diluent.

97. The pharmaceutical composition of claim 96, wherein the pharmaceutically acceptable diluent is artificial cerebral spinal fluid (aCSF), sterile saline, or PBS.

98. The pharmaceutical composition of claim 97, wherein the pharmaceutical composition consists essentially of the modified oligonucleotide, the oligomeric duplex, the antisense agent, or the population and PBS or aCSF.

99. A method comprising administering to a subject an oligomeric compound of any of claims 1-61, an oligomeric duplex of any of claims 62-85, an antisense agent of any of claims 86-89, a population of any of claims 90-95, or a pharmaceutical composition of any of claims 96-98.

100. A method of treating a disease associated with APOE comprising administering to a subject having or at risk for developing a disease associated with APOE a therapeutically effective amount of an oligomeric compound of any of claims 1-61, an oligomeric duplex of any of claims 62-85, an antisense agent of any of claims 86-89, a population of any of claims 90-95, or a pharmaceutical composition according to any of claims 96-98; and thereby treating the disease associated with APOE.

101. The method of claim 100, wherein the APOE-associated disease is Alzheimer's Disease.

102. The method of any of claims 99-101, wherein at least one symptom or hallmark of the APOE-associated disease is ameliorated.

103. The method of claim 102, wherein the symptom or hallmark is cognitive impairment, progressive memory loss, behavioral abnormality, dementia, difficulty performing daily activities, amyloid plaques, neurofibrillary tangles, or neuroinflammation.

104. The method of any of claims 100-103, wherein administering an oligomeric compound of any of claims 1-61, an oligomeric duplex of any of claims 62-85, an antisense agent of any of claims 86-89, a population of any of claims 90-95, or a pharmaceutical composition according to any of claims 96-98 reduces cognitive impairment, behavioral abnormality, dementia, difficulty performing daily activities, amyloid plaques, neurofibrillary tangles, or neuroinflammation, or slows memory loss in the subject.

105. The method of any of claims 99-104, wherein the subject is human.

106. A method of reducing expression of APOE in a cell comprising contacting the cell with an oligomeric compound of any of claims 1-61, an oligomeric duplex of any of claims 62-85, an antisense agent of any of claims 86-89, a population of any of claims 90-95, or a pharmaceutical composition according to any of claims 96-98.

107. The method of claim 106, wherein the cell is a neuron or a glial cell, optionally wherein the cell is an astrocyte or microglial cell.

108. The method of claim 106 or claim 107, wherein the cell is a human cell.

109. Use of an oligomeric compound of any of claims 1-61, an oligomeric duplex of any of claims 62-85, an antisense agent of any of claims 86-89, a population of any of claims 90-95, or a pharmaceutical composition according to any of claims 96-98 for treating a disease associated with APOE.

110. Use of an oligomeric compound of any of claims 1-61, an oligomeric duplex of any of claims 62-85, an antisense agent of any of claims 86-89, a population of any of claims 90-95, or a pharmaceutical composition according to any of claims 96-98 in the manufacture of a medicament for treating a disease associated with APOE.

111. The use of claim 109 or claim 110, wherein the APOE-associated disease is Alzheimer's Disease.
